SEQUENCE LISTING (8 sequences)

SEQ ID NO: 1 - DNA, 3978 bp, Artificial Sequence
  Synthetic Construct
atgctttggtgggaggaagtggaggactgctacgagagagaggacgtgcagaagaaaaccttcaccaagtgggtgaacgcccagttcagcaagttcggcaagcagcacatcgagaacctgttcagcgacctgcaggatggcaggagactgctggacctgctggagggcctgaccggccagaagctgcccaaggagaagggcagcaccagagtgcacgccctgaacaacgtgaacaaggccctgagagtgctgcagaacaacaacgtggacctggtgaacatcggcagcaccgacatcgtggacggcaaccacaagctgaccctgggcctgatctggaacatcatcctgcactggcaggtgaagaacgtgatgaagaacatcatggccggcctgcagcagaccaacagcgagaagatcctgctgagctgggtgaggcagagcaccagaaactacccccaggtgaacgtgatcaacttcaccacctcctggagcgacggcctggccctgaacgccctgatccacagccacagacccgacctgttcgactggaacagcgtggtgtgtcagcagagcgccacccagagactggagcacgccttcaacatcgccagataccagctgggcatcgagaagctgctggaccccgaggacgtggacaccacctaccccgacaagaaaagcatcctcatgtacattaccagcctgttccaggtgctgccccagcaggtgtccatcgaggccatccaggaagtggaaatgctgcccaggccccccaaagtgaccaaggaggagcacttccagctgcaccaccagatgcactacagccagcagatcacagtgagcctggcccagggctatgagagaaccagcagccccaagcccagattcaagagctacgcctacacccaggccgcctacgtgaccacctccgaccccaccagaagccccttccccagccagcacctggaggcccccgaggacaagagcttcggcagcagcctgatggagagcgaagtgaacctggacagataccagaccgccctggaggaagtgctgtcctggctgctgagcgccgaggacaccctgcaggcccagggcgagatcagcaacgacgtggaagtggtgaaggaccagttccacacccacgagggctacatgatggatctgaccgcccaccagggcagagtgggcaatatcctgcagctgggcagcaagctgatcggcaccggcaagctgagcgaggacgaggagaccgaagtgcaggagcagatgaacctgctgaacagcagatgggagtgcctgagagtggccagcatggagaagcagagcaacctgcacagagtgctgatggacctgcagaaccagaagctgaaggagctgaacgactggctgaccaagaccgaggagcggaccagaaagatggaggaggagcccctgggccccgacctggaggacctgaagagacaggtgcagcagcacaaagtgctgcaggaggacctggagcaggagcaggtgcgcgtgaacagcctgacccacatggtggtggtcgtggacgagagcagcggcgaccacgccacagccgccctggaagagcagctgaaagtgctgggcgacagatgggccaatatttgtaggtggaccgaggacagatgggtgctgctgcaggaccagcccgacctggcccctggcctgaccaccatcggcgccagccccacccagaccgtgaccctggtgacccagcccgtggtgacaaaggagaccgccatcagcaagctggagatgcccagctccctgatgctggaagtgcccacccaccgcctgctccagcagttccccctggacctggagaagttcctggcctggctgaccgaggccgaaaccaccgccaatgtgctccaggacgccactagaaaggagaggctgctggaggacagcaagggcgtgaaagagctgatgaagcagtggcaggatctgcagggcgaaatcgaggcccacaccgacgtgtaccacaacctggacgagaacagccagaagattctgaggagcctggagggcagcgacgacgccgtcctgctccagaggaggctggacaacatgaacttcaagtggagcgagctgcggaagaagagcctgaacatccggagccacctggaagccagcagcgaccagtggaagagactgcacctgagcctgcaggagctgctggtgtggctgcagctgaaggacgacgagctgagcagacaggcccccatcggcggcgacttccccgccgtgcagaagcagaacgacgtgcaccgggccttcaagagggagctgaaaaccaaggaacccgtgatcatgagcaccctggagacagtgcggatcttcctgaccgagcagcccctggagggactggagaagctgtaccaggagcccagagagctgccccccgaggagagagcccagaacgtgaccaggctgctgagaaagcaggccgaggaagtgaataccgagtgggagaagctgaatctgcacagcgccgactggcagagaaagatcgacgagaccctggagagactccaggaactgcaggaagccaccgacgagctggacctgaagctgagacaggccgaagtgatcaagggcagctggcagcctgtgggcgatctgctgatcgactccctgcaggatcacctggagaaagtgaaggccctgcggggcgagatcgcccccctgaaggagaatgtgagccacgtgaacgacctggccagacagctgaccaccctgggcatccagctgagcccctacaacctgagcacactggaggatctgaacacccggtggaaactgctgcaggtggccgtggaggatagagtgaggcagctgcacgaagcccacagagacttcggccctgcctcccagcacttcctgagcaccagcgtgcagggcccctgggagagagccatctcccccaacaaagtgccctactacatcaaccacgagacccagaccacctgctgggaccaccctaagatgaccgagctgtatcagagcctggccgacctgaacaatgtgcggttcagcgcctacagaaccgccatgaagctgcggagactgcagaaggccctgtgcctggatctgctgagcctgagcgccgcctgcgacgccctggaccagcacaacctgaagcagaatgaccagcccatggacatcctgcagatcatcaactgcctgaccacaatctacgaccggctggaacaggagcacaacaacctggtgaatgtgcccctgtgcgtggacatgtgcctgaattggctgctgaacgtgtacgacaccggcaggaccggcagaatccgcgtgctgagcttcaagaccggcatcatcagcctgtgcaaggcccacctggaggataagtaccgctacctgttcaagcaggtggccagcagcaccggcttctgcgatcagaggagactgggcctgctgctgcacgatagcatccagatccctaggcagctgggcgaagtggccagctttggcggcagcaacatcgagccctctgtgaggagctgcttccagttcgccaacaacaagcccgagatcgaggccgccctgttcctggactggatgaggctggagcctcagagcatggtgtggctgcctgtgctgcacagagtggccgccgccgagaccgccaagcaccaggccaagtgcaatatctgcaaggagtgccccatcatcggcttccggtacaggagcctgaagcacttcaactacgacatctgccagagctgctttttcagcggcagagtggccaagggccacaaaatgcactaccccatggtggagtactgcacccccaccacctccggcgaggatgtgagagacttcgccaaagtgctgaagaataagttccggaccaagcggtactttgccaagcaccccaggatgggctacctgcccgtgcagaccgtgctggaaggcgacaacatggagacctga

SEQ ID NO: 2 - protein, 1325 aa, Artificial Sequence
  Synthetic Construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDQPDLAPGLTTIGASPTQTVTLVTQPVVTKETAISKLEMPSSLMLEVPTHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMET

SEQ ID NO: 3 - protein, 736 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 4 - DNA, 4955 bp, Artificial Sequence
  Synthetic Construct
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtaccccactacgggtctaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagcggccgatccgccaccatgctttggtgggaggaagtggaggactgctacgagagagaggacgtgcagaagaaaaccttcaccaagtgggtgaacgcccagttcagcaagttcggcaagcagcacatcgagaacctgttcagcgacctgcaggatggcaggagactgctggacctgctggagggcctgaccggccagaagctgcccaaggagaagggcagcaccagagtgcacgccctgaacaacgtgaacaaggccctgagagtgctgcagaacaacaacgtggacctggtgaacatcggcagcaccgacatcgtggacggcaaccacaagctgaccctgggcctgatctggaacatcatcctgcactggcaggtgaagaacgtgatgaagaacatcatggccggcctgcagcagaccaacagcgagaagatcctgctgagctgggtgaggcagagcaccagaaactacccccaggtgaacgtgatcaacttcaccacctcctggagcgacggcctggccctgaacgccctgatccacagccacagacccgacctgttcgactggaacagcgtggtgtgtcagcagagcgccacccagagactggagcacgccttcaacatcgccagataccagctgggcatcgagaagctgctggaccccgaggacgtggacaccacctaccccgacaagaaaagcatcctcatgtacattaccagcctgttccaggtgctgccccagcaggtgtccatcgaggccatccaggaagtggaaatgctgcccaggccccccaaagtgaccaaggaggagcacttccagctgcaccaccagatgcactacagccagcagatcacagtgagcctggcccagggctatgagagaaccagcagccccaagcccagattcaagagctacgcctacacccaggccgcctacgtgaccacctccgaccccaccagaagccccttccccagccagcacctggaggcccccgaggacaagagcttcggcagcagcctgatggagagcgaagtgaacctggacagataccagaccgccctggaggaagtgctgtcctggctgctgagcgccgaggacaccctgcaggcccagggcgagatcagcaacgacgtggaagtggtgaaggaccagttccacacccacgagggctacatgatggatctgaccgcccaccagggcagagtgggcaatatcctgcagctgggcagcaagctgatcggcaccggcaagctgagcgaggacgaggagaccgaagtgcaggagcagatgaacctgctgaacagcagatgggagtgcctgagagtggccagcatggagaagcagagcaacctgcacagagtgctgatggacctgcagaaccagaagctgaaggagctgaacgactggctgaccaagaccgaggagcggaccagaaagatggaggaggagcccctgggccccgacctggaggacctgaagagacaggtgcagcagcacaaagtgctgcaggaggacctggagcaggagcaggtgcgcgtgaacagcctgacccacatggtggtggtcgtggacgagagcagcggcgaccacgccacagccgccctggaagagcagctgaaagtgctgggcgacagatgggccaatatttgtaggtggaccgaggacagatgggtgctgctgcaggaccagcccgacctggcccctggcctgaccaccatcggcgccagccccacccagaccgtgaccctggtgacccagcccgtggtgacaaaggagaccgccatcagcaagctggagatgcccagctccctgatgctggaagtgcccacccaccgcctgctccagcagttccccctggacctggagaagttcctggcctggctgaccgaggccgaaaccaccgccaatgtgctccaggacgccactagaaaggagaggctgctggaggacagcaagggcgtgaaagagctgatgaagcagtggcaggatctgcagggcgaaatcgaggcccacaccgacgtgtaccacaacctggacgagaacagccagaagattctgaggagcctggagggcagcgacgacgccgtcctgctccagaggaggctggacaacatgaacttcaagtggagcgagctgcggaagaagagcctgaacatccggagccacctggaagccagcagcgaccagtggaagagactgcacctgagcctgcaggagctgctggtgtggctgcagctgaaggacgacgagctgagcagacaggcccccatcggcggcgacttccccgccgtgcagaagcagaacgacgtgcaccgggccttcaagagggagctgaaaaccaaggaacccgtgatcatgagcaccctggagacagtgcggatcttcctgaccgagcagcccctggagggactggagaagctgtaccaggagcccagagagctgccccccgaggagagagcccagaacgtgaccaggctgctgagaaagcaggccgaggaagtgaataccgagtgggagaagctgaatctgcacagcgccgactggcagagaaagatcgacgagaccctggagagactccaggaactgcaggaagccaccgacgagctggacctgaagctgagacaggccgaagtgatcaagggcagctggcagcctgtgggcgatctgctgatcgactccctgcaggatcacctggagaaagtgaaggccctgcggggcgagatcgcccccctgaaggagaatgtgagccacgtgaacgacctggccagacagctgaccaccctgggcatccagctgagcccctacaacctgagcacactggaggatctgaacacccggtggaaactgctgcaggtggccgtggaggatagagtgaggcagctgcacgaagcccacagagacttcggccctgcctcccagcacttcctgagcaccagcgtgcagggcccctgggagagagccatctcccccaacaaagtgccctactacatcaaccacgagacccagaccacctgctgggaccaccctaagatgaccgagctgtatcagagcctggccgacctgaacaatgtgcggttcagcgcctacagaaccgccatgaagctgcggagactgcagaaggccctgtgcctggatctgctgagcctgagcgccgcctgcgacgccctggaccagcacaacctgaagcagaatgaccagcccatggacatcctgcagatcatcaactgcctgaccacaatctacgaccggctggaacaggagcacaacaacctggtgaatgtgcccctgtgcgtggacatgtgcctgaattggctgctgaacgtgtacgacaccggcaggaccggcagaatccgcgtgctgagcttcaagaccggcatcatcagcctgtgcaaggcccacctggaggataagtaccgctacctgttcaagcaggtggccagcagcaccggcttctgcgatcagaggagactgggcctgctgctgcacgatagcatccagatccctaggcagctgggcgaagtggccagctttggcggcagcaacatcgagccctctgtgaggagctgcttccagttcgccaacaacaagcccgagatcgaggccgccctgttcctggactggatgaggctggagcctcagagcatggtgtggctgcctgtgctgcacagagtggccgccgccgagaccgccaagcaccaggccaagtgcaatatctgcaaggagtgccccatcatcggcttccggtacaggagcctgaagcacttcaactacgacatctgccagagctgctttttcagcggcagagtggccaagggccacaaaatgcactaccccatggtggagtactgcacccccaccacctccggcgaggatgtgagagacttcgccaaagtgctgaagaataagttccggaccaagcggtactttgccaagcaccccaggatgggctacctgcccgtgcagaccgtgctggaaggcgacaacatggagacctgatgaggagctcgagaggcctaataaagagctcagatgcatcgatcagagtgtgttggttttttgtgtgagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 5 - DNA, 106 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtgg

SEQ ID NO: 6 - DNA, 113 bp, Artificial Sequence
  Synthetic Construct
cgcccgacgcccgggctttgcccgggcggcctcagtgagcgagcgagcgcgccagctggcgtaatagcgaagaggcccgcaccgatcgcccttcccaacagttgcgcagcctg

SEQ ID NO: 7 - DNA, 145 bp, Artificial Sequence
  Synthetic Construct
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 8 - DNA, 145 bp, Artificial Sequence
  Synthetic Construct
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

(19) United States
(12) Patent Application Publication
(10) Pub. No.: US 2024/0043869 A1
(43) Pub. Date: Feb. 8, 2024

(54) METHODS FOR PURIFICATION OF AAV VECTORS BY AFFINITY CHROMATOGRAPHY

(71) Applicant: PFIZER INC., New York, NY (US)

(72) Inventors: William S KISH, Holly Springs, NC (US); John R. LIGHTHOLDER, Franklin, MA (US); Matthew K. ROACH, Cary, NC (US); Tamara ZEKOVIC, Cary, NC (US)

(21) Appl. No.: 18/255,868

(22) PCT Filed: Dec. 20, 2021
(86) PCT No.: PCT/IB2021/062018

§ 371 Date: Jun. 4, 2023

Related U.S. Application Data
(60) Provisional application No. 63/212,457, filed on Jun. 18, 2021.
(60) Provisional application No. 63/129,934, filed on Dec. 23, 2020.

(51) Int. Cl. C12N 15/86; B01D 15/20; B01D 15/42; B01D 15/38; B01D 15/16

(52) U.S. Cl. CPC C12N 15/86; B01D 15/203; B01D 15/426; B01D 15/3823; B01D 15/166; C12N 2750/14143; C12N 2750/14151

(57) ABSTRACT

The present disclosure provides methods for purifying a recombinant AAV (rAAV) vector from a solution by affinity chromatography to produce an eluate enriched for AAV vectors (rAAV vectors).

### CROSS-REFERENCE TO RELATED APPLICATIONS

This application claims priority to U.S. Provisional Patent Application No. 63/212,457, filed Jun. 18, 2021 and U.S. Provisional Patent Application No. 63/129,934, filed Dec. 23, 2020, the contents of each of which are incorporated herein by reference in their entirety.

### SEQUENCE LISTING

The instant application contains a Sequence Listing which has been submitted electronically in ASCII format and is hereby incorporated by reference in its entirety. Said ASCII copy, created Nov. 17, 2021, is named PC072554A_Seq_Listing_ST25.txt and is 1,048,576 bytes in size.

### FIELD OF THE INVENTION

The present invention relates to the purification of AAV, and in particular recombinant AAV (rAAV) vectors by affinity chromatrography.

### BACKGROUND

Gene therapy, including those using a recombinant AAV (rAAV) vector to deliver a therapeutic transgene, has the potential to treat a wide range of serious diseases for which no cure, and in many cases, limited treatment exists (Wang et al. (2019) Nature Reviews 18:358-378). Manufacturing of gene therapy vectors is complex and requires specialized methods to purify the therapeutic rAAV vector from host cell impurities and from viral capsids that do not contain a complete vector genome encoding the therapeutic transgene. In addition to the development of a purification method that produces a clinical grade rAAV vector composition of high purity and with a good safety and efficacy profile, the purification method must also be scalable to high volume rAAV production levels to meet patient needs.

Chromatographic methods including affinity and/or ion exchange chromatography have proven useful for large-scale production of clinical grade rAAV, including separation of empty viral capsids from full rAAV vectors.

There remains a need for methods for preparation of clinical grade rAAV vectors (e.g., rAAV9) with optimal purity, potency and consistency. These methods include the purification of rAAV comprising a vector genome with therapeutic transgene at a scale necessary to meet the clinical need for treatment of disease (e.g., Duchenne Muscular Dystrophy (DMD), Friedreich's Ataxia (FA)).

### SUMMARY

The present disclosure provides affinity chromatography methods of purification of rAAV vectors including, but not limited to, the purification of rAAV vectors (e.g., rAAV9 vectors) from host cell proteins and host nucleic acids. Such purified rAAV vectors are suitable for the manufacturing and production of a drug product for administration to a human subject, such as a subject with DMD. Provided herein are methods of preparation of a chromatography eluate comprising rAAV vectors (e.g., from affinity chromatography) for further purification by a subsequent chromatography step, for example, anion exchange chromatography (AEX). The disclosure also provides methods for the regeneration of an affinity chromatography stationary phase that, advantageously, allows the stationary phase to be used in multiple chromatography runs while maintaining the integrity of the process (e.g., successful purification of rAAV vectors, etc.) and while reducing manufacturing costs. Accordingly, the purification methods disclosed herein are a key aspect of manufacturing methods, that in some embodiments, produce a rAAV vector composition.

Those skilled in the art will recognize, or be able to ascertain using no more than routine experimentation, many equivalents to the specific embodiments of the invention described herein. Such equivalents are intended to be encompassed by the following embodiments (E).
E1. A method of purifying a rAAV vector, the method comprising loading a solution comprising the rAAV vector on an affinity chromatography stationary phase and eluting the rAAV vector from the stationary phase with an elution buffer to produce an eluate.
E2. The method of E1, wherein the eluate is an affinity eluate.
E3. The method of E1 or E2, wherein the loading is performed at a linear velocity of 100 cm/hr to 550 cm/hr.
E4. The method of any one of E1-E3, wherein the loading is performed at a linear velocity of about 150 cm/hr to 200 cm/hr, about 250 cm/hr to 350 cm/hr or about 450 cm/hr to 550 cm/hr.
E5. The method of any one of E1-E4, wherein the loading is performed at a linear velocity of about 170 cm/hr, about 300 cm/hr, or about 500 cm/hr.
E6. The method of any one of E1-E5, wherein the loading is performed at a residence time of about 2 min/column volume (CV) to about 10 min/CV, e.g., from about 1 min/CV to about 5 min/CV, from about 2 min/CV to about 8 min/CV, from about 2 min/CV to about 4 min/CV, from about 3 min/CV to about 4 min/CV, from about 4 min/CV to about 5 min/CV, from about 5 min/CV to about 6 min/CV, from about 6 min/CV to about 7 min/CV, from about 7 min/CV to about 8 min/CV, about from 8 min/CV to about 9 min/CV or from about 9 min/CV to about 10 min/CV.
E7. The method of any one of E1-E6, wherein the loading is performed at a residence time of about 3 min/CV, about 3.5 min/CV, about 4 min/CV, about 4.5 min/CV, about 5 min/CV, about 5.5 min/CV, about 6 min/CV, about 6.5 min/CV, about 7 min/CV, about 7.5 min/CV or about 8 min/CV.
E8. The method of any one of E1-E7, wherein the solution comprising the rAAV vector is a clarified lysate.
E9. The method of E8, wherein the clarified lysate is prepared from a host cell culture.
E10. The method of E9, wherein the host cell culture comprises a host cell selected from the group consisting of HEK293, PER.C6, W138, MRC5, A549, HeLa, HepG2, Saos-2, HuH7, HT1080, VERO, COS-1, COS-7, MDCK, BHK21-F, HKCC, and CHO cells.
E11. The method of E9 or E10, wherein the host cell is a HEK293 cell.
E12. The method of any one of E1-E11, wherein the solution comprising the rAAV vector is loaded onto the stationary phase to achieve a challenge of 1×10¹² viral genomes (vg)/mL stationary phase to 1.5×10¹⁴ vg/mL stationary phase.
E13. The method of any one of E1-E12, wherein the solution comprising the rAAV vector is loaded onto the stationary phase to achieve a challenge of 2×10¹³ vg/mL stationary phase to 8×10¹³ vg/mL stationary phase.
E14. The method of any one of E1-E13, wherein the solution comprising the rAAV vector is loaded onto the stationary phase to achieve a challenge of 3×10¹³ vg/mL stationary phase to 7×10¹³ vg/mL stationary phase.
E15. The method of any one of E1-E14, wherein the solution comprising the rAAV vector is loaded onto the stationary phase to achieve a challenge of about 3.8×10¹³ vg/mL stationary phase, about 2.5×10¹³ vg/mL stationary phase, about 7.5×10¹³ vg/mL stationary phase or about 8×10¹³ vg/mL stationary phase.
E16. The method of any one of E1-E15, wherein the solution comprising the rAAV vector is loaded onto the stationary phase to achieve a challenge of ≤5×10¹⁶ vg/L stationary phase.
E17. The method of any one of E1-E16, wherein the stationary phase binds a rAAV capsid.
E18. The method of any one of E1-E17, wherein the stationary phase is an affinity chromatography stationary phase that binds an rAAV9 capsid and the eluate is an affinity eluate comprising an rAAV9 vector.
E19. The method of any one of E1-E18, wherein the stationary phase comprises one or more antigen-binding domains that bind an rAAV capsid.
E20. The method of any one of E1-E19, wherein the stationary phase comprises one or more single domain antibody that binds an rAAV capsid or a rAAV vector.
E21. The method of any one of E1-E20, wherein the stationary phase is an affinity chromatography stationary phase that binds an AAV9 capsid that comprises a VP1 protein comprising an amino acid sequence that is at least 80%, 85% 90%, 95%, 98%, 99% or 100% identical to SEQ ID NO:3.
E22. The method of any one of E1-E21, wherein the stationary phase comprises a camelid-derived single domain antibody (e.g., V_{H}H).
E23. The method of any one of E1-E22, wherein the stationary phase has a dynamic binding capacity of greater than 1.0×10¹³ vg/mL stationary phase.
E24. The method of any one of E1-E23, wherein the stationary phase has a dynamic binding capacity of about 1.0×10¹⁴ vg/mL stationary phase, optionally wherein the binding capacity is measured by ITR qPCR.
E25. The method of any one of E1-E24, wherein the stationary phase has a dynamic binding capacity of about 5.0×10¹³ vg/mL stationary phase, optionally wherein the binding capacity is measured by transgene qPCR.
E26. The method of any one of E1-E25, wherein the stationary phase is an affinity resin capable of binding a capsid of an AAV serotype of AAV1, AAV2, AAV3, AAV4, AAV5, AAV6, AAV7, AAV8, AAV9 or a combination thereof.
E27. The method of any one of E1-E26, wherein the stationary phase is an affinity resin capable of binding a capsid of an AAV serotype of AAV9.
E28. The method of any one of E1-E27, wherein the stationary phase is an affinity resin capable of binding a chimeric capsid.
E29. The method of any one of E1-E28, wherein the stationary phase is a resin comprising polystyrenedivinylbenzene beads, optionally wherein the beads are 50 μm and porous.
E30. The method of any one of E1-E29, wherein the stationary phase is POROS™ Capture Select™ AAVX affinity resin.
E31. The method of any one of E1-E30, wherein the stationary phase is POROS™ Capture Select™ AAV9 affinity resin.
E32. The method of any one of E1-E31, wherein the method further comprises a pre-use rinse of the stationary phase prior to loading the solution comprising the rAAV vector on the stationary phase.
E33. The method of E32, wherein the pre-use rinse comprises application of water (e.g., water for injection) to the stationary phase and removal of all or a portion of the water from the stationary phase.
E34. The method of E33, wherein about 2 CV to about 10 CV, e.g., about 2 CV, about 3 CV, about 4 CV, about 5 CV, about 6 CV, about 7 CV, about 8 CV, about 9 CV or about 10 CV of the water is applied to the stationary phase.
E35. The method of any one of E32-E34, wherein the pre-use rinse flows upward through the stationary phase.
E36. The method of any one of E32-E35, wherein the pre-use rinse removes a storage solution and optionally, wherein the storage solution is a solution comprising 15% to 20% ethanol.
E37. The method of any one of E1-E36, wherein the method further comprises pre-use sanitization of the stationary phase prior to loading the solution comprising the rAAV vector on the stationary phase.
E38. The method of E37, wherein the pre-use sanitization comprises application of a solution comprising phosphoric acid (e.g., about 0.1 N to about 0.2 N phosphoric acid) to the stationary phase and removal of all or a portion of the solution from the stationary phase.
E39. The method of E37 or E38, wherein the solution comprises about 0.132 N phosphoric acid at a pH of about 1.5 to about 2.5 (e.g., about 1.9).
E40. The method of E38 or E39, wherein about 2 CV to about 10 CV, e.g., about 2 CV, about 3 CV, about 4 CV, about 5 CV, about 6 CV, about 7 CV, about 8 CV, about 9 CV or about 10 CV of the solution is applied to the stationary phase.
E41. The method of any one of E38-E40, wherein the stationary phase is contacted with about 2.5 CV of the solution, followed by a hold period of about 45 minutes, followed by contacting the stationary phase with a second 2.5 CV of the solution.
E42. The method of any one of E38-E41, wherein the solution flows upward through the stationary phase.
E43. The method of any one of E37-E42, wherein the pre-use sanitization reduces bioburden.
E44. The method of any one of E1-E43, wherein the method further comprises a first equilibration of the stationary phase prior to loading the solution comprising the rAAV vector on the stationary phase.
E45. The method of E44, wherein the first equilbration comprises application of a buffer solution comprising a buffering agent (e.g., 50 mM to 150 mM Tris) to the stationary phase and removal of all or a portion of the buffer solution from the stationary phase.
E46. The method of E45, wherein the buffer solution comprises about 100 mM Tris at a pH of 7.5.
E47. The method of E45 or E46, wherein about 2 CV to about 10 CV, e.g., about 2 CV, about 3 CV, about 4 CV, about 5 CV, about 6 CV, about 7 CV, about 8 CV, about 9 CV or about 10 CV of the buffer solution is applied to the stationary phase.
E48. The method of any one of E44-E47, wherein the first equilibration of the stationary phase adjusts the pH of the stationary phase.
E49. The method of any one of E1-E48, wherein the method further comprises a second equilibration of the stationary phase after loading the solution comprising the rAAV vector on the stationary phase and before eluting the rAAV vector from the stationary phase, and optionally prior to a pre-elution wash.
E50. The method of E49, wherein the second equilbration comprises application of a buffer solution comprising a buffering agent (e.g., 50 mM to 150 mM Tris) to the stationary phase and removal of all or a portion of the buffer solution from the stationary phase.
E51. The method of E50, wherein the buffer solution comprises about 100 mM Tris at a pH of 7.5.
E52. The method of E50 or E51, wherein about 2 CV to about 10 CV, e.g., about 2 CV, about 3 CV, about 4 CV, about 5 CV, about 6 CV, about 7 CV, about 8 CV, about 9 CV or about 10 CV of the buffer solution is applied to the stationary phase.
E53. The method of any one of E1-E52, wherein the method further comprises a pre-elution wash after loading the solution comprising the rAAV vector on the stationary phase and before eluting the rAAV vector from the stationary phase.
E54. The method of E53, wherein the pre-elution wash comprises application of a pre-elution wash solution to the stationary phase and removal of all or a portion of the pre-elution wash solution from the stationary phase.
E55. The method of any one of E53-E54, wherein the pre-elution wash solution i) removes bound impurities from the stationary phase; ii) maintains rAAV-stationary phase ligand binding; iii) has a reduced pH to improve removal of proteins other than rAAV proteins, such as host cell proteins, or a combination thereof.
E56. The method of E54 or E55, wherein the pre-elution wash solution comprises a solvent and a buffering agent.
E57. The method of E56, wherein the solvent is selected from the group consisting of ethanol, isopropanol, propanol, butanol, and ethylene glycol.
E58. The method of E56 or E57, wherein the solvent is ethanol.
E59. The method of any one of E56-E58, wherein the concentration of the solvent is about 2% to about 60%, e.g., about 10% to about 40%, about 10% to about 30%, about 2% to about 10%, about 10% to about 25%, about 10% to about 20%, about 20% to about 30%, about 30% to about 40%, about 40% to about 50% or about 50% to about 60%.
E60. The method of any one of E56-E59, wherein the concentration of the solvent is about 15% to about 20% (e.g., about 17%, about 17.5%).
E61. The method of any one of E56-E60, wherein the solvent is ethanol and wherein the concentration of the ethanol is about 15% to about 20% (e.g. about 17%, about 17.5%).
E62. The method of any one of E56-E61, wherein the solvent is ethanol and wherein the concentration of the ethanol is about 17%.
E63. The method of any one of E56-E61, wherein the solvent is ethanol and wherein the concentration of the ethanol is about 17.5%.
E64. The method of any one of E56-E63, wherein the buffering agent is selected from the group consisting of sodium acetate, ammonium acetate, Tris (e.g., a mixture of Tris Base and Tris-HCl), BIS-Tris propane, diethanolamine, diethylamine, tricine, triethanolamine, bicine and a combination thereof.
E65. The method of any one of E56-E64, wherein the concentration of the buffering agent is about 10 mM to about 500 mM, e.g., about 50 mM to about 400 mM, about 100 mM to about 300 mM, about 10 mM to about 50 mM, about 50 mM to about 100 mM, about 100 mM to about 200 mM, about 50 mM to about 150 mM, about 200 mM to about 300 mM, about 300 mM to about 400 mM, or about 400 mM to about 500 mM.
E66. The method of any one of E56-E65, wherein the concentration of the buffering agent is about 50 mM to about 200 mM.
E67. The method of any one of E56-E66, wherein the buffering agent is sodium acetate.
E68. The method of any one of E56-E67, wherein the buffering agent is sodium acetate and wherein the concentration of the sodium acetate in the pre-elution wash solution is about 145 mM to about 155 mM (e.g., about 153 mM).
E69. The method of any one of E56-E68, wherein the buffering agent is sodium acetate and wherein the concentration of the sodium acetate in the pre-elution wash solution is about 150 mM.
E70. The method of any one of E56-E69, wherein the buffering agent is sodium acetate and wherein the concentration of the sodium acetate in the pre-elution wash solution is about 153 mM.
E71. The method of any one of E54-E70, wherein the pre-elution wash solution has a pH of about 4.5 to 8.0, e.g., about 5.0 to about 8.0, about 5.0 to about 6.0, about 5.0 to about 5.5, about 5.5 to about 6.0, about 6.0 to about 6.5, about 6.5 to about 7, about 7.0 to about 8.0, about 7.5 to about 8.0.
E72. The method of any one of E54-E71, wherein the pre-elution wash solution has a pH of 5 to 6 (e.g., about 5.6).
E73. The method of any one of E54-E72, wherein the pre-elution wash solution comprises 15% to 25% of ethanol, 100 mM to 200 mM of sodium acetate, and a pH of 5 to 6.
E74. The method of any one of E54-E73, wherein the pre-elution wash solution comprises about 15% to 20% ethanol, about 145 mM to about 155 mM sodium acetate, and a pH of 5 to 6.
E75. The method of any one of E54-E74, wherein the pre-elution wash solution comprises about 17.5% ethanol, about 153 mM sodium acetate, and a pH of about 5.6.
E76. The method of any one of E54-E75, wherein the pre-elution wash solution comprises about 17% ethanol, about 150 mM sodium acetate, and a pH of about 5.6.
E77. The method of any one of E54-E76, wherein 2 CV to 10 CV, e.g., 1 CV to 3 CV, 3 CV to 5 CV, 3 CV to 8 CV, 4 CV to 6 CV, 5 CV to 8 CV, or 8 CV to 10 CV, of pre-elution wash solution is applied to the stationary phase.
E78. The method of any one of E54-E77, wherein 4.5 CV to 5.5 CV of pre-elution wash solution is applied to the stationary phase.
E79. The method of any one of E54-E78, wherein about 5 CV of pre-elution wash solution is applied to the stationary phase.
E80. The method of any one of E54-E79, wherein flow velocity of the pre-elution wash solution is about 10 cm/hr to about 600 cm/hr, e.g., about 50 cm/hr to about 500 cm/hr, about 100 cm/hr to about 300 cm/hr, about 100 cm/hr to about 400 cm/hr, about 100 cm/hr to about 500 cm/hr, about 10 cm/hr to about 50 cm/hr, about 50 cm/hr to about 100 cm/hr, about 100 cm/hr to about 200 cm/hr, about 200 cm/hr to about 300 cm/hr, about 150 cm/hr to about 250 cm/hr, about 300 cm/hr to about 400 cm/hr, about 400 cm/hr to about 500 cm/hr, about 450 cm/hr to about 550 cm/hr, or about 500 cm/hr to about 600 cm/hr.
E81. The method of any one of E54-E80, wherein flow velocity of the pre-elution wash solution is about 150 cm/hr to about 200 cm/hr (e.g., about 170 cm/hr), about 150 cm/hr to about 250 cm/hr (e.g., about 200 cm/hr) or about 300 cm/hr to about 400 cm/hr (e.g., 345 cm/hr).
E82. The method of any one of E54-E81, wherein residence time of the pre-elution wash is about 2 min/CV to about 10 min/CV, e.g., about 1 min/CV to about 5 min/CV, about 3 min/CV to about 8 min/CV, about 2 min/CV to about 3 min/CV, about 3 min/CV to about 4 min/CV, about 4 min/CV to about 5 min/CV, about 5 min/CV to about 6 min/CV, about 6 min/CV to about 7 min/CV, about 7 min/CV to about 8 min/CV, about 8 min/CV to about 9 min/CV, or about 9 min/CV to about 10 min/CV.
E83. The method of any one of E54-E82, wherein residence time of the pre-elution wash solution is about 2 min/CV, about 3 min/CV, about 4 min/CV, about 4.3 min/CV, about 4.35 min/CV, about 4.5 min/CV, about 5 min/CV, about 5.5. min/CV, about 6 min/CV, about 7 min/CV, or about 8 min/CV.
E84. The method of any one E53-E83, wherein an eluate from the pre-elution wash has an A280 maximum peak height of at least 750 mAU/mm.
E85. The method of any one E53-E83, wherein an eluate from the pre-elution wash has an A280 maximum peak height of about 750 mAU/mm to about 1250 mAU/mm.
E86. The method of any one of E54-E85, wherein about 4.5 CV to about 5.5 CV (e.g., about 5 CV) of a pre-elution wash solution comprising about 15% to about 20% (e.g., about 17.5%) ethanol, about 150 mM to about 155 mM (e.g., about 153 mM) sodium acetate, pH 5 to 6 (e.g., about 5.6) is applied to the stationary phase, and optionally wherein flow velocity of the pre-elution wash solution is about 300 to about 400 cm/hr (e.g., about 345 cm/hr) and optionally wherein the residence time of the pre-elution wash solution is about 4 min/CV to about 5 min/CV (e.g., about 4.35 min/CV).
E87. The method of any one of E54-E86, wherein about 4.5 CV to about 5.5 CV (e.g., about 5.0 CV) of a pre-elution wash solution comprising about 15% to about 20% (e.g., about 17.5%) ethanol, about 150 mM to about 155 mM (e.g., about 153 mM) sodium acetate, pH 5 to 6 (e.g., about 5.6) is applied to the stationary phase, and optionally wherein flow velocity of the pre-elution wash solution is about 150 to about 250 cm/hr (e.g., about 200 cm/hr) and optionally wherein the residence time of the pre-elution wash solution is about 4.5 min/CV to about 5.5 min/CV (e.g., about 5.0 min/CV).
E88. The method of any one of E54-E87, wherein about 2.5 CV to about 3.5 CV (e.g., about 3.0 CV) of a pre-elution wash solution comprising about 15% to about 20% (e.g., about 17%) ethanol and about 145 mM to about 155 mM (e.g., about 150 mM), pH 5 to 6 (e.g., about 5.6) is applied to the stationary phase, and optionally wherein flow velocity of the pre-elution wash solution is about 150 to about 200 cm/hr (e.g., about 170 cm/hr) and optionally wherein the residence time of the pre-elution wash solution is about 2.5 min/CV to about 3.5 min/CV (e.g., about 3.0 min/CV).
E89. The method of any one of E1-E88, wherein the method further comprises a third equilibration of the stationary phase after loading the solution comprising the rAAV vector on the stationary phase and before eluting the rAAV vector from the stationary phase, and optionally after the pre-elution wash.
E90. The method of E89, wherein the third equilbration comprises application of a buffer solution (e.g., about 150 mM to about 160 mM sodium acetate) to the stationary phase and removal of all or a portion of the buffer solution from the stationary phase.
E91. The method of E90, wherein the buffer solution comprises about 153 mM sodium acetate at a pH of about 5.6.
E92. The method of E90 or E91, wherein about 2 CV to about 10 CV, e.g., about 2 CV, about 3 CV, about 4 CV, about 5 CV, about 6 CV, about 7 CV, about 8 CV, about 9 CV or about 10 CV of the buffer solution is applied to the stationary phase.
E93. The method of any one of E90-E92, wherein the third equilibrataion comprises application of about 5 CV of the buffer solution comprising about 153 mM sodium acetate at a pH of 5.6 to the stationary phase.
E94. The method of any one of E1-E93, wherein eluting the rAAV vector from the stationary phase comprises application of an elution buffer to the stationary phase and removing all or a portion of the elution buffer from the stationary phase.
E95. The method of E94, wherein the elution buffer the does not preferentially elute residual impurities from the stationary phase.
E96. The method of any one of E1-E95,
   i) wherein the elution buffer elutes the rAAV vector from the stationary phase;
   ii) wherein the elution buffer does not result in precipitation of an affinity eluate;
   iii) wherein the elution buffer maximizes an % vg recovery;
   iv) wherein the elution buffer does not interfere with binding of the rAAV vector to an anion exchange chromatography (AEX) stationary phase;
   v) wherein the elution buffer does not contain a trivalent anion;
   vi) wherein the elution buffer does not contain citrate ions, or a combination thereof.
E97. The method of any one of E1-E96, wherein the elution buffer does not interfere with binding of the rAAV vector to an AEX stationary phase such that 90% to 100% of the rAAV vector binds to the AEX stationary phase in a subsequent purification step.
E98. The method of any one of E1-E97, wherein the elution buffer comprises a component selected from the group consisting of a salt, an amino acid, a buffering agent, and any combination thereof.
E99. The method of E98, wherein the salt of the elution buffer is selected from the group consisting of sodium chloride, magnesium chloride, sodium sulfate, and any combination thereof.
E100. The method of E98 or E99, wherein the salt of the elution buffer is magnesium chloride.
E101. The method of any of E98-E100, wherein the concentration of the salt of the elution buffer is about 1 mM to about 500 mM, e.g., about 5 mM to 150 mM, about 10 mM to about 200 mM, about 25 M to about 150 mM, about 1 mM to about 10 mM, about 10 mM to about 50 mM, about 50 mM to about 100 mM, about 100 mM to about 200 mM, about 200 mM to about 300 mM, about 300 mM to about 400 mM, and about 400 mM to about 500 mM.
E102. The method of any one of E98-E101, wherein the concentration of the salt of the elution buffer is about 5 mM to about 150 mM.
E103. The method of any one of E98-E102, wherein the salt is magnesium chloride and wherein the concentration of the magnesium chloride in the elution buffer is about 10 mM to about 100 mM (e.g., about 25 mM).
E104. The method of any one of E98-E103, wherein the amino acid of the elution buffer is selected from the group consisting of histidine, arginine, citrulline, glycine, and any combination thereof.
E105. The method of any one of E98-E104, wherein the amino acid is glycine.
E106. The method of any one of E98-E105, wherein the concentration of the amino acid of the elution buffer is about 0 mM to about 500 mM, e.g., about 10 mM to about 400 mM, about 50 mM to about 300 mM, about 50 mM to about 150 mM, about 0 mM to about 10 mM, about 10 mM to about 50 mM, about 50 mM to about 100 mM, about 100 mM to about 200 mM, about 200 mM to about 300 mM, about 300 mM to about 400 mM, and about 400 mM to about 500 mM.
E107. The method of any one of E98-E106, wherein the concentration of the amino acid in the elution buffer is about 10 mM to about 200 mM.
E108. The method of any one of E98-E107, wherein the concentration of the amino acid in the elution buffer is about 50 mM to about 150 mM.
E109. The method of any one of E98-E108, wherein the concentration of the amino acid in the the elution buffer is about 75 mM to about 125 mM (e.g., about 100 mM).
E110. The method of any one of E98-E109, wherein the amino acid is glycine and wherein the concentration of the glycine in the elution buffer is about 75 mM to about 125 mM (e.g., about 100 mM).
E111. The method of any one of E98-E110, wherein the buffering agent is sodium citrate, sodium acetate, ammonium acetate, Tris (e.g., a mixture of Tris Base and Tris-HCl), BIS-Tris propane, diethanolamine, diethylamine, tricine, triethanolamine and/or bicine.
E112. The method of E98-E111, wherein the buffering agent of the elution buffer is sodium acetate.
E113. The method of any one of E98-E112, wherein the concentration of the buffering agent in the elution buffer is about 10 mM to about 500 mM, e.g., about 100 mM to about 400 mM, 145 mM to about 150 mM, about 200 mM to about 300 mM, about 10 mM to about 50 mM, about 50 mM to about 100 mM, about 75 mM to 250 mM, about 100 mM to about 200 mM, about 200 mM to about 300 mM, about 300 mM to about 400 mM, or about 400 mM to about 500 mM.
E114. The method of any one of E98-E113, wherein the concentration of the buffering agent in the elution buffer is about 145 mM to about 150 mM (e.g., about 148 mM).
E115. The method of any one of E98-E114, wherein the buffering agent is sodium acetate and wherein the concentration of the sodium acetate in the elution buffer is about 145 mM to about 150 mM (e.g., about 148 mM).
E116. The method of any one of E1-E115, wherein the elution buffer has a pH of about 2 to about 6, e.g., about 2 to 4, about 2.5 to about 3.5, about 2 to 2.5, about 2.5 to about 3, about 3 to about 3.5, about 2.5 to about 3.5, about 3.5 to about 4, about 4 to about 4.5, about 4.5 to about 5, about 5 to about 5.5 or about 5.5 to about 6.0.
E117. The method of any one of E1-E116, wherein the elution buffer has a pH of about 3, about 4, or about 5.
E118. The method of any one of E1-E116, wherein the elution buffer has a pH of about 2.5 to about 3.5 (e.g., about 3.5).
E119. The method of any one of E1-E118, wherein the elution buffer has a conductivity of about 1 mS/cm to about 40 mS/cm, e.g., about 1 mS/cm to about 20 mS/cm, about 5 mS/cm to about 40 mS/cm, about 15 mS/cm to about 40 mS/cm, about 10 mS/cm to about 15 mS/cm, about 15 mS/cm to about 20 mS/cm, about 20 mS/cm to about 25 mS/cm, about 25 mS/cm to about 30 mS/cm, about 30 mS/cm to about 35 mS/cm, or about 35 mS/cm to about 40 mS/cm.
E120. The method of any one of E1-E119, wherein the elution buffer has a conductivity of about 20 mS/cm to about 35 mS/cm.
E121. The method of any one of E1-E120, wherein the elution buffer is selected from the group consisting of i) 200 mM glycine, 50 mM MgCl₂, pH 3.5; ii) 100 mM glycine, 100 mM MgCl₂, 150 mM sodium acetate, pH 3.0; iii) 100 mM glycine, 50 mM MgCl₂, 150 mM sodium acetate, pH 3.0; iv) 100 mM glycine, 25 mM MgCl₂, 150 mM sodium acetate, pH 3.0; v) 100 mM glycine, 5 mM MgCl₂, 150 mM sodium acetate, pH 3.0; vi) 100 mM glycine, 25 mM MgCl₂, 148 mM sodium acetate, pH 3.0; vii) 100 mM glycine, 100 mM MgCl₂, pH 3.0; viii) 100 mM glycine, 100 mM MgCl₂, 100 mM sodium acetate, pH 3.0; ix) 100 mM glycine, 100 mM MgCl₂, 100 mM sodium acetate, 25 wt./v. % iodixanol, pH 3.0; x) 100 mM glycine, 100 mM MgCl₂, 100 mM sodium acetate, 25 wt. % propylene glycol, pH 3.0, and xi) 50 mM to 150 mM of glycine, 10 mM to 100 mM of MgCl₂, 50 mM to 150 mM of sodium acetate, and a pH of 2.5 to 3.5.
E122. The method of any one of E1-E121, wherein the elution buffer comprises about 75 mM to about 125 mM (e.g., about 100 mM) glycine, about 20 mM to about 30 mM (e.g., about 25 mM) MgCl₂, about 140 to about 150 mM (e.g., about 148 mM) sodium acetate, pH about 2.5 to about 3.5 (e.g., about 3.0).
E123. The method of any one of E1-E122, wherein the elution buffer comprises about 100 mM glycine, about 25 mM MgCl₂, about 148 mM sodium acetate, pH 3.0.
E124. The method of any one of E1-E123, wherein the elution buffer does not contain an anion that competes with binding of the rAAV vector to an AEX stationary phase.
E125. The method of E124, wherein the anion is a trivalent anion.
E126. The method of E124 or E125, wherein the anion is citrate.
E127. The method of any one of E1-E126, wherein 2 CV to 10 CV, e.g., 1 CV to 3 CV, 3 CV to 5 CV, 3 CV to 8 CV, 4 CV to 6 CV, 5 CV to 8 CV, or 8 CV to 10 CV, of elution buffer is applied to the stationary phase.
E128. The method of any one of E1-E127, wherein 4.5 CV to 5.5 CV (e.g., about 5 CV) of elution buffer is applied to the stationary phase.
E129. The method of any one of E1-E128, wherein the flow velocity of the elution buffer is about 10 cm/hr to about 600 cm/hr, e.g., about 50 cm/hr to about 500 cm/hr, about 100 cm/hr to about 300 cm/hr, about 100 cm/hr to about 400 cm/hr, about 100 cm/hr to about 500 cm/hr, about 10 cm/hr to about 50 cm/hr, about 50 cm/hr to about 100 cm/hr, about 100 cm/hr to about 200 cm/hr, about 200 cm/hr to about 300 cm/hr, about 300 cm/hr to about 400 cm/hr, about 250 cm/hr to about 350 cm/hr, about 400 cm/hr to about 500 cm/hr, about 450 cm/hr to about 550 cm/hr, or about 500 cm/hr to about 600 cm/hr.
E130. The method of any one of E1-E129, wherein the flow velocity of the elution buffer is about 50 cm/hr to about 100 cm/hr (e.g., about 64 cm/hr).
E131. The method of any one of E1-E130, wherein the flow velocity of the elution buffer is about 250 cm/hr to about 350 cm/hr (e.g., about 300 cm/hr).
E132. The method of any one of E1-E130, wherein the flow velocity of the eluction buffer is about 450 cm/hr to about 550 cm/hr (e.g., 500 cm/hr).
E133. The method of any one of E1-E132, wherein the residence time of the elution buffer is about 2 min/CV to about 10 min/CV, e.g., about 1 min/CV to about 5 min/CV, about 3 min/CV to about 8 min/CV, about 2 min/CV to about 3 min/CV, about 3 min/CV to about 4 min/CV, about 4 min/CV to about 5 min/CV, about 5 min/CV to about 6 min/CV, about 6 min/CV to about 7 min/CV, about 7 min/CV to about 8 min/CV, about 8 min/CV to about 9 min/CV, about 9 min/CV to about 10 min/CV.
E134. The method of any one of E1-E133, wherein residence time of the elution buffer solution is about 3 min/CV, about 4 min/CV, about 5 min/CV, about 5.5. min/CV, about 6 min/CV, about 7 min/CV, or about 8 min/CV.
E135. The method of any one of E1-E134, wherein about 4.5 CV to about 5.5 CV (e.g., about 5 CV) of the elution buffer comprising about 75 mM to about 125 mM (e.g., about 100 mM) glycine, about 20 mM to about 30 mM (e.g., about 25 mM) MgCl₂, about 140 to about 150 mM (about 148 mM) sodium acetate, pH about 2.5 to about 3.5 (e.g., about 3.0) is applied to the stationary phase, and optionally wherein the flow velocity of the elution buffer is about 450 to about 550 cm/hr (e.g., about 500 cm/hr) and optionally wherein the residence time of the elution buffer is about 2.5 min/CV to about 3.5 min/CV (e.g., about 3 min/CV).
E136. The method of any one of E1-E134, wherein about 4.5 CV to about 5.5 CV (e.g., about 5 CV) of the elution buffer comprising about 75 mM to about 125 mM (e.g., about 100 mM) glycine, about 20 mM to about 30 mM (e.g., about 25 mM) MgCl₂, about 140 to about 150 mM (about 148 mM) sodium acetate, pH about 2.5 to about 3.5 (e.g., about 3.0) is applied to the stationary phase, and optionally wherein the flow velocity of the elution buffer is about 250 cm/hr to about 350 cm/hr (e.g., about 300 cm/hr) and optionally wherein the residence time of the elution buffer is about 2.5 min/CV to about 3.5 min/CV (e.g., about 3 min/CV).
E137. The method of any one of E1-E134, wherein about 4.5 CV to about 5.5 CV (e.g., about 5 CV) of the elution buffer comprising about 75 mM to about 125 mM (e.g., about 100 mM) glycine, about 20 mM to about 30 mM (e.g., about 25 mM) MgCl₂, about 140 to about 150 mM (about 148 mM) sodium acetate, pH about 2.5 to about 3.5 (e.g., about 3.0) is applied to the stationary phase, and wherein the flow velocity of the elution buffer is about 50 cm/hr to about 100 cm/hr (e.g., about 64 cm/hr) and optionally wherein the residence time of the elution buffer is about 7.5 min/CV to about 8.5 min/CV (e.g., about 7.97 min/CV).
E138. The method of any one of E1-E137, further comprising collection of the eluate from the stationary phase, and wherein the eluate is an affinity eluate.
E139. The method of E138, wherein collection of the affinity eluate is started at an A280 of about 1 mAU/mm path length and stopped at about 35 mAU/mm path length.
E140. The method of E138, wherein collection of the affinity eluate is started at an A280 of about 2.5 mAU/mm path length and stopped at about 32.5 mAU/mm path length.
E141. The method of E138, wherein collection of the affinity eluate is started at an A280 of about 2.5 mAU/mm path length and stopped at an A280 of about 22.5 mAU/mm path length.
E142. The method of E138, wherein collection of the affinity eluate is started at an A280 of about 10 mAU and stopped at about 120 mAU (about 5 mm path length).
E143. The method of E138, wherein collection of the affinity eluate is started at an A280 of about 12.5 mAU and stopped at about 112.5 mAU (about 5 mm path length).
E144. The method of any one of E138-E143 wherein a volume of affinity eluate collected from the stationary phase is about 1 mL to about 3 L, about 10 mL to about 3 L, about 100 mL to about 3 L, about 200 mL to about 3 L, about 500 mL to about 3 L, about 100 mL to about 2 L, or about 100 mL to about 1 L.
E145. The method of any one of E138-E144, wherein a volume of affinity eluate collected from the stationary phase is about 0.1 CV to about 10 CV.
E146. The method of any one of E138-E145, wherein a volume of affinity eluate collected from the stationary phase is about 0.1 CV to about 8 CV, about 0.1 CV to about 5 CV, about 0.1 CV to about 1 CV, about 0.1 CV to about 0.5 CV, about 0.5 CV to about 8 CV, about 0.5 CV to about 5 CV, about 0.5 CV to about 3 CV, about 0.5 CV to about 1.5 CV, about 0.5 CV to about 1.0 CV, about 1 CV to about 8 CV, about 1 CV to about 5 CV, or about 1 CV to about 2 CV.
E147. The method of any one of E138-E146, wherein a volume of affinity eluate collected from the stationary phase is about 0.5 CV to about 1.0 CV.
E148. The method of any one of E138-E147, wherein a volume of affinity eluate collected from the stationary phase is about 1 CV to about 2 CV (e.g., 1.1 CV to 1.9 CV).
E149. The method of any one of E138-E148, wherein the affinity eluate is collected as a single fraction.
E150. The method of any one of E138-E149, wherein the affinity eluate is collected as more than one fraction.
E151. The method of E150, wherein the more than one affinity eluate fractions are pooled.
E152. The method of E1-E151, wherein the stationary phase is within a column.
E153. The method of E152, wherein the column has a volume of about 1 mL to about 30 L, about 1 mL to about 20 L, about 1 mL to about 10 L, about 1 mL to about 5 L, about 1 mL to about 1 L, about 1 mL to about 500 mL, about 1 mL to about 250 mL, about 1 mL to about 100 mL, about 1 mL to about 50 mL, about 1 mL to about 10 mL, about 500 mL to about 1 L, about 1 L to about 2 L, about 1 L to about 3 L, about 1 L to about 4 L, about 1 L to about 5 L, about 1 L to about 6 L, about 1 L to about 7 L, about 1 L to about 8 L, about 1 L to about 9 L, about 1 L to about 10 L, about 10 L to about 15 L, about 10 L to about 20 L, about 10 L to about 25 L, or about 10 L to about 30 L.
E154. The method of any one of E152-E153, wherein the volume of the column is about 1 L (e.g., about 0.997 L) with an inner diameter or about 10 cm and a bed height of about 13 cm (e.g., about 12.7 cm).
E155. The method of any one of E152-E153, wherein the volume of the column is about 2.6 L (e.g., about 2.67 L) with an inner diameter of about 20 cm and a bed height of about 8.5 cm.
E156. The method of any one of E152-E153, wherein the volume of the column is about 17.0 L (e.g., 17.663 L) with an inner diameter or about 30 cm and a bed height of about 25 cm.
E157. The method of of any one of E1532-E153, wherein the volume of the column is about 25 L (e.g., about 25.61 L) with an inner diameter of about 45 cm and a bed height of about 15 cm.
E158. The method of any one of E2-E157, wherein a % vg yield in the affinity eluate is about 50% to about 100%, about 60% to about 100%, about 70% to about 100%, about 80% to about 100%, about 85% to about 100%, about 90% to about 100%, about 95% to about 100%, or about 98% to about 100%.
E159. The method of E158, wherein the % vg yield of the affinity eluate is about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100%.
E160. The method of any one of E2-E158, wherein a vg per mL of affinity eluate is about 1.0×10¹² vg/mL to about 1.0×10¹⁴ vg/mL, about 5.0×10¹² vg/mL to about 1.0×10¹⁴ vg/mL, about 5.0×10¹² vg/mL to about 7.0×10¹³ vg/mL, about 1.0×10¹³ vg/mL to about 1.0×10¹⁴ vg/mL, about 1.0×10¹³ vg/mL to about 8.0×10¹³ vg/mL, or about 1.0×10¹³ vg/mL to about 5.0×10¹³ vg/mL.
E161. The method of any one of E158-E160, wherein the % vg yield of the affinity eluate, and/or vg per mL of the affinity eluate is measured by qPCR.
E162. The method of E161, wherein the qPCR measures copies of a transgene sequence.
E163. The method of E162, wherein the transgene sequence comprises a nucleic acid sequence that is at least 80%, 85% 90%, 95%, 98%, 99% or 100% identical to SEQ ID NO:1, or a fragment thereof.
E164. The method of E161, wherein the qPCR measures copies of an inverted terminal repeat (ITR) sequence.
E165. The method of E164, wherein the ITR sequence comprises a nucleic acid sequence that is at least 80%, 85% 90%, 95%, 98%, 99% or 100% identical to any one of SEQ ID NO:5-8, or a fragment thereof.
E166. The method of any one of E2-E165, wherein an A260/A280 ratio of the affinity eluate is greater than 0.8.
E167. The method of any one of E2-E166, wherein the A260/A280 ratio of the affinity eluate is about 0.8, about 0.81, about 0.82, about 0.83, about 0.84, about 0.85, about 0.86, about 0.87, about 0.88, about 0.89, about 0.9, about 0.91, about 0.92, about 0.93, about 0.94, about 0.95, about 0.96, about 0.97, about 0.98, about 0.99, about 1.0, about 1.1, about 1.11, about 1.12, about 1.13, about 1.14, about 1.15, about 1.16, about 1.17, about 1.18, about 1.19, about 1.20, about 1.21, about 1.22, about 1.23, about 1.24, about 1.25, about 1.26, about 1.27, about 1.28, about 1.29, about 1.30 or greater.
E168. The method of any one of E2-E167, wherein the A260/A280 of the affinity eluate collected is measured by size exclusion chromatography (SEC) with simultaneous absorbance measurement.
E169. The method of any one of E2-E168, wherein a % purity of the affinity eluate is about 80% to about 100%, about 85% to about 100%, about 90% to about 100%, about 95% to about 100% or about 98% to about 100% capsid protein of total protein in the eluate.
E170. The method of any one of E2-E169, wherein the % purity of the affinity eluate is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% capsid protein of total protein in the eluate.
E171. The method of any one of E169 or E170, wherein the % purity viral protein of total protein of the affinity eluate is measured by reverse phase HPLC, non-reducing.
E172. The method of any one of E169 or E170, wherein the % purity viral protein of total protein of the affinity eluate is measured by size exclusion chromatography (SEC).
E173. The method of any one of E2-E172, wherein % purity viral protein of total protein of the affinity eluate is increased as compared to the % purity capsid protein of total protein of the solution comprising the rAAV vector.
E174. The method of any one of E2-E173, wherein a viral particle titer of the affinity eluate is about 1×10¹¹ vp/mL to about 1×10¹⁵ vp/mL, about 1×10¹² vp/mL to about 1×10¹⁴ vp/mL, about 1×10¹³ vp/mL to about 1×10¹⁴ vp/mL, about 1×10¹³ vp/mL to about 9×10¹³ vp/mL or about 2×10¹³ vp/mL to about 8×10¹³ vp/mL.
E175. The method of any one of E2-E174, wherein a viral particle titer of the affinity eluate is about 1×10¹³ vp/mL, about 2×10¹³ vp/mL, about 3×10¹³ vp/mL, about 4×10¹³ vp/mL, about 5×10¹³ vp/mL, about 6×10¹³ vp/mL, about 7×10¹³ vp/mL, about 8×10¹³ vp/mL, about 9×10¹³ vp/mL or about 10×10¹³ vp/mL.
E176. The method of E174 or E175, wherein the viral particle titer of the affinity eluate is measured by size exclusion chromatography.
E177. The method of any one of E2-E176, wherein the affinity eluate has a pH of about 2.5 to about 4.5, about 3 to about 4, about 3.2 to about 3.6, or about 3.4 to about 3.6.
E178. The method of any one of E2-E177, wherein an amount of host cell DNA (HCDNA) present in the affinity eluate is about 50 ng/1×10¹⁴ vg to about 100 ng/1×10¹⁴ vg, about 50 ng/1×10¹⁴ vg to about 750 ng/1×10¹⁴ vg, about 50 ng/1×10¹⁴ vg to about 500 ng/1×10¹⁴ vg, about 50 ng/1×10¹⁴ vg to about 250 ng/1×10¹⁴ vg, or about 50 to about 100 ng/1×10¹⁴ vg.
E179. The method of any one of E2-E178, wherein an amount of HCDNA present in the affinity eluate is about 1 pg/1×10⁹ vg to about 25 pg/1×10⁹ vg, about 1 pg/1×10⁹ vg to about 20 pg/1×10⁹ vg, about 1 pg/1×10⁹ vg to about 15 pg/1×10⁹ vg, or about 1 pg/1×10⁹ vg to about 10 pg/1×10⁹ vg.
E180. The method of any one of E2-E179, wherein an amount of HCDNA present in the affinity eluate is about 1 pg/1×10⁹ vg, 2 pg/1×10⁹ vg, 3 pg/1×10⁹ vg, 4 pg/1×10⁹ vg, 5 pg/1×10⁹ vg, 6 pg/1×10⁹ vg, 7 pg/1×10⁹ vg, 8 pg/1×10⁹ vg, 9 pg/1×10⁹ vg, 10 pg/1×10⁹ vg, 15 pg/1×10⁹ vg or about 20 pg/1×10⁹ vg.
E181. The method of any one of E2-E180, wherein an amount of HCDNA present in the affinity eluate is about 1×10⁴ pg/mL to about 1×10⁶ pg/mL, about 1×10⁴ pg/mL to about 1×10⁵ pg/mL or 1×10⁴ pg/mL to about 5×10⁴ pg/mL.
E182. The method of any one of E2-E181, wherein an amount of HCDNA present in the affinity eluate is about 5×10⁴ pg/mL, 6×10⁴ pg/mL, 7×10⁴ pg/mL, 8×10⁴ pg/mL, 9×10⁴ pg/mL, 1×10⁵ pg/mL, 2×10⁵ pg/mL, 3×10⁵ pg/mL, 4×10⁵ pg/mL, 5×10⁵ pg/mL, 6×10⁵ pg/mL, 7×10⁵ pg/mL, 8×10⁵ pg/mL, 9×10⁵ pg/mL or 10×10⁵ pg/mL.
E183. The method of any one of E178-E182, wherein the amount of HCDNA present in the affinity eluate is measured by qPCR.
E184. The method of any one of E2-E183, wherein the amount of HCDNA in the affinity eluate is reduced as compared to the amount of HCDNA in the solution comprising the rAAV vector.
E185. The method of E184, wherein the reduction is a log reduction value of the amount of HCDNA in the affinity eluate as compared to the amount of HCDNA in the solution comprising the rAAV vector of about 1 to about −1.
E186. The method of any one of E2-E185, wherein an amount of host cell protein (HCP) present in the affinity eluate is about 1000 ng/1×10¹⁴ vg to about 20000 ng/1×10¹⁴ vg, about 1000 ng/1×10¹⁴ vg to about 15000 ng/1×10¹⁴ vg, about 1000 ng/1×10¹⁴ vg to about 10000 ng/1×10¹⁴ vg, about 1000 ng/1×10¹⁴ vg to about 5000 ng/1×10¹⁴ vg, about 1000 ng/1×10¹⁴ vg to about 2500 ng/1×10¹⁴ vg, or about 5000 ng/1×10¹⁴ vg to about 10000 ng/1×10¹⁴ vg.
E187. The method of any one of E2-E186, wherein an amount of HCP present in the affinity eluate is about 10 pg/1×10⁹ vg to about 4000 pg/1×10⁹ vg, 10 pg/1×10⁹ vg to about 3000 pg/1×10⁹ vg, 10 pg/1×10⁹ vg to about 2000 pg/1×10⁹ vg, 10 pg/1×10⁹ vg to about 1000 pg/1×10⁹ vg about 10 pg/1×10⁹ vg to about 800 pg/1×10⁹ vg, about 10 pg/1×10⁹ vg to about 600 pg/1×10⁹ vg, about 10 pg/1×10⁹ vg to about 500 pg/1×10⁹ vg, about 10 pg/1×10⁹ vg to about 250 pg/1×10⁹ vg, about 10 pg/1×10⁹ vg to about 100 pg/1×10⁹ vg, or about 50 pg/1×10⁹ vg to about 500 pg/1×10⁹ vg.
E188. The method of any one of E2-E187, wherein an amount of HCP present in the affinity eluate is about 10 pg/1×10⁹ vg, 20 pg/1×10⁹ vg, 30 pg/1×10⁹ vg, 40 pg/1×10⁹ vg, 50 pg/1×10⁹ vg, 60 pg/1×10⁹ vg, 70 pg/1×10⁹ vg, 80 pg/1×10⁹ vg, 90 pg/1×10⁹ vg, 100 pg/1×10⁹ vg, 200 pg/1×10⁹ vg, 300 pg/1×10⁹ vg, 400 pg/1×10⁹ vg, 500 pg/1×10⁹ vg, 600 pg/1×10⁹ vg, 700 pg/1×10⁹ vg, 800 pg/1×10⁹ vg, 900 pg/1×10⁹ vg or 1000 pg/1×10⁹ vg.
E189. The method of any one of E2-E188, wherein an amount of HCP present in the affinity eluate is about 1×10² ng/mL to about 1×10⁵ ng/mL, about 1×10² ng/mL to about 1×10⁴ ng/mg, 1×10² ng/mL to about 1×10³ ng/mL or about 1×10³ ng/mL to about 1×10⁴ ng/mL.
E190. The method of any one of E2-E189, wherein an amount of HCP present in the affinity eluate is about 1×10³ ng/mL, 2×10³ ng/mL, 3×10³ ng/mL, 4×10³ ng/mL, 5×10³ ng/mL, 6×10³ ng/mL, 7×10³ ng/mL, 8×10³ ng/mL, 9×10³ ng/mL, 10×10³ ng/mL or 20×10³ ng/mL.
E191. The method of any one of E186-E190, wherein the amount of HCP present in the affinity eluate is measured by a method selected from the group consisting of ELISA, Western blot, and silver staining.
E192. The method of any one of E2-E191, wherein amount of HCP in the affinity eluate is reduced as compared to the amount of HCP in the solution comprising the rAAV vector.
E193. The method of E192, wherein there is a log reduction value of the amount of HCP in the affinity eluate as compared to the amount of HCP in the solution comprising the rAAV vector of about 1 to about 10.
E194. The method of any one of E2-E193, wherein an amount of residual plasmid DNA present in the affinity eluate is about 1 pg/1×10¹ vg to about 100 pg/1×10⁹ vg, about 1 pg/1×10⁹ vg to about 90 pg/1×10⁹ vg, about 1 pg/1×10⁹ vg to about 80 pg/1×10⁹ vg, about 1 pg/1×10⁹ vg to about 70 pg/1×10⁹ vg, about 1 pg/1×10⁹ vg to about 60 pg/1×10⁹ vg, about 1 pg/1×10⁹ vg to about 50 pg/1×10⁹ vg or about 1 pg/1×10⁹ vg to about 40 pg/1×10⁹ vg.
E195. The method of any one of E2-E194, wherein an amount of residual plasmid DNA present in the affinity eluate is about 1×10⁴ pg/mL to about 1×10⁶ pg/mL, 1×10⁵ pg/mL to about 1×10⁶ pg/mL, 1×10⁵ pg/mL to about 9×10⁵ pg/mL, 1×10⁵ pg/mL to about 8×10⁵ pg/mL, 1×10⁵ pg/mL to about 8×10⁵ pg/mL, 1×10⁵ pg/mL to about 7×10⁵ pg/mL, 1×10⁵ pg/mL to about 6×10⁵ pg/mL or 1×10⁵ pg/mL to about 5×10⁵ pg/mL.
E196. The method of any one of E2-E195, wherein amount of residual plasmid DNA in the affinity eluate is reduced as compared to the amount of residual plasmid DNA in the solution comprising the rAAV vector.
E197. The method of E196, wherein there is a log reduction value of the amount of residual plasmid DNA in the affinity eluate as compared to the amount of residual plasmid DNA in the solution comprising the rAAV vector of about 1 to about −1.
E198. The method of any one of E194-E197, wherein the residual plasmid DNA is measured by qPCR.
E199. The method of any one of E2-E198, wherein an amount of residual affinity ligand present in the affinity eluate is about 1 pg/1×10⁹ vg to about 100 pg/1×10⁹ vg, about 1 pg/1×10⁹ vg to about 90 pg/1×10⁹ vg, about 1 pg/1×10⁹ vg to about 80 pg/1×10⁹ vg, about 1 pg/1×10⁹ vg to about 70 pg/1×10⁹ vg, about 1 pg/1×10⁹ vg to about 60 pg/1×10⁹ vg, about 1 pg/1×10⁹ vg to about 50 pg/1×10⁹ vg or about 1 pg/1×10⁹ vg to about 40 pg/1×10⁹ vg, optionally wherein the residual affinity ligand is measured by ELISA.
E200. The method of any one of E2-E199, wherein an amount of residual affinity ligand present in the affinity eluate is about 1 ng/mL to about 1000 ng/mL, 1 ng/mL to about 900 ng/mL, 1 ng/mL to about 800 ng/mL, 1 ng/mL to about 700 ng/mL, 1 ng/mL to about 600 ng/mL, 1 ng/mL to about 500/mL, 1 ng/mL to about 400 ng/mL or 1 ng/mL to about 300 ng/mL, optionally, wherein the residual affinity ligand is measured by ELISA.
E201. The method of any one of E2-E200, wherein infectivity ratio of the rAAV vector in the affinity eluate is about 5000 vg/IU to about 50000 vg/IU (e.g., about 12962, 17581, 20345 vg/IU).
E202. The method of any one of E2-E201, wherein the infectivity ratio of the rAAV vector in the affinity eluate is increased as compared to infectivity ratio of the rAAV vector in the solution comprising the rAAV vector.
E203. The method of E201 or E202, wherein the infectivity ratio of the rAAV vector in the affinity eluate is measured by a cell-based assay.
E204. The method of any one of E1-E203, wherein the rAAV vector comprises a capsid protein from a AAV serotype selected from the group consisting of AAV1, AAV2, AAV3 (including AAV3A and AAV3B), AAV4, AAV5, AAV6, AAV7, AAV8, AAV9, AAV10, AAV12, AAVrh8, AAVrh10, AAVrh39, AAVrh43, AAVrh74, AAVrh32.22, AAV1.1, AAV2.5, AAV6.1, AAV6.2, AAV6.3.1, AAV9.45, AAVShH10, HSC15/17, RHM4-1, RHM15-1, RHM15-2, RHM15-3/RHM15-5, RHM15-4, RHM15-6, AAVhu.26, AAV2i8, AAV29G, AAV2, AAV8G9, AAV-LK03, AAV2-TT, AAV2-TT-S312N, AAV3B-S312N, AAVavian, AAVbat, AAVbovine, AAVcanine, AAVequine, AAVprimate, AAVnon-primate, AAVovine, AAVmuscovy duck, AAVporcine4, AAVporcine5, AAVsnake NP4, NP22, NP66, AAVDJ, AAVDJ/8, AAVDJ/9, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14, AAVHSC15, AAVv66, AAVv33, AAVv37, AAVv40, AAVv67, AAVv70, AAVv72, AAVv84, AAVv86, AAVv87 and AAVv90.
E205. The method of any one of E1-E204, wherein the rAAV serotype is AAV9.
E206. The method of any one of E1-E205, wherein the rAAV vector comprises a VP1 protein comprising an amino acid sequence is at least 80%, 85% 90%, 95%, 98%, 99% or 100% identical to the amino acid sequence of SEQ ID NO:3.
E207. The method of any one of E1-E206, wherein the rAAV vector comprises a mini-dystrophin transgene.
E208. The method of E207, wherein the mini-dystrophin transgene comprises or consists of the nucleic acid sequence of SEQ ID NO:1.
E209. The method of any one of E207 or E208, wherein the mini-dystrophin transgene encodes a protein comprising or consisting of the amino acid sequence of SEQ ID NO:2 E210. The method of any one of E1-E209, wherein the rAAV vector comprises a nucleic acid comprising or consisting of the nucleic acid sequence of SEQ ID NO:4.
E211. The method of any one of E1-E210, further comprising regenerating the stationary phase.
E212. The method of E211, wherein regenerating the stationary phase comprises contacting the stationary phase with a first regeneration buffer, wherein rAAV vector protein, host cell impurities, and/or nucleic acid is released from the stationary phase by the contact with the first regeneration buffer, and optionally further comprising loading a solution comprising an rAAV vector on the stationary phase after removing at least a portion of first regeneration buffer.
E213. A method of regenerating a stationary phase for purification of a rAAV vector by affinity chromatography, the method comprising contacting the stationary phase with a first regeneration buffer, wherein rAAV vector protein, host cell impurities and/or nucleic acid is released from the stationary phase by the contact with the first regeneration buffer.
E214. The method of any one of E211-E213, wherein the method further comprises a fourth equilibration of the stationary phase prior to contacting the stationary phase with the first regeneration buffer.
E215. The method of E214, wherein the fourth equilibration comprises application of buffer solution (e.g., 150 mM to 160 mM sodium acetate) to the stationary phase and removal of all or a portion of the buffer solution from the stationary phase.
E216. The method of E215, wherein the buffer solution comprises about 153 mM sodium acetate at a pH of 5.6.
E217. The method of E215 or E216, wherein about 2 CV to about 10 CV, e.g., about 2 CV, about 3 CV, about 4 CV, about 5 CV, about 6 CV, about 7 CV, about 8 CV, about 9 CV or about 10 CV of the buffer solution is applied to the stationary phase.
E218. The method of any one of E213-E217, wherein a number of purification cycles that can be run on the stationary phase is increased by contacting the stationary phase with the first regeneration buffer as compared to the number of purification cycles that can be run on a stationary phase that is not contacted with the first regeneration buffer and
   wherein a purification cycle comprises loading a solution comprising a rAAV vector onto the stationary phase and eluting the rAAV vector from the stationary phase and
   wherein the % vg recovery of the rAAV vector in the eluate is 50% or greater.
E219. The method of E218, wherein the number of purifications cycles that can be run on the stationary phase that is contacted with the first regeneration buffer is greater than 2.
E220. The method of E218 or E219, wherein the number of purification cycles that can be run on the stationary phase that is contacted with the first regeneration buffer is 8 or more;
   wherein the number of purification cycles that can be run on the stationary phase that is contacted with the first regeneration buffer is 10 or more;
   wherein the % vg recovery of an eluate from a last purification cycle after contacting the stationary phase with the first regeneration buffer is not decreased more than 10% as compared to a % vg recovery of an eluate from a first purification cycle before the stationary phase is contacted with the first regeneration buffer;
   wherein an amount of unbound rAAV vector in a flow through of a last purification cycle after contacting the stationary phase with the first regeneration buffer is not increased more than 10% as compared to an amount of unbound rAAV vector in a flow through of a first purification cycle before the stationary phase is contacted with the first regeneration buffer;
   wherein a column pressure of a last purification cycle after contacting the stationary phase with the first regeneration buffer is not higher than 0.4 MPa;
   wherein a % purity of an eluate from a last purification cycle after contacting the stationary phase with the first regeneration buffer is not decreased more than 10% as compared to a % purity of an eluate from a first purification cycle before the stationary phase is contacted with the first regeneration buffer;
   wherein an amount of HCP of an eluate from a last purification cycle after contacting the stationary phase with the first regeneration buffer is not increased more than 10% as compared to an amount of HCP of an eluate from a first purification cycle before the stationary phase is contacted with the first regeneration buffer;
   wherein an amount of HCDNA of an eluate from a last purification cycle after contacting the stationary phase with the first regeneration buffer is not increased more than 10% as compared to an amount of HCDNA of an eluate from a first purification cycle before the stationary phase is contacted with the first regeneration buffer; and/or
   wherein an average A260/A280 of an eluate from a last purification cycle after contacting the stationary phase with the first regeneration buffer is not decreased more than 10% as compared to an average A260/A280 of an eluate from a first purification cycle before the stationary phase is contacted with the first regeneration buffer.
E221. The method of any one of E218-E220, wherein the number of purifications cycles that can be run on the stationary phase that is contacted with the first regeneration buffer is increased as compared to a stationary phase that is contacted with a buffer comprising guanidine HCl.
E222. The method of any one of E212-E221, wherein the first regeneration buffer comprises an acid, propylene glycol, and/or urea.
E223. The method of E222, wherein the acid of the first regeneration buffer is phosphoric acid, acetic acid, or an amino acid.
E224. The method of E222 or E223, wherein the concentration of the acid in the first regeneration buffer is about 0.05 N to about 1.5 N, e.g., about 0.05 N to about 1 N, about 0.05 N to about 0.75 N, about 0.05 N to about 0.5 N, about 0.05 N to about 0.25 N, about 0.05 N to about 0.15 N, about 0.05 N to about 0.075 N, about 0.05 N to about 0.1 N, about 0.1 N to about 0.15 N, about 0.05 N to about 0.06 N, about 0.06 N to about 0.07 N, about 0.07 N to about 0.08 N, about 0.08 N to about 0.09 N, about 0.09 N to about 0.1 N, about 0.1 N to about 0.11 N, about 0.11 N to about 0.12 N, about 0.12 N to about 0.13 N, about 0.13 N to about 0.14 N, about 0.14 N to about 0.15 N, about 0.15 N to about 0.2 N, about 0.2 N to about 0.3 N, about 0.3 N to about 0.4 N, about 0.4 N to about 0.5 N, about 0.45 N to about 0.55 N, about 0.5 N to about 0.6 N, about 0.6 N to about 0.7 N, about 0.7 N to about 0.8 N, about 0.8 N to about 0.9 N, about 0.9 N to about 1.0 N, about 1.0 N to about 1.1 N, about 1.1 N to about 1.2 N, about 1.2 N to about 1.3 N, about 1.3 N to about 1.4 N, about 1.4 N to about 1.5 N.
E225. The method of any one of E222-E224, wherein the concentration of the acid in the first regeneration buffer is about 0.10 N to about 0.15 N (e.g., about 0.132N, about 0.1N).
E226. The method of any one of E222-E225, wherein the acid is phosphoric acid.
E227. The method of any one of E222-E226, wherein the acid is phosphoric acid and the concentration of the phosphoric acid is about 0.13 N to about 0.14 N (e.g., about 0.132 N).
E228. The method of any one of E212-E227, wherein the first regeneration buffer has a pH of about 1 to about 4, e.g., about 1 to about 3, about 1 to about 2, about 1.5 to about 2.5, about 2 to about 3, about 3 to about 4, about 2 to about 4.
E229. The method of any one of E212-E228, wherein the first regeneration buffer has a pH of about 1, about 2, about 3, about 4.
E230. The method of any one of E212-E229, wherein the first regeneration buffer has a pH of about 2 (e.g., about 1.9).
E231. The method of any one of E222-E230, wherein the acid is phosphoric acid and the concentration of the phosphoric acid is about 0.10 N to about 0.15 N (e.g., about 0.132 N), and wherein the first regeneration buffer has a pH of about 1.5 to about 2.5 (e.g., about 1.9).
E232. The method of any one of E212-E231, wherein the stationary phase is contacted with 2 CV to 10 CV, e.g., 1 CV to 3 CV, 3 CV to 5 CV, 3 CV to 8 CV, 4 CV to 6 CV, 5 CV to 8 CV, or 8 CV to 10 CV, of first regeneration buffer.
E233. The method of any one of E212-E232, wherein the stationary phase is contacted with about 4.5 CV to about 5.5 CV (e.g., about 5 CV) of first regeneration buffer.
E234. The method of any one of E212-E233, the stationary phase is contacted with about 2.5 CV of first regeneration buffer, followed by a hold period of about 45 minutes, followed by contacting the stationary phase with a second 2.5 CV of a first regeneration buffer.
E235. The method of any one of E212-E234, wherein the first regeneration buffer flows upward through the stationary phase.
E236. The method of any one of E212-E235, wherein flow velocity of the first regeneration buffer is about 10 cm/hr to about 600 cm/hr, e.g., about 50 cm/hr to about 500 cm/hr, about 100 cm/hr to about 300 cm/hr, about 100 cm/hr to about 400 cm/hr, about 100 cm/hr to about 500 cm/hr, about 10 cm/hr to about 50 cm/hr, about 50 cm/hr to about 100 cm/hr, about 100 cm/hr to about 200 cm/hr, about 200 cm/hr to about 300 cm/hr, about 300 cm/hr to about 400 cm/hr, about 400 cm/hr to about 500 cm/hr, or about 500 cm/hr to about 600 cm/hr.
E237. The method of any one of E212-E235, wherein flow velocity of the first regeneration buffer is about 450 cm/hr to about 550 cm/hr (e.g., 500 cm/hr).
E238. The method of any one of E212-E235, wherein flow velocity of the first regeneration buffer is about 250 cm/hr to about 350 cm/hr (e.g., 300 cm/hr).
E239. The method of any one of E212-E235, wherein flow velocity of the first regeneration buffer is about 10 cm/hr to about 20 cm/hr (e.g., 14 cm/hr) E240. The method of any one of E212-E239, wherein residence time of the first regeneration buffer is about 2.5 min/CV to about 3.5 min/CV (e.g., 3 min/CV).
E241. The method of any one of E212-E239, wherein residence time of the first regeneration buffer is about 11 min/CV to about 12 min/CV (e.g., 11.5 min/CV).
E242. The method of any one of E212-E241, wherein the stationary phase is contacted with about 4.5 to about 5.5 CV (e.g., about 5 CV) of a first regeneration buffer comprising about 0.10 N to about 0.15 N (e.g., about 0.132 N) phosphoric acid, and wherein flow velocity of the first regeneration buffer is about 450 to about 550 cm/hr (e.g., about 500 cm/hr).
E243. The method of any one of E212-E241, wherein the stationary phase is contacted with about 4.5 to about 5.5 CV (e.g., about 5 CV) of a first regeneration buffer comprising about 0.10 N to about 0.15 N (e.g., about 0.132 N) phosphoric acid, and wherein flow velocity of the first regeneration buffer is about 250 to about 350 cm/hr (e.g., about 300 cm/hr).
E244. The method of any one of E212-E243, wherein the stationary phase is contacted with about 4.5 to about 5.5 CV (e.g., about 5 CV) of a first regeneration buffer comprising about 0.08 N to about 0.12 N (e.g., about 0.1 N) phosphoric acid, and wherein flow velocity of the first regeneration buffer is about 10 to about 20 cm/hr (e.g., about 14 cm/hr).
E245. The method of any one of E212-E244, further comprising a post-sanitization wash of the stationary phase after contacting the stationary phase with the first regeneration buffer.
E246. The method of E245, wherein the post-sanitization wash comprises application of buffer solution (e.g., 50 mM to 150 mM Tris) to the stationary phase and removal of all or a portion of the buffer solution from the stationary phase E247. The method of E246, wherein the buffer solution comprises about 100 mM Tris at a pH of 7.5 E248. The method of E246 or E247, wherein about 2 CV to about 10 CV, e.g., about 2 CV, about 3 CV, about 4 CV, about 5 CV, about 6 CV, about 7 CV, about 8 CV, about 9 CV or about 10 CV of the buffer solution is applied to the stationary phase.
E249. The method of any one of E246-E248, wherein the buffer solution flows downward through the stationary phase.
E250. The method of any one of E212-E249, further comprising contacting the stationary phase with a second regeneration buffer after contacting the stationary phase with the first regeneration buffer, and optionally after the post sanitization wash.
E251. The method of E250, wherein the second regeneration buffer comprises a detergent and a buffering agent.
E252. The method of E251, wherein the detergent is selected from the group consisting of sarkosyl, poloxamer 188 (P188), Triton X-100, polysorbate 80 (PS80), Brij-35, nonyl phenoxypolyethoxylethanol (NP-40), and a combination thereof.
E253. The method of E251-E252, wherein the detergent is sarkosyl.
E254. The method of any one of E251-E253, wherein percentage of detergent in the second regeneration buffer is about 0.1% to about 10%, e.g., about 0.1% to about 5%, about 0.5% to about 5%, about 0.5% to about 2.5%, about 0.5% to about 1.5%, about 0.1% to about 0.5%, about 0.5% to about 1.0%, about 1.0% to about 5.0%, about 5.0% to about 10%.
E255. The method of any one of E251-E254, wherein percentage of detergent is about 0.5% to about 1.5% (e.g., about 1.0%).
E256. The method of any one of E251-E255, wherein the detergent is sarkosyl and wherein the concentration of the sarkosyl is about 0.3% to about 1.5% (e.g., about 1.0%).
E257. The method of any one of E251-E256, wherein the buffering agent of the second regeneration buffer is selected from the group consisting of Tris (e.g., a mixture of Tris Base and Tris-HCl), BIS-Tris propane, diethanolamine, diethylamine, tricine, triethanolamine, and bicine.
E258. The method of any one of E251-E257, wherein the buffering agent in the second regeneration buffer is Tris.
E259. The method of any one of E251-E258, wherein concentration of the buffering agent in the second regeneration buffer is about 10 mM to about 500 mM, e.g., about 10 mM to about 400 mM, about 10 mM to about 300 mM, about 10 mM to about 200 mM, from about 50 mM to about 500 mM, about 50 mM to about 400 mM, about 50 mM to about 100 mM, about 10 mM to about 50 mM, about 50 mM to about 100 mM, about 50 mM to 150 mM, about 100 mM to about 200 mM, about 200 mM to about 300 mM, about 300 mM to about 400 mM, or about 400 mM to about 500 mM.
E260. The method of any one of E251-E259, wherein concentration of the buffering agent in the second regeneration buffer is about 50 mM to about 150 mM (e.g., about 100 mM).
E261. The method of any one of E251-E260, wherein the buffering agent in the second regeneration buffer is Tris and the concentration of Tris in the second regeneration buffer is about 50 mM to about 150 mM (e.g., about 100 mM).
E262. The method of any one of E250-E261, wherein the second regeneration buffer has a pH of about 7 to about 8, e.g., about 7.5.
E263. The method of any one of E250-E262, wherein the second regeneration buffer comprises about 0.5% to about 1.5% (e.g., about 1.0%) sarkosyl, about 50 mM to about 150 mM (e.g., about 100 mM) Tris and has a pH of about 7 to about 8 (e.g., about 7.5).
E264. The method of any one of E250-E263, wherein the stationary phase is contacted with 2 CV to 10 CV, e.g., 1 CV to 3 CV, 3 CV to 5 CV, 3 CV to 8 CV, 4 CV to 6 CV, 5 CV to 8 CV, or 8 CV to 10 CV, of the second regeneration buffer.
E265. The method of any one of E250-E264, wherein the stationary phase is contacted with about 4.5 CV to about 5.5 CV (e.g., about 5 CV) of the second regeneration buffer.
E266. The method of any one of E250-E265, wherein the second regeneration buffer flows upward through the stationary phase.
E267. The method of any one of E250-E266, wherein flow velocity of the second regeneration buffer is about 450 cm/hr to about 550 cm/hr (e.g., 500 cm/hr).
E268. The method of any one of E250-E266, wherein flow velocity of the second regeneration buffer is about 250 cm/hr to about 350 cm/hr (e.g., 300 cm/hr).
E269. The method of any one of E250-E266, wherein flow velocity of the second regeneration buffer is about 10 cm/hr to about 20 cm/hr (e.g., 14 cm/hr) E270. The method of any one of E250-E269, wherein residence time of the second regeneration buffer is about 2.5 min/CV to about 3.5 min/CV (e.g., 3 min/CV).
E271. The method of any one of E250-E269, wherein residence time of the regeneration buffer is about 11 min/CV to about 12 min/CV (e.g., 11.5 min/CV).
E272. The method of any one of E250-E271, wherein the stationary phase is contacted with about 4.5 to about 5.5 CV (e.g., about 5 CV) of a second regeneration buffer comprising about 0.5% to about 1.5% (e.g., about 1.0%) sarkosyl, about 50 mM to about 150 mM (e.g., about 100 mM) Tris, and has a pH of about 7 to about 8 (e.g., about 7.5), and wherein the flow velocity of the second regeneration buffer is about 450 to about 550 cm/hr (about 500 cm/hr).
E273. The method of any one of E250-E271, wherein the stationary phase is contacted with about 4.5 to about 5.5 CV (e.g., about 5 CV) of a second regeneration buffer comprising about 0.5% to about 1.5% (e.g., about 1.0%) sarkosyl, about 50 mM to about 150 mM (e.g., about 100 mM) Tris, and has a pH of about 7 to about 8 (e.g., about 7.5), and wherein the flow velocity of the second regeneration buffer is about 250 to about 350 cm/hr (about 300 cm/hr).
E274. The method of any one of E250-E271, wherein the stationary phase is contacted with about 4.5 to about 5.5 CV (e.g., about 5 CV) of a second regeneration buffer comprising about 0.5% to about 1.5% (e.g., about 1.0%) sarkosyl, about 50 mM to about 150 mM (e.g., about 100 mM) Tris, and has a pH of about 7 to about 8 (e.g., about 7.5), and wherein the flow velocity of the second regeneration buffer is about 10 to about 20 cm/hr (about 14 cm/hr).
E275. The method of any one of E250-E274, further comprising a post-regeneration wash of the stationary phase after contacting the stationary phase with the second regeneration buffer.
E276. The method of E275, wherein the post-regeneration wash comprises application of buffer solution to the stationary phase and removal of all or a portion of the buffer solution from the stationary phase E277. The method of E276, wherein the buffer solution comprises about 1 M to about 3 M (e.g., about 2 M) NaCl and about 50 mM to about 150 mM Tris (e.g., about 100 mM) Tris at a pH of about 7 to 8 (e.g., about 7.5).
E278. The method of E276 or E277, wherein about 2 CV to about 10 CV, e.g., about 2 CV, about 3 CV, about 4 CV, about 5 CV, about 6 CV, about 7 CV, about 8 CV, about 9 CV or about 10 CV of the buffer solution is applied to the stationary phase.
E279. The method of any one of E276-E278, wherein the buffer solution flows downward through the stationary phase.
E280. The method of any one of E212-E279, wherein residence time of the fourth equilibration buffer solution, first regeneration buffer, post-sanitization wash buffer solution, second regeneration buffer and/or post-regeneration wash buffer solution is about 1 min/CV to about 15 min/CV, e.g., about 2 min/CV to about 12 min/CV, about 5 min/CV to about 10 min/CV, about 1 min/CV to about 5 min/CV, about 3 min/CV to about 8 min/CV, about 2 min/CV to about 3 min/CV, about 3 min/CV to about 4 min/CV, about 4 min/CV to about 5 min/CV, about 5 min/CV to about 6 min/CV, about 6 min/CV to about 7 min/CV, about 7 min/CV to about 8 min/CV, about 8 min/CV to about 9 min/CV, about 9 min/CV to about 10 min/CV.
E281. The method of any one of E212-E280, wherein residence time of the fourth equilibration buffer solution, first regeneration buffer, post-sanitization wash buffer solution, second regeneration buffer and/or post-regeneration wash buffer solution is about 3 min/CV, about 4 min/CV, about 5 min/CV, about 5.5. min/CV, about 6 min/CV, about 7 min/CV, r about 8 min/CV, about 9 min/CV, about 9.5 min/CV, about 10 min/CV, about 10.5 min/CV, about 11 min/CV about 11.5 min/CV, about 12 min/CV about 12.5 min/CV, about 13 min/CV, about 13.5 min/CV, about 14 min/CV, about 14.5 min/CV or about 15 min/CV.
E282. The method of any one of E212-E281, wherein flow velocity of the fourth equilibration buffer solution, first regeneration buffer, post-sanitization wash buffer solution, second regeneration buffer and/or post-regeneration wash buffer solution is about 10 cm/hr to about 600 cm/hr, e.g., about 50 cm/hr to about 500 cm/hr, about 100 cm/hr to about 300 cm/hr, about 100 cm/hr to about 400 cm/hr, about 100 cm/hr to about 500 cm/hr, about 10 cm/hr to about 50 cm/hr, about 50 cm/hr to about 100 cm/hr, about 100 cm/hr to about 200 cm/hr, about 200 cm/hr to about 300 cm/hr, about 300 cm/hr to about 400 cm/hr, about 400 cm/hr to about 500 cm/hr, or about 500 cm/hr to about 600 cm/hr.
E283. The method of any one of E212-E282, wherein the first regeneration buffer, the second regeneration buffer, or both removes impurities from the stationary phase such that the regeneration buffer A280 peak area is greater than 900 mL*mAU and/or the regeneration buffer A260 peak area is greater than 500 mL*mAU.
E284. The method of any one of E1-E283, wherein maximum change in column pressure over the course of an affinity chromatography run is about 0.222 mPa to about 0.713 mPa when mobile phase flow is kept constant.
E285. The method of any one of E1-E284, wherein the method further comprises filtering the solution comprising the rAAV vector prior to loading on a stationary phase with a guard column, guard filter, or both a guard column and a guard filter.
E286. The method of E285, wherein the guard column comprises a strong anion exchange resin with a quaternary ammonium or hydroxyl anion (e.g., POROS™ XQ, POROS™ HQ), a benzyl ultra hydrophobic interaction chromatography (HIC) resin (e.g., POROS™ Benzyl Ultra), a octyl HIC resin (e.g., Capto Octyl), or an octylamine ligand resin (e.g., Capto 700) guard column.
E287. The method of E285 or E286, wherein the guard filter is a 0.2 μm nominal filter.
E288. The method of any one of E285 to E287, wherein the guard filter is a 0.2 μm nominal Pall™ EAV pre-column filter.
E289. A method of purifying a recombinant AAV (rAAV) vector, the method comprising:
   loading a solution comprising the rAAV vector on an affinity chromatography stationary phase;
   applying a pre-elution wash solution to the stationary phase, wherein the pre-elution wash solution comprises 15% to 25% ethanol and a buffering agent and has a pH of 5 to 6;
   eluting the rAAV vector from the stationary phase with an elution buffer, wherein the elution buffer comprises a salt, an amino acid and a buffering agent and has a pH of 2 to 4, to produce an affinity eluate.
E290. A method of purifying a rAAV vector, the method comprising
   loading a solution comprising the rAAV vector on a stationary phase;
   applying a pre-elution wash solution to the stationary phase, wherein the pre-elution wash comprises about 15% to 20% (e.g., about 17%, about 17.5%) ethanol and about 145 mM to about 155 mM (e.g., about 150 mM, about 153 mM) sodium acetate, pH 5 to 6 (e.g., about 5.6);
   eluting the rAAV vector from the stationary phase with an elution buffer, wherein the elution buffer comprises about 75 mM to about 125 mM (e.g., about 100 mM) glycine, about 20 mM to about 30 mM (e.g., about 25 mM) MgCl₂, about 140 to about 150 mM sodium acetate (about 148 mM), pH 2.5 to 3.5 (e.g., about 3.0);
   collecting an affinity eluate comprising the purified rAAV vector from the stationary phase.
E291. A method of purifying a rAAV vector, the method comprising
   pre-use rinse of a stationary phase, wherein pre-use rinse comprises application of water to the stationary phase;
   pre-use sanitization of the stationary phase, wherein pre-use sanitization comprises application of a solution comprising about 0.1N to about 0.2 N phosphoric acid (e.g., about 0.132N), pH 1.5 to 2.5 (e.g., about 1.9) to the stationary phase;
   a first equilibration of the stationary phase, wherein the first equilibration comprises application of a buffer solution comprising about 50 mM to about 150 mM (e.g., about 100 mM) of a buffering agent (e.g., Tris), pH 7 to 8 (e.g., about 7.5) to the stationary phase;
   loading a solution comprising the rAAV vector on a stationary phase;
   a second equilibration of the stationary phase, wherein the second equilibration comprises application of a buffer solution comprising about 50 mM to about 150 mM (e.g., about 100 mM) of a buffering agent (e.g., Tris), pH 7 to 8 (e.g., about 7.5) to the stationary phase;
   pre-elution wash of the stationary phase, wherein pre-elution wash comprises application of a pre-elution wash solution comprising about 15% to 20% (e.g., about 17%, about 17.5%) ethanol and about 145 mM to about 155 mM (e.g., about 150 mM, about 153 mM) sodium acetate, pH 5 to 6 (e.g., about 5.6) to the stationary phase;
   a third equilibration of the stationary phase, wherein the third equilibration comprises application of a buffer solution comprising about 150 mM to about 160 mM (e.g., about 153 mM) of a buffering agent (e.g., sodium acetate), pH 5 to 6 (e.g., about 5.6) to the stationary phase;
   eluting the rAAV vector from the stationary phase with an elution buffer, wherein the elution buffer comprises about 75 mM to about 125 mM (e.g., about 100 mM) glycine, about 20 mM to about 30 mM (e.g., about 25 mM) MgCl₂, about 140 to about 150 mM sodium acetate (about 148 mM), pH 2.5 to 3.5 (e.g., about 3.0); and/or
collecting an affinity eluate comprising the purified rAAV vector from the stationary phase.
E292. The method of E291, the method further comprising:
   a fourth equilibration of the stationary phase, wherein the fourth equilibration comprises application of a buffer solution comprising 150 mM to about 155 mM (e.g., about 153 mM) of a buffering agent (e.g., sodium acetate), pH 5 to 6 (e.g., about 5.6) to the stationary phase;
   contacting the stationary phase with a first regeneration buffer wherein the first regeneration buffer comprises about 0.10 N to about 0.15 N (e.g., about 0.132 N) phosphoric acid, pH 1.5 to 2.5 (e.g., about 1.9);
   a post-sanitization wash of the stationary phase, wherein the post-sanitization wash comprises application of a buffer solution comprising about 50 mM to about 150 mM (e.g., about 100 mM) of a buffering agent (e.g., Tris), pH 7 to 8 (e.g., about 7.5) to the stationary phase;
   contacting the stationary phase with a second regeneration buffer wherein the second regeneration buffer comprises about 0.5% to about 1.5% (e.g., about 1%) of a detergent (e.g., sarkosyl) and about 50 mM to about 150 mM (e.g., about 100 mM) of a buffering agent (e.g., Tris), pH 7 to 8 (e.g., about 7.5);
   a post-regeneration wash of the stationary phase, wherein the post-regeneration wash comprises application of a buffer solution comprising about 1 M to about 3 M (e.g., about 2 M) NaCl and about 50 mM to about 150 mM Tris (e.g., about 100 mM) Tris at a pH of about 7 to 8 (e.g., about 7.5);
   a post use flush comprising application of water for injection to the stationary phase; and/or
   contacting the stationary phase with a solution comprising 17.5% ethanol.
E293. The method of E292, wherein the method further comprises loading a solution comprising an rAAV vector on the affinity chromatography stationary phase after removing at least a portion of the solution comprising 17.5% ethanol.
E294. A method of regenerating a stationary phase, the method comprising
   equilibration of the stationary phase, wherein the equilibration comprises application of a buffer solution comprising 150 mM to about 155 mM (e.g., about 153 mM) of a buffering agent (e.g., sodium acetate), pH 5 to 6 (e.g., about 5.6) to the stationary phase;
   contacting the stationary phase with a first regeneration buffer wherein the first regeneration buffer comprises about 0.10 N to about 0.15 N (e.g., about 0.132 N) phosphoric acid, pH 1.5 to 2.5 (e.g., about 1.9);
   a post-sanitization wash of the stationary phase, wherein the post-sanitization wash comprises application of a buffer solution comprising about 50 mM to about 150 mM (e.g., about 100 mM) of a buffering agent (e.g., Tris), pH 7 to 8 (e.g., about 7.5) to the stationary phase;
   contacting the stationary phase with a second regeneration buffer wherein the second regeneration buffer comprises about 0.5% to about 1.5% (e.g., about 1%) of a detergent (e.g., sarkosyl) and about 50 mM to about 150 mM (e.g., about 100 mM) of a buffering agent (e.g., Tris), pH 7 to 8 (e.g., about 7.5);
   a post-regeneration wash of the stationary phase, wherein the post-regeneration wash comprises application of a buffer solution comprising about 1 M to about 3 M (e.g., about 2 M) NaCl and about 50 mM to about 150 mM Tris (e.g., about 100 mM) Tris at a pH of about 7 to 8 (e.g., about 7.5);
   a post use flush comprising application of water for injection to the stationary phase; and/or
   contacting the stationary phase with a solution comprising 17.5% ethanol.
E295. The method of E294, wherein rAAV vector protein, capsid protein, host cell impurities and/or nucleic acid is released from the stationary phase by contact with the first regeneration buffer, second regeneration buffer or both
E296. The method of E294 or E295, wherein the method further comprises loading a solution comprising an rAAV vector on the affinity chromatography stationary phase after removing at least a portion of the solution comprising 17.5% ethanol.
E297. A method of purifying a recombinant AAV (rAAV) vector, the method comprising: loading a solution comprising the rAAV vector on an affinity chromatography stationary phase; applying a pre-elution wash solution to the stationary phase, wherein the pre-elution wash solution comprises 15% to 25% ethanol and a buffering agent, and has a pH of 5 to 6; eluting the rAAV vector from the stationary phase with an elution buffer, wherein the elution buffer comprises a salt, an amino acid and a buffering agent and has a pH of 2 to 4, to produce an affinity eluate.
E298. The method of E297, wherein the affinity chromatography stationary phase is in a column.
E299. The method of any one of E297-E298, wherein the elution buffer comprises 5 mM to 150 mM of the salt, optionally wherein the salt is magnesium chloride.
E300. The method of any one of E298-E299, wherein the elution buffer comprises 50 mM to 150 mM of the amino acid, optionally wherein the amino acid is glycine.
E301. The method of any one of E297-E300, wherein the elution buffer comprises 75 mM to 250 mM of the buffering agent, optionally wherein the buffering agent is sodium acetate.
E302. The method of any one of E297-E301, wherein the elution buffer has a pH of 2.5 to 3.5.
E303. The method of any one of E297-E302, wherein the elution buffer comprises 50 mM to 150 mM of glycine, 10 mM to 100 mM of MgCl₂, 50 mM to 200 mM of sodium acetate, and a pH of 2.5 to 3.5, optionally wherein the elution buffer has a conductivity of 5 mS/cm to 40 mS/cm or 20 mS/cm to 35 mS/cm.
E304. The method of any one of E297-E303, wherein 2 column volumes (CV) to 10 CV or 4.5 CV to 5.5 CV of the elution buffer is applied to the stationary phase.
E305. The method of any one of E297-E304, wherein the elution buffer i) elutes the rAAV vector from the stationary phase, ii) does not elute residual impurities from the stationary phase; iii) does not result in precipitation of the affinity eluate; iv) maximizes a % vg recovery; v) does not interfere with binding of the rAAV vector to an anion exchange chromatography (AEX) stationary phase; vi) does not contain a trivalent anion; vii) does not contain citrate ions, or a combination thereof.
E306. The method of any one of E297-E305, wherein 0.1 CV to 10 CV of the affinity eluate is collected from the stationary phase.
E307. The method of any one of E297-E306, wherein the solution comprising the rAAV vector comprises host cell protein and host cell DNA.
E308. The method of any one of E297-E307, wherein the solution comprising the rAAV vector is loaded onto the stationary phase to achieve a challenge of 1×10¹² viral genomes (vg)/mL stationary phase to 1.5×10¹⁴ vg/mL stationary phase.
E309. The method of any one of E297-E308, wherein the rAAV vector comprises a capsid protein from an AAV serotype.
E310. The method of E309, wherein the rAAV vector comprises a capsid protein from a AAV serotype selected from the group consisting of AAV1, AAV2, AAV3 (including AAV3A and AAV3B), AAV4, AAV5, AAV6, AAV7, AAV8, AAV9, AAV10, AAV12, AAVrh8, AAVrh10, AAVrh39, AAVrh43, AAVrh74, AAVrh32.22, AAV1.1, AAV2.5, AAV6.1, AAV6.2, AAV6.3.1, AAV9.45, AAVShH10, HSC15/17, RHM4-1, RHM15-1, RHM15-2, RHM15-3/RHM15-5, RHM15-4, RHM15-6, AAVhu.26, AAV2i8, AAV29G, AAV2, AAV8G9, AAV-LK03, AAV2-TT, AAV2-TT-S312N, AAV3B-S312N, AAVavian, AAVbat, AAVbovine, AAVcanine, AAVequine, AAVprimate, AAVnon-primate, AAVovine, AAVmuscovy duck, AAVporcine4, AAVporcine5, AAVsnake NP4, NP22, NP66, AAVDJ, AAVDJ/8, AAVDJ/9, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14, AAVHSC15, AAVv66, AAVv33, AAVv37, AAVv40, AAVv67, AAVv70, AAVv72, AAVv84, AAVv86, AAVv87 and AAVv90.
E311. The method of any one of E297-E310, wherein the stationary phase comprises a macromolecule that binds an AAV capsid.
E312. The method of any one of E297-E311, wherein the pre-elution wash solution comprises 10 mM to 500 mM of the buffering agent, optionally wherein the buffering agent is sodium acetate.
E313. The method of any one of E297-E312, wherein the pre-elution wash solution comprises 15% to 25% of ethanol, 100 mM to 200 mM of sodium acetate, and has a pH of 5 to 6, optionally wherein 1 CV to 10 CV or 4.5 to 5.5 CV of the pre-elution wash solution is applied to the stationary phase.
E314. The method of any one of E297-E313, wherein the pre-elution wash i) removes bound impurities from the stationary phase; ii) maintains rAAV-stationary phase ligand binding; iii) has a reduced pH to improve removal of proteins other than rAAV proteins, such as host cell proteins, or a combination thereof.
E315. The method of any one of E297-E314, further comprising equilibration of the stationary phase i) prior to loading the solution comprising the rAAV vector on the stationary phase, ii) after loading the solution comprising the rAAV vector on the stationary phase, iii) prior to application of a pre-elution wash, iv) after application of a pre-elution wash v) prior to eluting the rAAV vector from the stationary phase with an elution buffer, vi) after eluting the rAAV vector from the stationary phase with an elution buffer, vii) prior to contacting the stationary phase with a first regeneration buffer, or a combination thereof.
E316. The method of E315, wherein equilibration comprises application of a buffer solution to the stationary phase and removal of all or a portion of the buffer solution from the stationary phase.
E317. The method of E316, wherein the buffer solution comprises Tris or sodium acetate.
E318. The method of E316 or E317, wherein the buffer solution comprises about 100 mM Tris at about pH 7.5 or about 153 mM sodium acetate at about pH 5.6.
E319. The method of any one of E297-E318, further comprising contacting the stationary phase with a first regeneration buffer after obtaining at least a portion of the affinity eluate.
E320. The method of E319, wherein the first regeneration buffer comprises 0.05 N to 1.5 N of an acid, optionally wherein the acid is phosphoric acid.
E321. The method of E319 or E320, wherein the first regeneration buffer comprises 0.10 N to 0.15 N phosphoric acid, and a pH of 1.5 to 2.5.
E322. The method of any one of E319-E321, wherein the stationary phase is contacted with 1 CV to 10 CV or 4.5 CV to 5.5 CV of the first regeneration buffer.
E323. The method of any one of E319-E322, wherein the method further comprises loading another solution comprising an rAAV vector on the affinity chromatography stationary phase after removing at least a portion of the first regeneration buffer.
E324. The method of any one of E319-E323, wherein the method further comprises applying a second amount of the pre-elution wash solution on the affinity chromatography stationary phase after removing at least a portion of the first regeneration buffer.
E325. The method of any one of E319-E324, wherein a number of purification cycles that can be run on the stationary phase that is contacted with the first regeneration buffer is 8 or more;
   wherein a number of purification cycles that can be run on the stationary phase that is contacted with the first regeneration buffer is 10 or more;
   wherein a % vg recovery of an eluate from a last purification cycle after contacting the stationary phase with the first regeneration buffer is not decreased more than 10% as compared to a % vg recovery of an eluate from a first purification cycle before the stationary phase is contacted with the first regeneration buffer;
   wherein an amount of unbound rAAV vector in a flow through of a last purification cycle after contacting the stationary phase with the first regeneration buffer is not increased more than 10% as compared to an amount of unbound rAAV vector in a flow through of a first purification cycle before the stationary phase is contacted with the first regeneration buffer; wherein a column pressure of a last purification cycle after contacting the stationary phase with the first regeneration buffer is not higher than 0.4 MPa;
   wherein a % purity of an eluate from a last purification cycle after contacting the stationary phase with the first regeneration buffer is not decreased more than 10% as compared to a % purity of an eluate from a first purification cycle before the stationary phase is contacted with the first regeneration buffer;
   wherein an amount of HCP of an eluate from a last purification cycle after contacting the stationary phase with the first regeneration buffer is not increased more than 10% as compared to an amount of HCP of an eluate from a first purification cycle before the stationary phase is contacted with the first regeneration buffer;
   wherein an amount of HCDNA of an eluate from a last purification cycle after contacting the stationary phase with the first regeneration buffer is not increased more than 10% as compared to an amount of HCDNA of an eluate from a first purification cycle before the stationary phase is contacted with the first regeneration buffer;
   wherein an average A260/A280 of an eluate from a last purification cycle after contacting the stationary phase with the first regeneration buffer is not decreased more than 10% as compared to an average A260/A280 of an eluate from a first purification cycle before the stationary phase is contacted with the first regeneration buffer or a combination thereof.
E326. The method of any one of E319-E325, wherein a number of purifications cycles that can be run on the stationary phase that is contacted with the first regeneration buffer is increased as compared to a stationary phase that is contacted with a buffer comprising guanidine HCl.
E327. The method of any one of E319-E326, further comprising contacting the stationary phase with a second regeneration buffer after contacting the stationary phase with the first regeneration buffer, wherein the second regeneration buffer is different than the first regeneration buffer.
E328. The method of E327, wherein the second regeneration buffer comprises 0.1% to 5% of a detergent, optionally wherein the detergent is sarkosyl.
E329. The method of E327-E328, wherein the second regeneration buffer comprises 50 mM to 150 mM of a buffering agent, optionally wherein the buffering agent is Tris.
E330. The method of any one of E327-E329, wherein the second regeneration buffer comprises 0.1% to 1.5% of sarkosyl, 50 mM to 150 mM of Tris, and a pH of 7 to 8, optionally wherein the stationary phase is contacted with 1 CV to 10 CV or 4.5 CV to 5.5 CV of the second regeneration buffer.
E331. The method of any one of E327-E330, wherein the method further comprises loading another solution comprising a rAAV vector on the affinity chromatography stationary phase after removing at least a portion of the second regeneration buffer.
E332. The method of any one of E327-E331, wherein the method further comprises applying a second amount of the pre-elution wash solution on the affinity chromatography stationary phase after removing at least a portion of the second regeneration buffer.
E333. A method of purifying a rAAV vector, the method comprising:
loading a solution comprising the rAAV vector on an affinity chromatography stationary phase in a column;
applying a pre-elution wash solution comprising 15% to 25% ethanol, 100 mM to 200 mM sodium acetate, and a pH of 5 to 6 to the stationary phase; and
eluting the rAAV vector from the stationary phase with an elution buffer comprising 50 mM to 150 mM glycine, 10 mM to 100 mM MgCl₂, 50 mM to 250 mM sodium acetate, and a pH of 2.5 to 3.5 to produce an affinity eluate containing the rAAV vector.
E334. The method of E333, wherein 1 CV to 10 CV or 4.5 CV to 5.5 CV of the pre-elution wash solution is applied to the stationary phase.
E335. The method of E333 or E334, wherein the pre-elution wash has a reduced pH to improve removal of a protein other than a rAAV protein from the stationary phase, wherein the pre-elution wash maintains rAAV-stationary phase ligand binding or both and optionally wherein the proteins other than rAAV protein is a host cell protein.
E336. The method of any one of E333-E335, wherein the elution buffer does not result in precipitation of an affinity eluate, wherein the elution buffer does not interfere with binding of the rAAV vector to an anion exchange chromatography (AEX) stationary phase or both.
E337. The method of any one of E333-E336, wherein a % vg recovery in the affinity eluate is 50% to 100%, optionally as measured by qPCR; wherein a vg per mL of the affinity eluate is about 1.0×10¹² vg/mL to about 1.0×10¹⁴ vg/mL, optionally as measured by qPCR; wherein a % purity of the affinity eluate is about 95% to about 100% capsid protein of total protein, optionally as measured by SEC or reverse phase HPLC, non-reducing, or a combination thereof.
E338. The method of any one of E333-E337, further comprising contacting the stationary phase with a first regeneration buffer after obtaining at least a portion of the affinity eluate.
E339. The method of any one of E333-E338, further comprising contacting the stationary phase with a second regeneration buffer after the first regeneration buffer, wherein the second regeneration buffer is different than the first regeneration buffer.
E340. A method of regenerating an affinity chromatography stationary phase, the method comprising: contacting the stationary phase with a first regeneration buffer comprising an acid and a pH of 1 to 4, wherein impurities are removed from the stationary phase.
E341. The method of E340, wherein the first regeneration buffer comprises 0.05 N to 1.5 N of the acid, optionally wherein the acid is phosphoric acid or acetic acid.
E342. The method of E340 or E341, wherein the first regeneration buffer comprises 0.10 N to 0.15 N phosphoric acid, and a pH of 1.5 to 2.5.
E343. The method of any one of E340-E342, wherein 1 CV to 10 CV or 4.5 CV to 5.5 CV of the first regeneration buffer is applied to the stationary phase, wherein the stationary phase is contacted with about half of the volume of a first regeneration buffer, followed by a hold period of about 45 minutes, followed by contacting the stationary phase with a second half of the volume of the first regeneration buffer.
E344. The method of any one of E340-E343, wherein the method further comprises loading a solution comprising an rAAV vector on the affinity chromatography stationary phase after removing at least a portion of the first regeneration buffer.
E345. The method of any one of E340-E344, wherein the method further comprises applying a pre-elution wash solution on the affinity chromatography stationary phase after removing at least a portion of the first regeneration buffer.
E346. The method of any one of E340-E345, further comprising contacting the stationary phase with a second regeneration buffer after the first regeneration buffer, wherein the second regeneration buffer is different than the first regeneration buffer.
E347. The method of E346, wherein the second regeneration buffer comprises a detergent and a buffering agent.
E348. The method of E346-E347, wherein the second regeneration buffer comprises 0.1% to 5% of the detergent, optionally wherein the detergent is sarkosyl.
E349. The method of any one of E346-E348, wherein the second regeneration buffer comprises 50 mM to 150 mM of the buffering agent, optionally wherein the buffering agent is Tris.
E350. The method of any one of E346-E349, wherein the second regeneration buffer comprises 0.1% to 1.5% of sarkosyl, 50 mM to 150 mM of Tris, and a pH of 7 to 8.
E351. The method of any one of E346-E350, wherein 1 CV to 10 CV or 4.5 CV to 5.5 CV of the second regeneration buffer is applied to the stationary phase.
E352. The method of any one of E350-E351, wherein the method further comprises loading a solution comprising an rAAV vector on the affinity chromatography stationary phase after removing at least a portion of the second regeneration buffer.
E353. The method of any one of E350-E352, wherein the method further comprises applying a pre-elution wash solution on the affinity chromatography stationary phase after removing at least a portion of the second regeneration buffer.

### BRIEF DESCRIPTION OF DRAWINGS

FIG. 1 depicts exemplary affinity elution chromatograms for elution buffers A and B.

FIG. 2 depicts exemplary overlay of HQ-17 and HQ-18 AEX elution profiles.

FIG. 3 depicts exemplary affinity elution chromatograms for HQ-21 and HQ-22 runs.

FIG. 4 depicts exemplary side-by-side comparison of HQ-21 and HQ-22 AEX elution profiles.

FIG. 5 depicts exemplary side-by-side comparison of HQ-24 and HQ-25 AEX elution profiles. The HQ-24 affinity elution buffer was 30 mM citrate, 100 mM glycine, 100 mM MgCl₂ pH 3.0. The HQ-25 affinity elution buffer was 150 mM acetate, 100 mM glycine, 100 mM MgCl₂ pH 3.0.

FIG. 6 depicts exemplary maximum delta column pressure over 12 purification cycles on a 15 cm bed height POROS™ CaptureSelect AAV9 column operated with a 3 min/CV residence time.

FIG. 7 depicts exemplary delta column pressure over 12 purification cycles on a 15 cm bed height POROS™ CaptureSelect AAV9 column operated with a 3 min/CV residence time.

FIG. 8 depicts exemplary column pressure over 6 purification cycles on a POROS™ CaptureSelect AAV9 column operated with an in-line EAV guard filter.

FIG. 9 depicts exemplary affinity elution chromatogram on a 2.7 L column.

FIG. 10 depicts exemplary affinity elution chromatogram on a 17.6 L column.

### DESCRIPTION

### I. Definitions

Unless otherwise defined, all technical and scientific terms used herein have the meaning commonly understood by one of ordinary skill in the art to which this invention belongs. The terminology used herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise. The following terms have the meanings given:

As used herein, the term “A260/A280” or “A260/A280 ratio” refers to the ratio of an absorbance measured at 260 nm and an absorbance measured at 280 nm. An A260/A280 ratio of a solution comprising rAAV capsids provides an estimation of the relative amounts of capsids with packaged DNA (i.e., full capsids and intermediate capsids) and without packaged DNA (i.e., empty capsids) that are present in the solution. For example, the higher the value, the greater the percentage of full and intermediate capsids present in the solution, such as an affinity eluate. Comparison of A260/A280 ratios between solutions allows for a relative estimation of capsid species, such that a solution with a higher A260/A280 has a greater percentage of full and intermediate capsids than a solution with a lower A260/A280 ratio. As used herein, an affinity eluate with an A260/A280 ratio of greater than 0.7 considered acceptable for use in further purification processes (e.g., anion exchange chromatography) to produce a rAAV vector composition suitable for use as a drug product. In some embodiments, an absorbance is measured using analytical size exclusion chromatography (SEC) in a high-performance liquid chromatography (HPLC) system, and measurement of the absorbance may be at one or more wavelengths (e.g., 260 nm and/or 280 nm).

As used herein, the term “about,” or “approximately” is used to indicate that a value includes the standard deviation of error for the device or method being employed to determine the value. In some embodiments, the term “about” can be added to any numeral recited herein to the extent the numeral would have a standard deviation of error when measuring.

As used herein, the term “affinity % vg recovery” refers to an amount of vector genome (vg) present in an affinity eluate as a percentage of the total amount of vg present in a load solution (e.g., clarified lysate) loaded on the stationary phase. For example, affinity % vg recovery=((amount of vg in an affinity eluate/amount of vg in a load solution)*100). An affinity eluate may be referred to as an affinity pool, representing the entire output from the affinity chromatography elution step. The amount of vg may be measured by any method known in the art, including but not limited to quantitative PCR (qPCR). QPCR can be used to measure vg by detecting and quantifying any nucleic acid sequence present in the vg, for example, an ITR sequence or a transgene sequence.

As used herein, the terms “adeno-associated virus” and/or “AAV” refer to a parvovirus with a linear single-stranded DNA genome and variants thereof. The term covers all subtypes and both naturally occurring and recombinant forms, except where required otherwise.

The canonical AAV wild-type genome comprises 4681 bases (Berns et al. (1987) Advances in Virus Research 32:243-307) and includes terminal repeat sequences (e.g., inverted terminal repeats (ITRs)) at each end which function in cis as origins of DNA replication and as packaging signals for the virus. The genome includes two large open reading frames, known as AAV replication (“AAV rep” or “rep”) and capsid (“AAV cap” or “cap”) genes, respectively. AAV rep and cap may also be referred to herein as AAV “packaging genes.” These genes code for the viral proteins involved in replication and packaging of the viral genome.

In wild type AAV, three capsid genes VP1, VP2 and VP3 overlap each other within a single open reading frame and alternative splicing leads to production of VP1, VP2 and VP3 capsid proteins (Grieger et al. (2005) J. Virol. 79(15):9933-9944). A single P40 promoter allows all three capsid proteins to be expressed at a ratio of about 1:1:10 for VP1, VP2, VP3, respectively, which complements AAV capsid production. More specifically, VP1 is the full-length protein, with VP2 and VP3 being increasingly shortened due to increasing truncation of the N-terminus. A well-known example is the capsid of AAV9 as described in U.S. Pat. No. 7,906,111, wherein VP1 comprises amino acid residues 1 to 736 of SEQ ID NO:123, VP2 comprises amino acid residues 138 to 736 of SEQ ID NO:123, and VP3 comprises amino acid residues 203 to 736 of SEQ ID NO:123. As used herein, the term “AAV Cap” or “cap” refers to AAV capsid proteins VP1, VP2 and/or VP3, and variants and analogs thereof. A second open reading frame of the capsid gene encodes an assembly factor, called assembly-activating protein (AAP), which is essential for the capsid assembly process (Sonntag et al. (2011) J. Virol. 85(23):12686-12697).

At least four viral proteins are synthesized from the AAV rep gene—Rep 78, Rep 68, Rep 52 and Rep 40—named according to their apparent molecular weights. As used herein, “AAV rep” or “rep” means AAV replication proteins Rep 78, Rep 68, Rep 52 and/or Rep 40, as well as variants and analogs thereof. As used herein, rep and cap refer to both wild type and recombinant (e.g., modified chimeric, and the like) rep and cap genes as well as the polypeptides they encode. In some embodiments, a nucleic acid encoding a rep will comprise nucleotides from more than one AAV serotype. For instance, a nucleic acid encoding a rep protein may comprise nucleotides from an AAV2 serotype and nucleotides from an AAV3 serotype (Rabinowitz et al. (2002) J. Virology 76(2):791-801).

As used herein the terms “recombinant adeno-associated virus vector,” “rAAV” and/or “rAAV vector” refer to an AAV capsid comprising a vector genome, unless specifically noted otherwise. The vector genome comprises a polynucleotide sequence that is not, at least in part, derived from a naturally-occurring AAV (e.g., a heterologous polynucleotide not present in wild type AAV), and the rep and/or cap genes of the wild type AAV genome have been removed from the vector genome. Where the rep and/or cap genes of the AAV have been removed (and/or ITRs from an AAV have been added or remain), the nucleic acid within the AAV is referred to as the “vector genome.” Therefore, the term rAAV vector encompasses both a rAAV viral particle that comprises a capsid but does not comprise a complete AAV genome; instead the recombinant viral particle can comprise a heterologous, i.e., not originally present in the capsid, nucleic acid, hereinafter referred to as a vector genome. Thus, a “rAAV vector genome” (or “vector genome”) refers to a heterologous polynucleotide sequence (including at least one ITR) that may, but need not, be contained within an AAV capsid. A rAAV vector genome may be double-stranded (dsAAV), single-stranded (ssAAV) or self-complementary (scAAV). Typically, a vector genome comprises a heterologous (to the original AAV from which it is derived) nucleic acid often encoding a therapeutic transgene, a gene editing nucleic acid, and the like.

As used herein, the terms “rAAV vector,” “rAAV viral particle” and/or “rAAV vector particle” refer to an AAV capsid comprised of at least one AAV capsid protein (though typically all of the capsid proteins, e.g., VP1, VP2 and VP3, or variant thereof, of a AAV are present) and containing a vector genome comprising a heterologous nucleic acid sequence, unless specifically noted otherwise. These terms are to be distinguished from an “AAV viral particle” or “AAV virus” that is not recombinant wherein the capsid contains a virus genome encoding rep and cap genes and which AAV virus is capable of replicating if present in a cell also comprising a helper virus, such as an adenovirus and/or herpes simplex virus, and/or required helper genes therefrom. Thus, production of a rAAV vector particle necessarily includes production of a recombinant vector genome using recombinant DNA technologies, as such, which vector genome is contained within a capsid to form a rAAV vector, rAAV viral particle, or a rAAV vector particle. In some embodiments, viral particles in a composition or solution, such as an affinity eluate, may be quantified and expressed as a viral particle titer (e.g., vp/mL). In some embodiments, a viral particle titer of a composition or solution is measured by size exclusion chromatography.

The genomic sequences of various serotypes of AAV, as well as the sequences of the inverted terminal repeats (ITRs), rep proteins, and capsid subunits are known in the art. Such sequences may be found in the literature or in public databases such as GenBank. See, e.g., GenBank Accession Numbers NC_002077 (AAV1), AF063497 (AAV1), NC_001401 (AAV2), AF043303 (AAV2), NC_001729 (AAV3), AF028705.1 (AAV3B), NC_001829 (AAV 4), U89790 (AAV4), NC_006152 (AAV5), AF028704 (AAV6), AF513851 (AAV7), AF513852 (AAV8), NC_006261 (AAV8), AY530579 (AAV9), AY631965 (AAV10), AY631966 (AAV11), and DQ813647 (AAV12); the disclosures of which are incorporated by reference herein. See also, e.g., Srivistava et al. (1983) J. Virology 45:555; Chiorini et al. (1998) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al. (1986) J. Virol. 58:921; Gao et al. (2002) Proc. Nat. Acad. Sci. USA 99: 11854; Moris et al. (2004) Virology 33:375-383; International Patent Publications WO 00/28061, WO 99/61601, WO 98/11244; WO 2013/063379, WO 2014/194132, WO 2015/121501; and U.S. Pat. Nos. 6,156,303 and 7,906,111.

As used herein, the term “AEX % vg recovery” refers to the amount of vg present in an eluate collected from an AEX column as a percentage of the total amount of vg present in a load solution (e.g., an affinity eluate or a diluted affinity eluate). For example, AEX % vg recovery=((amount of vg in an AEX eluate/amount of vg in load solution)*100). An AEX eluate may be referred to as an AEX pool, representing a portion of the output from the AEX elution step. The amount of vg may be measured by any method known in the art, including but not limited to quantitative PCR (qPCR). QPCR can be used to measure the vg by detecting and quantifying any nucleic acid sequence present in the vg, for example, an ITR sequence or a transgene sequence.

As used herein, the term “and/or” refers to and encompasses any and all possible combinations of one or more of the associated listed items, as well as the lack of combinations when interpreted in the alternative (“or”).

As used herein, the term “anion exchange chromatography,” or “AEX” refers to a chromatography process that employs a positively charged stationary phase (e.g., a resin) to separate substances (e.g., AAV capsids, DNA, protein, high molar mass species, amino acids) based on charge differences of the substances. AEX is useful for separating rAAV capsids from impurities based on charge differences at moderately acidic to alkaline pH (e.g., greater than pH 6). AEX can also separate empty capsids from rAAV vectors containing a complete, or essentially complete, vector genome (i.e., full capsid) by relying on charge differences between empty capsids and full capsids.

Without wishing to be bound by theory, the tightness of binding between an AAV capsid and an AEX chromatography stationary phase is related to the strength of the negative charge of the capsid, including the charge contribution from any nucleic acid within the capsid, solution pH and solution conductivity (Qu, G. et al., (2007) J. Virological Methods 140:183-192). In some embodiments, an AEX chromatography stationary phase is a resin (e.g., polystyrenedivinylbenzene particles modified with covalently bound quaternized polyethyleneimine, and optionally OH groups (e.g., POROS™ 50 HQ resin).

As used herein, the term “associated with” refers to with one another, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example, by means of hydrogen bonds, van der Waals interaction, hydrophobic interactions, magnetism, and a combination thereof.

As used herein, the terms “clarified lysate” and “supernatant from a cell lysate” refer to a solution collected following lysis of host cells from a host cell culture and clarification. The lysate may be clarified by any method known in the art, including but not limited to, sedimentation, centrifugation, flocculation, depth filtration, charged depth filtration and filtration using a filter containing diatomaceous earth media.

As used herein, the term “clean in place” or “CIP” refers to a step, phase or process whereby a stationary phase is contacted with a solution to remove impurities such as HCP, HCDNA and other host cell material. In some embodiments a clean in place step may also serve to sanitize a stationary phase. A clean in place step may be performed on a stationary phase more than once over the course of a chromatography run.

As used herein, the term “coding sequence” or “nucleic acid encoding” refers to a nucleic acid sequence which encodes a protein or polypeptide and denotes a sequence which is transcribed (in the case of DNA) and translated (in the case of mRNA) into a polypeptide in vitro or in vivo when placed under the control of (operably linked to) appropriate regulatory sequences. The boundaries of a coding sequence are generally determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences.

As used herein, the term “chimeric” refers to a viral capsid or particle, with capsid or particle sequences from different parvoviruses, preferably different AAV serotypes, as described in Rabinowitz et al., U.S. Pat. No. 6,491,907, the disclosure of which is incorporated in its entirety herein by reference. See also Rabinowitz et al. (2004) J. Virol. 78(9):4421-4432. In some embodiments, a chimeric viral capsid is an AAV2.5 capsid which has the sequence of the AAV2 capsid with the following mutations: 263 Q to A; 265 insertion T; 705 N to A; 708 V to A; and 716 T to N. The nucleotide sequence encoding such capsid is defined as SEQ ID NO: 15 as described in WO 2006/066066. Other preferred chimeric AAV capsids include, but are not limited to, AAV2i8 described in WO 2010/093784, AAV2G9 and AAV8G9 described in WO 2014/144229, and AAV9.45 (Pulicherla et al. (2011) Molecular Therapy 19(6):1070-1078), AAV-NP4, NP22 and NP66, AAV-LKO through AAV-LK019 described in WO 2013/029030, RHM4-1 and RHM15_1 through RHM5_6 described in WO 2015/013313, AAVDJ, AAVDJ/8, AAVDJ/9 described in WO 2007/120542.

As used herein, the term “chromatography stationary phase,” or “stationary phase” is used to refer to any substance that can be used for the separation of a product from another substance (e.g., an impurity). In some embodiments, a chromatography stationary phase is a resin, a media, a membrane, a membrane adsorber, fiber, or a monolith. In some embodiments, a chromatography stationary phase is a media that binds to AAV capsids under certain conditions. In some embodiments, a chromatography stationary phase is an affinity chromatography resin. In some embodiments, an affinity chromatography stationary phase is a resin comprising 50 μm porous polystyrenedivinylbenzene beads modified with a Camelid-derived single domain antibody (e.g., V_{H}H) ligand and with a dynamic binding capacity of 1.0×10¹⁴ vg/mL resin (e.g., POROS™ CaptureSelect™ AAV9 affinity resin, POROS™ CaptureSelect™ AAVX affinity resin). In some embodiments, a chromatography stationary phase is an ion exchange media (e.g., an anion exchange media (AEX), a cation exchange media). In some embodiments, a chromatography stationary phase is an AEX chromatography resin. In some embodiments, a chromatography stationary phase is POROS™ 50 HQ. In some embodiments, an AEX chromatography stationary phase is a resin comprising polystyrenedivinylbenzene particles modified with covalently bound quaternized polyethyleneimine, and optionally OH groups (e.g., POROS™ 50 HQ resin).

As used herein, the term “eluate” refers to fluid exiting from a chromatography stationary phase (e.g., a monolith, membrane, resin, fiber, media), (e.g., “eluting from the stationary phase”) comprised of a mobile phase and material that passed through the stationary phase or was displaced from the stationary phase. In some embodiments, a stationary phase includes, for example, a monolith, a membrane, a resin, fiber or a media. The mobile phase may be a solution that has been loaded onto a column and has flowed through the column (i.e., referred to as a “flow-through fraction” or an “unbound fraction”), an equilibration solution (e.g. an equilibration buffer) an isocratic elution solution, a gradient elution solution, a solution for regeneration of a stationary phase, a solution for sanitization of a stationary phase, a solution for washing, and a combination thereof. As used herein, the term “affinity eluate,” or “affinity pool” refers to an eluate from an affinity chromatography stationary phase and/or an affinity chromatography column. As used herein, the term “AEX eluate” or “AEX pool” refers to an eluate from an AEX stationary phase and/or an AEX column.

As used herein, the term “equilibration” refers to a step of a chromatography process whereby an equilibration buffer is applied to a stationary phase. The equilibration buffer ensures that particular conditions exist within the column (e.g., pH, conductivity) such that a target molecule (e.g., an AAV capsid) interacts effectively with a stationary phase ligand, and is bound by the ligand while other molecules, such as impurities, flow through, or around, the stationary phase. The flow through may occur during the load step, and/or during a subsequent wash step.

As used herein, the term “flanked,” refers to a sequence that is flanked by other elements and indicates the presence of one or more flanking elements upstream and/or downstream, i.e., 5′ and/or 3′, relative to the sequence. The term “flanked” is not intended to indicate that the sequences are necessarily contiguous. For example, there may be intervening sequences between a nucleic acid encoding a transgene and a flanking element. A sequence (e.g., a transgene) that is “flanked” by two other elements (e.g., ITRs), indicates that one element is located 5′ to the sequence and the other is located 3′ to the sequence; however, there may be intervening sequences there between.

As used herein, the term “flocculation” refers to the process by which fine particulates are caused to clump together to form a floc. The fine particles may include proteins, nucleic acids, lipoproteins, cellular fragments resulting from lysis of host cells. In some embodiments, a floc that forms in a liquid phase may float to the top of the liquid (creaming), settle to the bottom (sedimentation) of the liquid or be filtered from the liquid phase.

As used herein, the term “fragment” refers to a material or entity that has a structure that includes a discrete portion of the whole but lacks one or more moieties found in the whole. In some embodiments, a fragment consists of a discrete portion. In some embodiments, a fragment consists of or comprises a characteristic structural element or moiety found in the whole. In some embodiments, a polymer fragment comprises, or consists of, at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160,170, 180, 190, 200, 210, 220, 230, 240, 250, 275, 300, 325, 350, 375, 400, 425, 450, 475, 500 or more monomeric units (e.g., amino acid residues, nucleotides) found in the whole polymer.

rAAV vectors are referred to as “full,” a “full capsid,” “full vector” or a “fully packaged vector” when the capsid contains a complete vector genome, including a transgene. During production of rAAV vectors by host cells, vectors may be produced that have less packaged nucleic acid than the full capsids and contain, for example a partial or truncated vector genome. These vectors are referred to as “intermediates,” an “intermediate capsid,” a “partial” or a “partially packaged vector.” An intermediate capsid may also be a capsid with an intermediate sedimentation rate, that is a sedimentation rate between that of full capsids and empty capsids, when analyzed by analytical ultracentrifugation. Host cells may also produce viral capsids that do not contain any detectable nucleic acid material. These capsids are referred to as “empty(s),” or “empty capsids.” Full capsids may be distinguished from empty capsids based on A260/A280 ratios determined by SEC-HPLC, whereby the A260/A280 ratios have been previously calibrated against capsids (i.e., full, intermediate and empty) analyzed by analytical ultracentrifugation. Other methods known in the art for the characterization of capsids include CryoTEM, capillary isoelectric focusing and charge detection mass spectrometry. Calculated isoelectric points of ˜6.2 and ˜5.8 for empty and full AAV9 capsids, respectively have been reported (Venkatakrishnan et al., (2013) J. Virology 87.9:4974-4984).”

As used herein, the term “null capsid” refers to a capsid produced intentionally to lack a vector genome. Such null a capsid may be produced by transfection of a host cell with a rep/cap and a helper plasmid, but not a plasmid that comprises the transgene cassette sequence, also known as a vector plasmid.

As used herein, the term “functional” refers to a biological molecule in a form in which it exhibits a property and/or activity by which it is characterized. A biological molecule may have two functions (i.e., bifunctional) or many functions (i.e., multifunctional).

As used herein, the term “gene” refers to a polynucleotide containing at least one open reading frame that is capable of encoding a particular polypeptide or protein after being transcribed and translated. “Gene transfer” or “gene delivery” refers to methods or systems for reliably inserting foreign DNA into host cells. Such methods can result in transient expression of non-integrated transferred DNA, extrachromosomal replication and expression of transferred replicons (e.g. episomes), and/or integration of transferred genetic material into the genomic DNA of host cells.

As used herein, the term “heterologous” refers to a nucleic acid inserted into a vector (e.g., rAAV vector) for purposes of vector mediated transfer/delivery of the nucleic acid into a cell. Heterologous nucleic acids are typically distinct from the vector (e.g., AAV) nucleic acid, that is, the heterologous nucleic acid is non-native with respect to the viral (e.g., AAV) nucleic acid. Once transferred or delivered into a cell, a heterologous nucleic acid, contained within a vector, can be expressed (e.g., transcribed and translated if appropriate). Alternatively, a transferred or delivered heterologous nucleic acid in a cell, contained within the vector, need not be expressed. Although the term “heterologous” is not always used herein in reference to a nucleic acid, reference to a nucleic acid even in the absence of the modifier “heterologous” is intended to include a heterologous nucleic acid. For example, a heterologous nucleic acid would be a nucleic acid encoding a dystrophin polypeptide, or a fragment thereof, for example a codon optimized mini-dystrophin transgene described in WO 2017/221145, and incorporated herein by reference, for use in the treatment of Duchenne muscular dystrophy.

A further exemplary heterologous nucleic acid comprises a wild-type coding sequence, or a fragment thereof (e.g., truncated, internal deletion), of one of the following genes, and may or may not be codon-optimized:

| | | | | | |
|---|---|---|---|---|---|
| ABCA7 | COL17A1 | GBA | IDUA | PCSK9 | SGSH |
| ABCD1 | COL4A | GBE1 | IL2RG | PDE6C | SH3TC2 |
| ACAN | COL4A3 | GDAP1 | IMPDH1 | PDE6H | SLC25A13 |
| ADA | COL4A4 | GJB1 | ITGB2 | PINK1 | SLC25A15 |
| ADA2 | COL7A1 | GLA | ITGB4 | PKLR | SLC26A2 |
| ADAM10 | CPS1 | GLB1 | JAG1 | PMP22 | SMN |
| AGL | CRB1 | GLB1 | KDM6A | PON1 | SMPD1 |
| AIPL1 | CRX | GNAT2 | KMT2D | PPT1 | SNCA |
| APOB | CTNS | GNE | LAMA3 | PRKN | SORD |
| APOE4 | CTSD | GRN | LAMB3 | PRPF31 | SPATA7 |
| APP | CTSF | GRN | LAMC2 | PRPF8 | SPINK5 |
| ARG1 | CYBA | GRS | LAMP2 | PRPH2 | TGM1 |
| ARSA | CYBB | GUCA1B | LCA5 | PSEN1 | TPP1 |
| ARSB | CYP21A2 | GUCY2D | LDLR | PSEN2 | TULP1 |
| ASL | DDC | GYG1 | LPL | PYGL | UGT1A1 |
| ASS1 | DMD | HBA1 | LRAT | PYGM | VCP |
| ATF6 | DMPK | HBA2 | LRRK2 | RD3 | VEGF |
| ATP7B | DYSF | HBB | MFN2 | RDH12 | VEGFA |
| C9orf72 | F12 | HEXA | MPZ | RHO | VPS13C |
| CEP290 | F8 | HEXB | MTM1 | RP1 | VPS35 |
| CFTR | F9 | HGD | NAGLU | RPE65 | WAS |
| CHM | FANCA | HGH | NAGS | RPGR | WIPF1 |
| CHMP2B | FBLN5 | HGSNAT | NCF1 | RPGRIP1 | XPNPEP2 |
| CLN2 | FGF-1 | HINT1 | NCF2 | RS1 | BAG3 |
| CLN3 | FGFR2 | HMBS | NCF4 | SCL37A4 | ATP8B1 |
| CLN5 | FGFR3 | HNRNPA1 | NOTCH2 | SCN1A | ABCB11 |
| CLN6 | FXN | HNRNPA2B1 | OAT | SERPINA1 | ABCB4 |
| CNBP | G6PC | HTRA1 | OTC | SERPING1 | |
| CNGA3 | GAA | HTT | PAH | SGCA | |
| CNGB3 | GALNS | IDS | PARK7 | SGCG | |

As used herein, the term “homologous,” or “homology,” refers to two or more reference entities (e.g., nucleotide or polypeptide sequences) that share at least partial identity over a given region or portion. For example, when an amino acid position in two peptides is occupied by identical amino acids, the peptides are homologous at that position. Notably, a homologous peptide will retain activity or function associated with the unmodified or reference peptide and the modified peptide will generally have an amino acid sequence “substantially homologous” with the amino acid sequence of the unmodified sequence. When referring to a polypeptide, nucleic acid or fragment thereof, “substantial homology” or “substantial similarity,” means that when optimally aligned with appropriate insertions or deletions with another polypeptide, nucleic acid (or its complementary strand) or fragment thereof, there is sequence identity in at least about 95% to 99% of the sequence. The extent of homology (identity) between two sequences can be ascertained using computer program or mathematical algorithm. Such algorithms that calculate percent sequence homology (or identity) generally account for sequence gaps and mismatches over the comparison region or area. Exemplary programs and algorithms are provided below.

As used herein, the terms “host cell,” “host cell line,” and “host cell culture” are used interchangeably and refer to a cell into which an exogenous nucleic acid has been introduced, and includes the progeny of such a cell. A host cell includes a “transfectant,” “transformant,” “transformed cell,” and “transduced cell,” which includes the primary transfected, transformed or transduced cell, and progeny derived therefrom, without regard to the number of passages. In some embodiments, a host cell is a packaging cell for production of a rAAV vector.

As used herein, the term “host cell DNA” or “HCDNA” refers to residual DNA, derived from a host cell culture which produced a rAAV vector, and present in a chromatography fraction (e.g., an affinity eluate, an AEX eluate, a wash) or a chromatography load (e.g., an affinity load, an AEX load). Host cell DNA may be measured by methods know in the art such as qPCR to detect a sequence unique to the host cells. General DNA concentrations may be estimated using fluorescence dyes (e.g. PicoGreen® or SYBR® Green), absorbance measurement (e.g. at 260 nm, or 254 nm) or electrophoretic techniques (e.g. agarose gel electrophoresis, or capillary electrophoresis). An amount of HCDNA present in an eluate may be expressed relative to the amount of vg present in the eluate, for example, ng HCDNA/1×10¹⁴ vg or pg HCDNA/1×10¹ vg. An amount of HCDNA present in an eluate may be expressed relative to the amount of vg present in a volume of eluate, for example, pg HCDNA/mL eluate.

As used herein, the term “host cell protein” or “HCP” refers to residual protein, derived from a host cell culture which produced a rAAV vector, present in a chromatography fraction (e.g., an affinity eluate, an AEX eluate, a wash) or a chromatography load (e.g., an affinity load, an AEX load). Host cell protein may be measured by methods known in the art, such as ELISA. Host cell protein can be semi-quantitatively measured by various electrophoretic staining methods (e.g., silver stain SDS-PAGE, SYPRO® Ruby stain SDS-PAGE, and/or Western blot). An amount of HCP present in an eluate may be expressed relative to the amount of vg present, for example, ng HCP/1×10¹⁴ vg or pg HCP/1×10⁹ vg.

As used herein, the term “identity” or “identical to” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, 99% or more identical.

Calculation of the percent identity of two nucleic acid or polypeptide sequences, for example, can be performed by aligning two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of a reference sequence. Nucleotides at corresponding positions are then compared. When a position in a first sequence is occupied by the same residue (e.g., nucleotide or amino acid) as the corresponding position in a second sequence, then the molecules are identical at that position. The percent identity between two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm.

To determine percent identity, or homology, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wis., USA. Other techniques for alignment are described in Methods in Enzymology, vol. 266: Computer Methods for Macromolecular Sequence Analysis (1996), ed. Doolittle, Academic Press, Inc. Of particular interest are alignment programs that permit gaps in the sequence. Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Also of interest is the BestFit program using the local homology algorithm of Smith and Waterman (1981, Advances in Applied Mathematics 2: 482-489) to determine sequence identity. The gap generation penalty will generally range from 1 to 5, usually 2 to 4 and in some embodiments will be 3. The gap extension penalty will generally range from about 0.01 to 0.20 and in some instances will be 0.10. The program has default parameters determined by the sequences inputted to be compared. Preferably, the sequence identity is determined using the default parameters determined by the program. This program is available also from Genetics Computing Group (GCG) package, from Madison, WI, USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters: Mismatch Penalty: 1.00; Gap Penalty: 1.00; Gap Size Penalty: 0.33; and Joining Penalty: 30.0.

As used herein, the term “impurity” refers to any molecule other than the rAAV vector being purified that is also present in a solution comprising the rAAV vector being purified. Impurities include empty capsids, intermediate capsids, biological macromolecules such as DNA (e.g., host cell DNA), RNA, non-AAV proteins (e.g., host cell proteins), AAV aggregates, damaged AAV capsids, molecules that are part of an absorbent used for chromatography that may leach into a sample during prior purification steps, endotoxins, cell debris and chemicals from cell culture, including media components, plasmid DNA from transfection, an adventitious agent, bacteria and viruses.

As used herein, the term “infectivity ratio” or “IR” refers to the number of rAAV vector particles needed to infect a cell. In some embodiments, the cell is in an in vitro system. In some embodiments, the cell is a cell within, or taken from, a subject in need of treatment with the rAAV vector. Infectivity ratio may be measured by any method known in the art including a cell-based qPCR assay. Infectivity may be expressed as infectivity units (IU) per volume, IU/mL, or relative to the amount of vg present, IU/vg.

As used herein, the terms “inverted terminal repeat, “ITR,” “terminal repeat,” and “TR” refer to palindromic terminal repeat sequences at or near the ends of the AAV virus genome, comprising mostly complementary, symmetrically arranged sequences. These ITRs can fold over to form T-shaped hairpin structures that function as primers during initiation of DNA replication. They are also needed for viral genome integration into host genome, for the rescue from the host genome; and for the encapsidation of viral nucleic acid into mature virions. The ITRs are required in cis for vector genome replication and its packaging into viral particles. “5′ ITR” refer to the ITR at the 5′ end of the AAV genome and/or 5′ to a recombinant transgene. “3′ ITR” refers to the ITR at the 3′ end of the AAV genome and/or 3′ to a recombinant transgene. Wild-type ITRs are approximately 145 bp in length. A modified, or recombinant ITR, may comprise a fragment or portion of a wild-type AAV ITR sequence. One of ordinary skill in the art will appreciate that during successive rounds of DNA replication ITR sequences may swap such that the 5′ ITR becomes the 3′ ITR, and vice versa. In some embodiments, at least one ITR is present at the 5′ and/or 3′ end of a recombinant vector genome such that the vector genome can be packaged into a capsid to produce a rAAV vector (also referred to herein as “rAAV vector particle” or “rAAV viral particle”) comprising the vector genome.

As used herein, the term “isolated” refers to a substance or composition that is 1) designed, produced, prepared, and or manufactured by the hand of man and/or 2) separated from at least one of the components with which it was associated when initially produced (whether in nature and/or in an experimental setting). Generally, isolated compositions are substantially free of one or more materials with which they normally associate with in nature, for example, one or more protein, nucleic acid, lipid, carbohydrate and/or cell membrane. The term “isolated” does not exclude man-made combinations, for example, a recombinant nucleic acid, a recombinant vector genome (e.g., rAAV vector genome), a rAAV vector particle (e.g., such as, but not limited to, a rAAV vector particle comprising an AAV9 capsid) that packages, e.g., encapsidates, a vector genome and a pharmaceutical formulation. The term “isolated” also does not exclude alternative physical forms of the composition, such as hybrids/chimeras, multimers/oligomers, modifications (e.g., phosphorylation, glycosylation, lipidation), variants or derivatized forms, or forms expressed in host cells that are man-made.

Isolated substances or compositions may be separated from about 10%, about 20%, about 30%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure,” after having been combined with certain other components such as, for example, one or more carriers or excipients (e.g., buffer, solvent, water, etc.); in such embodiments, percent isolation or purity of the substance is calculated without including such carriers or excipients.

As used herein, the term “linear flow velocity” or “flow velocity” refers to the rate at which a solution flows through a stationary phase in a column as a function of volumetric flow rate and column radius and is expressed by the equation:

u=F/(π*r²),

where u=linear flow rate (cm/hr); F=volumetric flow rate (mL/hr); and r=column radius (cm). Linear flow velocity may be expressed as distance per time, for example, cm/hr.

As used herein, the term “load” or “load solution” refers to any material (e.g., a solution) containing a product of interest that is loaded onto a chromatography stationary phase. In some embodiments, a product of interest is a rAAV vector. In some embodiments, when a “load solution” is exposed to a chromatography stationary phase some components of the load solution bind to the stationary phase. In some embodiments, when a load solution is exposed to a chromatography stationary phase some components of the load solution do not bind to the chromatography stationary phase and flow through the column with the mobile phase as an “unbound fraction” or a “flow through.” In some embodiments, a load solution is a clarified lysate.

As used herein, the term “load chase” refers to a solution applied to a column after the load or load solution (as defined, infra) has been applied. A load chase serves to complete application of the load or load solution to the stationary phase, and to remove unbound material from the column.

As used herein, the term “modifier,” or “mobile phase modifier” is a component of the mobile phase that modifies the mobile phase in order to alter the chromatography. Such altering of the chromatography results in, for example, the removal, or washing off of, impurities from the stationary phase, or elution of a product or substance of interest from the stationary phase. Examples of “modifiers” include a salt, a buffering agent, a detergent, an amino acid (e.g., arginine, histidine, citrulline, glycine), an organic solvent (e.g., ethanol, ethylene glycol), a chaotropic agent (e.g., urea), or a displacer (also referred to as a selective elution agent).

As used herein, the terms “nucleic acid sequence,” “nucleotide sequence,” and “polynucleotide” refer interchangeably to any molecule composed of or comprising monomeric nucleotides connected by phosphodiester linkages. A nucleic acid may be an oligonucleotide or a polynucleotide. Nucleic acid sequences are presented herein in the direction from the 5′ to the 3′ direction. A nucleic acid sequence (i.e., a polynucleotide) of the present disclosure can be a deoxyribonucleic acid (DNA) molecule or ribonucleic acid (RNA) molecule and refers to all forms of a nucleic acid such as, double stranded molecules, single stranded molecules, small or short hairpin RNA (shRNA), micro RNA, small or short interfering RNA (siRNA), trans-splicing RNA, antisense RNA, messenger RNA, transfer RNA, ribosomal RNA. Where a polynucleotide is a DNA molecule, that molecule can be a gene, a cDNA, an antisense molecule or a fragment of any of the foregoing molecules. Nucleotides are indicated herein by a single letter code: adenine (A), guanine (G), thymine (T), cytosine (C), inosine (I) and uracil (U). A nucleotide sequence may be chemically modified or artificial. Nucleotide sequences include peptide nucleic acids (PNA), morpholinos and locked nucleic acids (LNA), as well as glycol nucleic acids (GNA) and threose nucleic acids (TNA). Each of these sequences is distinguished from naturally-occurring DNA or RNA by changes to the backbone of the molecule. Also, phosphorothioate nucleotides may be used. Other deoxynucleotide analogs include methylphosphonates, phosphoramidates, phosphorodithioates, N3′-P5′-phosphoramidates, and oligoribonucleotide phosphorothioates and their 2′-O-allyl analogs and 2′-O-methylribonucleotide methylphosphonates which may be used in a nucleotide sequence of the disclosure.

As used here, the term “nucleic acid construct,” refers to a non-naturally occurring nucleic acid molecule resulting from the use of recombinant DNA technology (e.g., a recombinant nucleic acid). A nucleic acid construct is a nucleic acid molecule, either single or double stranded, which has been modified to contain segments of nucleic acid sequences, which are combined and arranged in a manner not found in nature. A nucleic acid construct may be a “vector” (e.g., a plasmid, a rAAV vector genome, an expression vector, etc.), that is, a nucleic acid molecule designed to deliver exogenously created DNA into a host cell.

As used herein, the term “operably linked” refers to a linkage of nucleic acid sequence (or polypeptide) elements in a functional relationship. A nucleic acid is operably linked when it is placed into a functional relationship with another nucleic acid sequence. For instance, a promoter or other transcription regulatory sequence (e.g., an enhancer) is operably linked to a coding sequence if it affects the transcription of the coding sequence. In some embodiments, operably linked means that nucleic acid sequences being linked are contiguous. In some embodiments, operably linked does not mean that nucleic acid sequences are contiguously linked, rather intervening sequences are between those nucleic acid sequences that are linked.

As used herein, the term “percent purity,” “% purity,” or “% capsid purity” refers to a purity of a solution comprising full capsids, empty capsids and intermediate capsids. A percent purity is determined by methods known in the art, including reverse phase HPLC, non-reducing conditions (RP-HPLC, NR). The area under the chromatogram absorbance curve generated by RP-HPLC, NR that is attributable to capsids present in the solution, is expressed as a percentage of the total area under the absorbance curve. Percent purity may also be measured by SDS-PAGE or capillary electrophoresis.

As used herein, the term “percent vg yield,” “% vg yield” or “% vg recovery” refers to the amount of VG in an eluate collected from an affinity column (i.e., an affinity eluate or an affinity pool) as a percentage of the amount of VG present in the solution loaded onto the affinity column (e.g., a clarified lysate). For instance, % VG yield=((amount of VG in an affinity eluate)/(amount of VG in a clarified lysate))*100.

As used herein, the term “percent VG step yield” or “% VG step yield” refers to the amount of VG in a pooled eluate collected from an AEX column (i.e., an AEX pool) as a percentage of the amount of VG present in the affinity pool (also referred to herein as an affinity eluate) prior to dilution or filtration. For instance, % VG step yield=((amount of VG in AEX pool)/(amount of VG in affinity pool))*100.

As used herein, the term “pre-elution wash” refers to a step of a chromatography process that serves to remove impurities present in the mobile phase, impurities that have bound non-specifically to the stationary phase, and/or bound to the rAAV capsids, after a load solution has been applied to the stationary phase. Impurities that non-specifically bind to the stationary phase may be bound to the resin material and/or the ligand. For example, when purifying a rAAV vector by a chromatography process, a pre-elution wash solution is applied to a stationary phase after a solution comprising the rAAV vector has been loaded onto the stationary phase, to remove impurities (e.g., HCDNA, HCP) present in the mobile phase as well as those non-specifically bound to the stationary phase or rAAV capsids, while maintaining the interaction between the stationary phase (for example, via a ligand) and the rAAV vector.

As used herein, the term “pharmaceutically acceptable” and “physiologically acceptable” refers to a biologically acceptable formulation, gaseous, liquid or solid, or mixture thereof, which is suitable for one or more routes of administration, in vivo delivery or contact.

As used herein, the terms “polypeptide,” “protein,” “peptide” or “encoded by a nucleic acid sequence” (i.e., encode by a polynucleotide sequence, encoded by a nucleotide sequence) refer to full-length native sequences, as with naturally occurring proteins, as well as functional subsequences, modified forms or sequence variants so long as the subsequence, modified form or variant retains some degree of functionality of the native full-length protein. In methods and uses of the disclosure, such polypeptides, proteins and peptides encoded by the nucleic acid sequences can be but are not required to be identical to the endogenous protein that is defective, or whose expression is insufficient, or deficient in a subject treated with gene therapy.

As used herein, the term “recombinant,” refers to a vector, polynucleotide (e.g., a recombinant nucleic acid), polypeptide or cell that is the product of various combinations of cloning, restriction or ligation steps (e.g., relating to a polynucleotide or polypeptide comprised therein), and/or other procedure that results in a construct that is distinct from a product found in nature. A recombinant virus or vector (e.g., rAAV vector) comprises a vector genome comprising a recombinant nucleic acid (e.g., a nucleic acid comprising a transgene and one or more regulatory elements). The terms respectively include replicates of the original polynucleotide construct and progeny of the original virus construct.

As used herein, the term “regeneration” refers to a step, phase or process whereby a stationary phase is contacted with a solution which brings the stationary phase back to its original state by, for example, removal of impurities or removal of a chemical applied to the stationary phase during a prior chromatography step. In some embodiments, a step of regeneration precedes a step of loading a solution comprising a rAAV vector onto the stationary phase. In some embodiments, a step of regenerating may be performed on a stationary phase more than once over the course of a chromatography run.

As used herein, the term “residence time” or “contact time” refers to the time that a solution within a column is in contact with a stationary phase. Residence time may be expressed as time per column volume (CV), for example, minutes/CV.

As used herein, the term, “sanitization” refers to a step, phase or process whereby a stationary phase is contacted with a solution that serves to reduce bioburden within the column. A sanitization step may be performed on a stationary phase more than once over the course of a chromatography run.

As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments, a human subject is an adult, adolescent, or pediatric subject. In some embodiments, a subject is suffering from a disease, disorder or condition, e.g., a disease, disorder or condition that can be treated as provided herein. In some embodiments, a subject is suffering from a disease, disorder or condition associated with deficient or dysfunctional dystrophin, e.g., Duchenne muscular dystrophy. In some embodiments, a subject is susceptible to a disease, disorder, or condition. In some embodiments, a susceptible subject is predisposed to and/or shows an increased risk (as compared to the average risk observed in a reference subject or population) of developing a disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a human patient. In some embodiments, a subject is an individual to whom diagnosis and/or therapy is and/or has been administered (e.g., gene therapy for Duchenne muscular dystrophy). In some embodiments, a subject is a human patient with Duchenne muscular dystrophy.

Disease, disorders and conditions that can be treated using a rAAV vector purified according to the methods set forth herein include, for example a metabolic disease or disorder (e.g., Fabry disease, Gaucher disease, phenylketonuria, glycogen storage disease); a urea cycle disease or disorder (e.g., ornithine transcarbamylase deficiency); a lysosomal storage disease or disorder (e.g., metachromatic leukodystrophy, mucopolysaccharidosis); a liver disease or disorder (e.g., progressive familial intrahepatic cholestasis type 1-3); a blood disease or disorder (Hemophilia A, Hemophilia B, a thalassemia); a cancer (e.g., a carcinoma, a sarcoma, a blood cancer); a genetic disease or disorder (e.g., cystic fibrosis); or an infectious disease (e.g., HIV).

Diseases, disorders and conditions that can be treated using a rAAV vector purified according to the methods set forth herein include, for example: 21-hydroxylase-deficient congenital adrenal hyperplasia, achondrogenesis Type 1 B, achondroplasia, achromatopsia, acid sphingomyelinase deficiency (Niemann-Pick disease type A or B), acute intermittent porphyria, adenosine deaminase 2 deficiency, adenosine deaminase deficiency (e.g., severe combined immunodeficiency, X-linked), adrenoleukodystrophy (e.g., X-linked), age-related macular degeneration (e.g., neovascular, wet), Alagille syndrome, alkaptonuria, alpha-1 antitrypsin deficiency, alpha-thalassemia, Alport syndrome, Alzheimer disease, Apert syndrome, arginase deficiency, argininosuccinate lyase (ASL) deficiency, argininosuccinate synthase (ASS1) deficiency (citrullinemia type 1), aromatic L-amino acid decarboxylase deficiency, autosomal recessive congenital ichthyosis, Becker muscular dystrophy, beta-thalassemia, carbamoylphosphatase synthetase I deficiency, ceroid lipofuscinosis, Charcot-Marie-Tooth neuropathy, choroideremia, chronic granulomatous disease, citrin deficiency, Crigler-Najjar syndrome type 1 and 2, critical limb ischemia, cystic fibrosis, cystinosis, Danon disease, diabetic macular retinopathy, dominant inherited short stature, Dravet syndrome, Duchenne muscular dystrophy, dysferlinopathy (e.g., Miyoshi myopathy, limb-girdle muscular dystrophy 2B), dystrophic epidermolysis bullosa, Fabry disease, familial hypercholesterolemia, familial lipoprotein lipase deficiency, Fanconia anemia (e.g., Fanconia anemia A), Friedreich's ataxia, frontotemporal dementia, Gaucher disease, glycogen storage disease type 1A and 1 B (Von Gierke's disease), glycogen storage disease type Ill, glycogen storage disease type IV, glycogen storage disease type V, glycogen storage disease type VI, glycogen storage disease type XV, GM1 gangliosidosis, gyrate atrophy, hemophilia A, hemophilia B, hereditary angiodema, types I-Ill, Huntington's disease, inclusion body myositis, junctional epidermolysis bullosa, Kabuki Syndrome, Leber congenital amaurosis, leukocyte adhesion defect type 1, limb girdle muscular dystrophy, limb girdle muscular dystrophy type 2C (gamma-sarcoglycanopathy), limb girdle muscular dystrophy type 2D, metachromatic leukodystrophy, mucopolysaccharidosis Type 1, mucopolysaccharidosis type II (Hunter syndrome), mucopolysaccharidosis type IIIA, mucopolysaccharidosis type IIIB, mucopolysaccharidosis type IIIC, mucopolysaccharidosis type IIID, mucopolysaccharidosis type IVA (Morquio A syndrome), mucopolysaccharidosis type IVB (Morquio B syndrome), mucopolysaccharidosis type VI (Maroteaux-Lamy), myotonic dystrophy type 1, myotonic dystrophy type 2, N-acetylglutamate synthase (NAGS) deficiency, Netherton syndrome, neuronal ceroid lipofuscinosis, ornithine translocase deficiency, ornithine transcarbamylase deficiency disease, Parkinson's disease, phenylketonuria, Pompe, progressive familial intrahepatic cholestasis type 1-3, progressive myofibrillar myopathy, pyruvate kinase deficiency, retinitis pigmentosa, RPE65-related Leber congenital amaurosis, Sandhoff disease, sickle cell disease, spinal muscular atrophy, Tay Sachs disease, Wilson disease, Wiskott-Aldrich syndrome, Wiskott-Aldrich syndrome 2, X-linked adrenoleukodystrophy, X-linked chronic granulomatous disease, X-linked myotubular myopathy, X-linked retinitis pigmentosa, X-linked retinoschisis and X-linked severe combined immunodeficiency.

As used herein, the term “substantially” refers to the qualitative condition of exhibition of total or near-total extent or degree of a characteristic or property of interest. One of ordinary skill in the art will understand that biological and chemical phenomena rarely, if ever, go to completion and/or proceed to completeness or achieve or an absolute result. The term “substantially” is therefore used herein to capture the potential lack of completeness inherent in many biological and chemical phenomena.

As used herein, the term “therapeutic polypeptide” is a peptide, polypeptide or protein (e.g., enzyme, structural protein, transmembrane protein, transport protein) that may alleviate or reduce symptoms that result from an absence or defect in a protein in a target cell (e.g., an isolated cell) or organism (e.g., a subject). A therapeutic polypeptide or protein encoded by a transgene is one that confers a benefit to a subject, e.g., to correct a genetic defect, to correct a deficiency in a gene related to expression or function. Similarly, a “therapeutic transgene” is the transgene that encodes the therapeutic polypeptide. In some embodiments, a therapeutic polypeptide, expressed in a host cell, is an enzyme expressed from a transgene (i.e., an exogenous nucleic acid that has been introduced into the host cell). In some embodiments, a therapeutic polypeptide is a dystrophin protein, or fragment thereof, expressed from a therapeutic transgene transduced into a muscle cell (e.g., a skeletal muscle cell).

As used herein, the term “therapeutically effective amount” refers to an amount that produces the desired therapeutic effect for which it is administered. In some embodiments, the term refers to an amount that is sufficient, when administered to a population suffering from or susceptible to a disease, disorder or condition in accordance with a therapeutic dosing regimen, to treat the disease, disorder or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment.

As used herein, the term “transgene” is used to mean any heterologous polynucleotide for delivery to and/or expression in a host cell, target cell or organism (e.g., a subject). Such “transgene” may be delivered to a host cell, target cell or organism using a vector (e.g., rAAV vector). A transgene may be operably linked to a control sequence, such as a promoter. It will be appreciated by those of skill in the art that expression control sequences can be selected based on an ability to promote expression of the transgene in a host cell, target cell or organism. Generally, a transgene may be operably linked to an endogenous promoter associated with the transgene in nature, but more typically, the transgene is operably linked to a promoter with which the transgene is not associated in nature. An example of a transgene is a nucleic acid encoding a therapeutic polypeptide, for example a dystrophin polypeptide or fragment thereof, and an exemplary promoter is one not operable linked to a nucleotide encoding dystrophin in nature. Such a non-endogenous promoter can include a CBh promoter or a muscle specific promoter, among many others known in the art.

A nucleic acid of interest can be introduced into a host cell by a wide variety of techniques that are well-known in the art, including transfection and transduction.

“Transfection” is generally known as a technique for introducing an exogenous nucleic acid into a cell without the use of a viral vector. As used herein, the term “transfection” refers to transfer of a recombinant nucleic acid (e.g., an expression plasmid) into a cell (e.g., a host cell) without use of a viral vector. A cell into which a recombinant nucleic acid has been introduced is referred to as a “transfected cell.” A transfected cell may be a host cell (e.g., a CHO cell, Pro10 cell, HEK293 cell) comprising an expression plasmid/vector for producing a recombinant AAV vector. In some embodiments, a transfected cell (e.g., a packing cell) may comprise a plasmid comprising a transgene (e.g., a dystrophin transgene), a plasmid comprising an AAV rep gene and an AAV cap gene and a plasmid comprising a helper gene. Many transfection techniques are known in the art, which include, but are not limited to, electroporation, calcium phosphate precipitation, microinjection, cationic or anionic liposomes, and liposomes in combination with a nuclear localization signal.

As used herein, the term “transduction” refers to transfer of a nucleic acid (e.g., a vector genome) by a viral vector (e.g., rAAV vector) to a cell (e.g., a target cell, e.g., a muscle cell). In some embodiments, a gene therapy for Duchenne muscular dystrophy includes transducing a vector genome comprising a modified nucleic acid encoding dystrophin, or a fragment thereof, into a muscle cell. A cell into which a transgene has been introduced by a virus or a viral vector is referred to as a “transduced cell.” In some embodiments, a transduced cell is an isolated cell and transduction occurs ex vivo. In some embodiments, a transduced cell is a cell within an organism (e.g., a subject) and transduction occurs in vivo. A transduced cell may be a target cell of an organism which has been transduced by a recombinant AAV vector such that the target cell of the organism expresses a polynucleotide (e.g., a transgene, e.g., a modified nucleic acid encoding dystrophin, or a fragment thereof).

Cells that may be transduced include a cell of any tissue or organ type, or any origin (e.g., mesoderm, ectoderm or endoderm). Non-limiting examples of cells include liver (e.g., hepatocytes, sinusoidal endothelial cells), pancreas (e.g., beta islet cells, exocrine), lung, central or peripheral nervous system, such as brain (e.g., neural or ependymal cells, oligodendrocytes) or spine, kidney, eye (e.g., retinal), spleen, skin, thymus, testes, lung, diaphragm, heart (cardiac), muscle or psoas, or gut (e.g., endocrine), adipose tissue (white, brown or beige), muscle (e.g., fibroblasts, myocytes), synoviocytes, chondrocytes, osteoclasts, epithelial cells, endothelial cells, salivary gland cells, inner ear nervous cells or hematopoietic (e.g., blood or lymph) cells. Additional examples include stem cells, such as pluripotent or multipotent progenitor cells that develop or differentiate into liver (e.g., hepatocytes, sinusoidal endothelial cells), pancreas (e.g., beta islet cells, exocrine cells), lung, central or peripheral nervous system, such as brain (e.g., neural or ependymal cells, oligodendrocytes) or spine, kidney, eye (e.g., retinal), spleen, skin, thymus, testes, lung, diaphragm, heart (cardiac), muscle or psoas, or gut (e.g., endocrine), adipose tissue (white, brown or beige), muscle (e.g., fibroblast, myocytes), synoviocytes, chondrocytes, osteoclasts, epithelial cells, endothelial cells, salivary gland cells, inner ear nervous cells or hematopoietic (e.g., blood or lymph) cells.

In some embodiments, particular areas of a tissue or organ (e.g., muscle) may be transduced by a rAAV vector (e.g., a rAAV vector with a dystrophin, or portion of dystrophin, transgene) that is administered to the tissue or organ. In some embodiments, a muscle cell is transduced with a rAAV comprising a dystrophin transgene. In some embodiments, a skeletal muscle cell is transduced with a rAAV comprising a dystrophin transgene. In some embodiments, a cardiac muscle cell is transduced with a rAAV comprising a dystrophin transgene.

As used herein, the term “two-step % vg recovery” refers to a vg recovery following purification of rAAV from a solution comprising a rAAV vector by affinity chromatography and AEX chromatography. For example, a two-step vg recovery may be calculated by multiplying the affinity % vg recovery by the AEX % vg recovery.

As used herein, the term “upward flow” refers to the direction of flow of a mobile phase during a chromatographic separation such that the flow of the mobile phase is from the bottom of the column to the top of the column. In some embodiments, a mobile phase, such as pre-use water for injection (WFI), or a regeneration buffer, flows in a direction opposite that of a chromatographic separation step, such that the solution (e.g., water) flows from the bottom of the column to the top of the column, whereas during the chromatographic separation step (e.g., loading, washing or eluting) the solution flows from the top of the column to the bottom of the column.

As used herein, the term “vector” refers to a plasmid, virus (e.g., a rAAV), cosmid, or other vehicle that can be manipulated by insertion or incorporation of a nucleic acid (e.g., a recombinant nucleic acid). A vector can be used for various purposes including, e.g., genetic manipulation (e.g., cloning vector), to introduce/transfer a nucleic acid into a cell, to transcribe or translate an inserted nucleic acid in a cell. In some embodiments a vector nucleic acid sequence contains at least an origin of replication for propagation in a cell. In some embodiments, a vector nucleic acid includes a heterologous nucleic acid sequence, an expression control element(s) (e.g., promoter, enhancer), a selectable marker (e.g., antibiotic resistance), a poly-adenosine (polyA) sequence and/or an ITR. In some embodiments, when delivered to a host cell, the nucleic acid sequence is propagated. In some embodiments, when delivered to a host cell, either in vitro or in vivo, the cell expresses the polypeptide encoded by the heterologous nucleic acid sequence. In some embodiments, when delivered to a host cell, the nucleic acid sequence, or a portion of the nucleic acid sequence is packaged into a capsid. A host cell may be an isolated cell or a cell within a host organism. In addition to a nucleic acid sequence (e.g., transgene) which encodes a polypeptide or protein, additional sequences (e.g., regulatory sequences) may be present within the same vector (i.e., in cis to the gene) and flank the gene. In some embodiments, regulatory sequences may be present on a separate (e.g., a second) vector which acts in trans to regulate the expression of the gene. Plasmid vectors may be referred to herein as “expression vectors.”

As used herein, the term “vector genome” refers to a nucleic acid that that may, but need not, be packaged/encapsidated in an AAV capsid to form a rAAV vector. Typically, a vector genome includes a heterologous polynucleotide sequence (e.g., a transgene, regulatory elements, etc.) and at least one ITR. In cases where a recombinant plasmid is used to construct or manufacture a recombinant vector (e.g., rAAV vector), the vector genome does not include the entire plasmid but rather only the sequence intended for delivery by the viral vector. This non-vector genome portion of the recombinant plasmid is referred to as the “plasmid backbone,” which is important for cloning. selection and amplification of the plasmid, a process that is needed for propagation of recombinant viral vector production, but which is not itself packaged or encapsidated into a rAAV vector. Typically, the heterologous sequence to be packaged into the capsid is flanked by the ITRs such that when cleaved from the plasmid backbone, it is packaged into the capsid.

As used herein, the term “viral vector” generally refers to a viral particle that functions as a nucleic acid delivery vehicle and which comprises a vector genome (e.g., comprising a transgene which has replaced the wild type rep and cap) packaged within the viral particle (i.e., capsid) and includes, for example, lenti- and parvo-viruses, including AAV serotypes and variants (e.g., rAAV vectors). As noted elsewhere herein, a recombinant viral vector does not comprise a virus genome with a rep and/or a cap gene; rather, these sequences have been removed to provide capacity for the vector genome to carry a transgene of interest.

The present disclosure provides methods for purification of rAAV vectors (e.g., full rAAV vectors) from host cell harvests. In particular, the disclosure provides methods for purification of rAAV vectors (e.g., full rAAV vectors) from other nucleic acids and proteins (including empty capsids) produced by the host cell. Furthermore, the disclosure provides methods for the separation of empty capsids from full rAAV vectors (e.g., rAAV vectors comprising a vector genome). Each of these aspects of the disclosure is discussed further in the ensuing sections.

### II. AAV and rAAV Vectors

A. AAV

As discussed supra, “adeno-associated virus” and/or “AAV” refer to parvoviruses with a linear single-stranded DNA genome and variants thereof. The term covers all subtypes and both naturally occurring and recombinant forms, except where required otherwise. Parvoviruses, including AAV, are useful as gene therapy vectors as they can penetrate a cell and introduce a nucleic acid (e.g., transgene) into the nucleus. In some embodiments, the introduced nucleic acid (e.g., rAAV vector genome) forms circular concatemers that persist as episomes in the nucleus of transduced cells. In some embodiments, a transgene is inserted in specific sites in the host cell genome, for example at a site on human chromosome 19. Site-specific integration, as opposed to random integration, is believed to likely result in a predictable long-term expression profile. The insertion site of AAV into the human genome is referred to as AAVS1. Once introduced into a cell, polypeptides encoded by the nucleic acid can be expressed by the cell. Because AAV is not associated with any pathogenic disease in humans, a nucleic acid delivered by AAV can be used to express a therapeutic polypeptide for the treatment of a disease, disorder and/or condition in a human subject.

Multiple serotypes of AAV exist in nature with at least fifteen wild type serotypes having been identified from humans thus far (i.e., AAV1-AAV15). Over 150 unique AAV serotypes have been identified. Naturally occurring and variant serotypes are distinguished by having a protein capsid that is serologically distinct from other AAV serotypes. AAV type 1 (AAV1), AAV type 2 (AAV2), AAV type 3 (AAV3) including AAV type 3A (AAV3A) and AAV type 3B (AAV3B), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 12 (AAV12), AAVrh10, AAVrh74 (see WO 2016/210170), AAVrh32.22, AAV1.1, AAV2.5, AAV6.1, AAV6.2, AAV6.3.1, AAV9.45, AAVShH10, HSC15/17, RHM4-1 (SEQ ID NO:5 of WO 2015/013313), RHM15-1, RHM15-2, RHM15-3/RHM15-5, RHM15-4, RHM15-6, AAVhu.26, AAV2i8, AAV29G, AAV2, AAV8G9, AAV-LK03, AAV2-TT, AAV2-TT-S312N, AAV3B-S312N, avian AAV, bovine AAV, canine AAV, equine AAV, primate AAV, non-primate AAV, and ovine AAV, NP4, NP22, NP66, AAVDJ, AAVDJ/8, AAVDJ/9, among many others (see, e.g., Fields et al., “Virology”, volume 2, chapter 69 (4^{th} ed., Lippincott-Raven Publishers); U.S. Pat. No. 7,906,111; Gao et al. (2004) J. Virol. 78:6381; Morris et al. (2004) Virol. 33:375; WO 2013/063379; WO 2014/194132; WO 2015/121501; WO 2015/013313, all of which are hereby incorporated by reference). AAV variants isolated from human CD34+ cell include AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14 and AAVHSC15 (Smith et al. (2014) Molecular Therapy 22(9):1625-1634, which is hereby incorporated by reference). Naturally occurring AAVs isolated from human tissues by long-read sequencing include AAVv66 with tropism for the CNS as well as AAVv33, AAVv37, AAVv40, AAVv67, AAVv70, AAVv72, AAVv84, AAVv86, AAVv87 and AAVv90 (Hsu et al. (2020) Nat. Comm. 11:3279).

“Primate AAV” refers to AAV that infect primates, “non-primate AAV” refers to AAV that infect non-primate mammals, “bovine AAV” refers to AAV that infect bovine mammals, and so on. Serotype distinctiveness is determined on the basis of the lack of cross-reactivity between antibodies to one AAV as compared to another AAV. Such cross-reactivity differences are usually due to differences in capsid protein sequences and antigenic determinants (e.g., due to VP1, VP2, and/or VP3 sequence differences of AAV serotypes). However, some naturally occurring AAV or man-made AAV mutants (e.g., recombinant AAV) may not exhibit serological difference with any of the currently known serotypes. These viruses may then be considered a subgroup of the corresponding type, or more simply a variant AAV. Thus, as used herein, the term “serotype” refers to both serologically distinct viruses, e.g., AAV, as well as viruses, e.g., AAV, that are not serologically distinct but that may be within a subgroup or a variant of a given serotype.

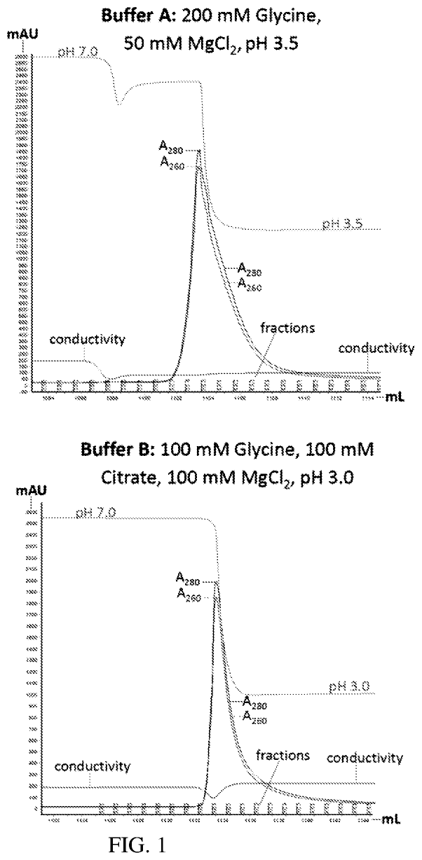

A comprehensive list and alignment of amino acid sequences of capsids of known AAV serotypes is provided by Marsic et al. (2014) Molecular Therapy 22(11):1900-1909, especially at supplementary FIG. 1; the entire publication is hereby incorporated by reference.

Genomic sequences of various serotypes of AAV, as well as sequences of the native inverted terminal repeats (ITRs), rep proteins, and capsid subunits are known in the art. Such sequences may be found in the literature or in public databases such as GenBank. See, e.g., GenBank Accession Numbers NC_002077 (AAV1), AF063497 (AAV1), NC_001401 (AAV2), AF043303 (AAV2), NC_001729 (AAV3), AF028705.1 (AAV3B), NC_001829 (AAV4), U89790 (AAV4), NC_006152 (AAV5), AF028704 (AAV6), AF513851 (AAV7), AF513852 (AAV8), NC_006261 (AAV8), AY530579 (AAV9), AY631965 (AAV10), AY631966 (AAV11), and DQ813647 (AAV12); the disclosures of which are incorporated by reference herein. See also, e.g., Srivistava et al. (1983) J. Virology 45:555; Chiorini et al. (1998) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al. (1986) J. Virol. 58:921; Gao et al. (2002) Proc. Nat. Acad. Sci. USA 99: 11854; Moris et al. (2004) Virology 33:375-383; International Patent Publications WO 00/28061, WO 99/61601, WO 98/11244; WO 2013/063379; WO 2014/194132; WO 2015/121501, and U.S. Pat. Nos. 6,156,303 and 7,906,111, which are hereby incorporated by reference. For illustrative purposes only, wild type AAV2 comprises a small (20-25 nm) icosahedral virus capsid of AAV composed of three proteins (VP1, VP2, and VP3; a total of 60 capsid proteins compose the AAV capsid) with overlapping sequences. The proteins VP1 (735 aa; Genbank Accession No. AAC03780), VP2 (598 aa; Genbank Accession No. AAC03778) and VP3 (533 aa; Genbank Accession No. AAC03779) exist in about a 1:1:10 ratio in the capsid. That is, for AAVs, VP1 is the full length protein and VP2 and VP3 are progressively shorter versions of VP1, with increasing truncation of the N-terminus relative to VP1. In one embodiment, of the method disclosed herein, a rAAV vector comprises an AAV9 VP1 comprising the amino acid sequence of SEQ ID NO:3.

B. Recombinant AAV (rAAV)

As discussed supra, a “recombinant adeno-associated virus” or “rAAV” is distinguished from a wild-type AAV by replacement of all or part of the viral genome with a non-native sequence. Incorporation of a non-native sequence within the virus defines the viral vector as a “recombinant” vector, and hence a “rAAV vector.” A rAAV vector can include a heterologous polynucleotide (e.g., human codon-optimized gene encoding human mini-dystrophin, e.g., SEQ ID NO:1) encoding a desired protein or polypeptide (e.g., a dystrophin polypeptide, or fragment thereof, e.g., SEQ ID NO:2). A recombinant vector sequence may be encapsidated or packaged into an AAV capsid and referred to as an “rAAV vector,” an “rAAV vector particle,” “rAAV viral particle” or simply a “rAAV.”

The present disclosure provides for methods of purifying a rAAV vector comprising a polynucleotide sequence not of AAV origin (e.g., a polynucleotide heterologous to AAV). The heterologous polynucleotide may be flanked by at least one, and sometimes by two, AAV terminal repeat sequences (e.g., inverted terminal repeats (ITRs)). The heterologous polynucleotide flanked by ITRs, also referred to herein as a “vector genome,” typically encodes a polypeptide of interest, or a gene of interest (“GOI”), such as a target for therapeutic treatment (e.g., a nucleic acid encoding dystrophin, or a fragment thereof, for the treatment of Duchenne muscular dystrophy). Delivery or administration of a rAAV vector to a subject (e.g. a patient) provides encoded proteins and peptides to the subject. Thus, a rAAV vector can be used to transfer/deliver a heterologous polynucleotide for expression for, e.g., treating a variety of diseases, disorders and conditions.

rAAV vector genomes generally retain 145 base ITRs in cis to the heterologous nucleic acid sequence that replaces the viral rep and cap genes. Such ITRs are necessary to produce a recombinant AAV vector; however, modified AAV ITRs and non-AAV terminal repeats including partially or completely synthetic sequences can also serve this purpose. ITRs form hairpin structures and function to, for example, serve as primers for host-cell-mediated synthesis of the complementary DNA strand after infection. ITRs also play a role in viral packaging, integration, etc. ITRs are the only AAV viral elements which are required in cis for AAV genome replication and packaging into rAAV vectors. A rAAV vector genome optionally comprises two ITRs which are generally at the 5′ and 3′ ends of the vector genome comprising a heterologous sequence (e.g., a transgene encoding a gene of interest, or a nucleic acid sequence of interest including, but not limited to, an antisense, and siRNA, a CRISPR molecule, among many others). A 5′ and a 3′ ITR may both comprise the same sequence, or each may comprise a different sequence. An AAV ITR may be from any AAV including by not limited to serotypes 1, 2, 3, 4, 5, 6, 7, 8, 9, 10 or 11 or any other AAV. An ITR is a sequence which mediates AAV genome replication and packaging.

A rAAV vector of the disclosure may comprise an ITR from an AAV serotype (e.g., wild-type AAV2, a fragment or variant thereof) that differs from the serotype of the capsid (e.g., AAV9 or other). Such a rAAV vector comprising at least one ITR from one serotype, but comprising a capsid from a different serotype, may be referred to as a hybrid viral vector (see U.S. Pat. No. 7,172,893). An AAV ITR may include the entire wild type ITR sequence, or be a variant, fragment, or modification thereof, but will retain functionality.

In some embodiments, a heterologous polypeptide comprises an ITR (e.g., an ITR from AAV2, but can comprise an ITR from any wild type AAV serotype, or a variant thereof) positioned at the left and right ends (i.e., 5′ and 3′ termini, respectively) of a vector genome. In some embodiments, a left (e.g., 5′) ITR comprises or consists of the nucleic acid sequence of SEQ ID NO:5 or SEQ ID NO:6. In some embodiments, a left (e.g., 5′) ITR comprises a nucleic acid sequence that is, or is at least, or is at most 80%, 85%, 90%, 95%, 98%, 99% or 100% identical to SEQ ID NO:5 or SEQ ID NO:6. In some embodiments, a right (e.g., 3′) ITR comprises or consists of a nucleic acid sequence of SEQ ID NO:5 or SEQ ID NO:6. In some embodiments, a right (e.g., 3′) ITR comprises a nucleic acid sequence that is, is at least, or is at most 80%, 85%, 90%, 95%, 98%, 99% or 100% identical to SEQ ID NO:5 or SEQ ID NO:6. Each ITR is in cis with but may be separated from each other, or other elements in the vector genome, by a nucleic acid sequence of variable length, such as a recombinant nucleic acid comprising a modified nucleic acid encoding dystrophin, or a fragment thereof, and regulatory elements. In some embodiments, ITRs are AAV2 ITRs, or variants thereof, and flank a dystrophin transgene. In some embodiments, a rAAV comprises a dystrophin transgene (e.g., comprising the nucleic acid sequence of SEQ ID NO:1) flanked by AAV2 ITRs (e.g., ITRs having the sequence as set forth in SEQ ID NO:5 or SEQ ID NO:6).

In some embodiments, a rAAV vector genome is linear, single-stranded, and flanked by AAV ITRs. Prior to transcription and translation of the heterologous gene, a single stranded DNA genome of approximately 4700 nucleotides must be converted to a double-stranded form by DNA polymerases (e.g., DNA polymerases within the transduced cell) using the free 3′-OH of one of the self-priming ITRs to initiate second-strand synthesis. In some embodiments, full length-single stranded vector genomes (i.e., sense and anti-sense) anneal to generate a full length-double stranded vector genome. This may occur when multiple rAAV vectors carrying genomes of opposite polarity (i.e., sense or anti-sense) simultaneously transduce the same cell. Regardless of how they are produced, once double-stranded vector genomes are formed, the cell can transcribe and translate the double-stranded DNA and express the heterologous gene.

The efficiency of transgene expression from a rAAV vector can be hindered by the need to convert a single stranded rAAV genome (ssAAV) into double-stranded DNA prior to expression. This step is circumvented by using a self-complementary AAV genome (scAAV) that can package an inverted repeat genome that can fold into double-stranded DNA without the need for DNA synthesis or base-pairing between multiple vector genomes (McCarty, (2008) Molec. Therapy 16(10):1648-1656; McCarty et al., (2001) Gene Therapy 8:1248-1254; McCarty et al., (2003) Gene Therapy 10:2112-2118). A limitation of a scAAV vector is that size of the unique transgene, regulatory elements and IRTs to be package in the capsid is about half the size (i.e., ˜2,500 nucleotides of which 2,200 nucleotides may be a transgene and regulatory elements, plus two copies of the ˜145 nucleotide ITRs) of a ssAAV vector genome (i.e., ˜4,900 nucleotides including two ITRs).

scAAV vector genomes are made by deleting the terminal resolution site (TRS) from one rAAV ITR of the expression plasmid, thereby preventing initiation of replication from that end (see U.S. Pat. No. 8,784,799). AAV replication within a host cell is initiated at the wild type ITR of the genome and continues through the mutant ITR without terminal resolution and then back across the genome to create a dimer. The dimer is a self-complementary genome with a mutant ITR in the middle, and wild-type ITRs at each end. In some embodiments, a mutant ITR with a deleted TRS is at the 5′ end of the vector genome. In some embodiments, a mutant ITR with a deleted TRS is at the 3′ end of the vector genome. In some embodiments, a mutant ITR comprises the nucleic acid sequence of SEQ ID NO:7 or SEQ ID NO:8.

Without wishing to be bound by theory, while the two halves of a scAAV genome are complementary, it is unlikely that there is substantial base pairing within the capsid as many of the bases are in contact with amino acid residues of the inner capsid shell and the phosphate backbone is sequestered toward the center (McCarty (2008) Molec. Therapy 16(10):1648-1656). It is likely that upon uncoating, the two halves of the scAAV genome anneal to form a dsDNA hairpin molecule, with a covalently closed ITR at one end and two open-ended ITRs on the other. The ITRs flank a double-stranded region encoding, among other things, the transgene, and regulatory elements in cis thereto.

A viral capsid of a rAAV vector may be from a wild type AAV or a variant AAV such as AAV1, AAV2, AAV3, AAV3A, AAV3B, AAV4, AAV5, AAV6, AAV7, AAV8, AAV9, AAV10, AAVrh10, AAVrh74 (see WO2016/210170), AAV12, AAV2i8, AAV1.1, AAV2.5, AAV6.1, AAV6.3.1, AAV9.45, RHM4-1 (SEQ ID NO:5 of WO 2015/013313), RHM15-1, RHM15-2, RHM15-3/RHM15-5, RHM15-4, RHM15-6, AAV hu.26, AAV1.1, AAV2.5, AAV6.1, AAV6.3.1, AAV9-45, AAV2i8, AAV29G, AAV2, AAV8G9, AAV-LK03, AAV2-TT, AAV2-TT-S312N, AAV3B-S312N, AAV avian AAV, bovine AAV, canine AAV, equine AAV, primate AAV, non-primate AAV, snake AAV, goat AAV, shrimp AAV, ovine AAV and variants thereof (see, e.g., Fields et al., “Virology”, volume 2, chapter 69 (4^{th} ed., Lippincott-Raven Publishers). Capsids may be derived from a number of AAV serotypes disclosed in U.S. Pat. No. 7,906,111; Gao et al. (2004) J. Virol. 78:6381; Morris et al. (2004) Virol. 33:375; WO 2013/063379; WO 2014/194132; and include true type AAV (AAV-TT) variants disclosed in WO 2015/121501, and RHM4-1, RHM15-1 through RHM15-6, and variants thereof, disclosed in WO 2015/013313, all of which are hereby incorporated by reference. Capsids may also be derived from AAV variants isolated from human CD34+ cell include AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14 and AAVHSC15 (Smith et al. (2014) Molecular Therapy 22(9):1625-1634). Naturally occurring AAVs isolated from human tissues by long-read sequencing include AAVv66 with tropism for the CNS as well as AAVv33, AAVv37, AAVv40, AAVv67, AAVv70, AAVv72, AAVv84, AAVv86, AAVv87 and AAVv90 (Hsu et al. (2020) Nat. Comm. 11:3279). One skilled in the art would know there are likely other AAV variants not yet identified that perform the same or similar function. A full complement of AAV cap proteins includes VP1, VP2, and VP3. The ORF comprising nucleotide sequences encoding AAV VP capsid proteins may comprise less than a full complement AAV Cap proteins or the full complement of AAV cap proteins may be provided.

In some embodiments, the present disclosure provides for the use of ancestral AAV vectors for use in therapeutic in vivo gene therapy. Specifically, in silico-derived sequences may be synthesized de novo and characterized for biological activities. Prediction and synthesis of ancestral sequences, in addition to assembly into a rAAV vector, may be accomplished using methods described in WO 2015/054653, the contents of which are incorporated by reference herein. Notably, rAAV vectors assembled from ancestral viral sequences may exhibit reduced susceptibility to pre-existing immunity in human populations as compared to contemporary viruses or portions thereof.

In some embodiments, a rAAV vector comprising a capsid protein encoded by a nucleotide sequence derived from more than one AAV serotype (e.g., wild type AAV serotypes, variant AAV serotypes) is referred to as a “chimeric vector” or “chimeric capsid” (See U.S. Pat. No. 6,491,907, the entire disclosure of which is incorporated herein by reference). In some embodiments, a chimeric capsid protein is encoded by a nucleic acid sequence derived from 2, 3, 4, 5, 6, 7, 8, 9, 10 or more AAV serotypes. In some embodiments, a recombinant AAV vector includes a capsid sequence derived from e.g., AAV1, AAV2, AAV3, AAV3A, AAV3B, AAV4, AAV5, AAV6, AAV7, AAV8, AAV9, AAV10, AAV11, AAVrh74, AAVrh10, AAV2i8, or variant thereof, resulting in a chimeric capsid protein comprising a combination of amino acids from any of the foregoing AAV serotypes (see, Rabinowitz et al. (2002) J. Virology 76(2):791-801). Alternatively, a chimeric capsid can comprise a mixture of a VP1 from one serotype, a VP2 from a different serotype, a VP3 from yet a different serotype, and a combination thereof. For example, a chimeric virus capsid may include an AAV1 cap protein or subunit and at least one AAV2 cap protein or subunit. A chimeric capsid can, for example include an AAV capsid with one or more B19 cap subunits, e.g., an AAV cap protein or subunit can be replaced by a B19 cap protein or subunit. For example, in one embodiment, a VP3 subunit of an AAV capsid can be replaced by a VP2 subunit of B19.

In some embodiments, chimeric vectors have been engineered to exhibit altered tropism or tropism for a particular tissue or cell type. The term “tropism” refers to preferential entry of the virus into certain cell or tissue types and/or preferential interaction with the cell surface that facilitates entry into certain cell or tissue types. AAV tropism is generally determined by the specific interaction between distinct viral capsid proteins and their cognate cellular receptors (Lykken et al. (2018) J. Neurodev. Disord. 10:16). Preferably, once a virus or viral vector has entered a cell, sequences (e.g., heterologous sequences such as a transgene) carried by the vector genome (e.g., a rAAV vector genome) are expressed.

A “tropism profile” refers to a pattern of transduction of one or more target cells, tissues and/or organs. For example, an AAV capsid may have a tropism profile characterized by efficient transduction of muscle cells with only low transduction of, for example, brain cells.

### III. Recombinant Nucleic Acids

Methods of the present disclosure include purification of a rAAV vector comprising a recombinant nucleic acid including modified nucleic acids as well as plasmids and vector genomes that comprise a modified nucleic acid. A recombinant nucleic acid, plasmid or vector genome may comprise regulatory sequences to modulate propagation (e.g., of a plasmid) and/or control expression of a modified nucleic acid (e.g., a transgene). Recombinant nucleic acids may also be provided as a component of a viral vector (e.g., a rAAV vector). Generally, a viral vector includes a vector genome comprising a recombinant nucleic acid packaged in a capsid.

A. Modified Nucleic Acids

A modified, or variant form, of a gene, nucleic acid or polynucleotide (e.g., a transgene) refers to a nucleic acid that deviates from a reference sequence. A reference sequence may be a naturally occurring, wild type sequence (e.g., a gene) and may include naturally occurring variants (e.g., splice variants, alternative reading frames). Those skilled in the art will be aware that reference sequences can be found in publicly available databases such as GenBank (ncbi.nlm.nih.gov/genbank). Modified/variant nucleic acids may have substantially the same, greater or lesser activity, function or expression as compared to a reference sequence. Preferably, a modified, or variant nucleic acid, as used interchangeably herein, exhibits improved protein expression, e.g., a protein encoded thereby is expressed at a detectably greater level in a cell compared with the level of expression of a protein provided by an endogenous gene (e.g., a wild type gene, a mutant gene) in an otherwise identical cell. In some embodiments, a modified, or variant nucleic acid, as used interchangeably herein, exhibits improved protein expression, e.g., a protein encoded thereby is expressed at a detectably greater level in a cell compared with the level of expression of a protein provided by an endogenous gene comprising a mutation in an otherwise identical cell.

Modifications to nucleic acids include one or more nucleotide substitutions (e.g., substitution of 1-3, 3-5, 5-10, 10-15, 15-20, 20-25, 25-30, 30-40, 40-50, 50-100 or more nucleotides), additions (e.g., insertion of 1-3, 3-5, 5-10, 10-15, 15-20, 20-25, 25-30, 30-40, 40-50, 50-100 or more nucleotides), deletions (e.g., deletion of 1-3, 3-5, 5-10, 10-15, 15-20, 20-25, 25-30, 30-40, 40-50, 50-100 or more nucleotides, deletion of a motif, domain, fragment, etc.) of a reference sequence. A modified nucleic acid may be about 50%, about 60%, about 70%, about 80%, about 85%, about 90%, about 92%, about 93%, about 94%, about 95%, about 96% about 97% about 98% or about 99% identical to a reference sequence.

A modified nucleic acid may encode a polypeptide with about 50%, about 60%, about 70%, about 80%, about 85%, about 90%, about 95%, about 98%, about 99% or 100% identity to a reference polypeptide. In some embodiments, a modified nucleic acid encodes a polypeptide with 100% identify to a reference polypeptide.

In some embodiments, a modified nucleic acid (e.g., transgene) encodes a wild-type protein. Such modified nucleic acid may be codon optimized. “Optimized” or “codon-optimized,” as referred to interchangeably herein, refers to a coding sequence that has been optimized relative to a wild type coding sequence or reference sequence (e.g., a coding sequence for a mini-dystrophin polypeptide, e.g., SEQ ID NO:2) to increase expression of the polypeptide, e.g., by minimizing usage of rare codons, decreasing the number of CpG dinucleotides, removing cryptic splice donor or acceptor sites, removing Kozak sequences, removing ribosomal entry sites, and the like. In some embodiments, a level of expression of a protein from a codon-optimized sequence is increased as compared to a level of expression of a protein from a wild type gene in an otherwise identical cell. In some embodiments, a level of expression of a protein from a codon-optimized sequence is not increased (e.g., expression is substantially similar) as compared to a level of expression of a protein from a wild-type gene in an otherwise identical cell. In some embodiments, a level of expression of a protein from a codon-optimized sequence is increased as compared to a level of expression of a protein from a mutant gene in an otherwise identical cell.

Examples of modifications include elimination of one or more cis-acting motifs and introduction of one or more Kozak sequences. In some embodiments, one or more cis-acting motifs are eliminated and one or more Kozak sequences are introduced.

Examples of cis-acting motifs that may be eliminated include internal TATA-boxes; chi-sites; ribosomal entry sites; ARE, INS, and/or CRS sequence elements; repeat sequences and/or RNA secondary structures; (cryptic) splice donor and/or acceptor sites, branch points; and restriction sites.

In some embodiments, a modified nucleic acid encodes a modified or variant polypeptide. A modified polypeptide (e.g., a codon optimized mini-dystrophin) encoded by a modified nucleic acid may retain all or a part of the function or activity of a polypeptide encoded by a wild type coding or reference sequence. In some embodiments, a modified polypeptide has one or more non-conservative or conservative amino acid changes. In some embodiments, certain domains that have been demonstrated to play a limited or no role in a function of a polypeptide are not present in a modified polypeptide (e.g., certain binding domains) (e.g., WO 2016/097219). Modified nucleic acids present in rAAV vectors may comprise fewer nucleotides than the wild type coding, or reference sequence, due to the packaging capacity of a rAAV capsid (e.g., shortened minidystrophin transgene, see WO 2001/83695; a B-domain deleted human Factor VIII transgene, see WO 2017/074526), and also include shortened transgenes that are both truncated and codon-optimized (e.g., a codon optimized mini-dystrophin transgene described in WO 2017/221145). In some embodiments, a polypeptide encoded by a modified nucleic acid has less than, the same, or greater, but at least a part of, a function or activity of a polypeptide encoded by a reference sequence.

Modified nucleic acids may have a modified GC content (e.g., the number of G and C nucleotides present in a nucleic acid sequence), a modified (e.g., increased or decreased) CpG dinucleotide content and/or a modified (e.g., increased or decreased) codon adaptation index (CAI) relative to a reference and/or wild-type sequence. See, e.g., WO 2017/077451 (discussing various considerations well-known in the art for codon-optimization of nucleic acid sequences of interest, including publicly available software for analyzing nucleic acid sequences for optimization). As used herein, modified refers to a decrease or an increase in a particular value, amount or effect.

In some embodiments, a GC content of a modified nucleic acid sequence of the present disclosure is increased relative to a reference and/or a wild-type gene or coding sequence. The GC content of a modified nucleic acid is at least 5%, at least 6%, at least 7%, at least 8%, at least 9%, at least 10%, at least 12%, at least 14%, at least 15%, at least 17%, at least 20%, at least 30%, at least 40%, at least 50%, at least 60%, at least 70% greater than GC content of a wild type coding sequence. In some embodiments, GC content is expressed as a percentage of G (guanine) and C (cytosine) nucleotides in the sequence.

In some embodiments, a codon adaptation index of a modified nucleic acid sequence of the present disclosure is at least 0.74, at least 0.76, at least 0.77, at least 0.80, at least 0.85, at least 0.86, at least 0.87, at least 0.90, at least 0.95 or at least 0.98.

In some embodiments, a modified nucleic acid sequence of the present disclosure has a reduced level of CpG dinucleotides, that being a reduction of about 10%, 20%, 30%, 50% or more, as compared to a wild type or reference nucleic acid sequence. In some embodiments, a modified nucleic acid has 1-5 fewer, 5-10 fewer, 10-15 fewer, 15-20 fewer, 20-25 fewer, 25-30 fewer, 30-40 fewer, 40-45 fewer or 45-50 fewer, or even fewer di-nucleotides than a reference sequence (e.g., a wild type sequence).

It is known that methylation of CpG dinucleotides plays an important role in the regulation of gene expression in eukaryotes. Specifically, methylation of CpG dinucleotides in eukaryotes essentially serves to silence gene expression through interfering with the transcriptional machinery. As such, because of the gene silencing evoked by methylation of CpG motifs, nucleic acids and vectors having a reduced number of CpG dinucleotides will provide for high and longer-lasting transgene expression level.

Modified nucleic acid sequences may include flanking restriction sites to facilitate subcloning into an expression vector. Many such restriction sites are well known in the art, and include, but are not limited to Aval, Swal, ApaL1 and Xmal.

The present disclosure includes a modified nucleic acid of SEQ ID NO:1 which encodes a functionally active fragment of the dystrophin polypeptide. A “functionally active” or “functional dystrophin polypeptide” indicates that the fragment provides the same or similar biological function and/or activity as a full-length dystrophin polypeptide. That is, the fragment provides the same function including, but not limited to, as a structural protein of myofilaments of a muscle fiber. The biological activity of a functional fragment of dystrophin encompasses reversing or preventing the neuromuscular phenotype associated with Duchenne muscular dystrophy.

Thus, one embodiment of the invention relates to a method of purifying a rAAV vector comprising a modified nucleic acid encoding a mini-dystrophin protein, the nucleic acid comprising, consisting essentially of, or consisting of the nucleic acid sequence of SEQ ID NO:1 or a sequence at least about 90% identical thereto. In some embodiments, the nucleic acid is, is at least, or is at most 90%, 91%, 92%, 93%, 94%, 95%, 96%, 97%, 98%, or 99% identical to the nucleic acid sequence of SEQ ID NO:1. In certain embodiments, the nucleic acid has a length that is within the capacity of a viral vector, e.g., a parvovirus vector, e.g., a rAAV vector. In some embodiments, the nucleic acid is, is atleast, or is at most 5000, 4900, 4800, 4700, 4600, 4500, 4400, 4300, 4200, 4100, or about 4000 nucleotides, or fewer.

In some embodiments, the nucleic acid encodes a mini-dystrophin protein comprising, consisting essentially of, or consisting of the amino acid sequence of SEQ ID NO:2 or a sequence that is, is at least, or is at most 90%, 91%, 92%, 93%, 94%, 95%, 96%, 97%, 98%, or 99% identical to the amino acid sequence of SEQ ID NO:2.

In some embodiments, a nucleic acid (e.g., SEQ ID NO:1) is part of a recombinant nucleic acid for production of dystrophin protein. The recombinant nucleic acid may further comprise regulatory elements useful for increasing expression of dystrophin.

B. Regulatory Elements

Methods of the present disclosure include purification of a rAAV vector comprising a recombinant nucleic acid including a modified nucleic acid encoding a polypeptide (e.g., mini-dystrophin) and various regulatory or control elements. Typically, regulatory elements are nucleic acid sequence(s) that influence expression of an operably linked polynucleotide. The precise nature of regulatory elements useful for gene expression will vary from organism to organism and from cell type to cell type including, for example, a promoter, enhancer, intron etc., with the intent to facilitate proper heterologous polynucleotide transcription and translation. Regulatory control can be affected at the level of transcription, translation, splicing, message stability, etc. Typically, a regulatory control element that modulates transcription is juxtaposed near the 5′ end of the transcribed polynucleotide (i.e., upstream). Regulatory control elements may also be located at the 3′ end of the transcribed sequence (i.e., downstream) or within the transcript (e.g., in an intron). Regulatory control elements can be located at a distance away from the transcribed sequence (e.g., 1 to 100, 100 to 500, 500 to 1000, 1000 to 5000, 5000 to 10,000 or more nucleotides). However, due to the length of an AAV vector genome, regulatory control elements are typically within 1 to 1000 nucleotides from the polynucleotide.

C. Promoter

As used herein, the term “promoter,” such as a “eukaryotic promoter,” refers to a nucleotide sequence that initiates transcription of a particular gene, or one or more coding sequences (e.g., an mini-dystrophin coding sequence), in eukaryotic cells (e.g., a muscle cell). A promoter can work with other regulatory elements or regions to direct the level of transcription of the gene or coding sequence(s). These regulatory elements include, for example, transcription binding sites, repressor and activator protein binding sites, and other nucleotide sequences known to act directly or indirectly to regulate the amount of transcription from the promoter, including, for example, attenuators, enhances and silencers. The promoter is most often located on the same strand and near the transcription start site, 5′ of the gene or coding sequence to which it is operably linked. A promoter is generally 100-1000 nucleotides in length. A promoter typically increases gene expression relative to expression of the same gene in the absence of a promoter.

As used herein, a “core promoter” or “minimal promoter” refers to the minimal portion of a promoter sequence required to properly initiate transcription. It may include any of the following: a transcription start site, a binding site for RNA polymerase and a general transcription factor binding site. A promoter may also comprise a proximal promoter sequence (5′ of a core promoter) that contains other primary regulatory elements (e.g., enhancer, silencer, boundary element, insulator) as well as a distal promoter sequence (3′ of a core promoter).

Examples of a suitable promoter include adenoviral promoters, such as the adenoviral major late promoter; heterologous promoters, such as the cytomegalovirus (CMV) promoter; the respiratory syncytial virus promoter; the Rous Sarcoma Virus (RSV) promoter; the albumin promoter; inducible promoters, such as the Mouse Mammary Tumor Virus (MMTV) promoter; the metallothionein promoter; heat shock promoters; the α-1-antitrypsin promoter; the hepatitis B surface antigen promoter; the transferrin promoter; the apolipoprotein A-1 promoter; chicken β-actin (CBA) promoter; the elongation factor 1a (EF1a) promoter; the hybrid form of the CBA promoter (CBh promoter); the CAG promoter (cytomegalovirus early enhancer element and promoter, the first exon, and the first intron of chicken beta-actin gene and the splice acceptor of the rabbit beta-globin gene) (Alexopoulou et al. (2008) BioMed. Central Cell Biol. 9:2); a creatine kinase promoter; and the human dystrophin gene promoter. In some embodiments of the present disclosure, a eukaryotic promoter sequence (e.g., a creatine kinase promoter) is operably linked to a modified nucleic acid encoding mini-dystrophin.

A promoter may be constitutive, tissue-specific or regulated. Constitutive promoters are those which cause an operably linked gene to be expressed essentially at all times. In some embodiments, a constitutive promoter is active in most eukaryotic tissues under most physiological and developmental conditions.

Regulated promoters are those which can be activated or deactivated. Regulated promoters include inducible promoters, which are usually “off,” but which may be induced to turn “on,” and “repressible” promoters, which are usually “on,” but may be turned “off.” Many different regulators are known, including temperature, hormones, cytokines, heavy metals and regulatory proteins. The distinctions are not absolute; a constitutive promoter may often be regulated to some degree. In some cases, an endogenous pathway may be utilized to provide regulation of the transgene expression, e.g., using a promoter that is naturally downregulated when the pathological condition improves.

A tissue-specific promoter is a promoter that is active in only specific types of tissues, cells or organs. Typically, a tissue-specific promoter is recognized by transcriptional activator elements that are specific to a particular tissue, cell and/or organ. For example, a tissue-specific promoter may be more active in one or several particular tissues (e.g., two, three or four) than in other tissues. In some embodiments, expression of a gene modulated by a tissue-specific promoter is much higher in the tissue for which the promoter is specific than in other tissues. In some embodiments, there may be little, or substantially no activity, of the promoter in any tissue other than the one for which it is specific. A promoter may be a tissue-specific promoter, such as the mouse albumin promoter, or the transthyretin promoter (TTR), which are active in liver cells. Other examples of tissue specific promoters include promoters from genes encoding skeletal α-actin, myosin light chain 2A, dystrophin, muscle creatine kinase which induce expression in skeletal muscle (Li et al. (1999) Nat. Biotech. 17:241-245). Liver specific expression may be induced using promoters from the albumin gene (Miyatake et al. (1997) J. Virol. 71:5124-5132), hepatitis B. virus core promoter (Sandig, et al. (1996) Gene Ther. 3:1002-1009) and alpha-fetoprotein (Arbuthnot et al., (1996) Hum. Gene. Ther. 7:1503-1514).

D. Enhancer

In another aspect, a modified nucleic acid encoding a therapeutic polypeptide further comprises an enhancer to increase expression of the therapeutic polypeptide. Typically, an enhancer element is located upstream of a promoter element but may also be located downstream or within another sequence (e.g., a transgene). An enhancer may be located 100 nucleotides, 200 nucleotides, 300 nucleotides or more upstream or downstream of a modified nucleic acid. An enhancer typically increases expression of a modified nucleic acid (e.g., encoding a therapeutic polypeptide) beyond the increased expression provided by a promoter element alone.

Many enhancers are known in the art, including, but not limited to, the cytomegalovirus major immediate-early enhancer. More specifically, the cytomegalovirus (CMV) MIE promoter comprises three regions: the modulator, the unique region and the enhancer (Isomura et al. (2003) J. Virol. 77(6):3602-3614). The CMV enhancer region can be combined with another promoter, or a portion thereof, to form a hybrid promoter to further increase expression of a nucleic acid operably linked thereto. For example, a chicken R-actin (CBA) promoter, or a portion thereof, can be combined with a CMV promoter/enhancer, or a portion thereof, to make a version of CBA termed the “CBh” promoter, which stands for chicken beta-actin hybrid promoter, as described in Gray et al. (2011) Human Gene Therapy 22:1143-1153. Like promoters, enhancers may be constitutive, tissue-specific or regulated.

In some embodiments of the present disclosure, a regulatory element comprises a hybrid enhancer and promoter, such as a synthetic hybrid enhancer and promoter derived from the creatine kinase (CK) gene which serves as a muscle specific transcription regulatory element (hCK) and which is operably linked to a modified nucleic acid encoding mini-dystrophin.

E. Fillers, Spacers and Stuffers

As disclosed herein, a recombinant nucleic acid intended for use in a rAAV vector may include an additional nucleic acid element to adjust the length of the nucleic acid to near, or at the normal size (e.g., approximately 4.7 to 4.9 kilobases), of the viral genomic sequence acceptable for AAV packaging into a rAAV vector (Grieger et al. (2005) J. Virol. 79(15):9933-9944). Such a sequence may be referred to interchangeably as filler, spacer or stuffer. In some embodiments, filler DNA is an untranslated (non-protein coding) segment of nucleic acid. In some embodiments, a filler or stuffer polynucleotide sequence is a sequence between about 1-10, 10-20, 20-30, 30-40, 40-50, 50-60, 60-70, 70-80, 80-90-90-100, 100-150, 150-200, 200-250, 250-300, 300-400, 400-500, 500-750, 750-1000, 1000-1500, 1500-2000, 2000-3000 or more in length.

AAV vectors typically accept inserts of DNA having a size ranging from about 4 kb to about 5.2 kb or about 4.1 to 4.9 kb for optimal packaging of the nucleic acid into the AAV capsid. In some embodiments, a rAAV vector comprises a vector genome having a total length between about 3.0 kb to about 3.5 kb, about 3.5 kb to about 4.0 kb, about 4.0 kb to about 4.5 kb, about 4.5 kb to about 5.0 kb or about 5.0 kb to about 5.2 kb. In some embodiments, a rAAV vector comprises a vector genome having a total length of about 4.5 kb. In some embodiments, a rAAV vector comprises a vector genome that is self-complementary. While the total length of a self-complementary (sc) vector genome in a rAAV vector is equivalent to a single-stranded (ss) vector genome (i.e., from about 4 kb to about 5.2 kb), the nucleic acid sequence (i.e., comprising the transgene, regulatory elements and ITRs) encoding the sc vector genome must be only half as long as a nucleic acid sequence encoding a ss vector genome in order for the sc vector genome to be packaged in the capsid.

F. Introns and Exons

In some embodiments, a recombinant nucleic acid includes, for example, an intron, exon and/or a portion thereof. An intron may function as a filler or stuffer polynucleotide sequence to achieve an appropriate length for vector genome packaging into a rAAV vector. An intron and/or an exon sequence can also enhance expression of a polypeptide (e.g., a transgene) as compared to expression in the absence of the intron and/or exon element (Kurachi et al. (1995) J. Biol. Chem. 270 (10):576-5281; WO 2017/074526). Furthermore, filler/stuffer polynucleotide sequences (also referred to as “insulators”) are well known in the art and include, but are not limited to, those described in WO 2014/144486 and WO 2017/074526.

An intron element may be derived from the same gene as a heterologous polynucleotide, or derived from a completely different gene or other DNA sequence (e.g., chicken R-actin gene, minute virus of mice (MVM)). In some embodiments, a recombinant nucleic acid includes at least one element selected from an intron and an exon derived from a non-cognate gene (i.e., not derived from the modified nucleic acid, e.g., transgene).

G. Polyadenylation Signal Sequence (polyA)

Further regulatory elements may include a stop codon, a termination sequence, and a polyadenylation (polyA) signal sequence, such as, but not limited to a bovine growth hormone poly A signal sequence (BHG polyA). A polyA signal sequence drives efficient addition of a poly-adenosine “tail” at the 3′ end of a eukaryotic mRNA which guides termination of gene transcription (see, e.g., Goodwin et al. (1992) J. Biol. Chem. 267(23):16330-16334). A polyA signal acts as a signal for the endonucleolytic cleavage of the newly formed precursor mRNA at its 3′ end and for addition to this 3′ end of an RNA stretch consisting only of adenine bases. A polyA tail is important for the nuclear export, translation and stability of mRNA. In some embodiments, a poly A is a SV40 early polyadenylation signal, a SV40 late polyadenylation signal, an HSV thymidine kinase polyadenylation signal, a protamine gene polyadenylation signal, an adenovirus 5 E1b polyadenylation signal, a growth hormone polyadenylation signal, a PBGD polyadenylation signal or an in silico designed polyadenylation signal.

In some embodiments, and optionally in combination with one or more other regulatory elements described herein, a polyA signal sequence of a recombinant nucleic acid is a polyA signal that is capable of directing and effecting the endonucleolytic cleavage and polyadenylation of the precursor mRNA resulting from the transcription of a modified nucleic acid encoding mini-dystrophin (e.g., SEQ ID NO:2).

In some embodiments, a rAAV 9 vector with tropism for muscle cells, contains a vector genome comprising AAV ITRs (e.g., AAV2 ITRs) and a recombinant nucleic acid comprising a modified (i.e., codon-optimized) nucleic acid encoding mini-dystrophin and at least one of the following regulatory elements: a promoter (e.g., a human CK promoter), a hybrid enhancer and promoter and a poly A (SEQ ID NO:4).

### IV. Assembly of Viral Vectors

A viral vector (e.g., rAAV vector) carrying a transgene (e.g., encoding mini-dystrophin) is assembled from a polynucleotide encoding a transgene, suitable regulatory elements and elements necessary for production of viral proteins which mediate cell transduction. Examples of a viral vector include but are not limited to adenoviral, retroviral, lentiviral, herpesvirus and adeno-associated virus (AAV) vectors, and in particular rAAV vector (as discussed, supra).

A vector genome component of a rAAV vector produced according to the methods of the disclosure include at least one transgene, e.g., a codon optimized mini-dystrophin transgene and associated expression control sequences for controlling expression of the modified nucleic acid encoding dystrophin, or a fragment thereof.

In an exemplary non-limiting embodiment, a vector genome includes a portion of a parvovirus genome, such as an AAV genome with rep and cap deleted and/or replaced by a modified nucleic acid (e.g., transgene, e.g., a codon optimized mini-dystrophin transgene) and its associated expression control sequences. A modified nucleic acid encoding dystrophin, or a fragment thereof, is typically inserted adjacent to one or two (i.e., is flanked by) AAV ITRs or ITR elements adequate for viral replication (Xiao et al. (1997) J. Virol. 71(2): 941-948), in place of the nucleic acid encoding viral rep and cap proteins. Other regulatory sequences suitable for use in facilitating tissue-specific expression of a codon optimized mini-dystrophin transgene in the target cell (e.g., muscle cell) may also be included.

A. Packaging Cell

One skilled in the art would appreciate that a rAAV vector comprising a transgene, and lacking virus proteins needed for viral replication (e.g., cap and rep), cannot replicate since such proteins are necessary for virus replication and packaging. Cap and rep genes may be supplied to a cell (e.g., a host cell, e.g., a packaging cell) as part of a plasmid that is separate from a plasmid supplying the vector genome with the transgene.

“Packaging cell” or “producer cell” means a cell or cell line which may be transfected with a vector, plasmid or DNA construct, and provides in trans all the missing functions which are required for the complete replication and packaging of a viral vector. The required genes for rAAV vector assembly include the vector genome (e.g., a codon optimized mini-dystrophin transgene, regulatory elements, and ITRs), AAV rep gene, AAV cap gene, and certain helper genes from other viruses such as, e.g., adenovirus. One of ordinary skill would understand that the requisite genes for AAV production can be introduced into a packaging cell in various ways including, for example, transfection of one or more plasmids. However, in some embodiments, some genes (e.g., rep, cap, helper) may already be present in a packaging cell, either integrated into the genome or carried on an episome. In some embodiments, a packaging cell expresses, in a constitutive or inducible manner, one or more missing viral functions.

Any suitable packaging cell known in the art may be employed in the production of a packaged viral vector. Mammalian cells or insect cells are preferred. Examples of cells useful for the production of a packaging cell in the practice of the disclosure include, for example, human cell lines, such as PER.C6, W138, MRC5, A549, HEK293 (which express functional adenoviral E1 under the control of a constitutive promoter), B-50 or any other HeLa cell, HepG2, Saos-2, HuH7, and HT1080 cell lines. Suitable non-human mammalian cell lines include, for example, VERO, COS-1, COS-7, MDCK, BHK21-F, HKCC or CHO cells.

In some embodiments, a packaging cell is capable of growing in suspension culture. In some embodiments, a packaging cell is capable of growing in serum-free media. For example, HEK293 cells are grow in suspension in serum free medium. In another embodiment, a packaging cell is a HEK293 cell as described in U.S. Pat. No. 9,441,206 and deposited as American Type Culture Collection (ATCC) No. PTA 13274. Numerous rAAV packaging cell lines are known in the art, including, but not limited to, those disclosed in WO 2002/46359.

A cell line for use as a packaging cell includes insect cell lines. Any insect cell which allows for replication of AAV and which can be maintained in culture can be used in accordance with the present disclosure. Examples include Spodoptera frugiperda, such as the Sf9 or Sf21 cell lines, Drosophila spp. cell lines, or mosquito cell lines, e.g., Aedes albopictus derived cell lines. A preferred cell line is the Spodoptera frugiperda Sf9 cell line. The following references are incorporated herein for their teachings concerning use of insect cells for expression of heterologous polypeptides, methods of introducing nucleic acids into such cells, and methods of maintaining such cells in culture: “Methods in Molecular Biology”, ed. Richard, Humana Press, NJ (1995); O'Reilly et al., “Baculovirus Expression Vectors: A Laboratory Manual”, Oxford Univ. Press (1994); Samulski et al. (1989) J. Virol. 63:3822-3828; Kajigaya et al. (1991) Proc. Nat'l. Acad. Sci. USA 88: 4646-4650; Ruffing et al. (1992) J. Virol. 66:6922-6930; Kimbauer et al. (1996) Virol. 219:37-44; Zhao et al. (2000) Virol. 272:382-393; and U.S. Pat. No. 6,204,059.

As a further alternative, viral vectors of the disclosure may be produced in insect cells using baculovirus vectors to deliver the rep/cap genes and rAAV template as described, for example, by Urabe et al. (2002) Human Gene Therapy 13:1935-1943. When using baculovirus production for AAV, in some embodiments, a vector genome is self-complementary. In some embodiments, a host cell is a baculovirus-infected cell (e.g., an insect cell) comprising, optionally, additional nucleic acids encoding baculovirus helper functions, thereby facilitating production of a viral capsid.

A packaging cell generally includes one or more viral vector functions along with helper functions and packaging functions sufficient to result in replication and packaging of the viral vector. These various functions may be supplied together, or separately, to the packaging cell using a genetic construct such as a plasmid or an amplicon, and they may exist extrachromosomally within the cell line, or integrated into the host cell's chromosomes. In some embodiments, a packaging cell is transfected with at least i) a plasmid comprising a vector genome comprising a transgene and AAV ITRs and further comprising at least one of the following regulatory elements: an enhancer, a promoter, an exon, an intron and a poly A and ii) a plasmid comprising a rep gene (e.g., AAV2 rep) and a cap gene (e.g., AAV9 or other cap).

In some embodiments, a host cell is supplied with one or more of the packaging or helper functions incorporated within, e.g., a host cell line with one or more vector functions incorporated extrachromosomally or integrated into the cell's chromosomal DNA.

B. Helper Function

AAV is a dependovirus in that it cannot replicate in a cell without co-infection of the cell by a helper virus. Helper functions include helper virus elements needed for establishing active infection of a packaging cell, which is required to initiate packaging of the viral vector. Helper viruses include, typically, adenovirus or herpes simplex virus. Adenovirus helper functions typically include adenovirus components adenovirus early region 1A (E1 a), E1 b, E2a, E4, and viral associated (VA) RNA. Helper functions (e.g., E1a, E1b, E2a, E4, and VA RNA) can be provided to a packaging cell by transfecting the cell with one or more nucleic acids encoding various helper elements. Alternatively, a host cell (e.g., a packaging cell) can comprise a nucleic acid encoding the helper protein. For instance, HEK293 cells were generated by transforming human cells with adenovirus 5 DNA and now express a number of adenoviral genes, including, but not limited to E1 and E3 (see, e.g., Graham et al. (1977) J. Gen. Virol. 36:59-72). Thus, those helper functions can be provided by the HEK 293 packaging cell without the need of supplying them to the cell by, e.g., a plasmid encoding them. In some embodiments, a packaging cell is transfected with at least i) a plasmid comprising a vector genome comprising a transgene and AAV ITRs and further comprising at least one of the following regulatory elements: an enhancer, a promoter, an exon, an intron and a poly A and ii) a plasmid comprising a rep gene (e.g., AAV2 rep) and a cap gene (e.g., AAV9 or other cap) and iii) a plasmid comprising a helper function.

Any method of introducing a nucleotide sequence carrying a helper function into a cellular host for replication and packaging may be employed, including but not limited to, electroporation, calcium phosphate precipitation, microinjection, cationic or anionic liposomes, a carrier molecule (e.g., polyethylenimine (PEI)) and liposomes in combination with a nuclear localization signal. In some embodiments, helper functions are provided by transfection using a virus vector, or by infection using a helper virus, standard methods for producing viral infection may be used.

The vector genome may be any suitable recombinant nucleic acid, such as a DNA or RNA construct and may be single stranded, double stranded, or duplexed (i.e., self-complementary as described in WO 2001/92551).

### V. Production of Packaged Viral Vector

Viral vectors can be made by several methods known to skilled artisans (see, e.g., WO 2013/063379). An exemplary non-limiting method is described in Grieger, et al. (2015) Molecular Therapy 24(2):287-297, the contents of which are incorporated by reference herein for all purposes. Briefly, efficient transfection of HEK293 cells is used as a starting point, wherein an adherent HEK293 cell line from a qualified clinical master cell bank is used to grow in animal component-free suspension conditions in shaker flasks and WAVE bioreactors that allow for rapid and scalable rAAV production. Using a triple transfection method (e.g., WO 96/40240), a HEK293 cell line suspension can generate greater than 1×10⁵ vector genome containing particles (VG)/cell, or greater than 1×10¹⁴ VG/L of cell culture, when harvested 48 hours post-transfection. More specifically, triple transfection refers a method whereby a packaging cell is transfected with three plasmids: one plasmid encodes the AAV rep and cap (e.g., AAV9 cap) genes, another plasmid encodes various helper functions (e.g., adenovirus or HSV proteins such as E1a, E1b, E2a, E4, and VA RNA, and another plasmid encodes a transgene (e.g., dystrophin, or a fragment thereof) and various elements to control expression of the transgene.

Single-stranded vector genomes are packaged into capsids as the plus strand or minus strand in about equal proportions. In some embodiments of a rAAV vector, a vector genome is in the plus strand polarity (i.e., the sense or coding sequence of the DNA strand). In some embodiments a rAAV vector, a vector is in the minus strand polarity (i.e., the antisense or template DNA strand). Given the nucleotide sequence of a plus strand in its 5′ to 3′ orientation, the nucleotide sequence of a minus strand in its 5′ to 3′ orientation can be determined as the reverse-complement of the nucleotide sequence of the plus strand.

To achieve the desired yields, a number of variables are optimized such as selection of a compatible serum-free suspension media that supports both growth and transfection, selection of a transfection reagent, transfection conditions and cell density.

A rAAV vector may be purified by methods standard in the art such as by column chromatography or cesium chloride gradients. Methods for purifying rAAV vectors are known in the art and include methods described in Clark et al. (1999) Human Gene Therapy 10(6):1031-1039; Schenpp et al. (2002) Methods Mol. Med. 69:427-443; U.S. Pat. No. 6,566,118 and WO 98/09657.

After rAAV vectors of the present disclosure have been produced and purified according to methods disclosed herein, they can be titered (e.g., the amount of rAAV vector in a sample can be quantified) to prepare compositions for administration to subjects, such as human subjects with Duchenne muscular dystrophy. rAAV vector titering can be accomplished using methods know in the art.

In some embodiments, the number of viral particles, including particles containing a vector genome and “empty” capsids that do not contain a vector genome, can be determined by electron microscopy, e.g., transmission electron microscopy (TEM). Such a TEM-based method can provide the number of vector particles (or virus particles in the case of wild type AAV) in a sample. In some embodiments, the amount of particles, containing a vector genome (full capsids), and “empty” capsids that do not contain a vector genome, can be determined by charge detection mass spectrometry, analytical ultracentrifugation (AUC), and/or measurement of absorbance at 260 nm and 280 nm to determine A260/A280 ratio.

In some embodiments, rAAV vector genomes can be titered using quantitative PCR (qPCR) using primers against sequences in the vector genome, for example ITR sequences (e.g., SEQ ID NO:5 or SEQ ID NO:6), and/or sequences in the transgene (or regulatory elements). By performing qPCR in parallel on dilutions of a standard of known concentration, such as a plasmid containing the sequence of the vector genome, a standard curve can be generated permitting the concentration of the rAAV vector to be calculated as the number of vector genomes (VG) per unit volume such as microliters or milliliters. By comparing the number of vector particles as measured by, e.g., SEC or ELISA, to the number of vector genomes in a sample, the percentage of empty capsids can be estimated. Because the vector genome contains the therapeutic transgene, vg/kg or vg/ml of a vector sample may be more indicative of the therapeutic amount of the vector that a subject will receive than the number of vector particles, some of which may be empty and not contain a vector genome. Once the concentration of rAAV vector genomes in the stock solution is determined, it can be diluted into or dialyzed against suitable buffers for use in preparing a composition (e.g., a drug substance) for administration to subjects (e.g., subjects with Duchenne muscular dystrophy).

### VI. AAV Vector Purification by Affinity Chromatography

A novel, purification strategy, based on affinity chromatography methods, may be used to generate high purity AAV vector preparations. In some embodiments, a method is used to generate high purity rAAV vector preparations. In some embodiments, such a method is universal with respect to AAV serotype and/or chimerism of a capsid. In some embodiments, a method is used to generate high purity rAAV vector preparations of various AAV serotypes and/or from chimeric capsids (e.g., AAV1, AAV2, AAV3 including AAV3A and AAV3B, AAV4, AAV5, AAV6, AAV7, AAV8, AAV9, AAV10, AAV12, AAVrh10, AAVrh74, avian AAV, bovine AAV, canine AAV, equine AAV, primate AAV, non-primate AAV, and ovine AAV, and recombinantly produced variants (e.g., capsid variants with insertions, deletions and substitutions, etc.), such as variants referred to as AAV2i8, NP4, NP22, NP66, AAVDJ, AAVDJ/8, AAVDJ/9, AAVLK03, RHM4-1, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14, and AAVHSC15). In some embodiments, a method can be applied to purify rAAV9 vector. Scalable manufacturing technology, as described herein, may be used to manufacture Good Manufacturing Practice (GMP) clinical and commercial grade rAAV vectors to treat disease (e.g., DMD).

In some instances, production of recombinant AAV vector (e.g. rAAV) for gene therapy requires purification of a rAAV vector from a host cell (e.g., host cell debris including but not limited to DNA, RNA, proteins, lipids, membrane and organelles of a host cell) that produced the vector, as well as removal of capsids that do not contain a complete vector genome (e.g., intermediate and/or empty capsids) and thus, do not comprise a therapeutic transgene.

Such purification methods generally comprise multiple steps including, for example, lysis of the host cell, precipitation of host cell protein and nucleic acids, separation of a AAV vector (e.g. rAAV) from host cell protein and nucleic acids, and separation of full rAAV vector (e.g. rAAV9) from empty capsids by column purification, low speed centrifugation, ultracentrifugation, normal flow filtration, ultrafiltration/diafiltration, or any combinations of these methods. Column purification may include, for example, ion exchange chromatography (e.g., anion, cation), affinity chromatography, size exclusion chromatography, multimodal chromatography, and/or hydrophobic interaction chromatography. Centrifugation methods may include, for example, ultracentrifugation or low speed centrifugation (e.g., for removal of solids and clarification). Filtration methods may include but are not limited to diafiltration, depth filtration, nominal filtration and/or absolute filtration.

In some embodiments, a rAAV vector can be purified by affinity chromatography. In some embodiments, a rAAV vector can be purified by affinity chromatography by, loading a solution comprising the rAAV vector onto a stationary phase, and eluting the rAAV vector from the stationary phase to produce an affinity eluate. In some embodiments, a rAAV vector comprises a capsid protein from a AAV serotype selected from the group consisting of AAV1, AAV2, AAV3 (including AAV3A and AAV3B), AAV4, AAV5, AAV6, AAV7, AAV8, AAV9, AAV10, AAV12, AAVrh8, AAVrh10, AAVrh39, AAVrh43, AAVrh74, AAVrh32.22, AAV1.1, AAV2.5, AAV6.1, AAV6.2, AAV6.3.1, AAV9.45, AAVShH10, HSC15/17, RHM4-1, RHM15-1, RHM15-2, RHM15-3/RHM15-5, RHM15-4, RHM15-6, AAVhu.26, AAV2i8, AAV29G, AAV2, AAV8G9, AAV-LK03, AAV2-TT, AAV2-TT-S312N, AAV3B-S312N, AAVavian, AAVbat, AAVbovine, AAVcanine, AAVequine, AAVprimate, AAVnon-primate, AAVovine, AAVmuscovy duck, AAVporcine4, AAVporcine5, AAVsnake NP4, NP22, NP66, AAVDJ, AAVDJ/8, AAVDJ/9, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14, AAVHSC15, AAVv66, AAVv33, AAVv37, AAVv40, AAVv67, AAVv70, AAVv72, AAVv84, AAVv86, AAVv87 and AAVv90. In some embodiments, an AAV serotype can be AAV9. In some embodiments, an AAV capsid comprises a VP1 protein comprising an amino acid sequence that is at least 80%, 85% 90%, 95%, 98%, 99% or 100% identical to SEQ ID NO:3. In some embodiments, an AAV capsid comprises a VP1 protein comprising or consisting of the amino acid sequence of SEQ ID NO:3. In some embodiments, a rAAV vector comprises a mini-dystrophin transgene. In some embodiments, a mini-dystrophin transgene comprises or consists of the nucleic acid sequence of SEQ ID NO:1. In some embodiments, a mini-dystrophin transgene encodes a protein comprising or consisting of the amino acid sequence of SEQ ID NO:2. In some embodiments, a rAAV vector comprises a nucleic acid comprising or consisting of the nucleic acid sequence of SEQ ID NO:4.

A. Affinity Chromatography Column

In some instances, a method herein uses affinity chromatography to purify rAAV vectors, or as a step in a purification process for rAAV vectors. Affinity chromatography uses a stationary phase with an affinity for a target macromolecule in a mobile phase. A stationary phase can have an affinity for a target macromolecule by containing a macromolecule bound in or to a stationary phase matrix, where a bound macromolecule has a specific binding affinity to the target macromolecule(s). Affinity chromatography employs specific macromolecular binding affinity between macromolecules to separate substances (e.g., AAV capsids, DNA, protein, high molar mass species, amino acids) in a mobile phase.

An affinity chromatography stationary phase can be present in a chromatography column. In some embodiments, a column can have a volume of about, at least about, or at most about 0.1, 0.5, 1, 2, 3, 4, 5, 6, 7, 8, 9, 10, 11, 12, 13, 14, 15, 15.5, 16, 16.5, 17, 17.5, 18, 18.5, 19, 19.5, 20, 21, 22, 23, 24, 25, 26, 27, 28, 29, 30, 35, 40, 45, 50, 55, 60, 70, 80, 90, 100, 105, 110, 115, 120, 125,130, 135, 140, 145, 146,147, 148, 149, 150, 151,152, 153, 154, 155, 156, 157, 158, 159, 160,170, 180, 190, 200, 300, 400, 500, 600, 700, 800, 900, 997, 1000, 1500, 2000, 2500, 3000, 3500, 4000, 4500, 5000, 6000, 7000, 8000, 9000, 10000, 11000, 12000, 13000, 14000, 15000, 16000, 17000, 18000, 19000, 20000, 30000, 40000, 50000, or 60000 mL (or any range derivable therein). In some embodiments, a column can have a volume of about 2670 mL. In some embodiments, a column can have a volume of about 17,663 mL. In some embodiments, a column can have a volume of about 25,610 mL. In some embodiments, a column can have an inner diameter of about, at least about, or at most about 1, 2, 3, 4, 5, 6, 7, 8, 9, 10, 11, 12, 13, 14, 15, 15.5, 16, 16.5, 17, 17.5, 18, 18.5, 19, 19.5, 20, 21, 22, 23, 24, 25, 26, 27, 28, 29, 30, 35, 40, 45, 50, 55, 60, 70, 80, 90, or 100 cm (or any range derivable therein). In some embodiments, a column can have a bed height of about, at least about, or at most about 1, 2, 3, 4, 5, 6, 7, 8, 9, 10, 11, 12, 13, 14, 15, 15.5, 16, 16.5, 17, 17.5, 18, 18.5, 19, 19.5, 20, 21, 22, 23, 24, 25, 26, 27, 28, 29, 30, 35, 40, 45, 50, 55, 60, 70, 80, 90, or 100 cm (or any range derivable therein).

B. Stationary Phase

In some embodiments, a stationary phase is an affinity chromatography stationary phase that binds an AAV capsid. In some embodiments, an affinity chromatography stationary phase is in a column. In some embodiments, a stationary phase used contains macromolecule(s) that can bind an AAV capsid. In some embodiments, a stationary phase used contains one or more antigen-binding domain that binds an AAV capsid. In some embodiments, a stationary phase used contains one or more antibody that can bind an AAV capsid. An AAV capsid can be a capsid of an rAAV vector. An AAV capsid can be of various AAV serotypes and/or from chimeric capsids (e.g., AAV1, AAV2, AAV3 (including AAV3A and AAV3B), AAV4, AAV5, AAV6, AAV7, AAV8, AAV9, AAV10, AAV12, AAVrh8, AAVrh10, AAVrh39, AAVrh43, AAVrh74, AAVrh32.22, AAV1.1, AAV2.5, AAV6.1, AAV6.2, AAV6.3.1, AAV9.45, AAVShH10, HSC15/17, RHM4-1, RHM15-1, RHM15-2, RHM15-3/RHM15-5, RHM15-4, RHM15-6, AAVhu.26, AAV2i8, AAV29G, AAV2, AAV8G9, AAV-LK03, AAV2-TT, AAV2-TT-S312N, AAV3B-S312N, AAVavian, AAVbat, AAVbovine, AAVcanine, AAVequine, AAVprimate, AAVnon-primate, AAVovine, AAVmuscovy duck, AAVporcine4, AAVporcine5, AAVsnake NP4, NP22, NP66, AAVDJ, AAVDJ/8, AAVDJ/9, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14, AAVHSC15, AAVv66, AAVv33, AAVv37, AAVv40, AAVv67, AAVv70, AAVv72, AAVv84, AAVv86, AAVv87 and AAVv90). In some embodiments, a stationary phase is an affinity chromatography stationary phase that binds a rAAV9 capsid. In some embodiments, a stationary phase comprises a macromolecule (e.g., an antibody) that binds an AAV capsid. In some embodiments, a stationary phase used contains an antibody that can bind a rAAV9 capsid.

In some embodiments, an antibody can be a monoclonal antibody, a polyclonal antibody, a recombinant antibody, a single domain antibody, etc., or a combination thereof. In some embodiments, an antibody can be a camelid-derived single domain antibody (e.g., V_{H}H).

In some embodiments, an affinity chromatography stationary phase matrix is a resin. In some instances, a matrix is an agarose, a cellulose, a dextran, a polyacrylamide, a latex, or a porous glass, or a combination thereof. In some instances, a matrix comprises porous polystyrenedivinylbenzene beads. In some instances, polystyrenedivinylbenzene beads are modified with a Camelid-derived single domain antibody (e.g., V_{H}H) ligand. In some embodiments, an affinity chromatography stationary phase is POROS™ CaptureSelect™ AAV9 affinity resin. In some embodiments, an affinity chromatography stationary phase is POROS™ CaptureSelect™ AAVX affinity resin.

In some aspects, a stationary phase has a dynamic binding capacity of greater than 1.0×10¹³ vg/mL stationary phase. In some aspects, a stationary phase has a dynamic binding capacity of about, at least about, or at most about 1.0×10¹³, 2.0×10¹³, 3.0×10¹³, 4.0×10¹³, 5.0×10¹³, 6.0×10¹³, 7.0×10¹³, 8.0×10¹³, 9.0×10¹³, 1.0×10¹⁴, 2.0×10¹⁴, 3.0×10¹⁴, 4.0×10¹⁴, 5.0×10¹⁴, 6.0×10¹⁴, 7.0×10¹⁴, 8.0×10¹⁴, 9.0×10¹⁴, or 1.0×10¹⁵ vg/mL stationary phase or greater (or any range derivable therein).

C. Loading of an Affinity Chromatography Stationary Phase

A method of purifying an AAV vector (e.g. rAAV) by affinity chromatography comprises a step of loading a solution comprising a AAV vector, e.g., a substance to be purified, onto an affinity chromatography stationary phase in a chromatography column. In some embodiments, a solution comprising a rAAV vector comprises host cell protein and host cell DNA. A step of loading may be performed by gravity feeding a load (e.g., a solution comprising the AAV vector) onto the column and/or pumping the load onto the column.

In some embodiments, a solution comprising an AAV vector can be loaded onto an affinity chromatography stationary phase at a linear velocity of about, at least about, or at most about 5, 10, 20, 30, 40, 50, 60, 70, 80, 90, 100, 150, 170, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 700, 750 or 800 cm/hr (or any range derivable therein). In some embodiments, loading of a solution comprising an AAV vector to a stationary phase can be performed at residence time on the stationary phase of about, at least about, or at most about 0.1, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 min/column volume (CV) of the solution (or any range derivable therein). In some embodiments, an amount of a solution comprising an AAV vector that is loaded onto a stationary phase is about, at least about, or at most about 0.05, 0.1, 0.5, 1, 2, 3, 4, 5, 6, 7, 8, 9, 10, 11, 12, 13, 14, 15, 15.5, 16, 16.5, 17, 17.5, 18, 18.5, 19, 19.5, or 20 CV (or any range derivable therein),

In some embodiments, a solution comprising a AAV vector can comprise “supernatant from a cell lysate” (also known as a “clarified lysate”), which as used herein, refers to a solution collected following sedimentation of lysed host cells from a host cell culture. In some embodiments, a solution comprising an AAV vector can comprise a “post-harvest solution,” which as used herein refers to a solution resulting from a cell lysis of host cells, wherein the solution has undergone flocculation, depth filtration, and/or nominal filtration. In some embodiments, an AAV vector can be purified from a solution having undergone at least one other purification or processing step (e.g., cell lysis, flocculation, filtration, dilution, pH adjustment, chromatography). In some embodiments, a clarified lysate has undergone at least one other purification or processing step (e.g., cell lysis, flocculation, filtration, dilution, pH adjustment, chromatography). In some embodiments, a post-harvest solution, has undergone at least one other purification or processing step (e.g., cell lysis, flocculation, filtration, dilution, pH adjustment, chromatography). In some embodiments, a solution comprising an rAAV vector (e.g., solution that is loaded to the affinity chromatography stationary phase), is obtained from at least one other purification or processing step (e.g., cell lysis, flocculation, filtration, dilution, pH adjustment, chromatography).

In some embodiments, a host cell culture can comprise a host cell selected from the group HEK293, PER.C6, W138, MRC5, A549, HeLa cell, HepG2, Saos-2, HuH7, HT1080, VERO, COS-1, COS-7, MDCK, BHK21-F, HKCC, and CHO cells. In some embodiments, a host cell can be a HEK293 cell.

In some embodiments, a solution comprising a rAAV vector (e.g. rAAV9) that is loaded on an affinity chromatography stationary phase can be loaded onto the stationary phase to achieve a challenge of about, at least about, or at most about 1×10¹², 5×10¹², 1×10¹³, 1.5×10¹³, 2×10¹³, 2.5×10¹³, 3×10¹³, 3.5×10¹³, 4×10¹³, 4.5×10¹³, 5×10¹³, 5.5×10¹³, 6×10¹³, 6.5×10¹³, 7×10¹³, 7.5×10¹³, 8×10¹³, 8.5×10¹³, 9×10¹³, 9.5×10¹³, 1×10¹⁴, 1.5×10¹⁴, 2×10¹⁴, 2.5×10¹⁴, 3×10¹⁴, 3.5×10¹⁴, 4×10¹⁴, 4.5×10¹⁴, 5×10¹⁴, 5.5×10¹⁴, 6×10¹⁴, 6.5×10¹⁴, 7×10¹⁴, 7.5×10¹⁴, 8×10¹⁴, 8.5×10¹⁴, 9×10¹⁴, 9.5×10¹⁴, or 1×10⁵ viral genomes (vg)/mL stationary phase (or any range derivable therein). In some embodiments, a solution comprising an rAAV vector is loaded onto a stationary phase to achieve a challenge of 1×10¹² viral genomes (vg)/mL stationary phase to 1.5×10¹⁴ vg/mL stationary phase. In some embodiments, a solution comprising an AAV vector can be loaded onto a stationary phase to achieve a challenge of ≤5×10¹³ vg/mL stationary phase.

In some embodiments, a method of purifying a AAV vector (e.g. rAAV) by affinity chromatography further comprises a pre-use rinse of a stationary phase prior to loading a solution comprising the AAV vector on the stationary phase. In some embodiments, a pre-use rinse removes a stationary phase storage solution (e.g., ethanol), and optionally prepares the stationary phase for equilibration. In some embodiments, a pre-use rinse comprises application of water (e.g., water for injection) to a stationary phase and removal of all or a portion of the water from the stationary phase. In some embodiments, about 2 CV to about 10 CV, e.g., about 2 CV, about 3 CV, about 4 CV, about 5 CV, about 6 CV, about 7 CV, about 8 CV, about 9 CV or about 10 CV of a pre-elution wash solution is applied to a stationary phase. In some embodiments, a pre-use rinse flows upward through a stationary phase in a column.

In some embodiments, a method of purifying an AAV vector (e.g., rAAV) by affinity chromatography further comprises pre-use sanitization of a stationary phase prior to loading a solution comprising the AAV vector on the stationary phase. In some embodiments, a pre-use sanitization comprises application of a solution comprising phosphoric acid (e.g., about 0.1 N to about 0.2 N phosphoric acid) to a stationary phase and removal of all or a portion of the solution comprising the phosphoric acid from the stationary phase. In some embodiments, a pre-use sanitization solution comprises about 0.132 N phosphoric acid at a pH of 1.9. In some embodiments, about 2 CV to about 10 CV, e.g., about 2 CV, about 3 CV, about 4 CV, about 5 CV, about 6 CV, about 7 CV, about 8 CV, about 9 CV or about 10 CV of a pre-use sanization solution is applied to a stationary phase. In some embodiments, application of a pre-use sanitization solution to a stationary phase reduces bioburden. In some embodiments, a pre-use sanitization solution flows upward through a stationary phase in a column.

In some embodiments, a method of purifying an AAV vector (e.g., rAAV) by affinity chromatography further comprises equilibration of the stationary phase. In some embodiments, a stationary phase is equilibrated by application of a buffer which is compatible with the solution to be loaded onto the stationary phase, for instance a clarified lysate, and the molecule to be purified, for instance a rAAV vector (e.g., rAAV9). In particular, a buffer which is compatible with a clarified lysate, and rAAV vector it contains, has a compatible pH (e.g. pH 7.5) and/or a compatible conductivity.

Equilibration of a stationary phase can be performed one or more times, for instance, 2, 3, 4, 5 6 or more times when purifying an AAV vector by affinity chromatography. In some embodiments, equilibration may be performed i) prior to loading the solution comprising the rAAV vector on the stationary phase, ii) after loading the solution comprising the rAAV vector on the stationary phase, iii) prior to application of a pre-elution wash, iv) after application of a pre-elution wash v) prior to eluting the rAAV vector from the stationary phase with an elution buffer, vi) after eluting the rAAV vector from the stationary phase with an elution buffer and/or vii) prior to contacting the stationary phase with a first regeneration buffer. In some embodiments, equilibration comprises application of a buffer solution to a stationary phase and removal of all or a portion of the buffer solution from the stationary phase. In some embodiments, an equilibribum buffer solution comprises Tris or sodium acetate. In some embodiments, a buffer solution for equilibration comprises about 50 mM to about 150 mM Tris (e.g., about 100 mM) at a pH of 7 to 8 (e.g., pH of about 7.5). In some embodiments, a buffer solution comprises about 100 mM to about 200 mM sodium acetate (e.g., about 153 mM sodium acetate) at a pH of 5 to 6 (e.g., pH of about 5.6). In some embodiments, a buffer solution comprises about 100 mM Tris at about pH 7.5. In some embodiments, a buffer solution comprises about 153 mM sodium acetate at about pH 5.6.

In some embodiments, a first equilibration of the stationary phase is performed prior to loading the solution comprising the rAAV vector on the stationary phase, optionally to render the stationary phase and surrounding aqueous phase compatible with the load solution.

In some embodiments, a second equilibration of the stationary phase is performed after loading the solution comprising the rAAV vector on the stationary phase and before eluting the rAAV vector from the stationary phase, and optionally prior to a pre-elution wash. Such second equilibration, which may be referred to as a “wash”, removes unbound impurities from the stationary phase, and from within the pores of the stationary phase, prior to application of a stronger wash or elution buffer.

In some embodiments, a third equilibration of the stationary phase is performed after loading the solution comprising the rAAV vector on the stationary phase and before eluting the rAAV vector from the stationary phase, and optionally after the pre-elution wash. In some embodiments, a a fourth equilibration of the stationary phase is performed prior to contacting the stationary phase with a first regeneration buffer.

In some embodiments, about, at least about, or at most about 0.5, 1, 1.5, 2, 2.5, 3, 3.5, 4, 4.5, 5, 5.5, 6, 6.5, 7, 7.5, 8, 8.5, 9, 9.5, 10, 15, 20, or 30 column volume (CV) (or any range derivable therein) of a equilibration buffer solution is applied to a stationary phase. In some embodiments, an equilibration buffer solution is applied to a stationary phase at a flow velocity of about, at least about, or at most about 1, 5, 10, 20, 30, 40, 50, 60, 70, 80, 90, 100, 110, 120, 130, 140, 150,160, 170, 180, 190, 200, 210, 220, 230, 240, 250, 260, 270, 280, 290, 300, 310, 320, 330, 340, 345, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 700, 750, 800, 900, 1000, 1500 or 2000 cm/hr (or any range derivable therein). In some embodiments, an equilibration buffer solution is applied to a stationary phase with a residence time about, at least about, or at most about 0.1, 0.5, 1, 1.5, 2, 2.5, 3, 3.5, 4, 4.1, 4.2, 4.3, 4.35, 4.4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 min/column volume (CV) (or any range derivable therein).

D. Pre-Elution Wash

In some embodiments, a method of purification of an AAV vector (e.g., rAAV vector) with affinity chromatography can comprises use of a pre-elution wash. A pre-elution wash can comprise application of a pre-elution wash solution to a stationary phase loaded with AAV vector, but prior to elution of the AAV vector off of the stationary phase with the elution buffer. In some embodiments, a pre-elution wash solution is applied to a stationary phase after loading a solution comprising the rAAV vector and before eluting the rAAV vector from the stationary phase with an elution buffer to produce an affinity eluate. In some instances, a pre-elution wash is at least partially, or completely, removed from a stationary phase before elution of an AAV vector off of the stationary phase with an elution buffer. Advantagously, a pre-elution wash will elute host cell proteins from a stationary phase before eluting a product (e.g., rAAV vector), thereby avoiding elution of host cell proteins with the product.

In some embodiments, a pre-elution wash can i) remove or sufficiently remove bound impurities from the stationary phase; ii) maintain or sufficiently maintain AAV bound to the stationary phase; and/or iii) have a reduced pH to improve removal of proteins other than AAV proteins, such as host cell proteins (HCP).

In some embodiments, a pre-elution wash solution can comprise a solvent and a buffering agent. In some embodiments, a pre-elution wash solution can be an aqueous solution comprising a solvent and a buffering agent. In some embodiments, a solvent can be ethanol, isopropanol, propanol, butanol, ethylene glycol, or any combination thereof. In some embodiments, a solvent can be ethanol. In some aspects, concentration of a solvent, such as ethanol, in a pre-elution wash solution can be about, at least about, or at most about 0.1, 1, 2, 3, 4, 5, 6, 7, 8, 9, 10, 11, 12, 13, 14, 15, 15.5, 16, 16.5, 17, 17.5, 18, 18.5, 19, 19.5, 20, 21, 22, 23, 24, 25, 26, 27, 28, 29, 30, 35, 40, 45, 50, 55, 60, 65, 70, 75, or 80 wt. % (or any range derivable therein). In some embodiments, a pre-elution buffer comprises a solvent and a buffering agent In some embodiments, a pre-elution buffer comprises a solvent that is ethanol. In some embodiments, a pre-elution buffer comprises about 17% or about 17.5% ethanol.

In some embodiments, a buffering agent can be sodium acetate, ammonium acetate, Tris (e.g., a mixture of Tris Base and Tris-HCl), BIS-Tris propane, diethanolamine, diethylamine, tricine, triethanolamine, bicine, or any combinations thereof. In some embodiments, concentration of a buffering agent, such as sodium acetate, in a pre-elution wash solution can be about, at least about, or at most about 1, 5, 10, 20, 30, 40, 50, 55, 60, 65, 70, 75, 80, 85, 90, 95, 100, 105, 110, 115, 120, 125,130, 135, 140, 150, 155,160, 170, 180, 190, 200, 300, 400, 500, 600, 700, 800, 900, 1000, 1500, or 2000 mM (or any range derivable therein). In some embodiments, a pre-elution wash solution comprises about, at least about, or at most about 1, 5, 10, 20, 30, 40, 50, 60, 70, 80, 90, 100, 105, 110, 115,120, 125, 130, 135, 140, 145, 146, 147, 148,149, 150, 151, 152, 153,154, 155, 156, 157, 158, 159, 160, 170, 180, 190, 200, or 300 mM (or any range derivable therein) of sodium acetate. In some embodiments, a pre-elution wash solution comprises 10 mM to 500 mM of a buffering agent, optionally wherein the buffering agent is sodium acetate. In some embodiments, a pre-elution wash solution comprises about 150 or about 153 mM sodium acetate.

In some embodiments, a pre-elution wash solution can have a pH of about, at least about, or at most about 3, 3.5, 4, 4.5, 5, 5.5, 5.6, 6, 6.5, 7, 7.5, 8, 8.5, 9, 9.5, or 10 (or any range derivable therein). In some embodiments, a pre-elution wash solution comprises a pH of about 5 to about 6. In some embodiments, a pre-elution wash solution comprises a pH of about 5.6.

In some embodiments, a pre-elution wash solution can comprise i) 2, 3, 4, 5, 6, 7, 8, 9, 10, 11, 12, 13, 14, 15, 15.5, 16, 16.5, 17, 17.5, 18, 18.5, 19, 19.5, 20, 21, 22, 23, 24, 25, 26, 27, 28, 29, 30, 35, 40, 45, or 50% (or any range derivable therein) of ethanol; ii) about, at least about, or at most about 100, 200, 300, 400, 500, 600, 700, 800, 900, 1000, 1500, 2000, 2500, or 3000 mM (or any range derivable therein) of sodium chloride; iii) about, at least about, or at most about 5, 10, 20, 30, 40, 50, 55, 60, 65, 70, 75, 80, 85, 90, 95, 100, 105, 110, 115, 120, 125, 130, 135, 140, 150, 155, 160, 170, 180, 190, 200, 300, 400, or 500 mM (or any range derivable therein) of Tris; and iv) a pH of about, at least about, or at most about 5, 5.5, 5.6, 6, 6.5, 7, 7.5, 8, 8.5, or 9 (or any range derivable therein). In some embodiments, a pre-elution wash solution can comprise i) 2, 3, 4, 5, 6, 7, 8, 9, 10, 11, 12, 13, 14, 15, 15.5, 16, 16.5, 17, 17.5, 18, 18.5, 19, 19.5, 20, 21, 22, 23, 24, 25, 26, 27, 28, 29, 30, 35, 40, 45, or 50% (or any range derivable therein) of ethanol; ii) about, at least about, or at most about 100, 200, 300, 400, 500, 600, 700, 800, 900, 1000, 1500, 2000, 2500, or 3000 mM (or any range derivable therein) of sodium chloride; iii) about, at least about, or at most about 5, 10, 20, 30, 40, 50, 60, 70, 80, 90, 100, 105, 110, 115, 120, 125, 130,135, 140, 145, 146, 147,148, 149, 150, 151, 152, 153, 154, 155, 156, 157, 158, 159, 160, 170, 180, 190, 200, or 300 mM (or any range derivable therein) of sodium acetate; and iv) a pH of about, at least about, or at most about 4, 4.5, 5, 5.5, 5.6, 6, 6.5, 7, 7.5, or 8 (or any range derivable therein). In some embodiments, a pre-elution wash solution can comprise i) 2, 3, 4, 5, 6, 7, 8, 9, 10, 11, 12, 13, 14, 15, 15.5, 16, 16.5, 17, 17.5, 18, 18.5, 19, 19.5, 20, 21, 22, 23, 24, 25, 26, 27, 28, 29, 30, 35, 40, 45, or 50% (or any range derivable therein) of ethanol; ii) about, at least about, or at most about 5, 10, 20, 30, 40, 50, 60, 70, 80, 90, 100, 105, 110, 115,120, 125, 130, 135, 140,145, 146, 147, 148, 149, 150, 151, 152, 153, 154, 155, 156, 157, 158, 159, 160, 170, 180, 190, 200, or 300 mM (or any range derivable therein) of sodium acetate; and iii) a pH of about, at least about, or at most about 4, 4.5, 5, 5.5, 5.6, 6, 6.5, 7, 7.5, or 8 (or any range derivable therein). In some embodiments a pre-elution wash solution comprises 15% to 25% of ethanol, 100 mM to 200 mM of sodium acetate, and has a pH of 5 to 6. In some embodiments. In some embodiments, a pre-elution wash solution comprises about 17.5% ethanol, about 153 mM sodium acetate and has a pH of about 5.6. In some embodiments, a pre-elution wash solution comprises about 17% ethanol, about 150 mM sodium acetate and has a pH of about 5.6.

In some embodiments, about, at least about, or at most about 0.5, 1, 1.5, 2, 2.5, 3, 3.5, 4, 4.5, 5, 5.5, 6, 6.5, 7, 7.5, 8, 8.5, 9, 9.5, 10, 15, 20, or 30 column volume (CV) (or any range derivable therein) of a pre-elution wash solution is applied to a stationary phase. In some embodiments, 1 CV to 10 CV or 4.5 to 5.5 CV of a pre-elution wash solution is applied to a stationary phase. In some embodiments, about 5 CV of a pre-elution wash solution is applied to a stationary phase. In some embodiments, a pre-elution wash solution is applied to a stationary phase at a flow velocity of about, at least about, or at most about 1, 5, 10, 20, 30, 40, 50, 60, 70, 80, 90, 100, 110, 120, 130, 140, 150,160, 170, 180, 190, 200, 210, 220, 230, 240, 250, 260, 270, 280, 290, 300, 310, 320, 330, 340, 345, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 700, 750, 800, 900, 1000, 1500 or 2000 cm/hr (or any range derivable therein). In some embodiments, a pre-elution wash solution is applied to a stationary phase with a residence time about, at least about, or at most about 0.1, 0.5, 1, 1.5, 2, 2.5, 3, 3.5, 4, 4.1, 4.2, 4.3, 4.35, 4.4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 min/column volume (CV) (or any range derivable therein).

In some embodiments, a pre-elution wash has an A280 peak height of about, at least about, or at most about 1500, 1600, 1700, 1800, 1900, 2000, 2100, 2200, 2300, 2400, or 2500 mAU when using a 2 mm path length after removal from the stationary phase.

E. Elution of the Affinity Chromatography Column

In some embodiments, the AAV (e.g. rAAV) vector can be eluted off of the stationary phase by application of an elution buffer to the stationary phase. In some embodiments, the elution buffer does not elute, or does not preferentially elute residual impurities from the stationary phase and/or results in relatively little or no precipitation in the affinity eluate. In some embodiments, the AAV (e.g. rAAV) vector is eluted off of the stationary phase after the pre-elution wash has been applied to the stationary phase, after the pre-elution wash has at least partially been removed from the stationary phase, or without the stationary phase having been contacted with the pre-elution wash.

In some embodiments, the elution buffer has any one of, any combination of, or all of the following properties i) elutes the AAV, such as rAAV from the stationary phase; ii) does not elute residual impurities from the stationary phase; iii) does not result in precipitation of an affinity eluate; iv) increases and/or maximizes an affinity % vg recovery; v) does not interfere, or does not sufficiently interfere, with binding of the rAAV vector, such as an rAAV9, in the eluate to an anion exchange (AEX) stationary phase. In some embodiments, the elution buffer does not contain a trivalent anion, such as citrate ions. In some embodiments, the elution buffer does not interfere, or does not sufficiently interfere, with binding of the rAAV vector, such as an rAAV9 vector, in an eluate to an AEX stationary phase in a subsequent purification step, such as about, at least about, 80, 85, 90, 91, 92, 93, 94, 95, 96, 97, 98, 99, or 100% of the rAAV vector in the eluate binds to the AEX stationary phase.

In some embodiments, the elution buffer comprises a salt, an amino acid, a buffering agent, or any combinations thereof.

In some embodiments, the salt in the elution buffer can be sodium chloride, magnesium chloride, sodium sulfate and a combination thereof. In some embodiments, the salt in the elution buffer can be magnesium chloride. In some embodiments, concentration of the salt, such as magnesium chloride, in the elution buffer can be about, at least about, or at most about 0.1, 0.5, 1, 5, 10, 20, 30, 40, 50, 60, 70, 80, 90, 100, 105, 110, 115, 120, 125, 130, 135, 140, 145, 146, 147, 148,149, 150, 151, 152, 153,154, 155, 156, 157, 158,159, 160, 170, 180, 190, 200, 300, 400, 500, 600, 700, 800, 900, 1000, 1500, 2000, 2500, 3000, 3500, 4000, 4500, or 5000 mM (or any range derivable therein). In some embodiments, concentration of magnesium chloride, in the elution buffer can be about, at least about, or at most about 0.1, 0.5, 1, 5, 10, 15, 20, 21, 22, 23, 24, 25, 26, 27, 28, 29, 30, 35, 40, 50, 60, 70, 80, 90, 100, 105, 110, 115, 120, 125, 130, 135,140, 145, 146, 147, 148,149, 150, 151, 152, 153,154, 155, 156, 157, 158, 159, 160, 170, 180, 190, 200, 250, or 300 mM (or any range derivable therein). In some embodiments, an elution buffer comprises 5 mM to 150 mM of a salt, and optionally, the salt is magnesium chloride. In some embodiments, an elution buffer comprises about 25 mM of magnesium chloride.

In some embodiments, the amino acid in the elution buffer can be histidine, arginine, citrulline, glycine, or any combinations thereof. In some embodiments, the amino acid in the elution buffer can be glycine. In some embodiments, concentration of the amino acid, such as glycine, in the elution buffer can be about, at least about, or at most about 0, 0.1, 0.5, 1, 5, 10, 20, 30, 40, 50, 60, 70, 75, 80, 85, 90, 95, 100, 105, 110, 115, 120, 125,130, 135, 140, 145, 146, 147, 148, 149, 150,151, 152, 153, 154, 155,156, 157, 158, 159, 160,170, 180, 190, 200, 300, 400, 500, 600, 700, 800, 900, 1000, 1500, 2000, 2500, 3000, 3500, 4000, 4500, or 5000 mM (or any range derivable therein). In some embodiments, an elution buffer comprises 50 mM to 150 mM of an amino acid, optionally the amino acid is glycine. In some embodiments, an elution buffer comprises about 100 mM of glycine.

In some aspects, the buffering agent in the elution buffer can be sodium acetate, ammonium acetate, sodium citrate, Tris (e.g., a mixture of Tris Base and Tris-HCl), BIS-Tris propane, diethanolamine, diethylamine, tricine, triethanolamine, bicine, or any combinations thereof. In some embodiments, concentration of the buffering agent, such as sodium acetate, in the elution buffer can be about, at least about, or at most about 0.1, 0.5, 1, 5, 10, 20, 30, 40, 50, 60, 70, 75, 80, 85, 90, 95, 100, 105, 110, 115,120, 125, 130, 135, 140,145, 146, 147, 148, 149, 150, 151, 152, 153, 154, 155, 156, 157, 158,159, 160, 170, 180, 190, 200, 300, 400, 500, 600, 700, 800, 900, 1000, 1500, 2000, 2500, 3000, 3500, 4000, 4500, or 5000 mM (or any range derivable therein). In some embodiments, concentration of sodium acetate in the elution buffer can be about, at least about, or at most about 50, 60, 70, 80, 90, 100, 105, 110, 115, 120, 125, 130, 135, 140,145, 146, 147, 148, 149,150, 151, 152, 153, 154,155, 156, 157, 158, 159, 160, 170, 180, 190, 200, 300, 400, or 500 mM (or any range derivable therein). In some embodiments, an elution buffer comprises 75 mM to 250 mM of a buffering agent, optionally the buffering agent is sodium acetate. In some embodiments, the elution buffer comprises about 148 mM of sodium acetate.

In some embodiments, the elution buffer can have a pH of about, at least about, or at most about 1, 1.5, 2, 2.5, 3, 3.5, 4, 4.5, 5, 5.5, 5.6, 6, 6.5, 7, 7.5, or 8 (or any range derivable therein). In some embodiments, an elution buffer has a pH of 2.5 to 3.5, for example about 3.0.

In some embodiments, the elution buffer can comprise i) about, at least about, or at most about 50, 60, 70, 75, 80, 85, 90, 95, 100, 105, 110, 115, 120, 125, 130, 135, 140, 145, 146, 147, 148, 149, 150,151, 152, 153, 154, 155,156, 157, 158, 159, 160, 170, 180, 190, 200, or 300 mM (or any range derivable therein) of glycine; ii) about, at least about, or at most about, 1, 5, 10, 15, 16, 17, 18, 19, 20, 21, 22, 23, 24, 25, 26, 27, 28, 29, 30, 40, 50, 60, 70, 80, 90, 100, 105, 110, 115, 120,125, 130, 135, 140, 145,146, 147, 148, 149, 150,151, 152, 153, 154, 155, 156, 157, 158, 159, 160, 170, 180, 190, 200, or 250 mM (or any range derivable therein) of MgCl₂; iii) about, at least about, or at most about 0, 0.1, 1, 5, 10, 20, 30, 40, 50, 60, 70, 80, 90, 100, 105, 110, 115, 120, 125, 130, 135, 140,145, 146, 147, 148, 149,150, 151, 152, 153, 154, 155, 156, 157, 158, 159, 160, 170, 180, 190, 200, 250, or 300 mM (or any range derivable therein) of sodium acetate; and iv) a pH of about, at least about, or at most about 1, 1.5, 2, 2.5, 3, 3.5, 4, 4.5, or 5 (or any range derivable therein). In some embodiments, an elution buffer comprises 50 mM to 150 mM of glycine, 10 mM to 100 mM of MgCl₂, 50 mM to 150 mM of sodium acetate, and a pH of 2.5 to 3.5. In some embodiments, an elution buffer comprises about 100 mM glycine, about 25 mM MgCl₂, about 148 mM sodium acetate, and has a pH of about 3.0.

In some embodiments, the elution buffer does not contain an anion that competes with binding of the AAV vector, such as a rAAV vector, to an AEX stationary phase. In some aspects, the elution buffer does not contain an trivalent anion that competes with binding of the AAV vector, such as a rAAV vector, to an AEX stationary phase. In some embodiments, the elution buffer does not contain a citrate anion.

In some embodiments, the elution buffer can have a conductivity of about, at least about, or at most about 1, 5, 10, 15, 20, 25, 30, 35, 40, 45, 50, 55, 60, 65, 70, 75, 80, 85, 90, 95, and 100 mS/cm (or any range derivable therein). In some embodiment, an elution buffer has a conductivity of 1 mS/cm to 40 mS/cm, 1 mS/cm to 30 mS/cm or 20 mS/cm to 35 mS/cm.

In some embodiments, about, at least about, or at most about 0.5, 1, 1.5, 2, 2.5, 3, 3.5, 4, 4.5, 5, 5.5, 6, 6.5, 7, 7.5, 8, 8.5, 9, 9.5, 10, 15, 20, or 30 column volume (CV) (or any range derivable therein) of the elution buffer is applied to the stationary phase. In some embodiments, the elution buffer is applied to the stationary phase at a flow velocity of about, at least about, or at most about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 700, 750, 800, 900, 1000, 1500, or 2000 cm/hr (or any range derivable therein). In some embodiments, about 2 CV to 10 CV or 4.5 CV to 5.5 CV of an elution buffer is applied to a stationary phase. In some embodiments, about 5 CV of an elution buffer is applied to a stationary phase.

In some embodiments, the elution buffer solution is applied to the stationary phase with a residence time about, at least about, or at most about 0.1, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 min/column volume (CV) (or any range derivable therein).

In some embodiments, elution of the AAV vector off of the stationary phase removes about, at least about, or at most about 60, 65, 70, 75, 80, 85, 90, 95, 96, 97, 98, 99, or 100% of the rAAV vector loaded onto the stationary phase (or any range derivable therein).

In some instances, the concentration of viral genomes (vg) per ml of the eluate, or a portion of the eluate, is about, at least about, or at most about 1.0×10¹³, 2.0×10¹³, 3.0×10¹³, 4.0×10¹³, 5.0×10¹³, 6.0×10¹³, 7.0×10¹³, 8.0×10¹³, 9.0×10¹³, 1.0×10¹⁴ vg/ml (or any range derivable therein).

In some instances, the % purity of the total proteins in the eluate, or a portion of the eluate, that are proteins from the target AAV vector is about, at least about, or at most about 60, 65, 70, 75, 80, 85, 90, 95, 96, 97, 98, 99, or 100% (or any range derivable therein).

In some instances, the amount of the host cell protein (HCP) in the eluate, or a portion of the eluate, is about, at least about, or at most about 1000, 2000, 3000, 4000, 5000, 6000, 7000, 8000, 9000, 10000, 11000, 12000, 13000, 14000, 15000, 16000, 17000, 18000, 19000, or 20000 ng/1×10¹⁴ vg (or any range derivable therein) or about, at least about, or at most about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 pg/1×10⁹ vg (or any range derivable therein).

In some instances, the amount of the host cell DNA (HCDNA) in the eluate, or a portion of the eluate, is about, at least about, or at most about 50, 60, 70, 80, 90, 100, 110, 120, 130, 140, 150, 160, 170,180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 ng/1×10¹⁴ vg (or any range derivable therein) or about, at least about, or at most about 1, 2, 3, 4, 5, 6, 7, 8, 9, 10, 11, 12, 13, 14, 15, 15.5, 16, 16.5, 17, 17.5, 18, 18.5, 19, 19.5, 20, 21, 22, 23, 24, or 25 pg/1×10⁹ vg (or any range derivable therein).

In some instances, the A260/A280 ratio of the eluate, or a portion of the eluate, is about, at least about, or at most about 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.5, 1.6, 1.7, 1.8, 1.9, or 2 (or any range derivable therein).

In some instances, the infectivity ratio of the affinity eluate is about, at least about, at most about, 8000, 10000, 11000, 12000, 12962, 13000, 14000, 15000, 16000, 17000, 17581, 18000, 19000, 20000, 20345, 22000, 25000, or 30000 vg/IU (or any range derivable therein).

F. Collection of the Affinity Eluate

In some embodiments, the method of purification of a rAAV vector, such as rAAV9 vector, with affinity chromatography can comprise collection of an affinity eluate from the stationary phase. In some embodiments, the affinity eluate can be collected as a single fraction. In some embodiments, the affinity eluate can be collected as more than one fraction. In some aspects, at least some of the, and/or portion of the more than one affinity fractions can be pooled.

In some aspects, collection of the affinity eluate can be started at about, at least about, or at most about 0.1, 0.5, 1, 2, 2.5, 3, 4, 5, 6, 7, 8, 9, 10, 11, 12, 12.5, 13, 14, 15, 16, 17, 18, 19, 20, 25, 30, 35, 40, 45, 50, 55, 60, 65, or 70 mAU (or any range derivable therein) at A280 using a 2 mm path length. In some aspects, collection of the affinity eluate can be stopped at about, at least about, or at most about 10, 11, 12, 12.5, 13, 14, 15, 16, 17, 18, 19, 20, 22.5, 25, 30, 35, 40, 45, 50, 55, 60, 65, 70, 75, 80, 85, 90, 95, 100, 105, 110,115, 120, 125, 130, 135, 140, 145, 146, 147,148, 149, 150, 151, 152,153, 154, 155, 156, 157, 158, 159, 160, 170, 180, 190, or 200 mAU (or any range derivable therein) at A280 using a 2 mm path length. In some aspects, collection of the affinity eluate can be started at about, at least about, or at most about 1, 2, 3, 4, 5, 6, 7, 8, 9, 10, 11, 12, 12.5, 13, 14, 15, 16, 17, 18, 19, 20, 22.5, 25, 30, 35, 40, 45, 50, 55, 60, 65, 70, 75, 80, 85, 90, 95, or 100 mAU/5 mm path length (or any range derivable therein) at A280. In some aspects, collection of the affinity eluate can be stopped at about, at least about, or at most about 25, 30, 35, 40, 45, 50, 55, 60, 65, 70, 75, 80, 85, 90, 95, 100, 105, 110, 115, 120,125, 130, 135, 140, 145,146, 147, 148, 149, 150,151, 152, 153, 154, 155, 156, 157, 158, 159, 160, 170, 180, 190, or 200 mAU/5 mm path length (or any range derivable therein) at A280. In some aspects, collection of the affinity eluate can be started at about, at least about, or at most about 0.1, 0.5, 1, 2, 2.5, 3, 4, 5, 6, 7, 8, 9, 10, 11, 12, 12.5, 13, 14, 15, 16, 17, 18, 19, 20, 25, 30, 35, 40, 45, 50, 55, 60, 65, or 70 mAU/mm (or any range derivable therein) at A280, and collection of the affinity eluate can be stopped at about, at least about, or at most about 01, 0.5, 1, 2, 2.5, 3, 4, 5, 6, 7, 8, 9, 10, 11, 12, 12.5, 13, 14, 15, 16, 17, 18, 19, 20, 25, 30, 35, 40, 45, 50, 55, 60, 65, or 70 mAU/mm (or any range derivable therein) at A280. In some instances, the collection is started and stopped based on the slope of the absorbance/time. In some instances, the mAU change/min at start time <mAU change/min at start time+1 sec, and mAU change/min at stop time >mAU change/min at stop time+1 sec, and wherein the collection is started at start time and the collection is stopped at stopped time. In some instances, the collection is started and/or stopped at predetermined times or eluate volumes.

In some aspects, a method of purifying an rAAV vector comprises loading a solution comprising the rAAV vector on an affinity chromatography stationary phase in a column; applying a pre-elution wash solution comprising 15% to 25% ethanol, 100 mM to 200 mM sodium acetate, and a pH of 5 to 6 to the stationary phase; and eluting the rAAV vector from the stationary phase with an elution buffer comprising 50 mM to 150 mM glycine, 10 mM to 100 mM MgCl₂, 50 mM to 250 mM sodium acetate, and a pH of 2.5 to 3.5 to produce an affinity eluate containing the rAAV vector. In some embodiments, a method of purifying an rAAV vector further comprises contacting a stationary phase with a first regeneration buffer after obtaining at least a portion of an affinity eluate. In some embodiments, a method of purifying an rAAV vector further comprises contacting the stationary phase with a second regeneration buffer after the first regeneration buffer, wherein the second regeneration buffer is different than the first regeneration buffer.

In some embodiments, volume of the affinity eluate collected from the stationary phase can be about, at least about, or at most about 0.1, 0.5, 1, 5, 10, 20, 30, 40, 50, 60, 70, 75, 80, 85, 90, 95, 100, 105, 110, 115, 120, 125,130, 135, 140, 145, 146,147, 148, 149, 150, 151, 152, 153, 154, 155,156, 157, 158, 159, 160,170, 180, 190, 200, 300, 400, 500, 600, 700, 800, 900, 1000, 1500, 2000, 2500, 3000, 3500, 4000, 4500, or 5000 mL (or any range derivable therein). In some embodiments, volume of the affinity eluate collected from the stationary phase can be about, at least about, or at most about 0.01, 0.1, 0.5, 1, 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5, 10, 15, 20, or 30 CV (or any range derivable therein). In some embodiments, wherein 0.1 CV to 10 CV of an affinity eluate is collected from the stationary phase.

In some embodiments, a % vg recovery of an affinity eluate collected is about, at least about, 75, 76, 77, 78, 79, 80, 81, 82, 83, 84, 85, 86, 87, 88, 89, 90, 91, 92, 93, 94, 95, 96, 97, 98, 99 or 100% (or any range derivable therein). In some embodiments, a % vg recovery of the affinity eluate can be measured by qPCR. In some embodiments, qPCR measures copies of a transgene sequence. In some embodiments, a transgene sequence can comprise of or consist of the nucleic acid sequence of SEQ ID NO:1. In some embodiments, qPCR measures copies of an inverted terminal repeat (ITR) sequence. In some embodiments, an ITR sequence comprises or consists of the nucleic acid sequence of any one of SEQ ID NO:5-8. In some embodiments, a % vg recovery of an affinity eluate is 50% to 100%, optionally as measured by qPCR.

In some embodiments, an affinity eluate collected can comprise about, at least about, or at most about 1.0×10¹², 5.0×10¹², 1.0×10¹³, 2.0×10¹³, 3.0×10¹³, 4.0×10¹³, 5.0×10¹³, 6.0×10¹³, 7.0×10¹³, 8.0×10¹³, 9.0×10¹³, 1.0×10¹⁴, 2.0×10¹⁴, 3.0×10¹⁴, 4.0×10¹⁴, 5.0×10¹⁴, 6.0×10¹⁴, 7.0×10¹⁴, 8.0×10¹⁴, 9.0×10¹⁴, or 1.0×10¹⁵ vg/mL (or any range derivable therein). In some embodiments, a vg per mL of an affinity eluate can be measured by qPCR. In some embodiments, qPCR measures copies of a transgene sequence. In some embodiments, a transgene sequence can comprise of or consist of the nucleic acid sequence of SEQ ID NO:1. In some embodiments, qPCR measures copies of an inverted terminal repeat (ITR) sequence. In some embodiments, an ITR sequence comprises or consists of the nucleic acid sequence of any one of SEQ ID NO:5-8. In some embodiments, a vg per mL of an affinity eluate is about 1.0×10¹² vg/mL to about 1.0×10¹⁴ vg/mL, optionally as measured by qPCR.

In some embodiments, a step of collecting eluate from an affinity chromatography column can comprises measurement of absorbance at 260 nm (A260) and/or absorbance at 280 nm (A280) of an eluate collected from a column. In some embodiments, measurement of absorbance (e.g., at A260 or A280) of an affinity eluate is performed in-line of the flow of the eluate. In some embodiments, A260/A280 ratio of an affinity eluate collected can be greater than 0.8. In some embodiments, A260/A280 ratio of an affinity eluate can be about, at least about, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.5, 1.6, 1.7, 1.8, 1.9, or 2 (or any range derivable therein). In some embodiments, an A260/A280 ratio of an affinity eluate can be measured by size exclusion chromatography (SEC) with simultaneous absorption detection.

In some embodiments, % purity of an affinity eluate collected is about, at least about, 75, 76, 77, 78, 79, 80, 81, 82, 83, 84, 85, 86, 87, 88, 89, 90, 91, 92, 93, 94, 95, 96, 97, 98, 99, or 100% (or any range derivable therein). In some embodiments, % purity of an affinity eluate is about 95% to about 100%, optionally as measured by SEC or reverse phase HPLC, non-reducing. In some embodiments, % purity of an affinity eluate can be measured by reverse phase HPLC. In some embodiments, % purity can be measured by SEC. In some embodiments, AAV capsids in an affinity eluate can be loaded to the HPLC column in non-reduced form to measure % purity in an affinity eluate. A % purity of an AAV vector, such as rAAV vector in an affinity eluate can be higher compared to the % purity of an AAV vector such as rAAV vector, in a solution comprising the AAV vector, such as rAAV vector.

In some embodiments, amount of host cell DNA (HCDNA) in the affinity eluate collected can be about, at most about, 5, 10, 20, 30, 40, 50, 60, 70, 80, 90, or 100 ng per 1.0×10¹⁴ vg (or any range derivable therein) in the affinity eluate. In some embodiments, amount of HCDNA in the affinity eluate collected can be about, at most about, 1, 2, 3, 4, 5, 6, 7, 8, 9, 10, 11, 12, 13, 14, 15, 16, 17, 18, 19, or 20 pg per 1.0×10⁹ vg (or any range derivable therein) in the affinity eluate. Amount HCDNA in the affinity eluate can be measured by qPCR. The amount of HCDNA per vg in the affinity eluate can be lower compared to the amount of HCDNA per vg in the solution comprising the AAV vector, such as rAAV vector.

In some embodiments, amount of host cell protein (HCP) in the affinity eluate collected can be about, at most about, 1000, 2000, 3000, 4000, 5000, 6000, 7000, 8000, 9000, 10000, 11000, 12000, 13000, 14000, 15000, 16000, 17000, 18000, 19000, or 20000 ng per 1.0×10¹⁴ vg (or any range derivable therein). In some embodiments, amount of HCP in the affinity eluate collected can be about, at most about, 10, 20, 30, 40, 50, 60, 70, 80, 90, 100, 105, 110, 115, 120, 125, 130, 135,140, 145, 146, 147, 148,149, 150, 151, 152, 153, 154, 155, 156, 157, 158, 159, 160, 170, 180, 190, 200, 300, 400, 500, 600, 700, 800, 900, 1000, pg per 1.0×10⁹ vg (or any range derivable therein). In some embodiments, amount of HCP in the affinity eluate can be measured by ELISA, Western blot, and/or silver staining. The amount of HCP per vg in the affinity eluate can be lower compared to the amount of HCP per vg in the solution comprising the AAV vector, such as rAAV vector.

In some embodiments, the infectivity ratio of the affinity eluate collected is about, at least about, at most about, 8000, 10000, 11000, 12000, 12962, 13000, 14000, 15000, 16000, 17000, 17581, 18000, 19000, 20000, 20345, 22000, 25000, or 30000 vg/IU (or any range derivable therein). In some embodiments, the infectivity ratio of the affinity eluate can be measured by a cell based assay. In some embodiments, the infectivity ratio of the affinity eluate can be increased as comparted to the infectivity ratio of the solution comprising the rAAV vector.

G. Method of Regenerating a Stationary Phase for Purification of a rAAV Vector by Affinity Chromatography

Following affinity elution and collection of at least one fraction, and/or at least a portion of the affinity eluate, additional steps may be performed to prepare the affinity chromatography column media for further loading and elution of rAAV vector, such as rAAV9 affinity purification runs. Such steps may include, for example, sanitization, equilibration, regeneration, flush and/or storage. One of skill in the art will understand that one or more steps may be performed, in varying order and frequency. In some embodiments, the stationary phase in the affinity chromatography column, following affinity elution and collection of at least one fraction and/or at least a portion of the affinity eluate, is regenerated for further rAAV loading and elution, such as rAAV9 affinity purification runs. One of ordinary skill in the art will also understand that methods of regenerating a stationary phase following affinity eluation and collection of at least one eluate fraction comprising a purified rAAV vector includes methods of removing impuities, including rAAV vector proteins, from the stationary phase.

In some embodiments, a method of regenerating a stationary phase for purification of a rAAV vector by affinity chromatography is described. In some embodiments, a method of regenerating a stationary phase for purification of a rAAV vector by affinity chromatography comprises contacting a stationary phase with a first regeneration buffer after elution and collection of rAAV vectors off of the stationary phase. In some embodiments, a method of regenerating a stationary phase for purification of a rAAV vector by affinity chromatography comprises contacting a stationary phase with a first regeneration buffer after obtaining at least a portion of the affinity eluate. In some embodiments, a number of purification cycles that can be run on the stationary phase is increased as compared to a number of purification cycles that can be run on a stationary phase that is not contacted with a first regeneration buffer. In some embodiments, a purification cycle comprises loading a solution comprising the rAAV vector onto the stationary phase and eluting the rAAV vector from the stationary phase.

In some embodiments, a number of purification cycles that can be run on a stationary phase that is contacted with a first regeneration buffer is increased as compared to a stationary phase that is contacted with a buffer comprising guanidine HCl. In some embodiments, the number of purification cycles that can be run on the stationary phase that is contacted with the first regeneration buffer can be about, at least about, 1, 2, 3, 4, 5, 6, 7, 8, 9, 10, 12, 14, 16, 18, or 20 (or any range derivable therein). In some embodiments, a number of purification cycles that can be run on a stationary phase that is contacted with a first regeneration buffer is 8 or more. In some embodiments, a number of purification cycles that can be run on a stationary phase that is contacted with a first regeneration buffer is 10 or more. In some embodiments, a purification cycle comprises one load onto the stationary phase and one elution of the stationary phase wherein the % vg recovery in the affinity eluate off of the stationary phase is 90% or greater of that which was loaded onto the stationary phase.

In some embodiments, a purification cycle that can be run is determined by the % vg recovery from the stationary phase into an eluate. In some embodiments, a purification cycle that can be run has a % vg recovery from the current cycle that is not decreased or not decreased by more than about 0.1, 0.5, 1, 2, 3, 4, 5, 6, 7, 8, 9, or 10% (or any range derivable therein) as compared to a % vg recovery of an eluate from a first purification cycle using that column. In some embodiments, a % vg recovery of an eluate from a last purification cycle after contacting a stationary phase with a first regeneration buffer is not decreased more than 10% as compared to a % vg recovery of an eluate from a first purification cycle before the stationary phase is contacted with the first regeneration buffer.

In some embodiments, a cycle that can be run has an amount of unbound AAV vector in a flow through of the current cycle that is not increased or not increased by more than about 0.1, 0.5, 1, 2, 3, 4, 5, 6, 7, 8, 9, or 10% (or any range derivable therein) as compared to an amount of unbound AAV vector in a flow through of a first purification cycle using that column. In some embodiments, an amount of unbound rAAV vector in a flow through of a last purification cycle after contacting a stationary phase with a first regeneration buffer is not increased more than 10% as compared to an amount of unbound rAAV vector in a flow through of a first purification cycle before the stationary phase is contacted with the first regeneration buffer.

In some embodiments, a cycle that can be run has a column pressure of the current cycle that is not increased or not increased by more than about 0.1, 0.5, 1, 2, 3, 4, 5, 6, 7, 8, 9, or 10% (or any range derivable therein) as compared to a column pressure of a first purification cycle using that column. In some embodiments, a column pressure of a last purification cycle after contacting a stationary phase with a first regeneration buffer is not higher than 0.4 MPa.

In some embodiments, a cycle that can be run has a % capsid purity of an eluate from the current cycle that is not decreased or not decreased by more than about 0.1, 0.5, 1, 2, 3, 4, 5, 6, 7, 8, 9, or 10% (or any range derivable therein) as compared to a % capsid purity of an eluate from a first purification cycle using that column. In some embodiments, a % purity of an eluate from a last purification cycle after contacting a stationary phase with a first regeneration buffer is not decreased more than 10% as compared to a % purity of an eluate from a first purification cycle before the stationary phase is contacted with the first regeneration buffer.

In some embodiments, a cycle that can be run has an amount of HCP in an eluate from the current cycle that is not increased or not increased by more than about 0.1, 0.5, 1, 2, 3, 4, 5, 6, 7, 8, 9, or 10% (or any range derivable therein) as compared to an amount of HCP in an eluate from a first purification cycle using that column. In some embodiments, an amount of HCP of an eluate from a last purification cycle after contacting the stationary phase with the first regeneration buffer is not increased more than 10% as compared to an amount of HCP of an eluate from a first purification cycle before the stationary phase is contacted with the first regeneration buffer

In some embodiments, a cycle that can be run has an amount of HCDNA in an eluate in the current cycle that is not increased or not increased by more than about 0.1, 0.5, 1, 2, 3, 4, 5, 6, 7, 8, 9, or 10% (or any range derivable therein) as compared to an amount of HCDNA in an eluate from a first purification cycle on that column. In some embodiments, an amount of HCDNA of an eluate from a last purification cycle after contacting the stationary phase with the first regeneration buffer is not increased more than 10% as compared to an amount of HCDNA of an eluate from a first purification cycle before the stationary phase is contacted with the first regeneration buffer.

In some embodiments, a cycle that can be run has an A260/A280 of an eluate from the current cycle that is not decreased or not decreased by more than about 0.1, 0.5, 1, 2, 3, 4, 5, 6, 7, 8, 9, or 10% (or any range derivable therein) as compared to an A260/A280 of an eluate from a first purification cycle using that column. In some embodiments, an average A260/A280 of an eluate from a last purification cycle after contacting the stationary phase with the first regeneration buffer is not decreased more than 10% as compared to an average A260/A280 of an eluate from a first purification cycle before the stationary phase is contacted with the first regeneration buffer.

In some embodiments, a cycle that can be run has a maximum change in column pressure using a constant flow rate over the course of an affinity elution of the stationary phase that is about, at most about, 0.222, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, or 0.713 mPa (or any range derivable therein).

In some aspects, a method of regenerating an affinity chromatography stationary phase comprises contacting the stationary phase with a first regeneration buffer comprising an acid at a pH of 1 to 4, and wherein impurities are removed from the stationary phase. In some embodiments, a first regeneration buffer comprises an acid. In some aspects, a concentration of an acid in a first regeneration buffer can be about, at least about, or at most about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.4, 0.5, 0.6, 0.7, 0.8, 0.9, 1, 1.1, 1.2, 1.3, 1.4, 1.5, 1.6, 1.7, 1.8, 1.9, or 2 N (or any range derivable therein). In some embodiment, an acid in a first regeneration buffer can be phosphoric acid and/or acetic acid. In some embodiments, an acid in a first regeneration buffer is phosphoric acid. In some embodiments, an acid in a first regeneration buffer is phosphoric acid and a concentration of the phosphoric acid is about, at least about, or at most about 0.05, 0.06, 0.07, 0.08, 0.09, 0.1, 0.11, 0.12, 0.13, 0.14, or 0.15 N (or any range derivable therein). In some embodiments, a first regeneration buffer comprises 0.05 N to 1.5 N of an acid, optionally wherein the acid is phosphoric acid. In some instances, a pH of a first regeneration buffer can be about, at least about, or at most about 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, or 3.5 (or any range derivable therein). In some embodiments, a first regeneration buffer comprises 0.10 N to 0.15 N phosphoric acid, and a pH of 1.5 to 2.5. In some embodiments, a first regeneration buffer comprises about 0.132 N phosphoric acid at about pH 1.9.

In some embodiments, a stationary phase is contacted with about, at least about, or at most about 0.5, 1, 1.5, 2, 2.5, 3, 3.5, 4, 4.5, 5, 5.5, 6, 6.5, 7, 7.5, 8, 8.5, 9, 9.5, 10, 15, 20, or 30 CV (or any range derivable therein) of a first regeneration buffer. In some embodiments, a stationary phase is contacted with 1 CV to 10 CV or 4.5 CV to 5.5 CV of a first regeneration buffer. In some embodiments, a stationary phase is contacted with about 5 CV of a first regeneration buffer. In some embodiments, a stationary phase is contacted with about, at least about, or at most about 0.5, 1, 1.5, 2, 2.5, 3, 3.5, 4, 4.5, 5, 5.5, 6, 6.5, 7, 7.5, 8, 8.5, 9, 9.5, 10, or 15 CV (or any range derivable therein) of a first regeneration buffer; followed by a hold period, followed by contacting the stationary phase with the 0.5, 1, 1.5, 2, 2.5, 3, 3.5, 4, 4.5, 5, 5.5, 6, 6.5, 7, 7.5, 8, 8.5, 9, 9.5, 10, or 15 CV (or any range derivable therein) of the first regeneration buffer again. In some embodiments, the hold period can be about, at least about, at most about 0.5, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 60, 65, 70, 80, 90, 150, 300, 450, or 600 min (or any range derivable therein). In some embodiments, a stationary phase is contacted with about half of the volume of a first regeneration buffer, followed by a hold period of about 45 minutes, followed by contactin the stationary phase with a second half of the volume of a first regeneration buffer. In some embodiments, a stationary phase is contacted with about 2.5 CV of a first regeneration buffer followed by a hold period of about 45 minutes, followed by contacting the stationary phase with about 2.5 CV of a first regeneration buffer, optionally, wherein the first regeneration buffer comprises about 0.132 N phosphoric acid at about pH 1.9.

In some embodiments, a first regeneration buffer flows upward through a stationary phase. In some embodiments, a first regeneration buffer is flowed upward through a column containing a stationary phase. In some embodiments, a first regeneration buffer is flowed through a stationary phase in a direction that is opposite to the direction that an elution buffered is flowed through the stationary phase. In some embodiments, a first regeneration buffer is flowed through a column containing tahe stationary phase in a direction that is opposite to the direction that an elution buffered is flowed through the column containing the stationary phase.

In some embodiments, a first regeneration buffer is applied to a stationary phase at a flow velocity of about, at least about, or at most about 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 60, 65, 70, 80, 90, 100, 110, 120, 130, 140, 150, 160, 170,180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 700, 750, 800, 900, 1000, 1500, or 2000 cm/hr (or any range derivable therein). In some embodiments, a first regeneration buffer is applied to a stationary phase with a residence time about, at least about, or at most about 0.1, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 min/column volume (CV) (or any range derivable therein). In some embodiments, a method of regenerating a stationary phase further comprises loading another solution comprising an rAAV vector on an affinity chromatography stationary phase after removing at least a portion of a first regeneration buffer. In some embodiments, a method of regenerating a stationary phase further comprises applying a second amount of a pre-elution wash solution on an affinity chromatography stationary phase after removing at least a portion of a first regeneration buffer.

In some embodiments, a method of regenerating a stationary phase for purification of a rAAV vector by affinity chromatography further comprises contacting the stationary phase with a second regeneration buffer after a first regeneration buffer. In some embodiments, a second regeneration buffer is different than a first regenteration buffer. In some embodiments, a second regeneration buffer can comprise a detergent and a buffering agent. In some embodiments, a detergent in a second regeneration buffer can be sarkosyl, poloxamer 188 (P188), Triton X-100, polysorbate 80 (PS80), Brij-35, nonyl phenoxypolyethoxylethanol (NP-40), or any combinations thereof. In some embodiments, a detergent can be sarkosyl. In some embodiments, a second regeneration buffer can comprise about, at least about, or at most about 0.1, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 22, 24, 26, 28, or 30% (or any range derivable therein) of the detergent, such as sarkosyl. In some embodiments, a second regeneration buffer comprises 0.1% to 5% of a detergent, optionally wherein the detergent is sarkosyl. In some embodiments, a second regeneration buffer comprises about 1% sarkosyl.

In some embodiments, the buffering agent in the second regeneration buffer can be Tris (e.g., a mixture of Tris Base and Tris-HCl), BIS-Tris propane, diethanolamine, diethylamine, tricine, triethanolamine, bicine, or any combinations thereof. In some embodiments, a second regeneration buffer can comprise about, at least about, or at most about 0.1, 0.5, 1, 5, 10, 20, 30, 40, 50, 60, 70, 75, 80, 85, 90, 95, 100, 105, 110, 115, 120, 125, 130, 135, 140, 145, 146,147, 148, 149, 150, 151,152, 153, 154, 155, 156,157, 158, 159, 160, 170, 180, 190, 200, 300, 400, 500, 600, 700, 800, 900, 1000, 1500, or 2000 mM of buffering agent, such as Tris. In some embodiments, a second regeneration buffer comprises 50 mM to 150 mM of a buffering agent, optionally wherein the buffering agent is Tris. In some embodiments, a second regeneration buffer comprises about 100 mM Tris.

In some embodiments, pH of the second regeneration buffer is about, at least about, or at most about 6, 6.5, 7, 7.1, 7.2, 7.3, 7.4, 7.5, 7.6, 7.7, 7.8, 7.9, 8, 8.5, or 9 (or any range derivable therein). In some embodiments, pH of a second regeneration buffer is about 7 to 8. In some embodiments, pH of a second regeneration buffer is about 7.5.

In some embodiments, a second regeneration buffer comprises about, at least about, or at most about 0.1, 0.5, 1, 1.5, 2, 2.5, 3, 3.5, 4, 4.5, 5, 5.5, 6, 6.5, 7, 7.5, 8, 8.5, 9, 9.5, or 10% (or any range derivable therein) of sarkosyl, about, at least about, or at most about 10, 20, 30, 40, 50, 60, 70, 75, 80, 85, 90, 95, 100, 105, 110, 115, 120, 125,130, 135, 140, 145, 146, 147, 148, 149, or 150 mM (or any range derivable therein) of Tris, and has a pH of about, at least about, or at most about 6, 6.5, 7, 7.1, 7.2, 7.3, 7.4, 7.5, 7.6, 7.7, 7.8, 7.9, 8, 8.5, or 9 (or any range derivable therein). In some embodiments, a second regeneration buffer wherein the second regeneration buffer comprises 0.1% to 1.5% of sarkosyl, 50 mM to 150 mM of Tris, and a pH of 7 to 8. In some embodiments, a second regeneration buffer comprises about 1% sarkosyl, about 100 mM Tris at about pH 7.5.

In some embodiments, a second regeneration buffer flows upward through a stationary phase. In some embodiments, a second regeneration buffer is flowed upward through a column containing a stationary phase. In some embodiments, a second regeneration buffer is flowed through a stationary phase in a direction that is opposite to the direction that an elution buffered is flowed through the stationary phase. In some embodiments, a second regeneration buffer is flowed through a column containing a stationary phase in a direction that is opposite to the direction that an elution buffered is flowed through the column containing the stationary phase.

In some embodiments, a stationary phase is contacted with about, at least about, or at most about 0.5, 1, 1.5, 2, 2.5, 3, 3.5, 4, 4.5, 5, 5.5, 6, 6.5, 7, 7.5, 8, 8.5, 9, 9.5, 10, 15, 20, or 30 CV (or any range derivable therein) of a second regeneration buffer. In some embodiments, a stationary phase is contacted with 1 CV to 10 CV or 4.5 CV to 5.5 CV of a second regeneration buffer. In some embodiments, a stationary phase is contacted with about 5 CV of a second regeneration buffer.

In some embodiments, a second regeneration buffer is applied to a stationary phase with a residence time about, at least about, or at most about 0.1, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 min/column volume (CV) (or any range derivable therein). In some embodiments, a second regeneration buffer is applied to a stationary phase at a flow velocity of about, at least about, or at most about 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 60, 65, 70, 80, 90, 100, 110, 120, 130, 140, 150, 160, 170,180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 700, 750, 800, 900, 1000, 1500, or 2000 cm/hr (or any range derivable therein).

In some embodiments, a first regeneration buffer, a second regeneration buffer, or both remove impurities from a stationary phase such that a i) A280 peak area in an elute from the first regeneration buffer, the second regeneration buffer, or both is about, at least about, 700, 800, 900, 1000, 1200, 1300, 1400, or 1500 mL*mAU (or any range derivable therein); and/or ii) A260 peak area in the elute from the first regeneration buffer, the second regeneration buffer, or both is about, at least about, 300, 400, 500, 600, 700, or 800 mL*mAU (or any range derivable therein).

In some embodiments, a method of regenerating a stationary phase further comprises loading another solution comprising an rAAV vector on an affinity chromatography stationary phase after removing at least a portion of a second regeneration buffer. In some embodiments, a method of regenerating a stationary phase further comprises applying a second amount of a pre-elution wash solution on an affinity chromatography stationary phase after removing at least a portion of a second regeneration buffer.

H. Additional Steps

In certain embodiments, a method of purification of AAV vector (e.g. rAAV) further comprises filtering a solution comprising an AAV vector prior to loading on a stationary phase with a guard column, guard filter, or both a guard column and a guard filter. In some embodiments, a guard column is a POROS™ XQ, POROS™ HQ, POROS™ Benzyl Ultra, Capto Octyl, or Capto 700 guard column. In some embodiments, a guard filter is a 0.2 μm nominal filter, such as 0.2 μm nominal PaII™ EAV pre-column filter. In some embodiments, a guard column, and/or a guard filter is not used.

The some embodiments, a method of AAV vector (e.g. rAAV) purification by affinity chromatography can include additional steps selected from pre-use flushing of a column media to displace a storage solution, pre-use sanitization of a column media, post-sanitization rinse of a column media, equilibration of a column media (e.g. during pre-use, between application of different solutions during use, and/or post use), post use application of storage media, or any combinations thereof. In some embodiments one or more additional steps can be excluded. One of skill in the art will also understand that the order of these steps may vary, and that certain steps may be performed more than once, and not necessarily in sequence.

In some embodiments, portions of, all of ranges described herein can be excluded. The % concentration mentioned herein can be wt. % and/or vol. % concentration.

### VII. Equivalents

The foregoing written specification is considered to be sufficient to enable one skilled in the art to practice the disclosure. The foregoing description and Examples detail certain exemplary embodiments of the disclosure. It will be appreciated, however, that no matter how detailed the foregoing may appear in text, the disclosure may be practiced in many ways and the disclosure should be construed in accordance with the appended claims and any equivalents thereof.

All references cited herein, including patents, patent applications, papers, text books, and the like, and the references cited therein, to the extent that they are not already, are hereby incorporated herein by reference in their entirety.

### VIII. Exemplary Embodiments

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

### EXAMPLES

### Example 1: Screening of Pre-Elution Washes for Optimal Conditions

A. Preparation of Affinity Load for Pre-Elution Wash Screening

HEK293 cells were grown in suspension culture and transfected with a 3-plasmid system to produce rAAV9 vector per standard methods know in the art (Grieger et al. (2016) Molecular Therapy 24(2):287-297). Cell suspension was obtained from a 50 L single-use bioreactor (SUB) for harvest. The cell harvest was treated with 0.5% triton X-100 (also known as Octoxynol-9) and mixed for 30 minutes to lyse the cells and release AAV9 including full capsids comprising a rAAV genome, intermediate capsids, and empty capsids. The lysate was flocculated, depth filtered, quenched, and 0.2 μm filtered to produce a clarified lysate that was loaded onto affinity chromatography columns as described below.

B. Pre-Elution Wash Screening via Affinity Chromatography

Three pre-elution washes (Wash 1, Wash 2, and Wash 3), described in Table 1, were studied to determine the optimal conditions to remove bound impurities while keeping AAV9 bound to a resin ligand. A POROS™ CaptureSelect AAV9 column (1 cm inner diameter, 10 cm bed height, and 8 mL column volume) was connected to an AKTA Avant 150 to be equilibrated, loaded with 2 L of 50 SUB clarified lysate, and washed. A 5 column volume (CV) wash before the elution was performed at the specified flow velocity and residence time shown in Table 1. Each method with different wash buffers were performed on different affinity columns to obtain replicates of each condition. A 3 mL elution volume and 40 mL wash volume were collected for each run and each condition. Collected pre-elution washes were evaluated for absorbance at 280 nm (A280), wherein higher peak height indicates higher levels of impurities. Affinity eluates were evaluated for viral genome recovery (% vg recovery), % purity, size exclusion chromatography (SEC) with simultaneous absorbance measurement for 260 nm/280 nm ratio (SEC A260/280), host cell DNA (HCDNA), host cell protein (HCP) removal, and infectivity ratio as shown in Table 2.

TABLE 1
Affinity Chromatography Method for Pre-Elution Wash Screening.

| | | | Residence | Flow |
|---|---|---|---|---|
| | | Column | Time | Velocity |
| Step | Solution | Volumes | (min/CV) | (cm/hr) |
| Equilibration | 100 mM Tris, pH 7.5 | 5 | 3 | 200 |
| 1 | | | | |
| Sample-Load | clarified lysate | 250 | 3 | 200 |
| | from 50 L SUB | | | |
| Equilibration | 100 mM Tris, pH 7.5 | 5 | 3 | 200 |
| 2 | | | | |
| Pre-Elution | (Wash 1) 17% ethanol, | 5 | 3 | 200 |
| Wash | 1M NaCl, 100 mM | | | |
| | Tris, pH 7.5; (Wash 2) | | | |
| | 17% ethanol, 1M | | | |
| | NaCl, 150 mM sodium | | | |
| | acetate, pH 5.6; or | | | |
| | (Wash 3) 17% ethanol, | | | |
| | 150 mM sodium | | | |
| | acetate, pH 5.6 | | | |
| | (Wash 4) No wash | | | |
| Equilibration | 150 mM sodium | 5 | 3 | 200 |
| 3 | acetate, pH 5.6 | | | |
| Elution | 150 mM sodium | 5 | 8 | 75 |
| | acetate, 25 mM | | | |
| | MgCl₂, 100 | | | |
| | mM glycine, pH 3.0 | | | |
| Equilibration | 150 mM sodium | 5 | 8 | 75 |
| 4 | acetate, pH 5.6 | | | |
| Regeneration | 0.1N phosphoric | 4 | 8 | 75 |
| 1 | acid, pH 1.9 | | | |
| Regeneration | 1% sarkosyl, 100 mM | 4 | 8 | 75 |
| 2 | Tris, pH 7.5 | | | |
| Equilibration | 100 mM Tris, pH 7.5 | 4 | 8 | 75 |
| 5 | | | | |
| Storage | 17% ethanol | 3 | 8 | 75 |

TABLE 2
Affinity Eluate Results from Pre-Elution Wash Screening.

| | Pre-Elution | Affinity Elution Peak Analytical Data | | | | | |
|---|---|---|---|---|---|---|---|
| | Wash A280 | % vg | SEC | % purity | HCDNA | HCP | |
| Pre-Elution Wash | Peak Height | recovery | A260/ | (RP-HPLC, | (ng/1 × | (ng/1 × | Infectivity |
| (Wash Name) | (mAU) | (qPCR) | 280 | NR) | 10¹⁴ vg) | 10¹⁴ vg) | Ratio (VG/IU) |
| No Wash (4) | 200 | 84.4 | 0.97 | 72.0 | 599 | 16,286 | 15,562 |
| 17% ethanol, 1M | 200 | 67.0 | 0.95 | 67.0 | 1014 | 17,793 | 55,305 |
| NaCl, 100 mM Tris, | | | | | | | |
| pH 7.5 (1) | | | | | | | |
| 17% ethanol, 1M | 500 | 78.8 | 0.96 | 92.0 | 746 | 8,826 | 16,256 |
| NaCl, 150 mM | | | | | | | |
| sodium acetate, pH | | | | | | | |
| 5.6 (2) | | | | | | | |
| 17% ethanol, 150 | 1800 | 80.2 | 0.94 | 92.0 | 634 | 15,981 | 12,962 |
| mM sodium | | | | | | | |
| acetate, pH 5.6 (3) | | | | | | | |
| 17% ethanol, 150 | 2000 | 92.0 | 0.95 | 94.0 | 117 | 7,771 | 17,581 |
| mM sodium | | | | | | | |
| acetate, pH 5.6 (3) | | | | | | | |
| 17% ethanol, 150 | 1900 | 86.0 | 0.97 | 92.0 | 398 | 7,481 | 20,346 |
| mM sodium | | | | | | | |
| acetate, pH 5.6 (3) | | | | | | | |

Elution buffer for all affinity runs: 150 mM sodium acetate, 100 mM glycine, 25 mM MgCl₂, pH 3.0

The affinity elution, with and without pre-elution wash steps, showed similar product quality as determined by SEC A260/280 ratio, HCDNA levels, and infectivity ratio. The affinity elution, without the wash step, and with wash 1, showed significantly lower % purity compared to the other tested conditions, as determined by reverse phase-high performance liquid chromatography, non-reducing (RP-HPLC, NR) (67% for wash 1 vs 92% for wash 3, Table 2). As shown in Table 2, the collected 17% ethanol, 150 mM sodium acetate, pH 5.6 wash (wash 3) had a A280 peak height 9- to 10-fold higher than no wash or wash 1, which indicated that higher levels of impurities were removed by this pre-elution wash. The 17% ethanol, 1 M NaCl, 150 mM sodium acetate, pH 5.6 wash (wash 2) had a A280 peak with only a 2.5-fold higher peak height than no wash or wash 1, as show in Table 2, which indicated that the sodium chloride, the additional component in wash 2 as compared to wash 3, was interfering with removal of impurities. Therefore, the 17% ethanol, 150 mM sodium acetate, pH 5.6 wash (wash 3) step was elected as the pre-elution wash for affinity chromatography due to its effectiveness in removing bound impurities while maintaining high product recovery in the elution step.

C. Summary

This Example describes the optimization of the pre-elution wash for the affinity chromatography capture step of rAAV9 vector comprising a mini-dystrophin transgene. An effective pre-elution wash removed bound impurities while maintaining rAAV9-resin ligand interactions. It was concluded that the 17% ethanol, 150 mM sodium acetate, pH 5.6 pre-elution wash would be used in the affinity chromatography process since it removed impurities while maintaining a consistently high % viral genome (vg) recovery and % purity.

### Example 2: MgCl₂ Concentration and PH Screening for Affinity Elution Buffer

A. Preparation of Affinity Load for Screening Study

HEK293 cells were grown in suspension culture and transfected with a 3-plasmid system to produce rAAV9 vector. Cell suspension was obtained from a 250 L SUB for harvest. Cell harvest was treated with 0.5% triton X-100 and mixed for 30 minutes to lyse the cells and release AAV9 including full capsids comprising a rAAV genome, intermediate capsids, and empty capsids. The lysate was flocculated, depth filtered, quenched, and 0.2 μm filtered to produce a clarified lysate that was loaded onto affinity chromatography columns as described below.

B. Initial Elution Buffer Screening Study (DoE #1): pH and MgCl₂ Concentration

Optimization of the affinity elution step is crucial for recovering the maximum amount of AAV9 from the affinity column. The elution buffer components can play a key role in ensuring that AAV9 is recovered from the affinity resin. In this first screening study, the factors tested were the pH (3.0, 4.0, and 5.0) and MgCl₂ concentration (0, 50, 100, and 200 mM) in the affinity elution buffer as shown in Table 3. The average load on the affinity column for these experiments was 3.8×10¹³ vg/mL resin. Two POROS™ CaptureSelect AAV9 affinity columns (1 cm inner diameter, 10 cm bed height, and 8 mL column volume) were used to conduct the design of experiments study that consisted of 12 runs (6 runs on each column).

TABLE 3
Design of Experiment (DoE) #1: Affinity
Elution Buffer Conditions and Data.

| | | | Elution | % vg | A260/280 | HCDNA |
|---|---|---|---|---|---|---|
| Run | | MgCl₂ | Conductivity | recovery | Ratio | (ng/1 × |
| Number | pH | (mM) | (mS/cm) | (qPCR) | (SEC) | 10¹⁴ vg) |
| 1 | 3.0 | 200 | 34.60 | 29.0 | 0.84 | 197 |
| 2 | 4.0 | 50 | 16.49 | 5.0 | 0.86 | 1112 |
| 3 | 5.0 | 0 | 12.59 | 0.1 | NA | 142 |
| 4 | 4.0 | 100 | 24.31 | 9.7 | 0.85 | 338 |
| 5* | 3.0 | 0 | 3.94 | 65.0 | 0.83 | 773 |
| 6 | 4.0 | 50 | 16.46 | 13.0 | 0.86 | 282 |
| 7 | 5.0 | 100 | 25.35 | 0.2 | 0.87 | 166 |
| 8* | 3.0 | 0 | 3.94 | 60.0 | 0.83 | 500 |
| 9 | 4.0 | 50 | 16.46 | 9.0 | 0.84 | 269 |
| 10 | 5.0 | 50 | 18.64 | 0.2 | 0.83 | 169 |
| 11 | 3.0 | 100 | 20.47 | 50.0 | 0.85 | 264 |
| 12 | 3.0 | 200 | 34.60 | 43.0 | 0.86 | 170 |

All elution buffers contain 100 mM glycine and 100 mM sodium citrate
*Precipitation was observed in Runs 5 and 8, both which used pH 3, 0 mM MgCl₂ elution buffers.

The affinity chromatography protocol that was used for these experiments is shown in Table 4 with the elution buffer composition being varied as shown in Table 3. For each run, the affinity eluate was collected and submitted for analytical testing that include % vg recovery (as measured by qPCR of inverted terminal repeats (ITR qPCR)), SEC A260/A280 ratio, and HCDNA (as measured by qPCR), which are shown in Table 3. The elution buffer with the highest product recovery as determined by % vg recovery contained 100 mM glycine, 100 mM sodium citrate, 0 mM MgCl₂ pH 3.0 (60%-65% vg recovery by qPCR, run numbers 5 and 8 in Table 3), but precipitation was observed. These elution samples were centrifuged with the supernatant taken for analytical testing. For the elution buffers at pH 3.0, there was a significant range of HCDNA present in the affinity eluate which was dependent on the MgCl₂ concentration. The HCDNA amount varied between 170 ng/1×10¹⁴ vg for the pH 3.0, 200 mM MgCl₂ elution buffer and 773 ng/1×10¹⁴ vg for the pH 3.0, 0 mM MgCl₂ elution buffer. Thus, MgCl₂ concentration influenced the HCDNA levels in the affinity eluate. The presence of MgCl₂ in the low pH elution buffers prevented precipitation. Elution buffers at pH 4.0 or 5.0 resulted in the lowest product recoveries, regardless of MgCl₂ concentration. The SEC A260/A280 ratio varied between 0.83-0.86 for all conditions which suggests that the elution fractions contained the same ratio of empty to full capsids (approximately 5%-10% full capsids). From this initial screening study, it was determined that the lower pH elution buffers and the presence of MgCl₂ are optimal conditions for the affinity elution buffer. This initial screening study informed the design of the next experiment (DOE #2) that studied similar parameters such as MgCl₂ concentration and pH.

TABLE 4
Affinity Chromatography Method for Affinity Elution Buffer Study

| | | | Residence | Flow |
|---|---|---|---|---|
| | | Column | Time | Velocity |
| Step | Solution | Volumes | (min/CV) | (cm/hr) |
| Equilibration | 100 mM Tris, pH 7.5 | 5 | 4 | 200 |
| 1 | | | | |
| Sample-Load | clarified lysate | 64 | 3 | 200 |
| Equilibration | 100 mM Tris, pH 7.5 | 5 | 3 | 200 |
| 2 | | | | |
| Pre-Elution | 20% ethanol, 1M | 5 | 3 | 200 |
| Wash | NaCl, 100 mM Tris, | | | |
| | pH 7.5 | | | |
| Equilibration | 100 mM sodium | 5 | 3 | 200 |
| 3 | citrate, pH 7.0 | | | |
| Elution | X glycine, 100 mM | 5 | 8 | 75 |
| | sodium citrate, X | | | |
| | MgCl₂ | | | |
| Equilibration | 100 mM sodium | 5 | 8 | 75 |
| 4 | citrate, pH 7.0 | | | |
| GuHCl Strip | 3M guanidine HCl, | 4 | 8 | 75 |
| | pH 5.05 | | | |
| Equilibration | 100 mM Tris, pH 7.5 | 4 | 4 | 75 |
| 5 | | | | |
| Storage | 20% ethanol | 3 | 4 | 75 |

C. Second Elution Buffer Screening Study (DoE #2): pH, MgCl₂ Concentration, and Glycine Concentration Evaluation

Based on the results obtained from the initial screening study (DoE #1), a second screening study (DoE #2) was developed to narrow the DoE and focus on key factors that affect the elution attributes. The factors tested in this secondary screening study were the pH (3.0, 3.5), MgCl₂ concentration (25, 50, 100, 200 mM), and glycine concentration (0, 100, 200 mM) as shown in Table 5. The average load on the affinity column for these experiments was 2.5×10¹³ vg/mL resin. Two POROS™ CaptureSelect AAV9 affinity columns (1 cm inner diameter, 10 cm bed height, and 8 mL column volume) were used for these experiments, with 7 runs on each column. The affinity chromatography protocol used for these experiments is outlined in Table 4, with the elution buffer conditions being varied. For each run, the affinity eluate was collected and submitted for various analytical testing that include % vg recovery measured by qPCR and SEC A260/A280, results are shown in Table 5. Overall, the % vg recovery was lower for this secondary screening study than that of the initial screening study due to lower resin challenge. The resin challenge (average load) was 3.8×10¹³ vg/mL and 2.5×10¹³ vg/mL of resin for the first (DoE #1) and second (DoE #2) study screen, respectively.

TABLE 5
DoE #2: Table for Affinity Elution Buffer Conditions and Data.

| | | | | Elution | % vg | SEC |
|---|---|---|---|---|---|---|
| Run | | MgCl₂ | glycine | Conductivity | recovery | A260/280 |
| Number | pH | (mM) | (mM) | (mS/cm) | (qPCR) | ratio |
| 1 | 3.5 | 50 | 100 | 14.16 | 24.4 | 0.85 |
| 2 | 3.0 | 100 | 100 | 20.47 | 42.8 | 0.86 |
| 3 | 3.0 | 200 | 100 | 34.60 | 30.5 | 0.85 |
| 4 | 3.5 | 25 | 0 | 10.26 | 33.3 | 0.85 |
| 5* | 3.0 | 50 | 200 | 12.00 | 47.7 | 0.85 |
| 6 | 3.5 | 50 | 100 | 14.16 | 31.1 | 0.85 |
| 7* | 3.0 | 50 | 0 | 12.45 | 46.3 | 0.85 |
| 8 | 3.5 | 100 | 0 | 22.18 | 29.3 | 0.84 |
| 9 | 3.5 | 50 | 100 | 14.16 | 33.4 | 0.85 |
| 10 | 3.0 | 200 | 100 | 34.6 | 35.1 | 0.85 |
| 11 | 3.5 | 25 | 200 | 9.92 | 49.9 | 0.85 |
| 12 | 3.0 | 100 | 100 | 20.47 | 35.2 | 0.85 |
| 13* | 3.0 | 25 | 100 | 8.34 | 55.5 | 0.85 |
| 14 | 3.5 | 100 | 200 | 20.26 | 29.0 | 0.85 |

All elution buffers contain 100 mM sodium citrate
*Precipitation was observed in column eluate. Eluate was centrifuged prior to sample submission. The supernatant was submitted for testing.

The elution buffer that consisted of 25 mM MgCl₂, 100 mM glycine, 100 mM sodium citrate, pH 3.0 (Table 5, Run 13) had the highest % vg recovery (55% by qPCR). However, it was observed that the affinity eluate had some precipitation which was determined to be caused by additional impurities co-eluting with AAV9 by silver stain SOS-PAGE analysis (data not shown). The SEC A260/280 ratios for all conditions were consistent suggesting similar full to empty capsid ratios in the affinity eluate. Comparing DoE #2 data (Table 5) with DoE #1 data (Table 3) showed that there was an optimal conductivity range of the elution buffer that prevented co-elution of residual impurities. As shown in Table 6, a conductivity range of 20-34 mS/cm elutes the AAV9 vector with high yield and reduces the elution of impurities and reduces precipitation. Conversely, elution with a lower conductivity (e.g., 4 mS/cm) leads to high yield, but also leads to impurity-based precipitation. This trend indicated that elution buffer conductivity as well as ionic strength played a role in reducing the co-elution of impurities. The most favorable condition was a lower MgCl₂ concentration, and an elution conductivity range of 20-34 mS/cm.

TABLE 6
DoE #1 and #2 Conductivity Data

| | | | | | | | | Precipitation |
|---|---|---|---|---|---|---|---|---|
| DoE | DoE | | | | sodium | Elution | % VG | Due to |
| Study | Run | | MgCl₂ | glycine | citrate | Conductivity | Recovery | Impurity* |
| # | # | pH | (mM) | (mM) | (mM) | (mS/cm) | (qPCR) | Co-elution |
| 1 | 5 | 3.0 | 0 | 100 | 100 | 4 | 65 | Yes |
| 1 | 11 | 3.0 | 100 | 100 | 100 | 20 | 50 | No |
| 1 | 12 | 3.0 | 200 | 100 | 100 | 34 | 43 | No |
| 2 | 10 | 3.0 | 200 | 100 | 100 | 34 | 35 | No |
| 2 | 12 | 3.0 | 100 | 100 | 100 | 20 | 35 | No |
| 2 | 13 | 3.0 | 25 | 100 | 100 | 8 | 56 | Yes |

*As judged by SDS-PAGE carried out on precipitate pellet and supernatant.

D. Summary

In this example, two DoEs were performed to narrow down the key elution buffer components that provide optimal elution attributes. The first DoE tested a broad range of pH values, (pH 3.0, 4.0, and 5.0) and MgCl₂ (0, 50, 100, and 200 mM) concentrations in 100 mM sodium citrate. From this first DoE, it was concluded that lower pH elution buffers as well as the presence of MgCl₂ improves the % vg recovery of the affinity eluate. The second DoE tested a narrower range of pH values (pH 3.0 and 3.5), MgCl₂ concentrations (25, 50, 100, and 200 mM), and glycine concentrations (25, 50, 100, and 200 mM) in 100 mM sodium citrate. The pH range was 3.0 and 3.5 due to results obtained from the first DoE. From the second DoE, it was concluded that pH 3.0, lower MgCl₂ concentration, and an elution conductivity of 20-34 mS/cm were all conditions that led to optimal % vg recovery from the affinity column.

### Example 3: Role of Sodium Citrate in Affinity Elution Buffer and its Effect on Anion Exchange Chromatography (AEX) Elution Profile

A. Preparation of Affinity Load for Elution Buffer Comparison Study

HEK293 cells were grown in suspension culture and transfected with a 3-plasmid system to produce rAAV9 vector. Cell suspension was obtained from a 50 L SUB for harvest. Cell harvest was treated with 0.5% triton X-100 and mixed for 30 minutes to lyse the cells and release AAV9 including full capsids comprising a rAAV genome, intermediate capsids, and empty capsids. The lysate was flocculated, depth filtered, quenched, and 0.2 μm filtered to produce a clarified lysate that was loaded onto affinity chromatography columns as described below.

B. Affinity Chromatography

The affinity elution buffers studied were 200 mM glycine, 50 mM MgCl₂, pH 3.5 (Buffer A) and 100 mM glycine, 100 mM sodium citrate, 100 mM MgCl₂, pH 3.0 (Buffer B). The affinity chromatography method for both elution buffers is shown in Table 7. A POROS™ CaptureSelect AAV9 affinity column (1 cm inner diameter, 10 cm bed height, and 8 mL column Table 7. Affinity Chromatography Method for Elution Buffer Comparison Residence Flow

TABLE 7
Affinity Chromatography Method for Elution Buffer Comparison

| | | | Residence | Flow |
|---|---|---|---|---|
| | | Column | Time | Velocity |
| Step | Solution | Volumes | (min/CV) | (cm/hr) |
| Equilibration | 100 mM Tris, pH 7.5 | 5 | 5 | 120 |
| 1 | | | | |
| Sample-Load | Clarified lysate | 125 | 2.9 | 207 |
| | from 50 L SUB | | | |
| Equilibration | 100 mM Tris, pH 7.5 | 5 | 5 | 120 |
| 2 | | | | |
| Pre-Elution | 20% ethanol, 1M | 5 | 5 | 120 |
| Wash | NaCl, 100 mM Tris, | | | |
| | pH 7.5 | | | |
| Equilibration | 100 mM sodium | 5 | 5 | 120 |
| 3 | citrate, pH 7.0 | | | |
| Elution | Buffer A: 200 mM | 5 | 8 | 75 |
| | glycine, 50 mM | | | |
| | MgCl₂, pH 3.5 | | | |
| | Buffer B: 100 mM | | | |
| | glycine, 100 mM | | | |
| | MgCl₂, 100 mM | | | |
| | sodium citrate, pH 3.0 | | | |
| Equilibration | 100 mM sodium | 5 | 8 | 75 |
| 4 | citrate, pH 7.0 | | | |
| GuHCl Strip | 3M guanidine HCl, | 4 | 8 | 75 |
| | pH 5.05 | | | |
| Equilibration | 100 mM Tris, pH 7.5 | 4 | 4 | 75 |
| 5 | | | | |
| Storage | 20% ethanol | 3 | 4 | 75 |

Elution fractions of 1 mL each were collected and then pooled for a total of 5 mL. For both runs, the pooled affinity eluate was neutralized with 1 M Tris pH 9.0. For elution buffer A, the neutralized affinity eluate was significantly turbid, so the solution was centrifuged. The supernatant was submitted for testing and used as the load for the subsequent anion exchange chromatography step. For Buffer B, the pooled affinity eluate was clear, and no precipitation was observed after neutralization. The analytical testing results for each affinity eluate are shown in Table 8. The elution buffers displayed similar % vg recovery and % purity profiles. The main difference between Buffer A and Buffer B was the presence of precipitation upon neutralization. The elution profile for Buffer A was broader which could indicate the co-elution of residual impurities as shown in FIG. 1. This broader elution profile was caused by the more gradual pH drop which may have resulted in the elution of impurities along with the rAAV capsids.

TABLE 8
Buffers A and B Affinity Eluate Results

| | % vg | % purity | A260/280 | Precipitation |
|---|---|---|---|---|
| Affinity Elution | recovery | (RP- | ratio | Post- |
| Buffer | (qPCR) | HPLC, NR) | (SEC) | Neutralization |
| Buffer A: 200 | 38 | 97.0 | 0.91 | Yes |
| mM glycine, 50 | | | | |
| mM MgCl₂, | | | | |
| pH 3.5 | | | | |
| Buffer B: 100 | 42 | 96.5 | 0.91 | No |
| mM glycine, 100 | | | | |
| mM MgCl₂, 100 | | | | |
| mM sodium | | | | |
| citrate, pH 3.0 | | | | |

C. Anion Exchange Chromatography (AEX) Using Buffer A and B Affinity Eluates

The affinity eluates from the affinity runs with Buffers A and B were each further processed on anion exchange columns as described below. The AEX step is utilized to separate the empty and full AAV9 capsids as well as to reduce remaining host cell impurities. The affinity elution buffer composition can affect how well the AAV9 binds to the AEX column during empty/full capsid separation. A POROS™ HO column (HO) (0.5 cm inner diameter, 5 cm bed height, and 1 mL column volume) was used for both runs. The affinity eluates from both runs were adjusted to pH 9.0 using 1 M Tris base and then diluted initially 10-fold into a buffer comprising 20 mM Tris, 2% PEG (8k), pH 9.0. The eluates were diluted further with 20 mM Tris, 2% PEG (8k), pH 9.0 until the conductivity was ≤2.0 mS/cm. 2% PEG 8k was included in the dilution buffer as a co-solvent to promote binding of AAV9 to the AEX column. The AEX method used for each run is detailed in Table 9.

TABLE 9
Anion Exchange Chromatography Method for HQ-17 and HQ-18

| | | | Residence | Flow |
|---|---|---|---|---|
| | | Column | Time | Velocity |
| Step | Solution | Volumes | (min/CV) | (cm/hr) |
| Equilibration | 20 mM Tris, 2% PEG | 10 | 0.5 | 611 |
| | 8k, pH 9.0 | | | |
| Sample Load | 50 L SUB affinity | 70 | 0.5 | 611 |
| | elution pool diluted | | | |
| | into 10 × 20 | | | |
| | mM Tris, 2% PEG 8k, | | | |
| | pH 9.0 | | | |
| Equilibration | 20 mM Tris, 2% PEG | 28 | 0.5 | 611 |
| | 8k, pH 9.0 | | | |
| Wash | 20 mM Tris, pH 9.0 | 10 | 0.5 | 611 |
| Elution | (1) 20 mM Tris, pH | 50 | 0.5 | 611 |
| Gradient | 9.0 | | | |
| (0-250 mM | (2) 20 mM Tris, 0.5M | | | |
| NaCl/50 CV, | NaCl, pH 9.0 | | | |
| hold 10 CV) | | | | |
| Regeneration | 2M NaCl, 25 mM | 10 | 0.5 | 611 |
| | Tris, pH 9.0 | | | |
| Sanitization | 0.5M NaOH | 10 | 6 | 51 |
| Equilibration | 20 mM Tris HCl, pH | 10 | 0.5 | 611 |
| | 9.0 | | | |
| Storage | 20% ethanol | 10 | 0.5 | 611 |

AAV9 was eluted from the AEX column using a 50 column volume (50 mL) linear gradient from 0 to 250 mM NaCl, in 20 mM Tris pH 9.0 buffer. The flow through as well as the elution fractions were all collected and neutralized with 50 mM sodium citrate, pH 3.6. The elution peak fractions that had a SEC A260/280 ratio 1.2 were pooled as shown in Tables 10 and 11. For the AEX run (HQ-18) Buffer A affinity eluate (200 mM glycine, 50 mM MgCl₂, pH 3.5), 2 elution fractions were pooled while for the AEX run (HQ-17) Buffer B affinity eluate (100 mM glycine, 100 mM MgCl₂, 100 mM sodium citrate, pH 3.0), 8 elution fractions were pooled.

TABLE 10
SEC A260/280 Ratios for HQ-17 AEX Elution Fractions
(Affinity Elution Buffer B: 100 mM glycine, 100 mM
MgCl₂, 100 mM sodium citrate, pH 3.0)

| Frac # | 20 | 21 | 22 | 29 | 30 | 31 | 32 | 33 | 34 | 35 | 36 |
|---|---|---|---|---|---|---|---|---|---|---|---|
| SEC | 1.23 | 1.28 | 1.28 | 1.29 | 1.28 | 1.28 | 1.27 | 1.23 | 0 | 0 | 0 |
| Pooled | Yes | Yes | Yes | Yes | Yes | Yes | Yes | Yes | No | No | No |

TABLE 11
SEC A260/280 Ratios for HQ-18 AEX Elution Fractions
(Affinity Elution Buffer A: 200 mM
glycine, 50 mM MgCl₂, pH 3.5)

| Frac # | 25 | 26 | 27 | 28 | 29 | 30 |
|---|---|---|---|---|---|---|
| SEC | 1.15 | 1.22 | 1.22 | 1.16 | 1.04 | 0.94 |
| Pooled | No | Yes | Yes | No | No | No |

The elution pools for both runs were submitted for analytical testing of % vg recovery, % purity, HCDNA, and analytical ultracentrifugation (AUC) as shown in Table 12.

TABLE 12
Analytical Results for HQ-17 and HQ-18 AEX Elution Pools

| | % vg in | % vg | % purity | HCDNA | | | |
|---|---|---|---|---|---|---|---|
| | Flow- | recovery | (RP- | (ng/1 × | % Capsid | | |
| Elution | Through | (ITR | HPLC, | 10¹⁴ | Species (AUC) | | |
| Pool | (Unbound) | qPCR) | NR) | vg) | Full | Interm. | Empty |
| HQ-17 | 54 | 7 | NA | NA | 43 | 33 | 24 |
| HQ-18 | 0 | 27 | 99.7 | 518 | 38 | 25 | 39 |

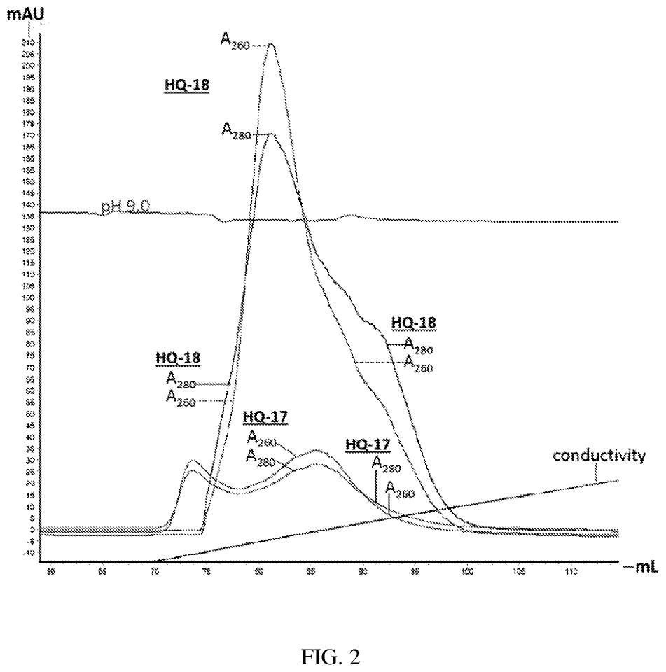

Minor load differences had dramatically different binding/elution profiles as shown in chromatogram overlay in FIG. 2. It was determined that citrate, a trivalent anion, outcompetes AAV9 with binding to the cationic AEX-HQ resin which leads to significantly different elution profiles and characteristics. HQ-17, which contained 8 mM citrate in the AEX load, resulted in 54% unbound VG. Conversely, HQ-18, which contained no citrate in the AEX load, completely bound all loaded VG (0% VG in flow through fraction). However, precipitation was seen after neutralization of the Buffer A (200 mM glycine, 50 mM MgCl₂, pH 3.5) which did not contain any citrate. This suggested that an anion was needed in the affinity elution buffer to prevent aggregation (and thus precipitation), but the anion could not be a strong anion like citrate, as it preferentially bound to the AEX resin as compared to AAV9 capsids.

D. Summary

In this example, two affinity elution buffers (Buffers A and B) were tested to determine their effect on affinity and AEX elution profiles. Buffer A did not contain sodium citrate while Buffer B contained 100 mM sodium citrate. Buffers A and B had similar affinity elution profiles with similar % vg recovery. The main difference was that upon neutralization of the Buffer A affinity eluate (which did not contain sodium citrate), precipitation was observed. Once the affinity eluate from each buffer run was neutralized, they were diluted and processed through AEX. The AEX eluate from the chromatography run which used the Buffer A affinity eluate (did not contain sodium citrate) had a much higher % vg recovery than the AEX eluate from the chromatography run which used the Buffer B affinity eluate (27% vs 7%). The AEX elution profiles for the Buffer A and Buffer B affinity eluates looked significantly different, with the AEX elution profile of Buffer A showing more optimal full/empty capsid enrichment.

It was concluded that sodium citrate interfered with the AAV9-AEX resin interaction, leading to reduced binding of AAV9 on the AEX resin. Therefore, citrate was removed from the affinity elution buffer and alternatives were studied.

### Example 4: Effect of Affinity Elution Buffers on AEX HQ Runs

A. Preparation of Affinity Load for Elution Buffer to AEX Run Study

HEK293 cells were grown in suspension culture and transfected with a 3-plasmid system to produce rAAV9 vector. Cell suspension was obtained from a wave bag for harvest. Cell harvest was treated with 0.5% tritonX-100 and mixed for 30 minutes to lyse the cells and release AAV9, including full capsids comprising an rAAV genome, intermediate capsids, and empty capsids. The lysate was flocculated, depth filtered, quenched, and 0.2 μm filtered to produce a clarified lysate that was loaded onto affinity chromatography columns as described below.

B. Affinity Chromatography to AEX Linking Study

In this linking study, affinity chromatography was carried out with various elution buffers, followed by dilution and empty/full capsid separations using anion exchange chromatography (AEX) on a POROS™ 50 HQ resin (referred to here as HQ). Once the top two performing affinity elution buffers were selected, large-scale studies were conducted to ensure that similar results could be ascertained. Six different affinity elution buffers were tested to determine their effects on the affinity and AEX elution profiles. The composition of these different elution buffers is provided in Table 13.

TABLE 13
Affinity Elution Buffer Compositions

| | | | | | Elution Buffer |
|---|---|---|---|---|---|
| Run | mM additional | glycine | MgCl₂ | | Conductivity |
| # | component (X) | (mM) | (mM) | pH | (mS/cm) |
| 19 | 0 | 100 | 100 | 3.0 | 19.4 |
| 20 | 100 sodium acetate | 100 | 100 | 3.0 | 19.6 |
| 21 | 30 sodium citrate | 100 | 100 | 3.0 | 18.8 |
| 22 | 150 sodium acetate | 100 | 100 | 3.0 | 19.8 |
| 23 | 100 sodium acetate + | 100 | 100 | 3.0 | 13.1 |
| | 25% iodixanol | | | | |
| N/A | 100 sodium acetate + | 100 | 100 | 3.0 | 8.7 |
| | 25% propylene glycol | | | | |

A total of 2 L of clarified cell lysate was loaded onto a POROS™ CaptureSelect AAV9 affinity column (1 cm inner diameter, 10 cm bed height, and 8 mL column volume). The affinity chromatography method for all six elution buffers is provided in Table 14.

TABLE 14
Affinity Chromatography Method for Six Elution Buffers

| | | | Residence | Flow |
|---|---|---|---|---|
| | | Column | Time | Velocity |
| Step | Solution | Volumes | (min/CV) | (cm/hr) |
| Equilibration | 100 mM Tris, pH 7.5 | 5 | 5 | 120 |
| 1 | | | | |
| Sample-Load | clarified lysate | 125 | 2.9 | 207 |
| | from Wave Bag | | | |
| Equilibration | 100 mM Tris, pH 7.5 | 5 | 5 | 120 |
| 2 | | | | |
| Pre-Elution | 20% ethanol, 1M | 5 | 5 | 120 |
| Wash | NaCl, 100 mM Tris, | | | |
| | pH 7.5 | | | |
| Equilibration | 100 mM sodium | 5 | 5 | 120 |
| 3 | citrate, pH 7.0 | | | |
| Elution | mM X, 100 mM | 5 | 8 | 75 |
| | glycine, 100 mM | | | |
| | MgCl₂, pH 3.0 | | | |
| Equilibration | 100 mM sodium | 5 | 8 | 75 |
| 4 | citrate, pH 7.0 | | | |
| GuHCl Strip | 3M guanidine HCl, | 4 | 8 | 75 |
| | pH 5.05 | | | |
| Equilibration | 100 mM Tris, pH 7.5 | 4 | 4 | 75 |
| 5 | | | | |
| Storage | 20% ethanol | 3 | 4 | 75 |

1 mL elution fractions were collected from the affinity column and pooled based on A280 values from Nanodrop analysis. The elution pool (950 μL of each fraction) was neutralized using 1 M Tris base, pH 9.0. If precipitation was observed after neutralization, then the elution pool was centrifuged, and the supernatant was retained for analytical testing. The analytical results for the affinity elution pool is shown in Table 15 for each run.

TABLE 15
Analytical Results for Affinity and AEX Elution Pools

| | Affinity Step Metrics | | | | | | | |
|---|---|---|---|---|---|---|---|---|
| | Precipitation | | | AEX Step Metrics | | | | |
| Run | Post- | % vg | % | % vg | % capsid species (AUC) | | | % |
| # | neutralization? | recovery | purity | recovery | Full | Interm. | Empty | purity |
| 19 | Yes | 60 | 97.8 | 35 | 42 | 20 | 38 | 99.4 |
| 20 | Yes | 57 | 97.9 | 39 | 41 | 33 | 26 | 99.1 |
| 21 | Yes | 51 | 97.4 | 51 | 41 | 38 | 21 | 98.9 |
| 22 | Yes | 54 | 97.0 | 54 | 54 | 33 | 13 | 98.9 |
| 23 | No | 62 | 80.8 | 39 | 39 | 26 | 29 | 99.2 |
| N/A | No | 49 | 86.9 | N/A | N/A | N/A | N/A | N/A |

The affinity eluate obtained for each of the affinity chromatography runs was then loaded onto an anion exchange chromatography column to assess empty/full separation. The affinity elution pool was diluted 9- to 11-fold into 20 mM Tris, 2% PEG 8k, pH 9.0 prior to loading on the AEX column. The dilution factor depended on the initial affinity eluate volume and the eluate was diluted until the conductivity was ≤2.0 mS/cm. The pH of each load was adjusted to 9.0 with the addition of 1 M Tris base. A POROS™ HO column (0.5 cm inner diameter, 5 cm bed height, and 1 mL column volume) was used for the analysis of the different affinity eluate by AEX.

The anion exchange chromatography method was consistent across all runs and is provided in Table 16.

TABLE 16
Anion Exchange Chromatography Method for
Six POROS ™ 50 HQ Runs

| | | | Residence | Flow |
|---|---|---|---|---|
| | | Column | Time | Velocity |
| Step | Solution | Volumes | (min/CV) | (cm/hr) |
| Equilibration | 20 mM Tris, 2% PEG | 10 | 0.5 | 611 |
| | 8k, pH 9.0 | | | |
| Sample Load | Wave bag, affinity | 70 | 0.5 | 611 |
| | elution pool diluted | | | |
| | into 20 mM Tris, | | | |
| | 2% PEG 8k, pH 9.0 | | | |
| Equilibration | 20 mM Tris, 2% PEG | 28 | 0.5 | 611 |
| | 8k, pH 9.0 | | | |
| Wash | 20 mM Tris, pH 9.0 | 10 | 0.5 | 611 |
| Elution | (1) 20 mM Tris, pH | 50 | 0.5 | 611 |
| Gradient | 9.0 | | | |
| (0-250 mM | (2) 20 mM Tris, 0.5M | | | |
| NaCl/50 CV, | NaCl, pH 9.0 | | | |
| hold 10 CV) | | | | |
| Regeneration | 2M NaCl, 25 mM | 10 | 0.5 | 611 |
| | Tris, pH 9.0 | | | |
| Sanitization | 0.5M NaOH | 10 | 6 | 51 |
| Equilibration | 20 mM Tris HCl, pH | 10 | 0.5 | 611 |
| | 9.0 | | | |
| Storage | 20% ethanol | 10 | 0.5 | 611 |

1 mL elution fractions from the AEX column were collected and submitted for SEC A260/280 to determine the elution pooling strategy. If the SEC A260/280 ratio was 1.2, then that fraction was included as part of the elution pool. For each run, the elution pool was submitted for further analytical testing to obtain % vg recovery (as measured by transgene qPCR), % purity (as measured by RP-HPLC, NR), HCP, HCDNA (as measured by qPCR), and % empty/intermediate/full capsids (measured by SEC-HPLC). Data are shown in Table 15.

Of the six affinity elution buffers tested, runs HQ-21 (30 mM sodium citrate, 100 mM glycine, 100 mM MgCl₂, pH 3.0) and HQ-22 (150 mM sodium acetate, 100 mM glycine, 100 mM MgCl₂, pH 3.0) were the top performing elution buffers. Upon dilution, both affinity elution buffers enabled high AAV9 binding to the POROS™ 50 HQ resin and high empty/full capsid separation. The key parameters that were indicative of high-performance were: two step % vg recovery (Affinity* AEX) of 26-29%, a % purity of 99% by RP-HPLC, NR, low HCDNA content (600 ng HCDNA/1×10¹⁴ vg), less than quantifiable levels of HCPs, and high percent full capsids (41%-54%) as measured by analytical ultracentrifugation (AUC).

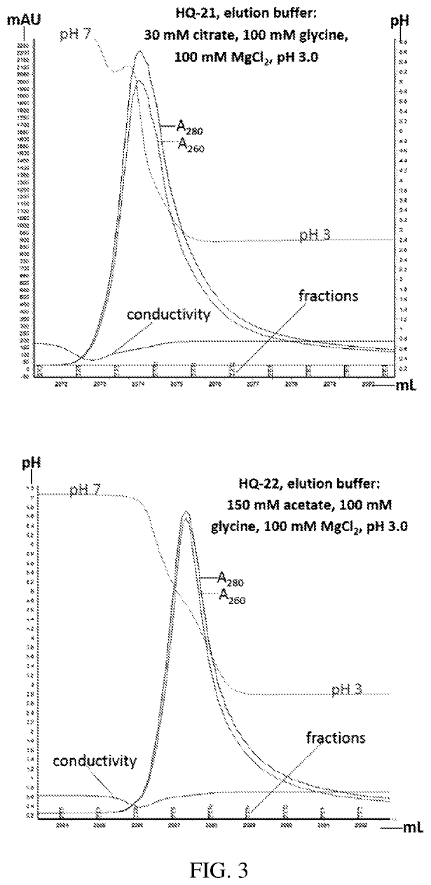

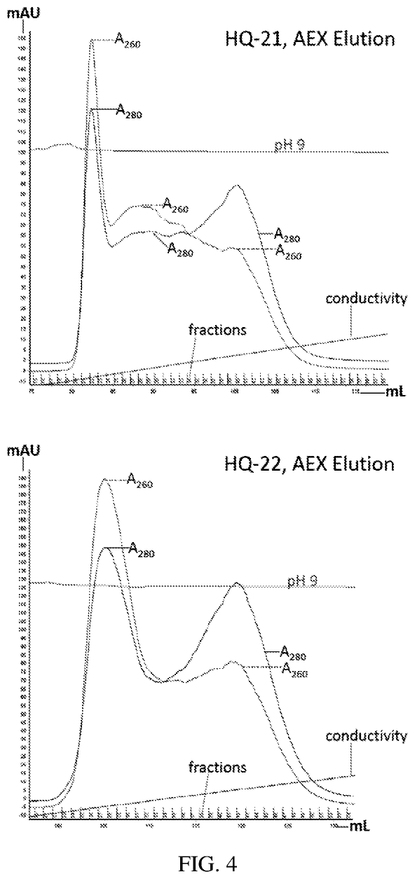

The affinity and AEX chromatograms for these two runs are shown in FIG. 3 and FIG. 4. Sharp and narrow affinity elution peaks for both runs are indicative of successful capture and elution of AAV9 with minimal co-elution of residual impurities. The bimodal AEX elution profiles for both runs shows sharp separation of empty and full capsids which was quantitatively confirmed with AUC data.

C. Scale-Up Affinity Chromatography to AEX Chromatography Linking Study

From the small-scale study, two different affinity elution buffers performed the best (30 mM sodium citrate, 100 mM glycine, 100 mM MgCl₂, pH 3.0 (“30 mM citrate” (HQ-21)) and 150 mM sodium acetate, 100 mM glycine, 100 mM MgCl₂, pH 3.0 (“150 mM acetate” (HQ-22))). Using these two affinity elution buffer candidates, a scale-up study was conducted to ensure that they would perform comparatively at a larger scale. A total of 7 L of 50 L SUB clarified lysate was loaded onto a POROS™ CaptureSelect AAV9 column (2.6 cm inner diameter, 19 cm bed height, and 100 mL column volume). The affinity chromatography method for this scale-up study is outlined in Table 17.

TABLE 17
Affinity Chromatography Method for Scale-Up Study

| | | | Residence | Flow |
|---|---|---|---|---|
| | | Column | Time | Velocity |
| Step | Solution | Volumes | (min/CV) | (cm/hr) |
| Equilibration 1 | 100 mM Tris pH 7.5 | 5 | 5 | 226 |
| Sample-Load | clarified lysate from | 100 | 2.9 | 390 |
| | Wave bag | | | |
| Equilibration 2 | 100 mM Tris pH 7.5 | 5 | 5 | 226 |
| Pre-Elution | 20% ethanol, 1M | 5 | 5 | 226 |
| Wash | NaCl, 100 mM Tris | | | |
| | pH 7.5 | | | |
| Equilibration 3 | 100 mM sodium | 5 | 5 | 226 |
| | citrate pH 7.0 | | | |
| Elution | HQ-24: 30 mM | 5 | 8 | 142 |
| | citrate, 100 mM | | | |
| | glycine, 100 mM | | | |
| | MgCl₂ pH 3.0 | | | |
| | HQ-25: 150 mM | | | |
| | acetate, 100 mM | | | |
| | glycine, 100 mM | | | |
| | MgCl₂ pH 3.0 | | | |
| Equilibration 4 | 100 mM sodium | 5 | 8 | 142 |
| | citrate pH 7.0 | | | |
| GuHCl Strip | 3M guanidine HCl | 4 | 8 | 142 |
| Equilibration 5 | 100 mM Tris pH 7.5 | 4 | 4 | 142 |
| Storage | 20% ethanol | 3 | 4 | 142 |

25 mL elution fractions were collected from the affinity column and pooled. The affinity elution pool was neutralized with 1 M Tris base, pH 9.0 and then submitted for analytical testing. The performance of the two affinity elution buffers at larger scale was similar in terms of yield and purity as shown in Table 18. The SEC A260/280 ratios (not shown) of the recovered vector indicated that neither of the affinity elution buffers increased enrichment of the full rAAV9 capsid as compared to the other buffer.

TABLE 18
Analytical Results for Scale-up Affinity to AEX Linking Studies

| | Affinity Step Metrics | | AEX Step Metrics | | | | |
|---|---|---|---|---|---|---|---|
| | % vg | | % vg step | % capsid species (AUC) | | | |
| Run# | recovery | % purity | yield | Full | Interm. | Empty | % purity |
| 24 | 55 | 96.6 | 63 | 47 | 41 | 13 | 99.9 |
| 25 | 59 | 95.9 | 44 | 26 | 20 | 54 | 99.9 |

The affinity elution pool from these two affinity chromatography runs were used as the starting material for AEX runs (HQ-24 and HQ-25). The affinity elution pools (48.8 mL) were diluted 30- to 40-fold in 100 mM Tris pH 9.0 until the conductivity was s 1.9 mS/cm to form the AEX load. If needed, the pH of the diluted load was adjusted to pH 9.0 using Tris base. The diluted AEX load was filtered through an EKV filter before loading onto a POROS™ 50 HQ column (1.2 cm inner diameter, 5 cm bed height, and 5.7 mL column volume). The AEX method used for these runs is outlined in Table 16. 1 mL elution fractions were collected and then pooled based on SEC A260/280 ratios. The AEX pooling strategy for runs HQ-24 and HQ-25 is shown in Tables 19 and 20, with the twelve fractions shown pooled from HQ-24 (used affinity elution buffer comprising 30 mM sodium citrate) and three fractions pooled from HQ-25 (used elution buffer comprising 150 mM sodium acetate).

TABLE 19
HQ-24 AEX SEC A260/280 Ratios (30 mM sodium citrate,
100 mM glycine, 100 mM MgCl₂, pH 3.0)

| Frac | | | | | | | | | | | | | |
|---|---|---|---|---|---|---|---|---|---|---|---|---|---|
| # | 5 | 6 | 7 | 8 | 9 | 10 | 11 | 12 | 13 | 14 | 15 | 16 | 17 |
| SEC | 1.25 | 1.28 | 1.29 | 1.29 | 1.30 | 1.30 | 1.30 | 1.30 | 1.30 | 1.30 | 1.29 | 1.26 | 1.22 |

TABLE 20
HQ-25 AEX SEC A260/280 Ratios (150 mM sodium
acetate, 100 mM glycine, 100 mM MgCl₂, pH 3.0)

| Frac # | 7 | 8 | 9 | 10 | 11 | 12 | 13 |
|---|---|---|---|---|---|---|---|
| SEC | 0.96 | 1.10 | 1.19 | 1.21 | 1.16 | 1.05 | 0.94 |
| Pooled? | No | No | Yes | Yes | Yes | No | No |

The AEX pools were submitted for % purity (as measured by RP-HPLC, NR) and % full/empty capsids (as measured by SEC-HPLC), the results of which are detailed in Table 18.

For the HQ-24 (30 mM citrate) run, 7% of the loaded vg was unbound from the AEX resin, while for the HQ-25 (150 mM acetate) run, only 3% of the loaded vg was unbound from the AEX resin. This result further corroborates what was observed in the small-scale study (runs HQ-17 and HQ-18), that is citrate, a trivalent anion, reduces AAV9 binding to the POROS™ 50 HQ resin. The HQ-24 (30 mM citrate) % vg step yield was 63% whereas the % vg step yield from the HQ-25 run (150 mM acetate) was 44%. HQ-24 (30 mM citrate) was able to reduce the % empty capsids six-fold (79% to 13%) while HQ-25 (150 mM acetate) was only able to reduce the % empty capsids 1.5-fold (79% to 54%). Both runs produced a very pure product pool of 99.9% by RP-HPLC, NR.

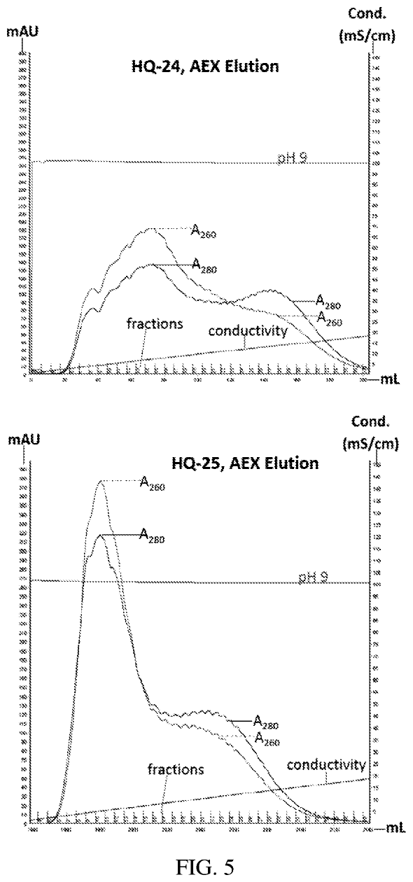

From these results, it was determined that the two affinity elution buffers performed comparably for the affinity chromatography step. For the AEX step, run HQ-24 (30 mM citrate) was unable to bind the same level of AAV9 to the AEX resin as was run HQ-25 (150 mM acetate) which led to distinct elution profiles as shown in FIG. 5. The small-scale and scale-up studies produced similar results and led to replacement of citrate with acetate in the affinity elution buffer. Additional studies optimized the MgCl₂ concentration in the affinity elution buffer that included acetate to improve the enrichment of % full capsids during the AEX step.

D. Summary

The previous example (Example 3) showed that 100 mM sodium citrate was not optimal for AEX elution so other affinity elution buffer components were tested. In this example, initially six different affinity elution buffers (different components added to a solution comprising 100 mM glycine, 100 mM MgCl₂, pH 3.0) were tested to determine their effect on the affinity and AEX elution profiles.

From this study, 30 mM sodium citrate, 100 mM glycine, 100 mM MgCl₂, pH 3.0 and 150 mM sodium acetate, 100 mM glycine, 100 mM MgCl₂, pH 3.0 performed the best in terms of % vg recovery for both affinity and AEX elution, as well as the % full capsids recovered in the AEX eluate. Based on this, a scale-up affinity chromatography to AEX linking study was performed using a 100 mL affinity column and these top two performing affinity elution buffers. For the large-scale study, the 150 mM sodium acetate, 100 mM glycine, 100 mM MgCl₂, pH 3.0 affinity elution buffer outperformed the buffer comprising 30 mM sodium citrate, 100 mM glycine, 100 mM MgCl₂, pH 3.0 buffer affinity elution buffer. Only about 3% of the loaded vg was unbound during the AEX step when the 150 mM sodium acetate affinity elution buffer was used whereas the about 7% of the loaded vg was unbound during the AEX step when the 30 mM citrate affinity elution buffer was used. This corroborates what was observed during the small-scale affinity to AEX linking study, and it was concluded that the 150 mM sodium acetate affinity elution buffer should be implemented with additional optimization to improve % full capsid enrichment during the AEX step.

### Example 5: Varying MgCl₂ Concentration in 150 mM Sodium Acetate Affinity Elution Buffer

A. Affinity Chromatography Step: Optimal MgCl₂ Concentration Study

The affinity elution buffer, 150 mM sodium acetate, 100 mM glycine pH 3.0, was formulated with 5 mM, 25 mM, 50 mM, and 100 mM MgCl₂ to study and optimize the MgCl₂ concentration. Sodium citrate buffer was not included in the affinity elution buffer due to its interference with binding of AAV9 onto the POROS™ 50 HQ resin during the anion exchange chromatography step. The objective of these affinity chromatography experiments was to identify the affinity chromatography elution buffer that would maximize vector recovery (based on qPCR assay) and would prevent vector precipitation after affinity elution.

A total of 1 L of clarified cell lysate was loaded onto a 4 mL POROS™ CaptureSelect AAV9 column (1 cm inner diameter and 5 cm bed height). The affinity chromatography method used for these experiments is detailed in Table 21.

TABLE 21
Affinity Chromatography Method for
MgCl₂ in 150 mM Sodium Acetate Study

| | | | Residence | Flow |
|---|---|---|---|---|
| | | Column | Time | Velocity |
| Step | Solution | Volumes | (min/CV) | (cm/hr) |
| Equilibration 1 | 100 mM Tris, pH | 5 | 3 | 100 |
| | 7.5 | | | |
| Sample-Load | clarified lysate | 250 | 3 | 100 |
| Equilibration 2 | 100 mM Tris, pH | 5 | 3 | 100 |
| | 7.5 | | | |
| Pre-Elution | 20% ethanol, 1M | 5 | 3 | 100 |
| Wash | NaCl, 100 mM Tris, | | | |
| | pH 7.5 | | | |
| Equilibration 3 | 150 mM sodium | 5 | 3 | 100 |
| | acetate, pH 5.6 | | | |
| Elution | 150 mM sodium | 5 | 8 | 37 |
| | acetate, 5 mM-100 | | | |
| | mM MgCl₂, 100 | | | |
| | mM glycine, pH 3.0 | | | |
| Equilibration 4 | 150 mM sodium | 5 | 8 | 37 |
| | acetate, pH 5.6 | | | |
| GuHCl Strip | 3M guanidine HCl, | 4 | 8 | 37 |
| | pH 5.05 | | | |
| Equilibration 5 | 100 mM Tris, pH | 4 | 3 | 100 |
| | 7.5 | | | |
| Storage | 20% ethanol | 3 | 3 | 37 |

A 5 mL elution fraction was collected from the affinity column and submitted for analytical testing with the key results shown in Table 22. Based on ITR qPCR results, increased % vg recovery was achieved when the elution buffer contained a lower concentration of MgCl₂, such as a 65% vg recovery for the buffer containing 25 mM MgCl₂ and a 58% vg recovery for the buffer containing 5 mM MgCl₂. However, precipitation was observed in the affinity elution containing 5 mM MgCl₂, 150 mM sodium acetate, 100 mM glycine, pH 3.0.

TABLE 22
Analytical Results from 150 mM sodium acetate/X mM MgCl₂ Study

| | | Affinity | | | | | |
|---|---|---|---|---|---|---|---|
| | | Step | | | | | |
| | Affinity | Results | AEX Step Results | | | | |
| Run | Elution | AF % vg | AEX % vg | % capsid species (AUC) | | | % |
| # | Buffer | recovery | recovery | Full | Interm. | Empty | purity |
| 29 | 150 mM sodium | 44 | 38 | 38 | 20 | 42 | 98.7 |
| | acetate/100 mM | | | | | | |
| | MgCl₂ | | | | | | |
| 30 | 150 mM sodium | 48 | 22 | 32 | 28 | 40 | 98.5 |
| | acetate/50 mM | | | | | | |
| | MgCl₂ | | | | | | |
| 31 | 150 mM sodium | 65 | 50 | 35 | 23 | 42 | 99.2 |
| | acetate/25 mM | | | | | | |
| | MgCl₂ | | | | | | |
| 32 | 150 mM sodium | 58 | 52 | 29 | 25 | 46 | 98.9 |
| | acetate/5 mM | | | | | | |
| | MgCl₂ | | | | | | |

B. AEX Chromatography Step for Optimal MgCl₂ Concentration Study

Next, studies were performed to determine the effect of MgCl₂ on AEX performance, and in particular, on AAV9 empty/full separation. Four affinity elution pools from the previously described affinity chromatography runs were utilized for the AEX chromatography step (runs 29-32). The objective of these AEX experiments was to evaluate the effect of the MgCl₂ concentration in the affinity elution buffer, and thus in the affinity eluate, on the AEX dilution step and empty/full capsid separation performance. The amount of MgCl₂ present in the AEX load can affect the binding of AAV9 to the POROS™ 50 HO resin and therefore impact empty/full capsid separation.

Each affinity elution pool (5 mL) was diluted with 20 mM Tris, pH 9.0, 2% PEG 8k until the conductivity reached about 1.9 mS/cm. If needed, the pH was adjusted to 9.0 using 1 M Tris base. The final pH, conductivity, and dilution factor were recorded for each AEX load. The pH ranged from 8.9 to 9.0, the conductivity ranged from 1.90 to 1.98 mS/cm, and the dilution factor ranged from 11- to 17-fold. The entire diluted sample was filtered through an EKV filter before being loaded onto a POROS™ SO HO column (0.5 cm inner diameter, 5 cm bed height, and 1 mL column volume). The AEX method used for these experiments is detailed in Table 23.

TABLE 23
AEX Chromatography Method for MgCl₂ titration
in 150 mM sodium acetate Study

| | | | Residence | Flow |
|---|---|---|---|---|
| | | Column | Time | Velocity |
| Step | Solution | Volumes | (min/CV) | (cm/hr) |
| Equilibration | 20 mM Tris, 2% PEG | 10 | 0.5 | 611 |
| | 8k, pH 9.0 | | | |
| Sample Load | affinity elution pool | 70 | 0.5 | 611 |
| | diluted into 20 mM | | | |
| | Tris, 2% PEG 8k, pH | | | |
| | 9.0 | | | |
| Equilibration | 20 mM Tris, 2% PEG | 28 | 0.5 | 611 |
| | 8k, pH 9.0 | | | |
| Wash | 20 mM Tris, pH 9.0 | 10 | 0.5 | 611 |
| Elution | (1) 20 mM Tris, pH | 50 | 0.5 | 611 |
| Gradient | 9.0 | | | |
| (0-250 mM | (2) 20 mM Tris, 0.5M | | | |
| NaCl/50 CV, | NaCl, pH 9.0 | | | |
| hold 10 CV) | | | | |
| Regeneration | 2M NaCl, 25 mM | 10 | 0.5 | 611 |
| | Tris, pH 9.0 | | | |
| Sanitization | 0.5M NaOH | 10 | 6 | 51 |
| Equilibration | 20 mM Tris HCl, pH | 10 | 0.5 | 611 |
| | 9.0 | | | |
| Storage | 20% ethanol | 10 | 0.5 | 611 |

One mL AEX elution fractions were collected and neutralized by addition of 50 mM sodium citrate, pH 3.6 at a volume that was 15% of the original fraction volume. Once the elution fractions were neutralized, they were submitted for SEC A260/280 analysis to determine the empty/full capsid ratios. The pooling strategy for these elution fractions is outlined in Tables 24-27. AEX elution fractions with SEC A260/280 ratios ≥1.15 were pooled. For run 29 (150 mM sodium acetate, 100 mM MgCl₂) and run 30 (150 mM sodium acetate, 50 mM MgCl₂), 3 consecutive elution fractions were pooled. For run 31 (150 mM sodium acetate, 25 mM MgCl₂) and run 32 (150 mM sodium acetate, 5 mM MgCl₂), 4 consecutive elution fractions were pooled.

TABLE 24
Run 29 (150 mM sodium acetate, 100 mM MgCl₂) AEX
SEC A260/280 Ratios for 4 mL Affinity Column

| Frac # | 2 | 3 | 4 | 5 | 6 | 7 |
|---|---|---|---|---|---|---|
| SEC | 1.1 | 1.21 | 1.24 | 1.18 | 1.04 | 0.9 |
| Pooled? | No | Yes | Yes | Yes | No | No |

TABLE 25
Run 30 (150 mM sodium acetate, 50 mM MgCl₂) AEX
SEC A260/280 Ratios for 4 mL Affinity Column

| Frac # | 2 | 3 | 4 | 5 | 6 | 7 |
|---|---|---|---|---|---|---|
| SEC | 1.12 | 1.22 | 1.24 | 1.18 | 1.01 | 0.89 |
| Pooled? | No | Yes | Yes | Yes | No | No |

TABLE 26
Run 31 (150 mM sodium acetate, 25 mM MgCl₂) AEX
SEC A260/280 Ratios for 4 mL Affinity Column

| Frac # | 2 | 3 | 4 | 5 | 6 | 7 |
|---|---|---|---|---|---|---|
| SEC | 1.17 | 1.22 | 1.25 | 1.15 | 1.03 | 0.95 |
| Pooled? | Yes | Yes | Yes | Yes | No | No |

TABLE 27
Run 32 (150 mM sodium acetate, 5 mM MgCl₂) AEX
SEC A260/280 Ratios for 4 mL Affinity Column

| Frac # | 2 | 3 | 4 | 5 | 6 | 7 | 8 |
|---|---|---|---|---|---|---|---|
| SEC | 1.15 | 1.21 | 1.21 | 1.15 | 1.03 | 0.98 | 0.94 |
| Pooled? | Yes | Yes | Yes | Yes | No | No | No |

The AEX elution pools for each run were submitted for analytical testing including % vg recovery as measured by ITR qPCR, % capsid species as measured by AUC, and % purity as measured by RP-HPLC, NR. Data is shown in Table 22.

In all four AEX runs, all loaded vg bound to the AEX column. In addition, the % purity (average % purity=98.8±0.3%) and product quality were highly similar (average % full+average % intermediate capsids=58±2% and average SEC A260/280 ratio=1.16±0.015). The MgCl₂ concentration did not impact the AEX empty/full capsid separation performance since all four AEX runs saw similar enrichment of full capsids. However, the concentration of MgCl₂ in the affinity elution buffer did impact the % vg recovery in both the affinity and AEX runs. The optimal affinity elution buffer was found to be 150 mM sodium acetate, 100 mM glycine, 25 mM MgCl₂, pH 3.0 since it resulted in % vg recovery of 65% and 50% for affinity and AEX unit operations, respectively (Table 22).

C. Preparation of Affinity Load for Varying MgCl₂ Concentration Study

HEK293 cells were grown in suspension culture and transfected with a 3-plasmid system to produce rAAV9 vector. Cell suspension was obtained from the bioreactor for harvest. Cell harvest was treated with 0.5% triton X-100 and mixed for 30 minutes to lyse the cells and release AAV9, including full capsids comprising an rAAV genome, intermediate capsids, and empty capsids. The lysate was flocculated, depth filtered, quenched, and 0.2 μm filtered to produce a clarified lysate that was loaded onto affinity chromatography columns as described below.

D. Scalability and Reproducibility of 150 mM Sodium Acetate/25 mM MgCl₂ Affinity Elution Buffer

Since the results described above were obtained on a small-scale affinity chromatography column (4 mL), additional experiments, at larger scales (8 mL and 40 mL columns), were conducted to evaluate the initial findings and test reproducibility. The larger scale affinity chromatography runs were completed using the 150 mM sodium acetate, 100 mM glycine, 25 mM MgCl₂, pH 3.0 affinity elution buffer to confirm the initial, small scale results. A total of five scaled-up affinity runs were performed with four runs on an 8 mL affinity column and a single run on a 40 mL affinity column as shown in Table 28.

TABLE 28
Scaled-Up Affinity Chromatography Runs With 150 mM sodium
acetate, 100 mM glycine, 25 mM MgCl₂ pH 3.0 Elution Buffer

| | Column | Elution Flow | Elution | % vg |
|---|---|---|---|---|
| Run | Volume | Velocity | Residence Time | recovery |
| Number | (mL) | (cm/hr) | (min/CV) | (ITR qPCR) |
| 1 | 8 | 75 | 8.0 | 96 |
| 2 | 8 | 75 | 8.0 | 85 |
| 3 | 8 | 75 | 8.0 | 89 |
| 4 | 8 | 75 | 8.0 | 83 |
| 5 | 40 | 150 | 8.0 | 69 |

Preparation of the affinity load was the same as was described previously in this example. A total of 1 L of clarified cell lysate was loaded onto a POROS™ CaptureSelect AAV9 column (1 cm inner diameter, 10 cm bed height, and 8 mL column volume). A similar affinity chromatography method was used as in the small-scale runs except for differing linear flow velocities. The elution pool was collected and submitted for ITR qPCR to determine % vg recovery. The average % vg recovery over all four 8 mL column volume affinity chromatography runs was 88% (ranging from 83% to 96%) as shown in Table 28.

A single scaled-up affinity chromatography run was performed on a 40 mL affinity column (run 5). Preparation of the affinity load was the same as was described previously in this example. A total of 4 L of clarified cell lysate was loaded onto a POROS™ CaptureSelect AAV9 column (1.6 cm inner diameter, 20 cm bed height, and 40 mL column volume). A similar affinity chromatography method was used as in the small-scale runs except for differing linear flow velocities. The affinity elution pool was collected and submitted for ITR qPCR to measure % vg recovery and RP-HPLC, NR to measure % purity. The % vg recovery was 69% (Table 28) and the % purity was 86%. From these eight scaled-up affinity chromatography runs, it was determined that the optimal affinity elution buffer at small-scale is also the optimal elution buffer at larger scale. % vg recoveries are comparable over all runs at the varying scales. Therefore, the optimal affinity elution buffer at all scales is 150 mM sodium acetate, 100 mM glycine, 25 mM MgCl₂, pH 3.0.

E. Summary

In this example, the MgCl₂ concentration was varied in the 150 mM sodium acetate affinity elution buffer to further optimize the % vg recovery and % full capsids purified at the AEX step. Four different MgCl₂ concentrations were tested. At the small-scale, it was determined that the 150 mM sodium acetate, 25 mM MgCl₂ affinity elution buffer performed the best, with a 65% vg recovery at the affinity chromatography step and 50% vg recovery at the AEX step. In addition, it provided the highest % full capsids at the AEX step out of all the buffers tested (35% full capsids). To confirm these results, and test reproducibility, a larger-scale study was conducted with 8 mL and 40 mL affinity columns. % vg recovery at the affinity chromatography step at these larger scales was comparable to that of the small-scale study which demonstrates the reproducibility and scalability of the 150 mM sodium acetate, 25 mM MgCl₂ affinity elution buffer results. Therefore, the affinity elution buffer selected was the buffer containing 150 mM sodium acetate, 100 mM glycine, 25 mM MgCl₂, pH 3.0.

### Example 6: Clean in Place (CIP) Screening Studies for Alternative Regeneration Buffers

A. Preparation of Affinity Load for CIP Screening Studies

HEK293 cells were grown in suspension culture and transfected with a 3-plasmid system to produce rAAV9 vector. Cell suspension was obtained from a 50 L SUB/FIN43 (Finesse bioreactor) bioreactor for harvest. Cell harvest was treated with 0.5% triton X-100 and mixed for 30 minutes to lyse the cells and release AAV9, including full capsids comprising an rAAV genome, intermediate capsids, and empty capsids. The lysate was flocculated, depth filtered, quenched, and 0.2 μm filtered to produce a clarified lysate that was loaded onto affinity chromatography columns as described below.

B. Initial Screening of Different Regeneration Buffers

The regeneration buffer comprising 3 M guanidine HCl, pH 5.05 did not completely clean the resin which reduced the number of purification cycles that could be run on the affinity chromatography column resin. To solve this issue, different regeneration buffers were screened to determine the optimal regeneration buffer that provided effective cleaning of the resin.

A total of 4 L of 50 L SUB clarified cell lysate was loaded onto a POROS™ CaptureSelect AAV9 column (1 cm inner diameter, 2.25 cm bed height, and 1.57 mL column volume). For all runs, the affinity chromatography method detailed in Table 29 was used.

TABLE 29
Affinity Chromatography Method for
the Initial CIP Screening Study

| | | | Residence | Flow |
|---|---|---|---|---|
| | | Column | Time | Velocity |
| Step | Solution | Volumes | (min/CV) | (cm/hr) |
| Equilibration 1 | 100 mM Tris, pH | 5 | 3 | 50 |
| | 7.5 | | | |
| Sample-Load | clarified lysate | 205 | 3 | 50 |
| | from 50 L SUB | | | |
| Equilibration 2 | 100 mM Tris, pH | 5 | 3 | 50 |
| | 7.5 | | | |
| Pre-Elution | 20% ethanol, 1M | 5 | 3 | 50 |
| Wash | NaCl, 100 mM Tris, | | | |
| | pH 7.5 | | | |
| Equilibration 3 | 100 mM sodium | 5 | 3 | 50 |
| | acetate, pH 5.6 | | | |
| Elution | 150 mM sodium | 5 | 8 | 19 |
| | acetate, 100 mM | | | |
| | MgCl₂, 100 mM | | | |
| | glycine, pH 3.0 | | | |
| Equilibration 4 | 100 mM sodium | 5 | 8 | 19 |
| | acetate, pH 5.6 | | | |
| Equilibration 5 | 100 mM Tris, pH | 10 | 8 | 19 |
| | 7.5 | | | |
| Regeneration A | Regeneration Buffer | 4 | 8 | 19 |
| | A (listed in Table | | | |
| | 30) | | | |
| Equilibration 6 | 100 mM Tris, pH | 10 | 4 | 38 |
| | 7.5 | | | |
| Regeneration B | Regeneration Buffer | 4 | 8 | 19 |
| | B (listed in Table | | | |
| | 30) | | | |
| Equilibration 7 | 100 mM Tris, pH | 10 | 4 | 38 |
| | 7.5 | | | |
| Storage | 20% ethanol | 3 | 4 | 38 |

Regeneration buffers A and B used in runs 1-7 are listed in Table 30. For run 7, 0.1 N phosphoric acid, pH 1.90 was used with a residence time of 34 min/CV.

TABLE 30
Regeneration Buffers Tested Per Affinity Run

| Run # | Regeneration Buffer A* | Regeneration Buffer B* |
|---|---|---|
| 1 | 3M guanidine HCl, pH 5.05 | 1M acetic acid, pH 2.36 |
| 2 | 1M acetic acid, pH 2.36 | 3M guanidine HCl, pH 5.05 |
| 3 | 200 mM glycine, 3M guanidine | 1M acetic acid, pH 2.36 |
| | HCl, pH 2.30 | |
| 4 | 400 mM glycine, 3M guanidine | 1M acetic acid, pH 2.36 |
| | HCl, pH 1.96 | |
| 5 | 400 mM glycine, 1M guanidine | 1M acetic acid, pH 2.36 |
| | HCl, pH 1.99 | |
| 6 | 8 min RT 0.1N phosphoric | 1M acetic acid, pH 2.36 |
| | acid, pH 1.90 | |
| 7 | 34 min RT 0.1N phosphoric | 1M acetic acid, pH 2.36 |
| | acid, pH 1.90 | |
| 8* | 0.5M phosphoric acid, pH 1.2 | 10 mM sarkosyl, 100 mM |
| | | Tris, pH 7.5 |
| 9* | 0.5M phosphoric acid, pH 1.2 | 10 mM sarkosyl, 100 mM |
| | | Tris, pH 7.5 |
| 10 | 25% propylene glycol, 100 mM | 10 mM sarkosyl, 100 mM |
| | Tris, pH 7.5 | Tris, pH 7.5 |
| 11 | 4M urea, 100 mM Tris, pH 7.5 | 10 mM sarkosyl, 100 mM |
| | | Tris, pH 7.5 |
| 12 | 10 mM sarkosyl, 100 mM | 8 min RT 0.1N phosphoric |
| | Tris, pH 7.5 | acid, pH 1.90 |
| 13 | 1M acetic acid, pH 2.4 | 10 mM sarkosyl, 100 mM |
| | | Tris, pH 7.5 |

*These runs have different starting materials (Run 8: FIN43, Run 9: 50SUB)

Samples from each step of the affinity chromatography method were collected. The regeneration buffer fractions were neutralized then submitted for ITR qPCR to determine % vg recovery. Results are shown in Table 31.

TABLE 31
qPCR Results from CIP Alternative Regeneration Buffer Study

| | % vg recovery | | |
|---|---|---|---|
| Run # | Affinity Elution Pool | Regen Buffer A | Regen Buffer B |
| 1 | 59.55 | 9.04 | 1.45 |
| 2 | 51.60 | 16.09 | 0.41 |
| 3 | 14.92 | 6.02 | 0.19 |
| 4 | 36.53 | 20.22 | 0.14 |
| 5 | 41.26 | 5.44 | 7.88 |
| 6 | 37.13 | 18.00 | 1.33 |
| 7 | 38.69 | 16.86 | 0.81 |
| 8 | 40.67 | 1.42 | below LOQ |
| 9 | N/A | N/A | N/A |
| 10 | 58.64 | below LOQ | 0.34 |
| 11 | 61.40 | 0.34 | 0.29 |
| 12 | 56.70 | 1.13 | below LOQ |
| 13 | 65.47 | 7.25 | below LOQ |

LOQ: limit of quantification

In addition to % vg recovery as measured by ITR qPCR, the regeneration buffer peak areas for each run were determined as shown in Table 32.

TABLE 32
Regeneration Buffer Peak Areas for each Run

| | Regeneration Buffer A | | Regeneration Buffer B | |
|---|---|---|---|---|
| | Peak Area | Peak Area | Peak Area | Peak Area |
| | 280 nm | 260 nm | 280 nm | 260 nm |
| Run # | (mL*mAU) | (mL*mAU) | (mL*mAU) | (mL*mAU) |
| 1 | 521 | 337 | 188 | 694 |
| 2 | 540 | 721 | 381 | 279 |
| 3 | 504 | 408 | 197 | 535 |
| 4 | 650 | 587 | 185 | 423 |
| 5 | 146 | 95 | 344 | 840 |
| 6 | 995 | 946 | 163 | 251 |
| 7 | 1128 | 1118 | 128 | 208 |
| 8 | 52 | 73 | 1259 | 552 |
| 9 | 46 | 107 | 1374 | 613 |
| 10 | 120 | 98 | 1416 | 665 |
| 11 | 326 | 344 | 588 | 579 |
| 12 | 2060 | 1054 | 26 | 30 |
| 13 | 215 | 425 | 1840 | 914 |

The data demonstrated that 0.1 N phosphoric acid, pH 1.90 was the optimal regeneration buffer among those tested. The second and third highest % vg recoveries were obtained from the 8 min/CV residence time, 0.1 N phosphoric acid (18% vg) run, and the 34 min/CV residence time, 0.1 N phosphoric acid (16.8% vg) run, respectively. The peak area comparisons for the regeneration buffers demonstrated that 0.1 N phosphoric acid, pH 1.90 with an 8 min/CV or 34 min/CV residence time performed better than other regeneration buffers by removing a greater amount of impurities and a greater amount of residual rAAV. The 34 min/CV residence time, 0.1 N phosphoric acid regeneration peak showed increased peak area, but it was not significant enough to warrant a change from an 8 min/CV residence time, which is consistent with other steps in the affinity chromatography method.

Ligand assay data showed that the first wash of new resin yielded 67.65 ng of column ligand in the first fraction following a wash with 0.1 N phosphoric acid, pH 1.90. This indicates that the first 0.1 N phosphoric acid, pH 1.90 washes off loosely attached ligand. To confirm these results, 0.1 N phosphoric acid, pH 1.90 was tested on various affinity load material (e.g., clarified lysates).

C. Second Screening of Alternative Regeneration Buffers

In scaled-up manufacturing, the affinity resin was useable for only 2-3 cycles when regeneration was performed with 3 M guanidine HCl, pH 5.05, which is not economical or efficient. Screening alternative regeneration buffers demonstrated that 0.1 N phosphoric acid, pH 1.90 performed the best. However, in order to ensure consistency of the performance of the 0.1 N phosphoric acid, pH 1.90, the results were confirmed using various affinity load materials.

The second screening of alternative regeneration buffers tested different regeneration buffers, as well as 0.1 N phosphoric acid. A total of 240 mL of 50 L SUB and FIN43 clarified cell lysates were loaded onto a POROS™ CaptureSelect AAV9 column (1 cm inner diameter, 2.8 cm bed height, and 2.2 mL column volume). For all runs, the affinity chromatography method detailed in Table 33 was used.

TABLE 33
Affinity Chromatography Method for
the Second CIP Screening Study

| | | | Residence | Flow |
|---|---|---|---|---|
| | | Column | Time | Velocity |
| Step | Solution | Volumes | (min/CV) | (cm/hr) |
| Equilibration 1 | 100 mM Tris, pH | 5 | 5 | 50 |
| | 7.5 | | | |
| Sample-Load | clarified lysate | 64 | 5 | 50 |
| | from 50 SUB | | | |
| Equilibration 2 | 100 mM Tris, pH | 5 | 5 | 50 |
| | 7.5 | | | |
| Pre-Elution | 17% ethanol, 1M | 5 | 5 | 50 |
| Wash | NaCl, 100 mM Tris, | | | |
| | pH 7.5 | | | |
| Equilibration 3 | 150 mM sodium | 5 | 5 | 50 |
| | acetate, pH 5.6 | | | |
| Elution | 150 mM sodium | 5 | 8 | 19 |
| | acetate, 25 mM | | | |
| | MgCl₂, 100 mM | | | |
| | glycine, pH 3.0 | | | |
| Equilibration 4 | 150 mM sodium | 5 | 8 | 19 |
| | acetate, pH 5.6 | | | |
| Regeneration A | Regeneration Buffer | 4 | 8 | 19 |
| | A (listed in Table | | | |
| | 30) | | | |
| Equilibration 5 | 100 mM Tris, pH | 5 | 8 | 38 |
| | 7.5 | | | |
| Regeneration B | Regeneration Buffer | 4 | 37 | 19 |
| | B (listed in Table | | | |
| | 30) | | | |
| Equilibration 6 | 100 mM Tris, pH | 5 | 5 | 38 |
| | 7.5 | | | |
| Storage | 17% ethanol | 3 | 5 | 38 |

Regeneration buffers A and B were selected from one of the following buffers: 1 M acetic acid, pH 2.4; 0.1 N phosphoric acid, pH 1.9; 0.5 M phosphoric acid, pH 1.2; 4 M urea in 100 mM Tris, pH 7.5; 25% propylene glycol in 100 mM Tris, pH 7.5; and 10 mM sarkosyl in 100 mM Tris, pH 7.5. The specific alternative regeneration buffers tested for each run are outlined in Table 30 (runs 8-13). Samples from each step of the affinity chromatography method were collected. The regeneration buffer fractions were neutralized then submitted for ITR qPCR to determine % vg recovery as shown in Table 31 (runs 8-13). In addition to ITR qPCR, the regeneration buffer peak areas for each run were determined as shown in Table 32 (runs 8-13). The data suggested that none of the regeneration buffers significantly reduced the 10 mM sarkosyl wash peak which was used as a positive control for effective cleaning of the resin in these experiments. A 10 mM sarkosyl wash generated consistent large A280 and A260 peaks indicative of removal of impurities and residual rAAV from the resin, and was thus tested through lifecycle studies to determine if using sarkosyl was a viable path forward.

D. Summary

In this example, two screening studies were conducted to test alternative regeneration buffers for the CIP step in the affinity chromatography process. 3 M guanidine HCl, pH 5.05 which had been used did not effectively clean the resin which lead to a limited lifecycle for the affinity resin. In the initial screening study, 0.1 N phosphoric acid, pH 1.90 was the best CIP regeneration buffer since it had one of the higher % vg recoveries and a higher regeneration peak area which indicated a greater level of removal of impurities and a greater level of removal of residual rAAV.

A second screening study was performed to show consistency with the initial screening results when different affinity load material was used. This second screening study demonstrated that none of the regeneration buffers tested were able to reduce the 10 mM sarkosyl wash peak area, which indicated that the resin was not effectively cleaned by the various regeneration buffers tested. Additional studies were conducted to determine an optimal CIP step.

### Example 7: Affinity Resin Cycling Study with 3 Min Residence Time and 15 cm Bed Height

A. Preparation of Affinity Load for Lifecycle Study

HEK293 cells were grown in suspension culture and transfected with a 3-plasmid system to produce rAAV9 vector. Cell harvest was treated with 0.5% triton X-100 and mixed for 30 minutes to lyse the cells and release AAV9, including full capsids comprising an rAAV genome, intermediate capsids, and empty capsids. The lysate was flocculated, depth filtered, quenched, and 0.2 μm filtered to produce a clarified lysate that was loaded onto affinity chromatography columns as described below.

B. POROS™ CaptureSelect AAV9 Affinity Resin Cycling Study

Previous process development cycling studies utilized affinity chromatography methods with residence times that ranged from 3 minutes to 36 minutes. At the manufacturing scale, the affinity chromatography operational interface (SKID) cannot accommodate the range of flow rates that are associated with those residence times. Therefore, this cycling study employed a 3-minute residence time across all affinity steps so that manufacturing scale chromatography SKID could be utilized.

Twelve purification cycles were performed on a POROS™ CaptureSelect AAV9 affinity column (0.66 cm inner diameter, 15 cm bed height, and 5.1 mL column volume). For each run, a total of 270 mL of FIN92 clarified lysate was loaded onto the affinity column with a resin challenge of 7.5×10¹³ vg/mL resin. The load turbidity was measured to be about 4.7±0.3 NTU prior to filtering with a 0.2 μm Pall™ EAV filter. The affinity load bag containing filtered clarified lysate was connected in line with the affinity column. During runs, the affinity load bag was placed in a refrigerator set to 2-8° C.

The affinity chromatography method utilized for this lifecycle study is detailed in Table 34.

TABLE 34
Affinity Lifecycle Study Chromatography Method

| | | | Residence | Flow |
|---|---|---|---|---|
| | | Column | Time | Velocity |
| Step | Solution | Volumes | (min/CV) | (cm/hr) |
| Equilibration 1 | 100 mM Tris. pH | 5 | 3.0 | 300 |
| | 7.5 | | | |
| Sample-Load | clarified lysate | 53 | 3.0 | 300 |
| | from FIN92 | | | |
| Equilibration 2 | 100 mM Tris. pH | 5 | 3.0 | 300 |
| | 7.5 | | | |
| Pre-Elution | 17% ethanol, 150 | 5 | 3.0 | 300 |
| Wash | mM sodium acetate, | | | |
| | pH 5.6 | | | |
| Equilibration 3 | 150 mM sodium | 5 | 3.0 | 300 |
| | acetate, pH 5.6 | | | |
| Elution | 150 mM sodium | 5 | 3.0 | 300 |
| | acetate, 25 mM | | | |
| | MgCl₂, 100 mM | | | |
| | glycine, pH 3.0 | | | |
| Equilibration 4 | 150 mM sodium | 5 | 3.0 | 300 |
| | acetate, pH 5.6 | | | |
| Regeneration 1 | 0.1N phosphoric | 5 | 3.0 | 300 |
| | acid, pH 1.9 | | | |
| Equilibration 5 | 100 mM Tris, pH | 5 | 3.0 | 300 |
| | 7.5 | | | |
| Regeneration 2 | 1% sarkosyl, 100 | 5 | 3.0 | 300 |
| | mM Tris, pH 7.5 | | | |
| Wash | 2M NaCl, 100 mM | 5 | 3.0 | 300 |
| | Tris, pH 7.5 | | | |
| Storage | 17% ethanol | 3 | 3.0 | 300 |

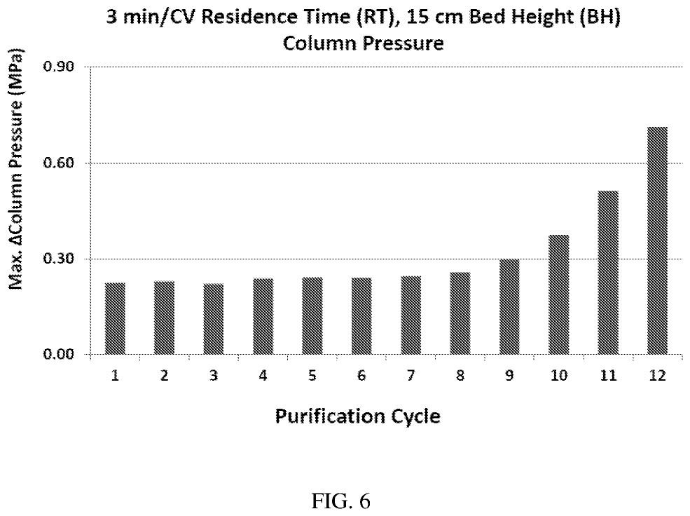

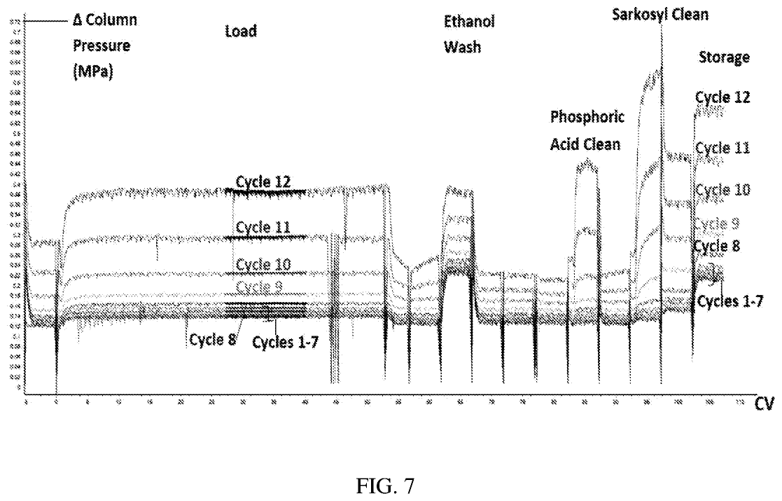

All affinity chromatography steps had a residence time of 3 minutes/CV. The collection gates for the affinity elution are specified as starting at 50 mAU and stopping at 45 mAU at 280 nm with a 2 mm path length. With this affinity elution collection criteria, a single elution fraction could be collected without having to collect and pool multiple elution fractions. The regeneration steps (0.1 N phosphoric acid, pH 1.90 and 1% sarkosyl, 100 mM Tris, pH 7.5) included a 48-minute hold to facilitate manufacturing operations. Column pressure was monitored and recorded during the pre-elution ethanol wash step, the sarkosyl regeneration step, and the ethanol storage step. Maximum delta column pressures for each run are shown in FIG. 6 and column pressure profiles for each run of the lifecycle study are shown in FIG. 7. The affinity elution from each of the twelve purification cycles was collected and submitted for analytical testing with the results shown in Table 35.

TABLE 35
Analytical Results for Affinity Elution, Run # 1-12, for Affinity Lifecycle Study

| | % vg | % purity | HCP | HCDNA | SEC | Max ΔCol |
|---|---|---|---|---|---|---|
| Run | recovery | (RP-HPLC, | (ng/1 × 10¹⁴ | (ng/1 × 10¹⁴ | A260/280 | Pressure |
| # | (ITR qPCR) | NR) | vg) | vg) | ratio | (MPa) |
| 1 | 81.3 | 97.4 | 2495 | 563 | 1.02 | 0.225 |
| 2 | 78.9 | 96.9 | 2666 | 724 | 1.02 | 0.230 |
| 3 | 74.0 | 97.0 | 2896 | 623 | 1.02 | 0.222 |
| 4 | 78.2 | 96.4 | 3470 | 557 | 1.03 | 0.239 |
| 5 | 97.4 | 96.5 | 2722 | 308 | 1.03 | 0.242 |
| 6 | 85.9 | 95.8 | 2995 | 299 | 1.03 | 0.241 |
| 7 | 84.0 | 95.4 | 4278 | 365 | 1.03 | 0.246 |
| 8 | 89.5 | 94.3 | 4184 | 332 | 1.03 | 0.258 |
| 9 | 74.6 | 94.6 | 5189 | 648 | 1.03 | 0.298 |
| 10 | 85.3 | 94.6 | 4557 | 653 | 1.03 | 0.375 |
| 11 | 83.5 | 93.0 | 5001 | 635 | 1.03 | 0.513 |
| 12 | 80.8 | 93.4 | 6019 | 587 | 1.03 | 0.713 |

The delta column pressure increased during the purification cycling from about 0.2 MPa to 0.7 MPa. Certain steps in the affinity chromatography method exhibited higher pressure than others. During runs 1-7, the ethanol wash step exhibited the highest pressure. During runs 8-10, the ethanol storage step was the step with the highest delta column pressure, reaching 0.258 MPa to 0.375 MPa. Lastly, during runs 11 and 12, the sarkosyl cleaning step was the step with the highest delta column pressure, reaching 0.513 MPa and 0.713 MPa, respectively. These significant pressure increases during the lifecycle study indicated that impurities were not effectively removed from the column by the regeneration steps and were building up on the resin. Significant pressure increases during a run indicated that the affinity resin was beginning to foul and that the resin could not be utilized for any further affinity runs.

Cycling with POROS™ CaptureSelect AAV9 gave an average % vg yield of 83±6% over the 12 purification cycles. The column maintained high binding capacity throughout the study, as no quantifiable unbound vg was detected during runs 1-10, and less than 2% unbound vg was detected during runs 11 and 12. This indicated that the % vg yield and affinity resin binding capacity were not significantly affected, even after twelve consecutive purification runs.

Over the course of the twelve runs, the residual HCP level increased from 2495 ng/1×10¹⁴ vg (run 1) to 6019 ng/1×10¹⁴ vg (run 12) and the percent purity decreased by about 4%. Collectively, these results indicated that the affinity column maintained its ability to bind AAV over the course of the twelve runs, but that the column resin became fouled with impurities with each successive run, leading to an increase in column pressure and residual HCP.

Overall, this lifecycle study indicated that about 8-10 consecutive affinity runs can be successfully performed using a single POROS™ CaptureSelect AAV9 column before observing significant increases in column pressure and residual HCP levels, and a decrease in percent purity.

C. Summary

In this example, an affinity resin lifecycle study using a 15 cm bed height column was conducted with a 3-minute residence time for each step. A total of twelve runs were performed on the same column and resin. Column pressure and % vg recovery were measured throughout the study. Across all 12 runs, % vg recovery stayed consistent, but column pressure spiked around runs 9-10. This increase in column pressure was due to buildup of impurities on the resin which were not effectively removed from the resin between runs. A decrease in percent purity was also observed. Therefore, about 8-10 runs could be successfully performed on a single column before affinity resin fouling was observed.

### Example 8: Use of Guard Column Pre-Affinity Chromatography for Removal of Impurities

A. Preparation of Guard Column Load

HEK293 cells were grown in suspension culture and transfected with a 3-plasmid system to produce rAAV9 vector. Cell suspension was obtained from a 50 L SUB bioreactor for harvest. Cell harvest was treated with 0.5% triton X-100 and mixed for 30 minutes to lyse the cells and release AAV9, including full capsids comprising an rAAV genome, intermediate capsids, and empty capsids. The lysate was flocculated, depth filtered, quenched, and 0.2 μm filtered to produce a clarified lysate that was loaded onto affinity chromatography columns as described below.

B. Guard Column Studies for Impurity Removal Pre-Affinity Column

Guard columns are protective columns that are placed before the analytical column, and in this study, a guard column was placed before the affinity column. The flow through from the guard column was then fed into the affinity column. The purpose of the guard column is to remove any impurities and precipitates from the affinity load and prevent them from reaching the affinity column, while at the same time, minimizing product loss. Impurities/precipitates bound to the affinity resin can lead to increased column pressure, as well as a reduction in the overall lifecycle of the resin. This results in ineffective use of the resin, and increased process cost.

With less impurities loaded onto the affinity column; it is likely that the regeneration peak from the affinity column would be diminished. Five different guard columns (POROS™ XQ, POROS™ HQ, POROS™ Benzyl Ultra, Capto Octyl, and Capto 700) were tested in the affinity chromatography method to determine if the 10 mM sarkosyl wash peak area would be reduced, indicating that a lesser amount of impurities were loaded onto the column. The POROS™ HQ and XQ resins are anion exchange resins while Capto Octyl, POROS™ Benzyl Ultra, and Capto 700 are hydrophobic interaction resins so these two resin groups target a wide range of impurities that may be present in the clarified lysate.

Each guard column utilized a different chromatography procedure as shown in Tables 36-39.

TABLE 36
8 mL POROS ™ XQ and HQ Guard Column Method

| | | | Residence | Flow |
|---|---|---|---|---|
| | | Column | Time | rate |
| Step | Solution | Volumes | (min/CV) | (mL/min) |
| Initial Clean | 0.5M NaOH | 5 | 3 | 2.6 |
| Equilibration | 25 mM Tris HCl, pH | 5 | 3 | 2.6 |
| | 7.5 - 1M NaCl | | | |
| Equilibration | 25 mM Tris HCl, pH | 5 | 3 | 2.6 |
| | 7.5 - 0.1M NaCl | | | |
| Sample | clarified lysate | 125 | 3 | 2.6 |
| | from 50 L SUB | | | |
| Wash 1 | 25 mM Tris HCl, pH | 5 | 3 | 2.6 |
| | 7.5 - 0.1M NaCl | | | |
| Wash 2 | 25 mM Tris HCl, pH | 5 | 3 | 2.6 |
| | 7.5 - 1M NaCl | | | |
| Regeneration | 0.5M NaOH | 5 | 3 | 2.6 |
| Equilibration | 25 mM Tris HCl, pH | 5 | 3 | 2.6 |
| | 7.5 - 0.1M NaCl | | | |
| Storage | 20% ethanol | 5 | 3 | 2.6 |

TABLE 37
8 mL POROS ™ Benzyl Ultra Guard Column Method

| | | | Residence | Flow |
|---|---|---|---|---|
| | | Column | Time | Velocity |
| Step | Solution | Volumes | (min/CV) | (cm/hr) |
| Equilibration | 25 mM Tris, 0.1M | 3 | 3 | 2.6 |
| | NaCl, pH 7.5 | | | |
| Load | clarified lysate | 125 | 3 | 2.6 |
| | from 50 L SUB | | | |
| Wash | 25 mM Tris, 0.5M | 3 | 3 | 2.6 |
| | NaCl, pH 7.5 | | | |
| Elution | 0.5M NaCl to 0.1M | 20 | 3 | 2.6 |
| | NaCl linear gradient, | | | |
| | 25 mM Tris, pH 7.5 | | | |
| Equilibration | 25 mM Tris, 0.1M | 3 | 3 | 2.6 |
| | NaCl, pH 7.5 | | | |
| Equilibration | H2O | 3 | 3 | 2.6 |
| CIP | 0.5M NaOH | 5 | 3 | 2.6 |
| Equilibration | H2O | 3 | 3 | 2.6 |
| CIP | 1M acetic acid, pH 2.0 | 5 | 3 | 2.6 |
| Equilibration | H2O | 3 | 3 | 2.6 |
| Storage | 0.1M NaOH | 3 | 3 | 2.6 |

TABLE 38
8 mL Capto Octyl Guard Column Method

| | | | Residence | Flow |
|---|---|---|---|---|
| | | Column | Time | rate |
| Step | Solution | Volumes | (min/CV) | (mL/min) |
| Equilibration | 25 mM Tris, 0.1M | 3 | 3 | 3.1 |
| | NaCl, pH 7.5 | | | |
| Load | clarified lysate | 125 | 3 | 3.1 |
| | from 50 L SUB | | | |
| Wash | 25 mM Tris, 0.5M | 3 | 3 | 3.1 |
| | NaCl, pH 7.5 | | | |
| Elution | 0.5M NaCl to 0.1M | 20 | 3 | 3.1 |
| | NaCl linear gradient, | | | |
| | 25 mM Tris, pH 7.5 | | | |
| Equilibration | 25 mM Tris HCl, pH | 3 | 3 | 3.1 |
| | 7.5 - 0.1M NaCl | | | |
| Equilibration | H₂O | 3 | 3 | 3.1 |
| CIP | 0.5M NaOH | 5 | 3 | 3.1 |
| Equilibration | H₂O | 3 | 3 | 3.1 |
| CIP | 1M acetic acid, pH 2.0 | 5 | 3 | 3.1 |
| Equilibration | H₂O | 3 | 3 | 3.1 |
| Storage | 0.1M NaOH | 3 | 3 | 3.1 |

TABLE 39
4.7 mL Capto 700 Guard Column Method

| | | | Residence | Flow |
|---|---|---|---|---|
| | | Column | Time | rate |
| Step | Solution | Volumes | (min/CV) | (mL/min) |
| Equilibration 1 | H₂O | 3 | 3 | 1.9 |
| Equilibration 2 | 20 mM Tris, pH 7.5 | 5 | 3 | 1.9 |
| Load | clarified lysate from | 49 | 3 | 1.9 |
| | 50 L SUB | | | |
| Wash 1 | 20 mM Tris, pH 7.5 | 5 | 3 | 1.9 |
| Wash 2 | 20 mM Tris, 150 | 10 | 3 | 1.9 |
| | mM NaCl, pH 7.5 | | | |
| CIP | 1M NaOH, 17% | 5 | 15 | 0.31 |
| | 2-propanol | | | |
| Equilibration 3 | 20 mM Tris, pH 7.5 | 5 | 3 | 1.9 |
| Equilibration 4 | H₂O | 3 | 3 | 1.9 |
| Storage | 17% ethanol | 5 | 3 | 1.9 |

A total of 1 L of 50 L SUB clarified lysate was loaded onto an 8 mL POROS™ XQ, HQ, Benzyl Ultra, and Capto Octyl guard column while 230 mL of 50 L SUB clarified lysate was loaded onto the 4.7 mL Capto 700 guard column. A total of six runs were performed for this study: one run using each of the guard columns and a control run with no guard column. The flow through from each guard column (240 mL) was loaded onto a POROS™ CaptureSelect AAV9 affinity column (1 cm inner diameter, 2.8 cm bed height, and 2.2 mL column volume). The post-guard column, affinity chromatography method used for these runs is detailed in Table Table 40.

TABLE 40
2.2 mL POROS ™ CaptureSelect AAV9
Affinity Chromatography Method

| | | | Residence |
|---|---|---|---|
| | | Column | Time |
| Step | Solution | Volumes | (min/CV) |
| Equilibration 1 | 100 mM Tris, pH 7.5 | 5 | 5 |
| Sample-Load | 50 L SUB guard column FT | 64* | 5 |
| Equilibration 2 | 100 mM Tris, pH 7.5 | 5 | 5 |
| Pre-Elution | 17% ethanol, 1M NaCl, 100 mM | 5 | 5 |
| Wash | Tris, pH 7.5 | | |
| Equilibration 3 | 150 mM sodium acetate, pH 5.6 | 5 | 5 |
| Elution | 150 mM sodium acetate, 25 mM | 5 | 8 |
| | MgCl₂, 100 mM glycine, pH 3.0 | | |
| Equilibration 4 | 150 mM sodium acetate, pH 5.6 | 5 | 8 |
| Regeneration | 0.1N phosphoric acid, pH 1.9 | 4 | 37 |
| Equilibration 5 | 100 mM Tris, pH 7.5 | 5 | 8 |
| Regeneration | 1% sarkosyl, 100 mM Tris, | 4 | 8 |
| | pH 7.5 | | |
| Equilibration 6 | 100 mM Tris, pH 7.5 | 5 | 5 |
| Storage | 17% ethanol | 3 | 5 |

*For Capto 700, the Sample-Load CV is 105

Peak areas for each regeneration step in the affinity chromatography method are provided in Table 41. Samples were obtained from each step of the guard column and POROS™ CaptureSelect AAV9 column procedures and submitted for qPCR analysis with results shown in Table 42. Based on qPCR results, there was no significant product loss when a guard column was included in the affinity chromatography method, demonstrating that the AAV capsids flowed through the guard columns that were tested, without binding to the media.

TABLE 41
Regeneration Peak Areas for Each Guard Column Run

| | 0.1N phosphoric | | 1% sarkosyl | |
|---|---|---|---|---|
| | acid Peak | | Peak | |
| | Areas (mL*mAU) | | Areas (mL*mAU) | |
| Guard Col Run | A280 Peak | A260 Peak | A280 Peak | A260 Peak |
| Control | 46 | 107 | 1374 | 613 |
| Benzyl Ultra | 144 | 232 | 1594 | 995 |
| POROS ™ HQ | 90 | 389 | 1865 | 839 |
| POROS ™ XQ | 70 | 106 | 1901 | 912 |
| Capto Octyl | 323 | 169 | 2045 | 980 |
| Capto 700 | 281 | 255 | 2381 | 1208 |

TABLE 42
qPCR Results for Guard Column Studies

| | vg titer | Volume | Total | % vg |
|---|---|---|---|---|
| Sample Name | (vg/mL) | (mL) | vgs | recovery |
| Benzyl Ultra (BU)-SM | 6.44E11 | 1000 | 6.44E14 | NA |
| BU-FT | 8.65E11 | 1000 | 8.65E14 | 134.33 |
| BU-AC-SM | 6.76E11 | 240 | 1.62E14 | NA |
| BU-AC-elution | 3.25E13 | 3.5 | 1.14E14 | 70.11 |
| BU-AC-phosphoric acid | 2.51E11 | 8.8 | 2.20E12 | 1.36 |
| BU-AC-sarkosyl | 2.03E10 | 8.8 | 1.78E11 | 0.11 |
| HQ-SM | 7.41E11 | 1000 | 7.41E14 | NA |
| HQ-FT | 6.80E11 | 1000 | 6.80E14 | 91.73 |
| HQ-AC-SM | 6.85E11 | 240 | 1.64E14 | NA |
| HQ-AC-elution | 4.38E13 | 3.5 | 1.53E14 | 93.21 |
| HQ-AC-phosphoric acid | 1.60E12 | 8.8 | 1.41E13 | 8.57 |
| HQ-AC-sarkosyl | <9.77E9 | 8.8 | below | 0.00 |
| | | | LOQ | |
| XQ-SM | 6.44E11 | 1000 | 6.44E14 | NA |
| XQ-FT | 6.56E11 | 1000 | 6.56E14 | 101.83 |
| XQ-AC-SM | 6.70E11 | 240 | 1.61E14 | NA |
| XQ-AC-elution | 2.51E13 | 3.5 | 8.79E13 | 54.65 |
| XQ-AC-phosphoric acid | 4.31E11 | 8.8 | 3.80E12 | 2.36 |
| XQ-AC-sarkosyl | <9.77E9 | 8.8 | below | 0.00 |
| | | | LOQ | |
| Capto Octyl (CO)-SM | 4.91E11 | 1000 | 4.91E14 | NA |
| CO-FT | 5.03E11 | 1000 | 5.03E14 | 102.44 |
| CO-AC-SM | 5.04E11 | 240 | 1.21E14 | NA |
| CO-AC-elution | 1.52E13 | 3.45 | 5.24E13 | 43.35 |
| CO-AC-phosphoric acid | 4.57E11 | 11 | 5.03E12 | 4.16 |
| CO-AC-sarkosyl | 0 | 11 | 0 | 0.00 |
| Capto 700 (C700)-SM | 5.72E11 | 230 | 1.32E14 | NA |
| C700-FT | 8.30E11 | 230 | 1.19E14 | 90.3 |
| C700-AC-SM | 7.46E11 | 192 | 1.43E14 | NA |
| C700-AC-elution | 1.36E13 | 4.5 | 6.12E13 | 42.73 |
| C700-AC-phosphoric | 1.38E12 | 11 | 1.52E13 | 10.60 |
| acid | | | | |
| C700-AC-sarkosyl | 3.01E10 | 11 | 3.31E11 | 0.23 |

SM: starting material (clarified lysate),
FT: flow through from column,
BU: Benzyl Ultra guard column,
AC: affinity chromatography,
LOQ: limit of quantification

The % vg recoveries of the affinity elution ranged from about 40%-90%. For all runs, the 0.1 N phosphoric acid, pH 1.90 regeneration step was effective in stripping the resin of any remaining bound AAV9. The 1% sarkosyl step for each run showed little to no AAV9 remaining, indicating effective removal of residual AAV9 from the resin. For all affinity columns loaded with clarified lysate processed on a guard column, the regeneration peak areas were similar to the control peak areas indicating the guard columns did not effectively adsorb impurities that were later removed at least in part by the phosphoric acid and/or sarkosyl washes.

The primary indicator of cleaning performance of the POROS™ CaptureSelect AAV9 resin was the area of the 1% sarkosyl peak. All the runs that used a guard column had higher 1% sarkosyl peak areas than that of the control run indicating that the guard columns were not effective in preventing deposition of impurities on the resin as shown in Table 41. Impurities remained on the resin even after the guard column was implemented which lead to the large 1% sarkosyl peak areas. Therefore, guard columns were not utilized in the final affinity chromatography process for AAV9.

C. Summary

In this example, five different guard columns were tested to determine if they would reduce the level of impurities loaded onto the affinity column and improve the life span of the column resin. The guard column was placed before the affinity column and the guard column flow through was loaded onto the affinity column. No significant AAV9 loss was observed across the guard columns which indicated that AAV9 flows through the column without binding. Based on the regeneration peak areas from the affinity chromatography process, a significant reduction in peak area was not observed, indicating that the guard columns were not effective for the removal of impurities from the affinity load. Therefore, guard columns are not effective in reducing affinity resin fouling over the course of an affinity chromatography method.

### Example 9: Use of Pre-Affinity Guard Filters to Improve Affinity Resin Lifecycle

A. Preparation of EAV Guard Filter Load

HEK293 cells were grown in suspension culture and transfected with a 3-plasmid system to produce rAAV9 vector. Cell suspension was obtained from HCD68 and 50 L SUB bioreactors for harvest. Cell harvest was treated with 0.5% triton X-100 and mixed for 30 minutes to lyse the cells and release AAV9, including full capsids comprising an rAAV genome, intermediate capsids, and empty capsids. The lysate was flocculated, depth filtered, quenched, and 0.2 μm filtered to produce a clarified lysate that was loaded onto affinity chromatography columns as described below.

B. Use of EAV Guard Filter In-Line with POROS™ CaptureSelect AAV9 Affinity Resin

In previous runs, fouling of the POROS™ CaptureSelect AAV9 resin with impurities resulting from flocculation occurred. This reduced the Vifecycle of the affinity resin which is an expensive component of large-scale AAV manufacturing processes. The purpose of this study was to determine if the use of a 0.2 μm nominal PaII™ EAV pre-column filter could extend the current lifecycle of the POROS™ CaptureSelect AAV9 affinity resin.

The EAV filter was placed in-line with the affinity column so that the filtrate was loaded directly onto the column. A total of 3 L of HCD68 or 50 L SUB clarified lysate was passed through a 260 cm² EAV filter with a volumetric load of 115 L/m². The filtrate was then loaded onto a POROS™ CaptureSelect AAV9 affinity column (1.6 cm inner diameter, 11 cm bed height, and 22 mL column volume). The affinity chromatography method utilized for this experiment is outlined in Table 43.

TABLE 43
EAV Guard Filter Study Affinity Chromatography Method

| | | | Residence | Flow |
|---|---|---|---|---|
| | | Column | Time | Velocity |
| Step | Solution | Volumes | (min/CV) | (cm/hr) |
| Equilibration 1 | 100 mM Tris, pH | 5 | 3 | 200 |
| | 7.5 | | | |
| Sample-Load | HCD-68 or 50 L | 136 | 3 | 200 |
| | SUB clarified | | | |
| | lysate (In-line | | | |
| | EAV filtered) | | | |
| Equilibration 2 | 100 mM Tris, pH | 5 | 3 | 200 |
| | 7.5 | | | |
| Pre-Elution | 20% ethanol, 1M | 5 | 3 | 200 |
| Wash | NaCl, 100 mM Tris, | | | |
| | pH 7.5 | | | |
| Equilibration 3 | 100 mM sodium | 5 | 3 | 200 |
| | citrate, pH 7.0 | | | |
| Elution | 100 mM sodium | 3 | 8 | 76 |
| | citrate, 25 mM | | | |
| | MgCl₂, 100 | | | |
| | mM glycine, pH 3.0 | | | |
| Equilibration 4 | 100 mM sodium | 4 | 4 | 76 |
| | citrate, pH 7.0 | | | |
| Regeneration | 3M guanidine HCl, | 4 | 4 | 76 |
| | pH 5.05 | | | |
| Equilibration 5 | 100 mM Tris, pH | 3 | 4 | 150 |
| | 7.5 | | | |
| Storage | 20% ethanol | 3 | 4 | 150 |

Five mL elution fractions were collected and neutralized using 1 M Tris base, pH 9.0. The volume of 1 M Tris base, pH 9.0 was dependent on the initial pH of the elution fraction. The elution fractions were pooled and submitted for analytical testing including transgene qPCR and SEC A260/A280 ratio as shown in Table 44.

TABLE 44
Analytical Results from the EAV Guard Filter Study

| | | | Load | SEC | Elution Peak | Average Load |
|---|---|---|---|---|---|---|
| Run | | % vg | Turbidity | A260/A280 | Area (mL* | Pre-Column |
| # | Source | yield | (NTU) | ratio | mAU) | Pressure (MPa) |
| 1 | HCD-68 | 32 | 19.8 | 0.93 | 3629 | 0.33 |
| 2 | 50SUB | 32 | 8.0 | 1.07 | 3158 | 0.34 |
| 3 | 50SUB | 40 | 11.7 | 1.07 | 3294 | 0.34 |
| 4 | 50SUB | 38 | 37.3 | 1.07 | 3125 | 0.35 |
| 5 | 50SUB | 41 | 33.2 | 1.07 | 3219 | 0.36 |
| 6 | 50SUB | 43 | 38.3 | 1.07 | 3581 | 0.49 |

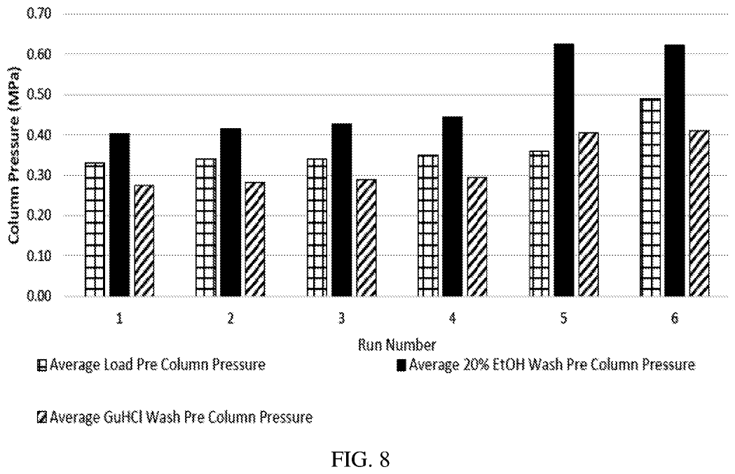

As shown in FIG. 8, column pressure was monitored and recorded over the six runs performed on the same affinity column. % vg recovery and SEC A260/A280 ratios were similar across all six runs with an average recovery of about 38%. The amount of AAV9 eluting from the column during each run was consistent, as shown by the elution peak areas normalized by load volume.

Based on these results, it was determined that the EAV guard filter pre-affinity column does not significantly enhance the affinity column lifecycle. The lifecycle with the EAV guard filter was about 4-5 runs. The fifth run showed a significant pressure increase in the ethanol pre-elution wash step of about 0.2 MPa. By the sixth run, the pressure increase was even more pronounced indicating that impurities were building up on the resin and thus, had not been effectively removed with the EAV guard filter. Therefore, the use of an in-line EAV guard filter was not implemented in the final affinity chromatography process.

C. Summary

In this example, the use of a 0.2 μm Pall™ EAV guard filter was tested to determine if it would remove residual flocculation from the affinity load before it was loaded onto the affinity column. Residual flocculation agent lead to fouling of the affinity resin with impurities and reduced its lifecycle. Different source materials were tested to determine if the guard filter was successful with different loads. The affinity resin lifecycle with the guard filter was about four to five runs and there was no significant reduction in regeneration peak areas. Therefore, a guard filter was not used in the final affinity chromatography process.

### Example 10: Elution and CIP Bed Height and Residence Time Optimization for Affinity Chromatography

A. Preparation of Affinity Load for the Elution Bed Height and Residence Time Study

HEK293 cells were grown in suspension culture and transfected with a 3-plasmid system to produce rAAV9 vector. Cell suspension was obtained from a FIN43 bioreactor for harvest. Cell harvest was treated with 0.5% triton X-100 and mixed for 30 minutes to lyse the cells and release AAV9, including full capsids comprising an rAAV genome, intermediate capsids, and empty capsids. The lysate was flocculated, depth filtered, quenched, and 0.2 μm filtered to produce a clarified lysate that was loaded onto affinity chromatography columns as described below.

B. Elution and CIP Bed Height and Residence Time Study

The purpose of this study was to determine if POROS™ CaptureSelect AAV9 affinity chromatography elution, CIP bed height and residence time affects % vg recovery, % purity, and A260/A280 ratio of the affinity elution. In order to test this, three different column bed heights (10, 15, and 20 cm) and three different residence times (3, 5.5, and 8 minute/CV) were tested as outlined in Table 45.

TABLE 45
Design of Elution Bed Height and Residence Time Study

| Run | Bed Height | Residence Time | Load Volume |
|---|---|---|---|
| # | (cm) | (min/CV) | (mL) |
| 1 | 10 | 8 | 192 |
| 2 | 20 | 8 | 384 |
| 3 | 10 | 3 | 192 |
| 4 | 10 | 3 | 192 |
| 5 | 15 | 8 | 288 |
| 6 | 15 | 3 | 288 |
| 7 | 10 | 5.5 | 192 |
| 8 | 15 | 5.5 | 288 |
| 9 | 20 | 3 | 384 |
| 10 | 20 | 5.5 | 384 |

The affinity load volume was determined by keeping the resin challenge at 8.00×10¹³ vg/mL of resin and using the starting material transgene titer. Three different POROS™ CaptureSelect AAV9 columns were used for the multiple runs (3.4, 5.1, and 6.8 mL column volumes). The general affinity chromatography methods for 3 minute/CV, 5.5 minute/CV, and 8 minute/CV residence times are shown in Tables 46-48.

TABLE 46
3 min RT Affinity Chromatography Method

| | | | Residence |
|---|---|---|---|
| | | Column | Time |
| Step | Solution | Volumes | (min/CV) |
| Equilibration 1 | 100 mM Tris, pH 7.5 | 5 | 3.0 |
| Sample-Load | clarified lysate from 50 L SUB | 56 | 3.0 |
| Equilibration 2 | 100 mM Tris, pH 7.5 | 5 | 3.0 |
| Pre-Elution | 17% ethanol, 1M NaCl, 100 mM | 5 | 3.0 |
| Wash | Tris, pH 7.5 | | |
| Equilibration 3 | 150 mM sodium acetate, pH 5.6 | 5 | 3.0 |
| Elution | 150 mM sodium acetate, 25 mM | 5 | 3.0 |
| | MgCl₂, 100 mM glycine, pH 3.0 | | |
| Equilibration 4 | 150 mM sodium acetate, pH 5.6 | 5 | 3.0 |
| Regeneration | 0.1N phosphoric acid, pH 1.9 | 20 | 3.0 |
| Equilibration 5 | 100 mM Tris, pH 7.5 | 5 | 3.0 |
| Regeneration | 1% sarkosyl, 100 mM Tris, | 20 | 3.0 |
| | pH 7.5 | | |
| Regeneration | 2M NaCl, 100 mM Tris, pH 7.5 | 5 | 3.0 |
| Storage | 17% ethanol | 5 | 3.0 |

TABLE 47
5.5 min RT Affinity Chromatography Method

| | | | Residence |
|---|---|---|---|
| | | Column | Time |
| Step | Solution | Volumes | (min/CV) |
| Equilibration 1 | 100 mM Tris, pH 7.5 | 5 | 3.0 |
| Sample-Load | clarified lysate from 50 L SUB | 56 | 3.0 |
| Equilibration 2 | 100 mM Tris, pH 7.5 | 5 | 3.0 |
| Pre-Elution | 17% ethanol, 1M NaCl, 100 mM | 5 | 3.0 |
| Wash | Tris, pH 7.5 | | |
| Equilibration 3 | 150 mM sodium acetate, pH 5.6 | 5 | 3.0 |
| Elution | 150 mM sodium acetate, 25 mM | 5 | 5.5 |
| | MgCl₂, 100 mM glycine, pH 3.0 | | |
| Equilibration 4 | 150 mM sodium acetate, pH 5.6 | 5 | 5.5 |
| Regeneration | 0.1N phosphoric acid, pH 1.9 | 11 | 5.5 |
| Equilibration 5 | 100 mM Tris, pH 7.5 | 5 | 5.5 |
| Regeneration | 1% sarkosyl, 100 mM Tris, | 11 | 5.5 |
| | pH 7.5 | | |
| Salt Chase/Post | 2M NaCl, 100 mM Tris, pH 7.5 | 5 | 5.5 |
| Regeneration | | | |
| Wash | | | |
| Storage | 17% ethanol | 5 | 5.5 |

TABLE 48
8 min RT Affinity Chromatography Method

| | | | Residence |
|---|---|---|---|
| | | Column | Time |
| Step | Solution | Volumes | (min/CV) |
| Equilibration 1 | 100 mM Tris, pH 7.5 | 5 | 3.0 |
| Sample-Load | clarified lysate from 50 L SUB | 56 | 3.0 |
| Equilibration 2 | 100 mM Tris, pH 7.5 | 5 | 3.0 |
| Pre-Elution | 17% ethanol, 1M NaCl, 100 mM | 5 | 3.0 |
| Wash | Tris, pH 7.5 | | |
| Equilibration 3 | 150 mM sodium acetate, pH 5.6 | 5 | 3.0 |
| Elution | 150 mM sodium acetate, 25 mM | 5 | 8.0 |
| | MgCl₂, 100 mM glycine, pH 3.0 | | |
| Equilibration 4 | 150 mM sodium acetate, pH 5.6 | 5 | 8.0 |
| Regeneration | 0.1N phosphoric acid, pH 1.9 | 5 | 36.0 |
| Equilibration 5 | 100 mM Tris, pH 7.5 | 5 | 8.0 |
| Regeneration | 1% sarkosyl, 100 mM Tris, | 5 | 36.0 |
| | pH 7.5 | | |
| Salt Chase | 2M NaCl, 100 mM Tris, pH 7.5 | 5 | 8.0 |
| Storage | 17% ethanol | 5 | 8.0 |

The elution pool for each run was collected and submitted for analytical testing for % vg recovery, HCP, HCDNA, % purity, and SEC A260/A280 ratio as shown in Table 49.

TABLE 49
Analytical Summary for Affinity Elution Pools

| | | | HCP | HCDNA | SEC |
|---|---|---|---|---|---|
| | % vg | % | (ng/1 × | (ng/1 × | A260/A280 |
| AC Elution | recovery | purity | 10¹⁴ vg) | 10¹⁴ vg) | ratio |
| 10 cm - 8 min | 57 | 96.8 | 7010 | 365 | 0.96 |
| 20 cm - 8 min | 49 | 98.0 | 4793 | 404 | 0.96 |
| 10 cm - 3 min (1) | 52 | 96.1 | 3793 | 306 | 0.96 |
| 10 cm - 3 min (2) | 52 | 94.0 | 3439 | 256 | 0.97 |
| 15 cm - 8 min | 48 | 97.9 | 3092 | 314 | 0.96 |
| 15 cm - 3 min | 70 | 97.2 | 2430 | 305 | 0.96 |
| 10 cm - 5.5 min | 62 | 95.1 | 3754 | 338 | 0.96 |
| 15 cm - 5.5 min | 45 | 96.8 | 4076 | 487 | 0.96 |
| 20 cm - 3 min | 57 | 97.2 | 4025 | 503 | 0.96 |
| 20 cm - 5.5 min | 52 | 96.8 | 5319 | 524 | 0.96 |

Ten affinity chromatography runs were conducted in order to determine if bed height, between 10 cm-20 cm, and residence time of elution and CI P, between 3 minute/CV-8 minute/CV, significantly affected % vg recovery, % purity, impurity levels (HCP, HCDNA), and A260/A280 ratio. There were no noticeable differences in HCP, HCDNA, % purity, aggregation, or A260/A280 ratio content among the bed heights and residence times tested. There was a slight difference in % vg recovery indicating that residence times of 3 minute/CV and 5.5 minute/CV for the elution and CIP steps may be optimal. Residence times of 3 and 5.5 min/CV for the elution did not result in decreased product quality or yield. The 3-minute/CV and 5.5-minute/CV residence time runs utilized 5 column volumes for all steps except for the 0.1 N phosphoric acid, pH 1.9 and the 1% sarkosyl, 100 mM Tris, pH 7.5 steps. These steps targeted enough volume to allow for 60 minutes of total contact time. The CIP peaks at all residence times were consistent in shape, peak area, and retention volume. Lower residence times could be used to decrease process times. Based on this study, a 3 minute/CV residence time was determined to be the optimal residence time for the affinity elution and CIP steps along with a bed height of 15 cm.

C. Summary

In this example, three different elution and CIP residence times and bed heights were studied to determine how they affect % vg recovery, % purity, and A260/A280 ratio of the affinity eluate. There were no significant differences in HCP and HCDNA levels as well % purity. 3-minute/CV and 5.5-minute/CV residence times had a slightly higher % vg recovery. Therefore, to decrease process times, a 3-minute residence time for elution and CIP was implemented.

### Example 11. Pilot and Manufacturing Scales of the Affinity Chromatography Process

A. Preparation of Clarified Lysate or Affinity Load at Large Scale

In a single use bioreactor (SUB), HEK293 cells were grown in suspension culture and transfected with a 3-plasmid system to produce rAAV9 vector. 10% triton X-100 was added to the SUB to achieve a final concentration of 0.5% and agitated for 30 minutes in order to lyse the cells and release AAV9, including full capsids compromising an rAAV genome, intermediate capsids and empty capsids. The lysate was flocculated and then filtered to produce a clarified lysate. The clarified lysate was put through a 0.2 μm pre-column filter prior to loading the lysate onto the POROS™ CaptureSelect AAV9 affinity resin.

B. Optimized Scale-Up to the 250 and 2000 L DMD Affinity Chromatography Scales

An optimized affinity chromatography method was scaled-up for rAAV9 capture and HCP removal for downstream processing of AAV vectors produced in 250 L and 2000 L SUBs. At the 250 L scale, a 2.67 L POROS™ CaptureSelect AAV9 column (20 cm (diameter)×8.5 (high) cm) was loaded with a target resin challenge of 8×10¹³ vg/mL resin (by ITR qPCR). At the 2000 L scale, a 17.663 L (30 cm (d)×25 cm (h)) or 25.610 L (45 cm (d)×15+/−1 cm (h)) POROS CaptureSelect AAV9 column was loaded with a target loading capacity of ≤5×10¹⁶ vg/L resin.

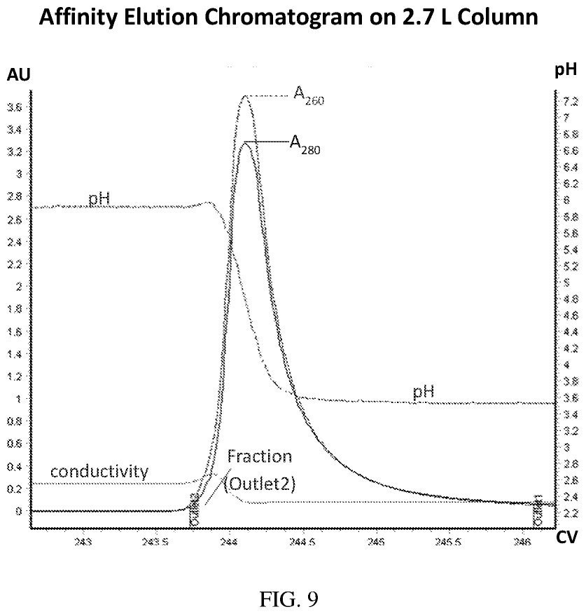

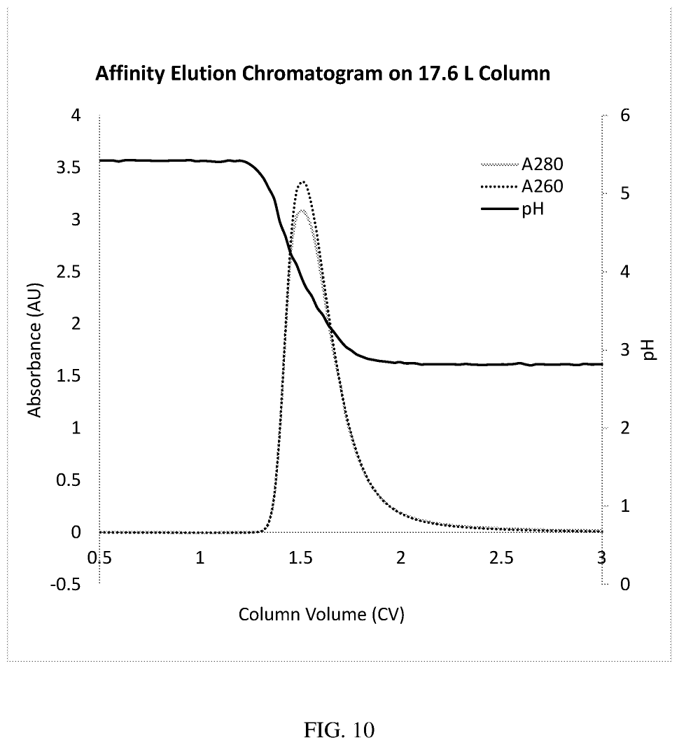

The affinity chromatography methods for the 250 L and 2000 L scales are provided in Tables 50-52. FIG. 9 and FIG. 10 provide chromatograms of representative affinity chromatography runs carried out at the 250 L and 2000 L scales, respectively.

For the 250 L scale, the elution UV collection criteria started at a UV280 of 25 mAU and ended at 65 mAU (2 mm path length) with a total of 0.5 to 1.0 CVs of elution collected. For the 2000 L scale, the elution UV collection criteria started at a UV280 of 12.5 mAU and ended at 112.5 mAU (5 mm path length) with a total of 1.1 to 1.9 CVs of elution collected. Analytical results for the 250 L and 2000 L scales are shown in Tables 53-55. The affinity chromatography process for the purification of rAAV vector comprising a mini-dystrophin transgene could be scaled up to the 250 L and 2000 L scales while maintaining product quality. Tables 56 and 57 show a significant log reduction of HO protein (3.0-4.4 LRV) from the clarified lysate at the 2000L scale and using either the 17.663 L or the 25.610 L columns.

TABLE 50
250 L Affinity Chromatography Method

| | | | linear | |
|---|---|---|---|---|
| | | | flow | residence |
| | | column | velocity | tme |
| step* | solution | volume | (cm/hr) | (min/CV) |
| Equilibration 1 | 100 mM Tris, pH 7.5 | 5 | 170 | 3 |
| Product Load | clarified lysate | NA | 170 | 3 |
| Equilibration 2 | 100 mM Tris, pH 7.5 | 5 | 170 | 3 |
| Pre-Elution | 17% ethanol, 150 mM | 5 | 170 | 3 |
| Wash | sodium acetate, pH 5.6 | | | |
| Equilibration 3 | 153 mM sodium | 5 | 170 | 3 |
| | acetate, pH 5.6 | | | |
| Elution | 148 mM sodium | 5 | 170 | 3 |
| | acetate, 25 mM | | 64 | 7.97 |
| | MgCl₂, 100 | | | |
| | mM glycine, pH 3.0 | | | |
| Equilibration 4 | 153 mM sodium | 5 | 170 | 3 |
| | acetate, pH 5.6 | | 64 | 7.97 |
| Regeneration | 0.1N phosphoric acid, | 5 | 14 | 11.5 |
| (upward flow) | pH 1.9 | | 64 | 7.97 |
| | | | 170 | 3 |
| Post- | 100 mM Tris, pH 7.5 | 5 | 64 | 7.97 |
| Sanitization | | | 170 | 3 |
| Wash | | | | |
| Regeneration | 1% sarkosyl, 100 mM | 5 | 14 | 11.5 |
| (upward flow) | Tris, pH 7.5 | | 64 | 7.97 |
| | | | 170 | 3 |
| Post- | 2M NaCl, 100 mM | 5 | 64 | 7.97 |
| Regeneration | Tris, pH 7.5 | | 170 | 3 |
| Wash | | | | |
| Storage | 17% ethanol | 3 | 64 | 7.97 |
| | | | 170 | 3 |

*all steps performed with downward flow unless indicated

TABLE 51
2000 L Affinity Chromatography Method
(17.665 L; 30 cm × 25 cm column)

| | | | linear | |
|---|---|---|---|---|
| | | | flow | residence |
| | | column | velocity | time |
| step* | solution | volume | (cm/hr) | (min/CV) |
| Pre-use rinse | Water for injection | 5 | 360 | 4.17 |
| (upward flow) | (WFI) | | | |
| Equilibration 1 | 100 mM Tris, pH 7.5 | 5 | 500 | 3 |
| Product load | clarified lysate | na | 500 | 3 |
| Equilibration 2 | 100 mM Tris, pH 7.5 | 5 | 500 | 3 |
| Pre-elution | 17.5% ethanol, 153 | 5 | 345 | 4.35 |
| wash | mM sodium acetate, | | | |
| | pH 5.6 | | | |
| Equilibration 3 | 153 mM sodium | 5 | 345 | 4.35 |
| | acetate, pH 5.6 | | | |
| Elution | 148 mM sodium | 5 | 500 | 3 |
| | acetate, 25 mM MgCl₂, | | | |
| | 100 mM glycine, pH 3.0 | | | |
| Equilibration 4 | 153 mM sodium | 5 | 500 | 3 |
| | acetate, pH 5.6 | | | |
| Regeneration | 0.132N phosphoric | 5^{#} | 500 | 3 |
| (upward flow) | acid, pH 1.9 | | | |
| Post- | 100 mM Tris, pH 7.5 | 5 | 500 | 3 |
| sanitization | | | | |
| wash | | | | |
| Regeneration | 1% sarkosyl, 100 mM | 5 | 500 | 3 |
| (upward flow) | Tris, pH 7.5 | | | |
| Post- | 2M NaCl, 100 mM | 5 | 500 | 3 |
| regeneration | Tris, pH 7.5 | | | |
| wash | | | | |
| Post-use rinse | WFI | 5 | 360 | 4.17 |
| Storage | 17.5% ethanol | 3 | 188 | 7.98 |

*all steps performed with downward flow unless indicated;
^{#}2.5 CV, hold for 45 minutes 2.5 CV.

TABLE 52
2000 L Affinity Chromatography Method (25.610 L;
45 cm × 15 +/− 1 cm column)

| | | | linear | |
|---|---|---|---|---|
| | | | flow | residence |
| | | column | velocity | time |
| step* | solution | volume | (cm/hr) | (min/CV) |
| Pre-use rinse | Water for injection | 5 | 300 | 3 |
| (upward flow) | (WFI) | | | |
| Pre-use | 0.132N phosphoric | 5# | | |
| sanitization | acid, pH 1.9 | | | |
| (upward flow) | | | | |
| Equilibration 1 | 100 mM Tris, pH 7.5 | 5 | 300 | 3 |
| Product load | clarified lysate | na | 300 | 3 |
| Equilibration 2 | 100 mM Tris, pH 7.5 | 5 | 300 | 3 |
| Pre-elution | 17.5% ethanol, 153 | 5 | 200 | 5 |
| wash | mM sodium acetate, | | | |
| | pH 5.6 | | | |
| Equilibration 3 | 153 mM sodium acetate, | 5 | 200 | 5 |
| | pH 5.6 | | | |
| Elution | 148 mM sodium acetate, | 5 | 300 | 3 |
| | 25 mM MgCl₂, | | | |
| | 100 mM glycine, pH 3.0 | | | |
| Equilibration 4 | 153 mM sodium acetate, | 5 | 300 | 3 |
| | pH 5.6 | | | |
| Regeneration | 0.132N phosphoric acid, | 5^{#} | 300 | 3 |
| (upward flow) | pH 1.9 | | | |
| Post- | 100 mM Tris, pH 7.5 | 5 | 300 | 3 |
| sanitization | | | | |
| wash | | | | |
| Regeneration | 1% sarkosyl, 100 mM | 5 | 300 | 3 |
| (upward flow) | Tris, pH 7.5 | | | |
| Post- | 2M NaCl, 100 mM | 5 | 300 | 3 |
| regeneration | Tris, pH 7.5 | | | |
| wash | | | | |
| Post-use rinse | WFI | 5 | 300 | 3 |
| Storage | 17.5% ethanol | 3 | 300 | 3 |

*all steps performed with downward flow unless indicated;
#2.5 CV, hold for 45 minutes 2.5 CV.

TABLE 53
250 L Pilot Analytical Results on Affinity Elution Pools.

| Batch ID | Run 1 | Run 2 | Run 3 | Run 4 | Run 5 | Run 6 |
|---|---|---|---|---|---|---|
| vg titer | 8.28E+12 | 5.77E+13 | 5.77E+13 | 4.19E+13 | 4.03E+13 | 4.94E+13 |
| (transgene) | | | | | | |
| (vg/mL) | | | | | | |
| HC DNA | — | 437,989 | 350,388 | 81,223 | 161,782 | 278,046 |
| (pg/mL) | | | | | | |
| HCP (ng/ml) | — | 11,470 | 4,2333 | 10,098 | 2,061 | 2,993 |
| Residual | — | 0.43 | 1.35 | 1.6 | — | — |
| affinity ligand | | | | | | |
| (pg/1E9 vg | | | | | | |
| % vg yield | 27.70% | 89.30% | 85.50% | 67% | 77.3% | 91.80% |
| (recovery) | | | | | | |

TABLE 54
2000L analytical results on Affinity Elution Pools.

| | 30 cm (d) × 25 cm (h) Affinity Column, 17,663 mL CV | | | | | |
|---|---|---|---|---|---|---|
| Test Method | Run 1 | Run 2 | Run 3 | Run 4 | Run 5 | Run 6 |
| Vg titer (transgene) | 7.25E+12 | 1.70E+13 | 1.83E+13 | 3.18E+13 | 2.70E+13 | 2.60E+13 |
| (vg/mL) | | | | | | |
| SEC_ AAV | 99.2 | 99.55 | 100 | 100 | 99.4 | 99.1 |
| (monomer) (% | | | | | | |
| purity) | | | | | | |
| SEC_ AAV (viral | 1.82E+13 | 5.42E+13 | 2.72E+13 | 3.78E+13 | 6.22E+13 | 5.88E+13 |
| particle titer ) | | | | | | |
| (vp/mL) | | | | | | |
| SEC_ AAV | 1.1 | 1.04 | 1.09 | 1.11 | 1.05 | 1.09 |
| (A260/280 ratio ) | | | | | | |
| HC DNA (pg/1E9 | 9.11 | 10.77 | 7.18 | 3.26 | 6.93 | 6.41 |
| vg) | | | | | | |
| HC DNA (pg/mL) | 6.60E+04 | 1.83E+05 | 1.31E+05 | 1.04E+05 | 1.87E+05 | 1.67E+05 |
| HC DNA Log | 0.004 | −0.090 | 0.146 | 0.377 | 0.210 | −0.038 |
| reduction value | | | | | | |
| Residual plasmid | 35 | 30 | 26 | 16 | 30 | 26 |
| DNA (pg/1E9) | | | | | | |
| Residual plasmid | 2.53E+05 | 5.07E+05 | 4.73E+05 | 5.23E+05 | 8.04E+05 | 6.87E+05 |
| DNA (pg/mL) | | | | | | |
| Residual plasmid | 0.859 | 0.783 | 0.723 | 0.780 | 0.519 | 0.835 |
| DNA Log reduction | | | | | | |
| value | | | | | | |
| HCP (pg/1E9 vg) | 330.75 | 338.96 | 497.52 | 339.33 | 212.9 | 306.21 |
| HCP (ng/mL) | 2.40E+03 | 5.76E+03 | 9.10E+03 | 1.08E+03 | 5.75E+03 | 7.96E+03 |
| HCP Log reduction | 3.528 | 3.56 | 3.020 | 3.340 | 3.379 | 3.081 |
| value | | | | | | |
| Residual affinity | 17.55 | 4.17 | 31.51 | 13.08 | 20.51 | 14.65 |
| ligand (pg/1E9 vg) | | | | | | |
| Residual affinity | 54.76 | 70.86 | 516.6 | 416 | 533.8 | 381 |
| ligand (ng/mL) | | | | | | |
| pH | not | not | not | not | not | 3.251 |
| | tested | tested | tested | tested | tested | |
| % vg yield (% | 69 | 58 | 84 | 102 | 75 | 67 |
| recovery) | | | | | | |

TABLE 55
2000 L analytical results on Affinity Elution Pools.

| | 45 cm (d) × 16 cm (h), 25,610 mL CV | | | | |
|---|---|---|---|---|---|
| | Run 1 | Run 2 | Run 3 | Run 4 | Run5 |
| Vg titer (transgene) | 3.79E+13 | 2.40E+13 | 2.33E+13 | 2.40E+13 | 3.81E+13 |
| (vg/mL) | | | | | |
| SEC_AAV (monomer) | 98.2 | 98.2 | 100 | 99.6 | 100 |
| (% purity) | | | | | |
| SEC_AAV (viral | 8.79E+13 | 7.31E+13 | 5.74E+13 | 7.64E+13 | 6.21E+13 |
| particle titer) (vp/mL) | | | | | |
| SEC_AAV (A260/280 | 1.08 | 1.05 | 1.02 | 0.98 | 1.07 |
| ratio) | | | | | |
| HC DNA (pg/1E9 vg) | 5.15 | 8.21 | 7.61 | 12.03 | 4.81 |
| HC DNA (pg/mL) | 1.67E+05 | 1.95E+05 | 1.77E+05 | 3.49E+05 | 1.83E+05 |
| HC DNA Log reduction | −0.152 | 0.116 | −0.116 | −0.237 | 0.117 |
| value | | | | | |
| Residual plasmid DNA | 4 | 10 | 34 | 34 | 33 |
| (pg/1E9) | | | | | |
| Residual plasmid DNA | 1.46E+05 | 2.39E+05 | 8.02E+05 | 9.83E+05 | 1.25E+05 |
| (pg/mL) | | | | | |
| Residual plasmid DNA | 0.978 | 0.829 | 0.046 | 0.060 | −0.197 |
| Log reduction value | | | | | |
| HCP (pg/1E9 vg) | 47.3 | 195.22 | 123.23 | 108.31 | 93.76 |
| HCP (ng/ml) | 1.79E+03 | 4.69E+03 | 2.87E+03 | 3.14E+03 | 3.57E+03 |
| HCP Log reduction | 4.320 | 4.085 | 4.289 | 4.163 | 4.174 |
| value | | | | | |
| Residual affinity | 5.79 | 22.16 | 20.6 | 9.48 | 7.14 |
| ligand (pg/1E9 vg) | | | | | |
| Residual affinity | 219.3 | 531.75 | 480 | 274.88 | 272.03 |
| ligand (ng/mL) | | | | | |
| pH | 3.457 | 3.527 | 3.544 | 3.531 | 3.570 |
| % vg yield (% | 75 | 56 | 75 | 73 | 87 |
| recovery) | | | | | |

TABLE 56
HC DNA and HC protein Log Reduction Value (LRV)

| Process | 30 cm (d) × 25 cm (h) Affinity Column, 17.663 L CV | | | | | |
|---|---|---|---|---|---|---|
| Intermediate | Run 1 | Run 2 | Run 3 | Run 4 | Run 5 | Run 6 |

HC DNA (total ng)

| | | | | | | |
|---|---|---|---|---|---|---|
| Clarified lysate | 2.31 | 2.82 | 3.23 | 5.04 | 6.45 | 3.47 |
| (1E+06) | | | | | | |
| Affinity eluate (1E+06) | 2.29 | 3.46 | 2.31 | 2.11 | 3.98 | 3.27 |
| HC DNA LRV | 0.004 | −0.090 | 0.146 | 0.377 | 0.210 | 0.025 |

HC protein (total ng)

| | | | | | | |
|---|---|---|---|---|---|---|
| Clarified lysate | 2.81 | 3.95 | 1.67 | 4.81 | 2.93 | 2.4 |
| (1E+11) | | | | | | |
| Affinity eluate (1E+8) | 0.832 | 1.09 | 1.60 | 2.20 | 1.22 | 1.89 |
| HCP LRV | 3.528 | 3.56 | 3.02 | 3.34 | 3.379 | 3.104 |

TABLE 57
HC DNA and HC protein Log Reduction Value (LRV)

| Process | 45 cm (d) × 16 cm ( h), 25.610 L CV | | | | | |
|---|---|---|---|---|---|---|
| Intermediate | Run 1 | Run 2 | Run 3 | Run 4 | Run 5 | Run 6 |

HC DNA (total ng)

| | | | | | | |
|---|---|---|---|---|---|---|
| Clarified lysate | 3.51 | 4.76 | 3.75 | 1.96 | 3.34 | 4.04 |
| (1E+06) | | | | | | |
| Affinity eluate (1E+06) | 3.83 | 3.35 | 2.87 | 2.56 | 5.76 | 3.09 |
| HC DNA LRV | 0.038 | 0.152 | 0.116 | −0.116 | −0.237 | 0.117 |

HC protein (total ng)

| | | | | | | |
|---|---|---|---|---|---|---|
| Clarified lysate | 2.2 | 6.43 | 8.32 | 8.05 | 7.55 | 8.97 |
| (1E+11) | | | | | | |
| Affinity eluate (1E+8) | 1.83 | 0.308 | 0.684 | 0.414 | 0.518 | 0.601 |
| HCP LRV | 3.081 | 4.42 | 4.085 | 4.289 | 4.163 | 4.174 |

We claim:

1. A method of purifying a recombinant AAV (rAAV) vector, the method comprising: loading a solution comprising the rAAV vector on an affinity chromatography stationary phase; applying a pre-elution wash solution to the stationary phase, wherein the pre-elution wash solution comprises 15% to 25% ethanol and a buffering agent, and has a pH of 5 to 6; eluting the rAAV vector from the stationary phase with an elution buffer, wherein the elution buffer comprises a salt, an amino acid and a buffering agent and has a pH of 2 to 4, to produce an affinity eluate.

2. The method of claim 1, wherein the affinity chromatography stationary phase is in a column.

3. The method of any one of claims 1-2, wherein the elution buffer comprises 5 mM to 150 mM of the salt, optionally wherein the salt is magnesium chloride.

4. The method of any one of claims 1-3, wherein the elution buffer comprises 50 mM to 150 mM of the amino acid, optionally wherein the amino acid is glycine.

5. The method of any one of claims 1-4, wherein the elution buffer comprises 75 mM to 250 mM of the buffering agent, optionally wherein the buffering agent is sodium acetate.

6. The method of any one of claims 1-5, wherein the elution buffer has a pH of 2.5 to 3.5.

7. The method of any one of claims 1-6, wherein the elution buffer comprises 50 mM to 150 mM of glycine, 10 mM to 100 mM of MgCl₂, 50 mM to 200 mM of sodium acetate, and a pH of 2.5 to 3.5, optionally wherein the elution buffer has a conductivity of 5 mS/cm to 40 mS/cm or 20 mS/cm to 35 mS/cm.

8. The method of any one of claims 1-7, wherein 2 column volumes (CV) to 10 CV or 4.5 CV to 5.5 CV of the elution buffer is applied to the stationary phase.

9. The method of any one of claims 1-8, wherein the elution buffer i) elutes the rAAV vector from the stationary phase, ii) does not elute residual impurities from the stationary phase; iii) does not result in precipitation of the affinity eluate; iv) maximizes a % vg recovery; v) does not interfere with binding of the rAAV vector to an anion exchange chromatography (AEX) stationary phase; vi) does not contain a trivalent anion; vii) does not contain citrate ions, or a combination thereof.

10. The method of any one of claims 1-9, wherein 0.1 CV to 10 CV of the affinity eluate is collected from the stationary phase.

11. The method of any one of claims 1-10, wherein the solution comprising the rAAV vector comprises host cell protein and host cell DNA.

12. The method of any one of claims 1-11, wherein the solution comprising the rAAV vector is loaded onto the stationary phase to achieve a challenge of 1×10¹² viral genomes (vg)/mL stationary phase to 1.5×10¹⁴ vg/mL stationary phase.

13. The method of any one of claims 1-12, wherein the rAAV vector comprises a capsid protein from an AAV serotype.

14. The method of claim 13, wherein the rAAV vector comprises a capsid protein from a AAV serotype selected from the group consisting of AAV1, AAV2, AAV3 (including AAV3A and AAV3B), AAV4, AAV5, AAV6, AAV7, AAV8, AAV9, AAV10, AAV12, AAVrh8, AAVrh10, AAVrh39, AAVrh43, AAVrh74, AAVrh32.22, AAV1.1, AAV2.5, AAV6.1, AAV6.2, AAV6.3.1, AAV9.45, AAVShH10, HSC15/17, RHM4-1, RHM15-1, RHM15-2, RHM15-3/RHM15-5, RHM15-4, RHM15-6, AAVhu.26, AAV2i8, AAV29G, AAV2, AAV8G9, AAV-LK03, AAV2-TT, AAV2-TT-S312N, AAV3B-S312N, AAVavian, AAVbat, AAVbovine, AAVcanine, AAVequine, AAVprimate, AAVnon-primate, AAVovine, AAVmuscovy duck, AAVporcine4, AAVporcine5, AAVsnake NP4, NP22, NP66, AAVDJ, AAVDJ/8, AAVDJ/9, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14, AAVHSC15, AAVv66, AAVv33, AAVv37, AAVv40, AAVv67, AAVv70, AAVv72, AAVv84, AAVv86, AAVv87 and AAVv90.

15. The method of any one of claims 1-14, wherein the stationary phase comprises a macromolecule that binds an AAV capsid.

16. The method of any one of claims 1-15, wherein the pre-elution wash solution comprises 10 mM to 500 mM of the buffering agent, optionally wherein the buffering agent is sodium acetate.

17. The method of any one of claims 1-16, wherein the pre-elution wash solution comprises 15% to 25% of ethanol, 100 mM to 200 mM of sodium acetate, and has a pH of 5 to 6, optionally wherein 1 CV to 10 CV or 4.5 to 5.5 CV of the pre-elution wash solution is applied to the stationary phase.

18. The method of any one of claims 1-17, wherein the pre-elution wash i) removes bound impurities from the stationary phase; ii) maintains rAAV-stationary phase ligand binding; iii) has a reduced pH to improve removal of proteins other than rAAV proteins, such as host cell proteins, or a combination thereof.

19. The method of any one of claims 1-18, further comprising equilibration of the stationary phase i) prior to loading the solution comprising the rAAV vector on the stationary phase, ii) after loading the solution comprising the rAAV vector on the stationary phase, iii) prior to application of a pre-elution wash, iv) after application of a pre-elution wash v) prior to eluting the rAAV vector from the stationary phase with an elution buffer, vi) after eluting the rAAV vector from the stationary phase with an elution buffer, vii) prior to contacting the stationary phase with a first regeneration buffer, or a combination thereof.

20. The method of claim 19, wherein equilibration comprises application of a buffer solution to the stationary phase and removal of all or a portion of the buffer solution from the stationary phase.

21. The method of claim 20, wherein the buffer solution comprises Tris or sodium acetate.

22. The method of claim 19 or 20, wherein the buffer solution comprises about 100 mM Tris at about pH 7.5 or about 153 mM sodium acetate at about pH 5.6.

23. The method of any one of claims 1-22, further comprising contacting the stationary phase with a first regeneration buffer after obtaining at least a portion of the affinity eluate.

24. The method of claim 23, wherein the first regeneration buffer comprises 0.05 N to 1.5 N of an acid, optionally wherein the acid is phosphoric acid.

25. The method of claim 23 or 24, wherein the first regeneration buffer comprises 0.10 N to 0.15 N phosphoric acid, and a pH of 1.5 to 2.5.

26. The method of any one of claims 23-25, wherein the stationary phase is contacted with 1 CV to 10 CV or 4.5 CV to 5.5 CV of the first regeneration buffer.

27. The method of any one of claims 23-26, wherein the method further comprises loading another solution comprising an rAAV vector on the affinity chromatography stationary phase after removing at least a portion of the first regeneration buffer.

28. The method of any one of claims 23-27, wherein the method further comprises applying a second amount of the pre-elution wash solution on the affinity chromatography stationary phase after removing at least a portion of the first regeneration buffer.

29. The method of any one of claims 23-28, wherein a number of purification cycles that can be run on the stationary phase that is contacted with the first regeneration buffer is 8 or more;
wherein a number of purification cycles that can be run on the stationary phase that is contacted with the first regeneration buffer is 10 or more;
wherein a % vg recovery of an eluate from a last purification cycle after contacting the stationary phase with the first regeneration buffer is not decreased more than 10% as compared to a % vg recovery of an eluate from a first purification cycle before the stationary phase is contacted with the first regeneration buffer;
wherein an amount of unbound rAAV vector in a flow through of a last purification cycle after contacting the stationary phase with the first regeneration buffer is not increased more than 10% as compared to an amount of unbound rAAV vector in a flow through of a first purification cycle before the stationary phase is contacted with the first regeneration buffer;
wherein a column pressure of a last purification cycle after contacting the stationary phase with the first regeneration buffer is not higher than 0.4 MPa;
wherein a % purity of an eluate from a last purification cycle after contacting the stationary phase with the first regeneration buffer is not decreased more than 10% as compared to a % purity of an eluate from a first purification cycle before the stationary phase is contacted with the first regeneration buffer;
wherein an amount of HCP of an eluate from a last purification cycle after contacting the stationary phase with the first regeneration buffer is not increased more than 10% as compared to an amount of HCP of an eluate from a first purification cycle before the stationary phase is contacted with the first regeneration buffer;
wherein an amount of HCDNA of an eluate from a last purification cycle after contacting the stationary phase with the first regeneration buffer is not increased more than 10% as compared to an amount of HCDNA of an eluate from a first purification cycle before the stationary phase is contacted with the first regeneration buffer;
wherein an average A260/A280 of an eluate from a last purification cycle after contacting the stationary phase with the first regeneration buffer is not decreased more than 10% as compared to an average A260/A280 of an eluate from a first purification cycle before the stationary phase is contacted with the first regeneration buffer, or a combination thereof.

30. The method of any one of claims 23-29, wherein a number of purifications cycles that can be run on the stationary phase that is contacted with the first regeneration buffer is increased as compared to a stationary phase that is contacted with a buffer comprising guanidine HCl.

31. The method of any one of claims 23-30, further comprising contacting the stationary phase with a second regeneration buffer after contacting the stationary phase with the first regeneration buffer, wherein the second regeneration buffer is different than the first regeneration buffer.

32. The method of claim 31, wherein the second regeneration buffer comprises 0.1% to 5% of a detergent, optionally wherein the detergent is sarkosyl.

33. The method of claim 31 or 32, wherein the second regeneration buffer comprises 50 mM to 150 mM of a buffering agent, optionally wherein the buffering agent is Tris.

34. The method of any one of claims 31-33, wherein the second regeneration buffer comprises 0.1% to 1.5% of sarkosyl, 50 mM to 150 mM of Tris, and a pH of 7 to 8, optionally wherein the stationary phase is contacted with 1 CV to 10 CV or 4.5 CV to 5.5 CV of the second regeneration buffer.

35. The method of any one of claims 31-34, wherein the method further comprises loading another solution comprising a rAAV vector on the affinity chromatography stationary phase after removing at least a portion of the second regeneration buffer.

36. The method of any one of claims 31-35, wherein the method further comprises applying a second amount of the pre-elution wash solution on the affinity chromatography stationary phase after removing at least a portion of the second regeneration buffer.

37. A method of purifying a rAAV vector, the method comprising:
loading a solution comprising the rAAV vector on an affinity chromatography stationary phase in a column;
applying a pre-elution wash solution comprising 15% to 25% ethanol, 100 mM to 200 mM sodium acetate, and a pH of 5 to 6 to the stationary phase; and
eluting the rAAV vector from the stationary phase with an elution buffer comprising 50 mM to 150 mM glycine, 10 mM to 100 mM MgCl₂, 50 mM to 250 mM sodium acetate, and a pH of 2.5 to 3.5 to produce an affinity eluate containing the rAAV vector.

38. The method of claim 37, wherein 1 CV to 10 CV or 4.5 CV to 5.5 CV of the pre-elution wash solution is applied to the stationary phase.

39. The method of claim 37 or 38, wherein the pre-elution wash has a reduced pH to improve removal of a protein other than a rAAV protein from the stationary phase, wherein the pre-elution wash maintains rAAV-stationary phase ligand binding or both and optionally wherein the proteins other than rAAV protein is a host cell protein.

40. The method of any one of claims 37-39, wherein the elution buffer does not result in precipitation of an affinity eluate, wherein the elution buffer does not interfere with binding of the rAAV vector to an anion exchange chromatography (AEX) stationary phase or both.

41. The method of any one of claims 37-40, wherein a % vg recovery in the affinity eluate is 50% to 100%, optionally as measured by qPCR; wherein a vg per mL of the affinity eluate is about 1.0×10¹² vg/mL to about 1.0×10¹⁴ vg/mL, optionally as measured by qPCR; wherein a % purity of the affinity eluate is about 95% to about 100% capsid protein of total protein, optionally as measured by SEC or reverse phase HPLC, non-reducing, or a combination thereof.

42. The method of any one of claims 37-41, further comprising contacting the stationary phase with a first regeneration buffer after obtaining at least a portion of the affinity eluate.

43. The method of any one of claims 37-42, further comprising contacting the stationary phase with a second regeneration buffer after the first regeneration buffer, wherein the second regeneration buffer is different than the first regeneration buffer.

44. A method of regenerating an affinity chromatography stationary phase, the method comprising: contacting the stationary phase with a first regeneration buffer comprising an acid and a pH of 1 to 4, wherein impurities are removed from the stationary phase.

45. The method of claim 44, wherein the first regeneration buffer comprises 0.05 N to 1.5 N of the acid, optionally wherein the acid is phosphoric acid or acetic acid.

46. The method of claim 44 or 45, wherein the first regeneration buffer comprises 0.10 N to 0.15 N phosphoric acid, and a pH of 1.5 to 2.5.

47. The method of any one of claims 44-46, wherein 1 CV to 10 CV or 4.5 CV to 5.5 CV of the first regeneration buffer is applied to the stationary phase, wherein the stationary phase is contacted with about half of the volume of a first regeneration buffer, followed by a hold period of about 45 minutes, followed by contacting the stationary phase with a second half of the volume of the first regeneration buffer.

48. The method of any one of claims 44-47, wherein the method further comprises loading a solution comprising an rAAV vector on the affinity chromatography stationary phase after removing at least a portion of the first regeneration buffer.

49. The method of any one of claims 44-48, wherein the method further comprises applying a pre-elution wash solution on the affinity chromatography stationary phase after removing at least a portion of the first regeneration buffer.

50. The method of any one of claims 44-49, further comprising contacting the stationary phase with a second regeneration buffer after the first regeneration buffer, wherein the second regeneration buffer is different than the first regeneration buffer.

51. The method of claim 50, wherein the second regeneration buffer comprises a detergent and a buffering agent.

52. The method of claim 50 or 51, wherein the second regeneration buffer comprises 0.1% to 5% of the detergent, optionally wherein the detergent is sarkosyl.

53. The method of any one of claims 50-52, wherein the second regeneration buffer comprises 50 mM to 150 mM of the buffering agent, optionally wherein the buffering agent is Tris.

54. The method of any one of claims 50-53, wherein the second regeneration buffer comprises 0.1% to 1.5% of sarkosyl, 50 mM to 150 mM of Tris, and a pH of 7 to 8.

55. The method of any one of claims 50-54, wherein 1 CV to 10 CV or 4.5 CV to 5.5 CV of the second regeneration buffer is applied to the stationary phase.

56. The method of any one of claims 50-55, wherein the method further comprises loading a solution comprising an rAAV vector on the affinity chromatography stationary phase after removing at least a portion of the second regeneration buffer.

57. The method of any one of claims 50-56, wherein the method further comprises applying a pre-elution wash solution on the affinity chromatography stationary phase after removing at least a portion of the second regeneration buffer.
